(19) United States
(12) Patent Application Publication
(10) Pub. No.: US 2026/0265195 A1
(43) Pub. Date: Sep. 10, 2026

(54) SARM1 MODULATORS, PREPARATIONS, AND USES THEREOF

(71) Applicant: SIRONAX LTD, Grand Cayman (KY)

(72) Inventors: Zhen SUN, Shanghai (CN); Lianzhu LIU, Shanghai (CN); Yanping XU, Beijing (CN); Yingtao LIU, Shanghai (CN); Jinbao WU, Beijing (CN); Baochuan SUN, Beijing (CN); Ming JIANG, Beijing (CN); Zhaolan ZHANG, Beijing (CN); Jianguang HAN, Beijing (CN)

(21) Appl. No.: 19/138,050

(22) PCT Filed: Dec. 15, 2023
(86) PCT No.: PCT/CN2023/139051

§ 371 Date: Jun. 11, 2025

(30) Foreign Application Priority Data
Dec. 15, 2022 (WO) PCT/CN2022/139210

(51) Int. Cl. C07D 209/42; A61K 31/404; A61K 31/415; A61K 31/421; A61K 31/425; A61K 31/4439; A61K 31/5375; C07B 59/00; C07D 401/04; C07D 401/12; C07D 403/06; C07D 403/12; C07D 405/04; C07D 405/12; C07D 405/14; C07D 409/12; C07D 413/12; C07D 417/04

(52) U.S. Cl. CPC C07D 209/42; A61K 31/404; A61K 31/415; A61K 31/421; A61K 31/425; A61K 31/4439; A61K 31/5375; C07B 59/002; C07D 401/04; C07D 401/12; C07D 403/06; C07D 403/12; C07D 405/04; C07D 405/12; C07D 405/14; C07D 409/12; C07D 413/12; C07D 417/04; C07B 2200/05

(57) ABSTRACT

This disclosure provides compounds of Formula (1), compositions comprising the same, and methods of using the same, including uses in modulating SARM1 and treating various diseases and conditions, e.g., those caused by axonal degeneration.

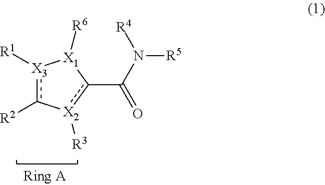

### CROSS-REFERENCE TO RELATED APPLICATION

This application claims the priority to International Application No. PCT/CN2022/139210, filed 15 Dec. 2022, which is incorporated herein by reference in its entirety.

### FIELD OF THE DISCLOSURE

The present disclosure relates to compounds that modulate SARM1, compositions comprising the compounds, methods of preparing the compounds, and methods of using the compounds to treat various diseases or conditions, e.g., those caused by axonal degeneration.

### BACKGROUND OF THE DISCLOSURE

Axonal degeneration causes disease progression and accumulation of disability in many degenerative diseases of the peripheral nervous system (PNS) and central nervous systems (CNS), such as multiple sclerosis, Parkinson's disease, and amyotrophic lateral sclerosis (ALS), or acute conditions such as traumatic brain injury. (Hughes 2021 (R. Hughes et al., Small Molecule SARM1 Inhibitors Recapitulate the SARM1−/−Phenotype and Allow Recovery of a Metastable Pool of Axons Fated to Degenerate, Cell Rep. 2021 Jan. 5; 34 (1): 108588.); Bosanac 2021 (T. Bosanac et al., Pharmacological SARM1 inhibition protects axon structure and function in paclitaxel-induced peripheral neuropathy, Brain, Vol. 144, Issue 10, 2021, pages 3226-3238)). Therefore, axonal protection is an important neuroprotective approach to treatment of chronic and acute CNS and PNS neurodegenerative disorders. (Hughes 2021; Bosanac 2021).

SARM1 (Sterile Alpha and TIR Motif-containing 1) is a unique member of the Myd88 family of adaptor proteins and is considered a major driver of an evolutionarily conserved program of axonal degeneration downstream of chemical, inflammatory, mechanical, or metabolic insults to the axon. (Hughes 2021; Bosanac 2021). SARM1 has been recognized as a central mediator of axonal degeneration in a number of diseases or conditions, including ALS, Parkinson's disease, multiple sclerosis, traumatic brain injury, and diabetic neuropathy, as well as chemotherapy induced peripheral neuropathy (CIPN), which is a major cause of morbidity and the main cause of dose reductions and discontinuations in cancer treatment. (Hughes 2021; Bosanac 2021). SARM1 is a compelling target to treat neurodegeneration characterized by axonopathies of the peripheral and central nervous systems.

SARM1 contains a mitochondrial targeting sequence, an N-terminal domain with armadillo repeats (ARM), two sterileα-motif (SAM) domains, and a Toll/interleukin-1 receptor (TIR) domain (Gerdts 2013 (J. Gerdts et al., Sarm1-mediated axon degeneration requires both SAM and TIR interactions. J Neurosci. 2013 Aug. 14; 33 (33): 13569-80.)) The SARM1 TIR domain is a NAD⁺ hydrolase (NADase), which converts the NAD⁺ to ADPR or cADPR and NAM (Sporny 2019 (M. Sporny et al., Structural Evidence for an Octameric Ring Arrangement of SARM1. J Mol Biol. 2019 Sep. 6; 431 (19): 3591-3605.)). This NADase activity is essential for its axonal degenerative function. (Bosanac 2021). The activity of SARM1 also depends on the oligomerization formed through the SAM domains (Sporny 2019) and is autoinhibited by the ARM domain (Chen (2021) (C. Shen et al., Multiple domain interfaces mediate SARM1 autoinhibition. Proc Natl Acad Sci USA. 2021 Jan. 26; 118 (4).)).

Certain SARM1 inhibitors are disclosed by Bosanac 2021, Hughes 2021, Sporny 2020 (M. Sporny et al, Structural basis for SARM1 inhibition and activation under energetic stress. Elife. 2020 Nov. 13; 9: e62021. doi: 10.7554/eLife. 62021. PMID: 33185189; PMCID: PMC7688312.), WO 2018/057989 A1, WO 2020/081923 A1, WO 2021/142006 A1, WO2021207302A1, and WO2021207308A1. Certain dipeptidyl peptidase inhibitors (e.g., biphenyl or phenyl benzo imidazole derivatives) are disclosed in US 2005/0272765 A1. Certain benzyl benzoxazol derivatives as Met-kinase inhibitors are disclosed in WO 2008/148449 A1. Certain dihydroisoquinolinone derivatives and combinatorial libraries thereof are described in WO 01/14879. Certain compositions for promoting readthrough of premature termination codons, and methods of using the same are described in WO 2017/049409. Certain nitrogen-containing heterocyclic compounds having nematicidal properties, preparations and uses thereof are described in CN 108276352.

The present disclosure describes SARM1 inhibitors that can be used to prevent axonal degeneration in peripheral and central axonopathies and to provide a transformational disease-modifying treatment for the related diseases or conditions.

### SUMMARY OF THE DISCLOSURE

One aspect of this disclosure provides a compound selected from compounds of Formulae 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, and 6-2f (e.g., Compounds 1 to 240) disclosed herein, a tautomer thereof, a solvate or stereoisomer of the compound or the tautomer, or a pharmaceutically acceptable salt of the foregoing, which can be employed in the treatment of various diseases or conditions, such as diseases or conditions caused by axonal degeneration. For example, disclosed herein is a compound of the following structural Formula 1:

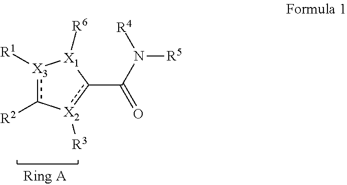

a tautomer thereof, a solvate or stereoisomer of the compound or the tautomer, or a pharmaceutically acceptable salt of the foregoing, wherein:
X₁ is C, N, or O; X₂ is C or N; X₃ is Cor N;
R¹ and R²:
are independently selected from H, optionally substituted C₁-C₄ alkyl, and optionally substituted C₃-C₆ cycloalkyl, or
join to form a 5- to 7-membered ring such that the 5- to 7-membered ring and Ring A together form Fused Ring A selected from Fused Ring A-1, Fused Ring A-2, Fused Ring A-3, and Fused Ring A-4 shown below:

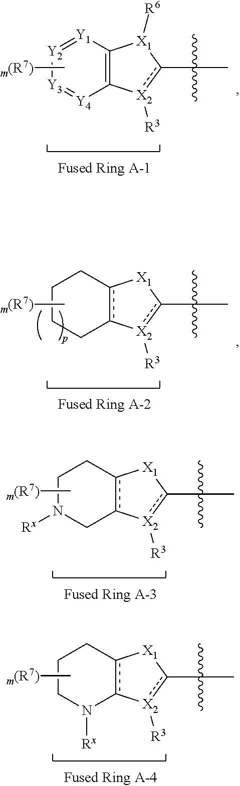

wherein:
Y₁, Y₂, Y₃, and Y₄ are each independently selected from C and N;
p is an integer selected from 0, 1, and 2;
R⁷ is selected from halogen, —CN, —CONH₂, —SO₂Me, optionally substituted 3- to 6-membered cycloalkyl, optionally substituted 4- to 6-membered heterocyclyl, optionally substituted phenyl, optionally substituted 5- to 6-membered heteroaryl, optionally substituted C₁-C₄ alkyl, ═O (provided that Fused Ring A is not Fused Ring A-1), and —OR^{s}, wherein R^{s} is optionally substituted C₁-C₄ alkyl, and m is an integer selected from 0, 1, 2, and 3;
R^{x} is selected from H, optionally substituted 3- to 5-membered cycloalkyl, optionally substituted C₁-C₄ alkyl, and —C(═O)R^{y}, wherein R^{y} is optionally substituted 3- to 5-membered cycloalkyl or optionally substituted C₁-C₄ alkyl;
R³:
is selected from absent, H, —CN, —C(═O)NH₂, and optionally substituted C₁-C₄ alkyl;
R⁴:
is selected from H, optionally substituted C₁-C₆ alkyl, optionally substituted 3- to 5-membered cycloalkyl, optionally substituted 3- to 6-membered heterocyclyl, optionally substituted 5- to 6-membered heteroaryl,
—R^{a}C(═O)R^{b}, —R^{a}NHC(═O)R^{b}, —R^{a}NR^{c}R^{d}, —R^{a}S(═O)R^{b}, —R^{a}S(═O)₂R^{b},

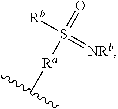

R^{a}, for each occurrence, is independently selected from C₁-C₈ alkyl optionally substituted by 1-3 groups selected from —OH and halogen;
R^{b}, for each occurrence, is independently selected from H, —OH, —O(C₁-C₃ alkyl), 4- to 6-membered heterocyclyl, and C₁-C₆ alkyl optionally substituted by 1-3 groups selected from —OH and halogen;
R^{c} and R^{d}, for each occurrence, are independently selected from H and C₁-C₆ alkyl, or
R^{c} and R^{d} join to form a 3- to 6-membered cycloalkyl or a 3- to 6-membered heterocyclyl wherein the 3- to 6-membered cycloalkyl or 3- to 6-membered heterocyclyl of R^{c} and R^{d} is optionally substituted by 1-3 groups selected from —OH and halogen;
R⁵:
is selected from optionally substituted C₁-C₇ alkyl, optionally substituted phenyl, optionally substituted 5- to 6-membered heteroaryl, optionally substituted 5- to 6-membered cycloalkyl, and optionally substituted 5- to 6-membered heterocyclyl; and
R⁶ is H, optionally substituted C₁-C₄ alkyl, or CH₂CONH₂, provided that R⁶ is absent
when X₁ is O.

In one aspect of the disclosure, the compounds of the Formulae disclosed herein are selected from Compounds 1 to 240 shown in Table 1, a tautomer thereof, a solvate or stereoisomer of the compound or the tautomer, or a pharmaceutically acceptable salt of the foregoing.

In some embodiments, the disclosure provides pharmaceutical compositions comprising a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 1 to 240 shown below, a tautomer thereof, a solvate or stereoisomer of the compound or the tautomer, or a pharmaceutically acceptable salt of the foregoing, and a pharmaceutically acceptable carrier. These compositions may further comprise an additional active pharmaceutical agent.

Another aspect of the disclosure provides methods of treating a disease or condition, comprising administering to a subject in need thereof, a therapeutically effective amount of a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, wherein the disease or condition is selected amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis a metabolic disease or disorder such as diabetic neuropathy, nonalcoholic fatty liver disease (NAFLD) and nonalcoholic steatohepatitis (NASH) and a peripheral neuropathy like CIPN induced by various drugs.

A further aspect of the disclosure provides methods of treating a disease or condition caused by axonal degeneration, or neuronal damage mediated by SARM1 comprising administering to a subject in need thereof, a therapeutically effective amount of a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) and disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In some embodiments, the methods of treatment comprise administering to a subject in need thereof, a compound selected from Compounds 1 to 240 shown below, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In some embodiments, the methods of treatment comprise administration of an additional active pharmaceutical agent to the subject in need thereof, either in the same pharmaceutical composition as a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or in a separate composition. In some embodiments, the methods of treatment comprise administering a compound selected from Compounds 1 to 240 shown below, a tautomer thereof, a solvate or stereoisomer of the compound or the tautomer, or a pharmaceutically acceptable salt of the foregoing with an additional active pharmaceutical agent either in the same pharmaceutical composition or in a separate composition. When administered as a separate composition, the additional therapeutic agent may be administered prior to, at the same time as, or following administration of the compound, tautomer, solvate, stereoisomer, or a pharmaceutically acceptable salt disclosed herein.

Also disclosed herein are methods of modulating, e.g., inhibiting, SARM1 in a subject in need thereof, comprising contacting the subject with a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt. In some embodiments, the methods of modulating, e.g., inhibiting, SARM1 in a subject in need thereof comprise contacting the subject with a compound selected from Compounds 1 to 240 shown below, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

Also disclosed herein are methods of inhibiting or preventing axonal degeneration in a subject in need thereof, comprising contacting the subject with a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt. In some embodiments, the methods of inhibiting or preventing axonal degeneration or neuronal damage mediated by SARM1 in a subject in need thereof comprise contacting the subject with a compound selected from Compounds 1 to 240 shown below, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “alkyl” refers to a hydrocarbon group selected from linear and branched saturated hydrocarbon groups, containing 1-20, e.g., 1-18, 1-12, 1-10, 1-8, 1-6, 1-4, or 1-3, carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of an alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl groups. Lower alkyl contains 1-8, preferably 1-6, more preferably 1-4 carbon atoms, and more preferably 1-3 carbon atoms.

The term “alkenyl” refers to a hydrocarbon group selected from linear and branched hydrocarbon groups, comprising at least one C═C double bond and 2-20, e.g., 2-18, 2-12, 2-10, 2-8, 2-6, or 2-4, carbon atoms. Examples of the alkenyl group include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups. Lower alkenyl contains 2-8, preferably 2-6, and more preferably 2-4 carbon atoms.

The term “alkynyl” refers to a hydrocarbon group selected from linear and branched hydrocarbon groups, comprising at least one C≡C triple bond and 2-20, e.g., 2-18, 2-12, 2-10, 2-8, 2-6, or 2-4, carbon atoms. Examples of the alkynyl group include ethynyl, 1-propynyl, 2-propynyl (propargyl), 1-butynyl, 2-butynyl, and 3-butynyl groups. Lower alkynyl contains 2-8, preferably 2-6, and more preferably 2-4 carbon atoms.

The term “heteroalkyl” refers to an alkyl group, as defined herein, in which one or more of the constituent carbon atoms have been replaced by a heteroatom, e.g., nitrogen, oxygen, or sulfur, e.g., CH₃CH₂OH, CH₃CH₂OCH₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “cycloalkyl” refers to a hydrocarbon group selected from saturated and partially unsaturated cyclic hydrocarbon groups, e.g., monocyclic and polycyclic (e.g., bicyclic and tricyclic) groups. For example, the cycloalkyl group may be of 3-12, 3-10, 3-8, 3-6, 3-4, or 5-6 carbon atoms. Even further for example, the cycloalkyl group may be a monocyclic group of 3-12, 3-8, 3-6, 3-4, or 5-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4, 4], [4, 5], [5, 5], [5, 6], and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e., partially unsaturated), but is not fully conjugated, and is not an aromatic ring, as “aromatic ring” is defined herein.

The term “heterocyclic” or “heterocycle” or “heterocyclyl” refers to a ring selected from 3- to 12-membered, e.g., 3- to 6-membered, 3- to 5-membered, 4- to 5-membered, or 5- to 6-membered, monocyclic, bicyclic, and tricyclic, saturated and partially unsaturated rings comprising at least one carbon atom in addition to 1, 2, 3, or 4 heteroatoms, selected from, e.g., oxygen, sulfur, nitrogen, and silicon. “Heterocycle” also refers to a 5- to 7-membered heterocyclic ring comprising at least one heteroatom selected from N, O, and S fused with 5-, 6-, and/or 7-membered cycloalkyl, carbocyclic aromatic, or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e., partially unsaturated). A heterocycle may be substituted with oxo. The point of the attachment may be carbon or heteroatom in the heterocyclic ring. A heterocycle is not a heteroaryl as defined herein.

Examples of heterocycles include, but are not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl, 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl, and 1,1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4, 4], [4, 5], [5, 5], [5, 6], and [6,6] ring systems as mentioned above; a fused bicyclic aryl ring such as 7- to 12-membered bicyclic aryl ring systems as mentioned above, a fused tricyclic aryl ring such as 10- to 15-membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “heteroatom” means one or more of oxygen, sulfur, nitrogen, phosphorus, and silicon, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR⁺ (wherein R is, e.g., an optionally substituted alkyl group) (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds. A double bond may be depicted as == (two solid lines). The depiction of [symbol] (a solid line and a dashed line), as used herein, denotes a bond that may be a double bond or a single bond.

The term “alkoxy” as used herein, refers to an alkyl group, as defined above, wherein one carbon of the alkyl group is replaced by an oxygen atom, provided that the oxygen atom is linked between two carbon atoms.

The term “halogen” includes F, Cl, Br, and I, i.e., fluoro, chloro, bromo, and iodo, respectively.

As used herein, a “CN,” “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n+2]p orbital electrons, wherein n is an integer of 0 to 6. A “non-aromatic” ring refers to a carbocyclic or heterocyclic that does not meet the requirements set forth above for an aromatic ring, and can be either completely or partially saturated. Non-limiting examples of aromatic rings include aryl and heteroaryl rings that are further defined as follows. An “aromatic ring” may be depicted as a cycle with conjugated double bonds, such as

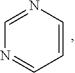

or as a cycle with an inside circle, such as

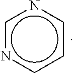

The term “aryl” herein refers to a group selected from: monocyclic carbocyclic aromatic rings, for example, phenyl; bicyclic ring systems such as 7-12 membered, e.g., 9-10 membered, bicyclic ring systems wherein at least one ring is carbocyclic and aromatic, selected, for example, from naphthalene, indane, and 1, 2, 3, 4-tetrahydroquinoline; and tricyclic ring systems such as 10-15 membered tricyclic ring systems wherein at least one ring is carbocyclic and aromatic, for example, fluorene.

For example, the aryl group may be a 6-membered carbocyclic aromatic ring fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group selected from: 5- to 7-membered, e.g., 5- to 6-membered, aromatic, monocyclic rings comprising 1, 2, 3, or 4 heteroatoms selected from N, O, and S, with the remaining ring atoms being carbon; 8- to 12-membered bicyclic rings comprising 1, 2, 3, or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3, or 4 heteroatoms, selected from N, O, and S, with the remaining ring atoms being carbon and wherein at least one ring is aromatic and at least one heteroatom is present in an aromatic ring.

For example, the heteroaryl group may be a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinolinyl.

The term “acyl” refers to a substituent group where a point of attachment in the substituent group is a carbonyl. Exemplary acyl groups include, but are not limited to, —C(═O)R′, —C(═O)NR′R″, or —C(═O)OR′, wherein R′ and R″ are independently selected from hydrogen, alkyl, alkenyl, alkynyl, heteroalkyl, heteroalkenyl, heteroalkynyl, cycloalkyl, aryl, heterocyclyl, or heteroaryl, any of which may be further substituted by one or more substituents.

Some of the compounds may exist with different points of attachment of hydrogen, referred to as “tautomers.” For example, compounds including carbonyl —CH₂C(O)-groups (keto forms) may undergo tautomerism to form hydroxyl —CH═C(OH)-groups (enol forms). Both keto and enol forms, individually as well as mixtures thereof, are also intended to be included where applicable.

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereoisomers using optically active resolving agents. Racemic mixtures of chiral compounds of the disclosure can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereoisomers, and conversion to the pure stereoisomers, and (3) separation of the substantially pure or enriched stereoisomers directly under chiral conditions.

The term “substantially pure” in the context of stereoisomers means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Unless otherwise indicated, structures depicted herein are meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the compounds disclosed herein are within the scope of the disclosure. Unless otherwise stated, all tautomeric forms of the compounds of the disclosure are within the scope of the disclosure.

The disclosure provides pharmaceutically acceptable salts of the disclosed compounds, tautomers, solvates, and stereoisomers. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, magnesium, aluminum, lithium, and ammonium. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, benzenesulfonic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate (i.e., caprate), caprylate, acrylate, formate, isobutyrate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium salts. Further non-limiting examples of pharmaceutically acceptable salts include salts of ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

If a compound is obtained as an acid addition salt, the free base can be obtained by basifying a solution of the acid addition salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The compounds, tautomers, solvates, stereoisomers, and pharmaceutically acceptable salts of the disclosure may also contain unnatural proportions of atomic isotopes at one or more of the atoms that constitute such compounds. For example, —CD₃, —CD₂H or —CDH₂ contains one or more deuteriums in place of hydrogen. For example, the compounds may be radiolabeled with radioactive isotopes, such as for example tritium (³H), iodine-125 (¹²⁵I), or carbon-14 (¹⁴C). All isotopic variations of the compounds of the disclosure, whether radioactive or not, are intended to be encompassed within the scope of the disclosure.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position.

Combinations of chemical components, e.g., substituents, ring structures, linkers, and/or heteroatoms, envisioned by this disclosure are those that result in the formation of stable or chemically feasible compounds.

In some embodiments, substituents are independently selected from optionally substituted heteroatom and optionally substituted, optionally hetero-, optionally cyclic C₁-C₁₈ hydrocarbyl, particularly wherein the optionally substituted, optionally hetero-, optionally cyclic C₁-C₁₈ hydrocarbyl is optionally-substituted, optionally hetero-, optionally cyclic alkyl, alkenyl or alkynyl, or optionally-substituted, optionally hetero-, aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In some embodiments, substituents are independently selected from: halogen, —R′, —OR′, ═O, ═NR′, ═N—OR′, —NR′R″, —SR′, —SiR′R″R″, —OC(═O)R′, —C(═O)R′, —CO₂R′, —C(═O)NR′R″, —OC(═O)NR′R″, —NR″C(═O)R′, —NR′—C(═O)NR″R″, —NR′—SO₂NR″R″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN, —NO₂, —N₃, —CH(Ph)₂, perfluoro (C₁-C₄) alkoxy, and perfluoro (C₁-C₄)alkyl, in a number ranging from zero to three, with those groups having zero, one, or two substituents being particularly preferred. R′, R″, and R′″ each independently refer to hydrogen, unsubstituted C₁-C₈ alkyl and heteroalkyl, C₁-C₈ alkyl and heteroalkyl substituted with one to three halogens, unsubstituted aryl, aryl substituted with one to three halogens, unsubstituted alkyl, alkoxy, or thioalkoxy groups, or aryl-(C₁-C₄)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl. When the aryl group is 1,2,3,4-tetrahydronaphthalenyl, it may be substituted with a substituted or unsubstituted C₃-C₇ spirocycloalkyl group. The C₃-C₇ spirocycloalkyl group may be substituted in the same manner as defined herein for “cycloalkyl.”

In some embodiments, substituents are selected from: halogen, —R′, —OR′, ═O, —NR′R″, —SR′, —SiR′R″R″, —OC(═O)R′, —C(═O)R′, —CO₂R′, —C(═O)NR′R″, —OC(═O)NR′R″, —NR″C(═O)R′, —NR″CO₂R′, —NR′—SO₂NR″R″, —S(═O)R′, —SO₂R′, —SO₂NR′R″, —NR″SO₂R, —CN, —NO₂, perfluoro C₁-C₄ alkoxy and perfluoro C₁-C₄ alkyl, where R′ and R″ are as defined above.

In some embodiments, substituents are independently selected from substituted or unsubstituted heteroatom, substituted or unsubstituted, 0-3 heteroatom-containing C₁-C₆ alkyl (e.g., C₁-C₃ alkyl or C₁-C₂ alkyl), substituted or unsubstituted, 0-3 heteroatom-containing C₂-C₆ alkenyl (e.g., C₂-C₄ alkenyl), substituted or unsubstituted, 0-3 heteroatom-containing C₂-C₆ alkynyl (e.g., C₂-C₄ alkynyl), or substituted or unsubstituted, 0-3 heteroatom-containing C₆-C₁₄ aryl (e.g., C₅-C₆ aryl), wherein each heteroatom is independently oxygen, phosphorus, sulfur, or nitrogen.

In some embodiments, substituents are independently selected from aldehyde, aldimine, alkanoyloxy, alkoxy, alkoxycarbonyl, alkyloxy, alkyl, alkenyl, alkynyl, amine, azo, halogen, carbamoyl, carbonyl, carboxamido, carboxyl, cyanyl, ester, haloformyl, hydroperoxyl, hydroxyl, imine, isocyanide, isocyante, N-tert-butoxycarbon yl, nitrate, nitrile, nitrite, nitro, nitroso, phosphate, phosphono, sulfide, sulfonyl, sulfo, sulfhydryl, thiol, thiocyanyl, trifluoromethyl, and trifluromethyl ether (OCF₃) groups.

Preferred substituents are disclosed herein and exemplified in the tables, structures, examples, and claims, and may be applied across different compounds of this disclosure. For example, substituents of a given compound may be combinatorically used with other compounds.

It may be advantageous to separate reaction products from one another and/or from starting materials. The desired products of each step or series of steps are separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example, reverse-phase and normal phase; size exclusion; ion exchange; high, medium, and low pressure liquid chromatography methods and apparatus; small scale analytical; simulated moving bed (“SMB”) and preparative thin or thick layer chromatography, as well as techniques of small scale thin layer and flash chromatography. One skilled in the art may apply such techniques to achieve a desired separation.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH), and sodium methoxide (NaOMe; NaOCH₃).

The term “subject” refers to an animal including a human.

The term “therapeutically effective amount” refers to the amount of a compound that produces a desired effect for which it is administered (e.g., improvement in a disease or condition, lessening the severity of a disease or condition, and/or reducing progression of a disease or condition, e.g., ALS, Parkinson's disease, multiple sclerosis, traumatic brain injury, diabetic neuropathy, and CIPN. The disease or condition may be caused by axonal degeneration. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, e.g., ALS, Parkinson's disease, multiple sclerosis, traumatic brain injury, diabetic neuropathy, and CIPN. The disease or condition may be caused by axonal degeneration. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages, for example, a range of +10% with respect to a specific point value) that is recognized by one of ordinary skill in the art.

### II. Compounds and Compositions

In a 1^{st} embodiment, a compound of this disclosure is a compound of the following structural Formula 1:

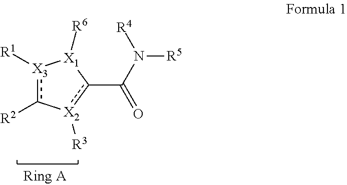

a tautomer thereof, a solvate or stereoisomer of the compound or the tautomer, or a pharmaceutically acceptable salt of the foregoing, wherein:
X₁ is C, N, or O; X₂ is C or N; X₃ is C or N;
R¹ and R²:
are independently selected from H, optionally substituted C₁-C₄ alkyl (C₁ alkyl, C₂ alkyl, or C₃ alkyl), and optionally substituted C₃-C₆ cycloalkyl(C₃, C₄, C₅, or C₆ cycloalkyl), or
join to form a 5- to 7-membered (5-, 6-, or 7-membered) ring such that the 5- to 7-membered (5-, 6-, or 7-membered) ring and Ring A together form Fused Ring A selected from Fused Ring A-1, Fused Ring A-2, Fused Ring A-3, and Fused Ring A-4 shown below:

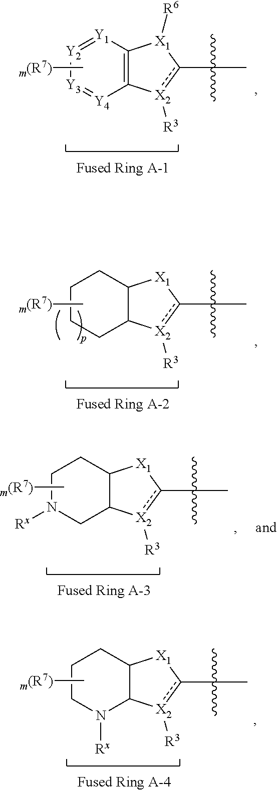

wherein:
Y₁, Y₂, Y₃, and Y₄ are each independently selected from C and N;
p is an integer selected from 0, 1, and 2;
R⁷ is selected from halogen (e.g., F, Cl, or Br), —CN, —CONH₂, —SO₂Me, optionally substituted 3-6 membered (3-, 4-, 5-, or 6-membered) cycloalkyl, optionally substituted 4-6 membered (4-, 5-, or 6-membered) heterocyclyl (e.g., containing, 1, 2, or 3 heteroatoms such as N, S, or O), optionally substituted phenyl, optionally substituted 5-6 membered heteroaryl (e.g., containing, 1, 2, or 3 heteroatoms such as N, S, or O), optionally substituted C₁-C₄ alkyl, ═O (provided that Fused Ring A is not Fused Ring A-1), and —OR^{s}, wherein R^{s} is optionally substituted C₁-C₄ alkyl, and m is an integer selected from 0, 1, 2, and 3 (examples of R⁷ includes F, Cl, Br, CH₃, CH₂CH₃, or OCH₃);
R^{x} is selected from H, optionally substituted 3- to 5-membered (3-, 4-, or 5-membered) cycloalkyl, optionally substituted C₁-C₄ alkyl, and —C(═O)R^{y}, wherein R^{y} is optionally substituted 3-5 membered cycloalkyl or optionally substituted C₁-C₄ alkyl;
R³:
is selected from absent, H, —CN, —C(═O)NH₂, and optionally substituted C₁-C₄ alkyl (e.g., —CH₂OH, —CH₂CH₂OH, —CH₂CH₂OCH₃, —CH₂CN, or —CH₂C(═O)NH₂);
R⁴:
is selected from H, optionally substituted C₁-C₆ alkyl, optionally substituted 3- to 5-membered cycloalkyl, optionally substituted 3- to 6-membered heterocyclyl, optionally substituted 5- to 6-membered heteroaryl, —R^{a}C(═O)R^{b}, —R^{a}NHC(═O)R^{b}, —R^{a}NR^{c}R^{d}, —R^{a}S(═O)R^{b}, —R^{a}S(═O)₂R^{b},

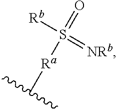

—R^{a}NHS(═O)₂R^{b}, and —R^{a}C(═O)NR^{c}R^{d}, wherein:
R^{a}, for each occurrence, is independently selected from C₁-C₈ alkyl optionally substituted by 1-3 groups selected from —OH and halogen;
R^{b}, for each occurrence, is independently selected from H, —OH, —O(C₁-C₃ alkyl), 4- to 6-membered heterocyclyl, and C₁-C₆ alkyl optionally substituted by 1-3 groups selected from —OH and halogen;
R^{c} and R^{d}, for each occurrence, are independently selected from H and C₁-C₆ alkyl, or
R^{c} and R^{d} join to form a 3- to 6-membered cycloalkyl or a 3- to 6-membered heterocyclyl wherein the 3- to 6-membered cycloalkyl or 3- to 6-membered heterocyclyl of R^{c} and R^{d} is optionally substituted by 1-3 groups selected from —OH and halogen;
R⁵:
is selected from optionally substituted C₁-C₇ alkyl (C₁, C₂, C₃, C₄, C₅, C₆, or C₇ alkyl), optionally substituted phenyl, optionally substituted 5- to 6-membered heteroaryl, optionally substituted 5- to 6-membered cycloalkyl, and optionally substituted 5- to 6-membered heterocyclyl; and
R⁶ is H, optionally substituted C₁-C₄ alkyl, or CH₂CONH₂, provided that R⁶ is absent
when X₁ is O.

Combinations of substituents as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed.

In a 2^{nd} embodiment, a compound of the disclosure is a compound of the following structural Formula 2:

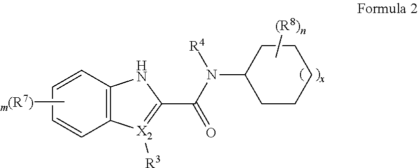

a tautomer thereof, a solvate or stereoisomer of the compound or the tautomer, or a pharmaceutically acceptable salt of the foregoing, wherein x is an integer selected from 0 and 1; R⁸, for each occurrence, is independently selected from C₁-C₄ alkyl (optionally substituted with 1 to 3 groups selected from —OH and halogen), halogen, CN, —O(C₁-C₄ alkyl), and —C(═O)O(C₁-C₄ alkyl); and n is an integer selected from 0, 1, 2, and 3, all other variables not specifically defined herein are as defined in any one of the suitable preceding embodiments. In some embodiments,
R³ is selected from absent, H, CN, —C(═O)NH₂, and C₁-C₃ alkyl, wherein the C₁-C₃ alkyl of R³ is optionally substituted with a group selected from OH, —O(C₁-C₂ alkyl), CN, —C(═O)NH₂, —NH₂, and —NHR^{p1}, wherein
R^{p1} is selected from —C(═O)(C₁-C₂ alkyl) and 5- to 6-membered heteroaryl, wherein the 5- to 6-membered heteroaryl of R^{p1} is optionally substituted with 1-2 groups selected from C₁-C₃ alkyl, —C(═O)OH, —C(═O)NH₂, and phenyl optionally substituted with 1 to 2 groups selected from halogen;
R⁴ is selected from H, C₁-C₃ alkyl, and 3- to 4-membered heterocyclyl, wherein the C₁-C₃ alkyl of R⁴ is optionally substituted with a group selected from OH, O(C₁-C₃ alkyl), 3- to 4-membered heterocyclyl, and —NR^{p}R^{q}, and wherein
the 3- to 4-membered heterocyclyl of the C₁-C₃ alkyl of R⁴ is optionally substituted with a group selected from C₁-C₃ alkyl, and wherein R^{p} and R^{q} are each independently selected from H and C₁-C₂ alkyl;
and
R⁷ is selected from halogen, —CN, —CONH₂, 4- to 6-membered heterocyclyl, 5- to 6-membered heteroaryl, phenyl, 3-5 membered cycloalkyl, C₁-C₄ alkyl (optionally substituted by 1-3 groups selected from halogen and OH), ═O (provided that Fused Ring A is not Fused Ring A-1), O(C₁-C₄ alkyl), and m is an integer selected from 0, 1, and 2.

In a 3^{rd} embodiment, a compound of the disclosure is a compound of the following structural Formula 2-1:

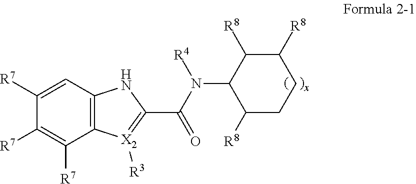

a tautomer thereof, a solvate or stereoisomer of the compound or the tautomer, or a pharmaceutically acceptable salt of the foregoing, wherein
X₂ is Cor N;
R³ is selected from absent, H, —CN, —C(═O)NH₂, and C₁-C₂ alkyl, wherein the C₁-C₂ alkyl of R³ is optionally substituted with a group selected from OH, —O(C₁-C₂ alkyl), CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —CN, —C(═O)OH, and —C(═O)NH₂;
R⁴ is selected from H, 3- to 5-membered cycloalkyl, 3- to 5-membered heterocyclyl, and C₁-C₃ alkyl optionally substituted with a group selected from OH, CN, and 3- to 5-membered heterocyclyl;
R⁷, for each occurrence, is independently selected from H, halogen, 3-4 membered cycloalkyl, and C₁-C₃ alkyl optionally substituted with 1 to 3 groups selected from —OH, halogen, and O(C₁-C₄ alkyl);
R⁸, for each occurrence, is independently selected from H, C₁-C₂ alkyl (optionally substituted with 1 to 3 groups selected from —OH and halogen), halogen, CN, —O(C₁-C₄ alkyl), and —C(═O)O(C₁-C₄ alkyl); and
x is an integer selected from 0 and 1; and
all other variables not specifically defined herein are as defined in any one of the suitable preceding embodiments.

In a 4^{th} embodiment, a compound of the disclosure is a compound of the following structural Formula 2-2:

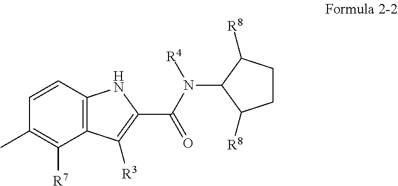

a tautomer thereof, a solvate or stereoisomer of the compound or the tautomer, or a pharmaceutically acceptable salt of the foregoing, wherein
R³ is selected from H, —CN, —C(═O)NH₂, and C₁-C₂ alkyl, wherein the C₁-C₂ alkyl of R³ is optionally substituted with a group selected from OH, —O(C₁-C₂ alkyl), —CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —CN, —C(═O)OH, and —C(═O)NH₂;
R⁴ is methyl or CH₂CH₂OH;
R⁷ is selected from H, methyl, 3-4 membered cycloalkyl, and C₁-C₃ alkyl optionally substituted with 1 to 3 groups selected from —OH and halogen; and
R⁸, for each occurrence, is independently selected from H, and C₁-C₂ alkyl optionally substituted with 1 to 3 groups selected from —OH and halogen; and all other variables not specifically defined herein are as defined in any one of the suitable preceding embodiments.

In a 5^{th} embodiment, a compound of the disclosure is a compound of the following structural Formula 2-3:

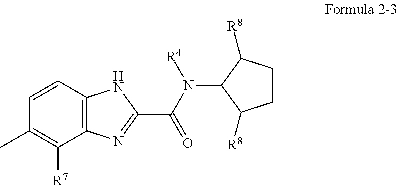

a tautomer thereof, a solvate or stereoisomer of the compound or the tautomer, or a pharmaceutically acceptable salt of the foregoing, wherein
R⁴ is methyl or CH₂CH₂OH;
R⁷ is selected from H, methyl, 3-4 membered cycloalkyl, and C₁-C₃ alkyl optionally substituted with 1 to 3 groups selected from —OH and halogen;
R⁸, for each occurrence, is independently selected from H and C₁-C₂ alkyl optionally substituted with 1 to 3 groups selected from —OH and halogen; and all other variables not specifically defined herein are as defined in any one of the suitable preceding embodiments.

In a 6^{th} embodiment, a compound of the disclosure is a compound of the following structural Formula 2-4:

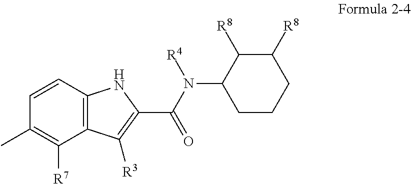

a tautomer thereof, a solvate or stereoisomer of the compound or the tautomer, or a pharmaceutically acceptable salt of the foregoing, wherein
R³ is selected from H, CN, —C(═O)NH₂, and C₁-C₂ alkyl, wherein
the C₁-C₂ alkyl of R³ is optionally substituted with a group selected from OH, —O(C₁-C₂ alkyl), CN, —C(═O)NH₂, and —NHR^{p1}, wherein
R^{p1} is selected from H, —C(═O)(C₁-C₂ alkyl), and 5- to 6-membered heteroaryl,
wherein the 5- to 6-membered heteroaryl of R^{p1} is optionally substituted with 1-2 groups selected from C₁-C₃ alkyl, —C(═O)OH, and —C(═O)NH₂;
R⁴ is methyl or CH₂CH₂OH;
R⁷ is selected from H, methyl, and 3-4 membered cycloalkyl optionally substituted with 1 to 3 groups selected from —OH and halogen, and
R⁸, for each occurrence, is independently selected from H and C₁-C₂ alkyl optionally substituted with 1 to 3 groups selected from —OH and halogen; and all other variables not specifically defined herein are as defined in any one of the suitable preceding embodiments.

In a 7^{th} embodiment, a compound of the disclosure is a compound of the following structural Formula 3:

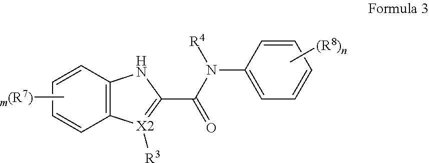

a tautomer thereof, a solvate or stereoisomer of the compound or the tautomer, or a pharmaceutically acceptable salt of the foregoing, wherein R⁸, for each occurrence, is independently selected from C₁-C₄ alkyl (optionally substituted with 1 to 3 groups selected from —OH and halogen), halogen, —CN, —O(C₁-C₄ alkyl), and —C(═O)O(C₁-C₄ alkyl); and n is an integer selected from 0, 1, 2, and 3; and all other variables not specifically defined herein are as defined in any one of the suitable preceding embodiments.

In some embodiments,
R³ is selected from absent, H, CN, —C(═O)NH₂, and C₁-C₃ alkyl, wherein the C₁-C₃ alkyl of R³ is optionally substituted with a group selected from OH, —O(C₁-C₂ alkyl), CN, —C(═O)NH₂, —NH₂, and —NHR^{p1}, wherein
R^{p1} is selected from —C(═O)(C₁-C₂ alkyl) and 5- to 6-membered heteroaryl, wherein the 5- to 6-membered heteroaryl of R^{p1} is optionally substituted with 1-2 groups selected from C₁-C₃ alkyl, —C(═O)OH, —C(═O)NH₂, and phenyl optionally substituted with 1 to 2 groups selected from halogen;
R⁴ is selected from H, C₁-C₃ alkyl, and 3- to 4-membered heterocyclyl, wherein the C₁-C₃ alkyl of R⁴ is optionally substituted with a group selected from OH, O(C₁-C₃ alkyl), 3- to 4-membered heterocyclyl, and —NR^{p}R^{q}, and wherein
the 3- to 4-membered heterocyclyl of the C₁-C₃ alkyl of R⁴ is optionally substituted with a group selected from C₁-C₃ alkyl, and wherein R^{p} and R^{q} are each independently selected from H and C₁-C₂ alkyl;
and
R⁷ is selected from halogen, —CN, —CONH₂, 4- to 6-membered heterocyclyl, 5- to 6-membered heteroaryl, phenyl, 3-5 membered cycloalkyl, C₁-C₄ alkyl (optionally substituted by 1-3 groups selected from halogen and OH), ═O (provided that Fused Ring A is not Fused Ring A-1), O(C₁-C₄ alkyl), and m is an integer selected from 0, 1, and 2.

In a 8^{th} embodiment, a compound of the disclosure is a compound of the following structural Formula 3-1:

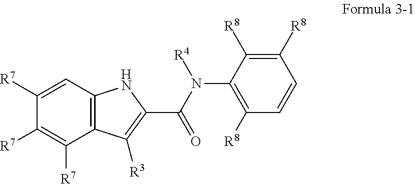

a tautomer thereof, a solvate or stereoisomer of the compound or the tautomer, or a pharmaceutically acceptable salt of the foregoing, wherein
R³ is selected from H, —CN, —C(═O)NH₂, and C₁-C₃ alkyl, wherein
the C₁-C₃ alkyl of R³ is optionally substituted with a group selected from OH, —O(C₁-C₂ alkyl), CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —C(═O)OH, and —C(═O)NH₂;
R⁴ is selected from H, 3- to 5-membered cycloalkyl, 3- to 5-membered heterocyclyl, and C₁-C₃ alkyl optionally substituted with a group selected from OH, CN, and 3- to 5-membered heterocyclyl;
R⁷, for each occurrence, is independently selected from H, halogen, 3-4 membered cycloalkyl, and C₁-C₃ alkyl optionally substituted with a group selected from —OH, halogen, and O(C₁-C₄ alkyl); and
R⁸, for each occurrence, is independently selected from H, C₁-C₂ alkyl (optionally substituted with 1 to 3 groups selected from —OH and halogen), halogen, CN, —C(═O)O(C₁-C₄ alkyl), and O(C₁-C₄ alkyl); and
all other variables not specifically defined herein are as defined in any one of the suitable preceding embodiments.

In a 9^{th} embodiment, a compound of the disclosure is a compound of the following structural Formula 3-2:

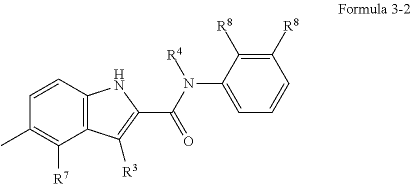

a tautomer thereof, a solvate or stereoisomer of the compound or the tautomer, or a pharmaceutically acceptable salt of the foregoing, wherein
R³ is selected from H, —CN, —C(═O)NH₂, and C₁-C₂ alkyl, wherein
the C₁-C₂ alkyl of R³ is optionally substituted with a group selected from OH, —O(C₁-C₂ alkyl), CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —C(═O)OH, and —C(═O)NH₂;
R⁴ is selected from H, 3- to 5-membered cycloalkyl, 3- to 5-membered heterocyclyl, and C₁-C₃ alkyl optionally substituted with a group selected from OH, CN, and 3- to 5-membered heterocyclyl;
R⁷ is selected from H, methyl, and 3-4 membered cycloalkyl, and
R⁸, for each occurrence, is independently selected from H, C₁-C₂ alkyl (optionally substituted with 1 to 3 groups selected from —OH and halogen), halogen, and O(C₁-C₄ alkyl); and all other variables not specifically defined herein are as defined in any one of the suitable preceding embodiments.

In a 10^{th} embodiment, a compound of the disclosure is a compound of the following structural Formula 3-3:

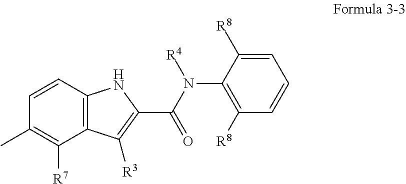

a tautomer thereof, a solvate or stereoisomer of the compound or the tautomer, or a pharmaceutically acceptable salt of the foregoing, wherein
R³ is selected from H, —CN, —C(═O)NH₂, and C₁-C₂ alkyl, wherein
the C₁-C₂ alkyl of R³ is optionally substituted with a group selected from OH, —O(C₁-C₂ alkyl), —CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —C(═O)OH, and —C(═O)NH₂;
R⁴ is selected from H, 3- to 5-membered cycloalkyl, 3- to 5-membered heterocyclyl, and C₁-C₃ alkyl optionally substituted with a group selected from OH, CN, and 3- to 5-membered heterocyclyl;
R⁷ is selected from H, methyl, and 3-5 membered cycloalkyl, and
R⁸, for each occurrence, is independently selected from H, C₁-C₂ alkyl (optionally substituted with 1 to 3 groups selected from —OH and halogen), halogen, and O(C₁-C₄ alkyl); and
all other variables not specifically defined herein are as defined in any one of the suitable preceding embodiments.

In a 11^{th} embodiment, a compound of the disclosure is a compound of the following structural Formula 4:

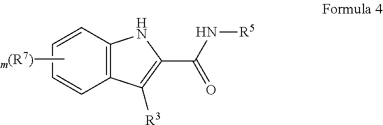

a tautomer thereof, a solvate or stereoisomer of the compound or the tautomer, or a pharmaceutically acceptable salt of the foregoing, and all other variables not specifically defined herein are as defined in any one of the suitable preceding embodiments.

In a 12^{th} embodiment, a compound of the disclosure is a compound of the following structural Formula 4-1:

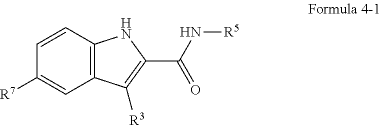

a tautomer thereof, a solvate or stereoisomer of the compound or the tautomer, or a pharmaceutically acceptable salt of the foregoing, wherein
R³ is C₁-C₂ alkyl optionally substituted with —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —C(═O)OH, —C(═O)NH₂, and phenyl optionally substituted with halogen;
R⁵ is selected from C₁-C₆ alkyl, wherein the C₁-C₆ alkyl of R⁵ is optionally substituted with —C(═O)NHR^{p2}, wherein R^{p2} is selected from C₁-C₃ alkyl and phenyl; and
R⁷ is selected from C₁-C₂ alkyl optionally substituted with 1 to 3 groups selected from —OH, halogen, and O(C₁-C₄ alkyl); and
all other variables not specifically defined herein are as defined in any one of the suitable preceding embodiments.

In a 13^{th} embodiment, in a compound, tautomer, a solvate or stereoisomer of the compound or the tautomer, or pharmaceutically acceptable salt of this disclosure, R¹ and R² do not join to form Fused Ring A, and R¹ and R² are each independently selected from H, C₁-C₄ alkyl, and 3- to 6-membered cycloalkyl; and all other variables not specifically defined herein are as defined in any one of the suitable preceding embodiments.

In a 14^{th} embodiment, in a compound, tautomer, a solvate or stereoisomer of the compound or the tautomer, or pharmaceutically acceptable salt of this disclosure, R¹ and R² do not join to form Fused Ring A, and R¹ and R² are each independently selected from H, methyl, and C₂-C₃ alkyl (such as —CH(CH₃)₂); and all other variables not specifically defined herein are as defined in any one of the suitable preceding embodiments.

In a 15^{th} embodiment, a compound of the disclosure is a compound of the following structural Formula 5:

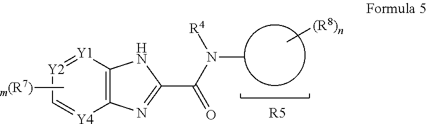

a tautomer thereof, a solvate or stereoisomer of the compound or the tautomer, or a pharmaceutically acceptable salt of the foregoing, wherein
Y₁, Y₂, and Y₄ are each independently selected from C and N;
R⁵ is phenyl, 5- to 6-membered heteroaryl, 5- to 6-membered cycloalkyl, or 5- to 6-membered heterocyclyl, wherein R⁵ is substituted with n groups of R⁸; and
R⁸, for each occurrence, is independently selected from halogen, —CN, —CONH₂, —SO₂Me, —C(═O)CH₃, C₁-C₄ alkyl (optionally substituted with 1-3 groups selected from —OH, halogen, —SO₂Me, and 4- to 6-membered heterocyclyl), 4- to 6-membered heterocyclyl (optionally substituted with 1-3 groups selected from —OH and halogen), ═O, and —O(C₁-C₃ alkyl), wherein the C₁-C₃ alkyl of the —O(C₁-C₃ alkyl) is optionally substituted with 1-3 groups selected from —OH and halogen; and n is an integer selected from 0, 1, 2, 3, and 4; and
all other variables not specifically defined herein are as defined in any one of the suitable preceding embodiments.

In a 16^{th} embodiment, a compound of the disclosure is a compound of the following structural Formula 6-1, 6-2, 6-3, or 6-4:

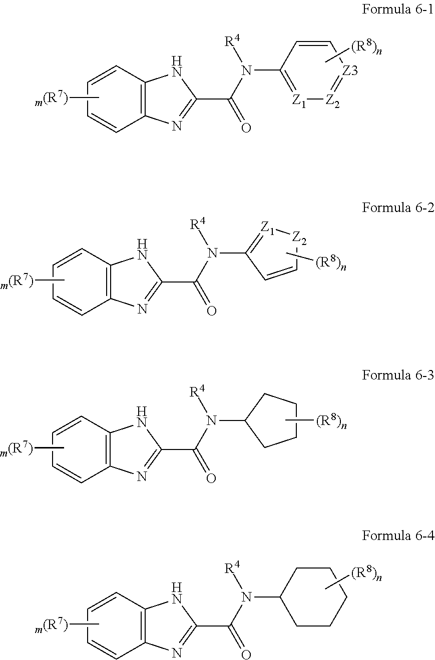

a tautomer thereof, a solvate or stereoisomer of the compound or the tautomer, or a pharmaceutically acceptable salt of the foregoing, wherein
Z₁, Z₂, and Z₃ are each independently selected from C and N;
R⁸, for each occurrence, is independently selected from halogen, —CN, C₁-C₂ alkyl, —O(C₁-C₂ alkyl), and ═O, wherein the C₁-C₂ alkyl of the —O(C₁-C₂ alkyl) is optionally substituted with 1-3 groups selected from halogen; and
all other variables not specifically defined herein are as defined in any one of the suitable preceding embodiments.

In a 17^{th} embodiment, a compound of the disclosure is a compound of the following structural Formula 6-1a:

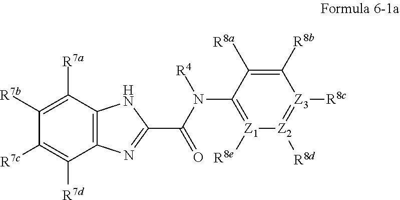

a tautomer thereof, a solvate or stereoisomer of the compound or the tautomer, or a pharmaceutically acceptable salt of the foregoing, wherein
Z₁, Z₂, and Z₃ are each independently selected from C and N;
R^{7a} is selected from H and halogen;
R^{7b} is selected from H and halogen;
R^{7c} is selected from H, halogen, C₁-C₄ alkyl (optionally substituted by 1-3 groups selected from OH, —CN, —CONH₂ and halogen),

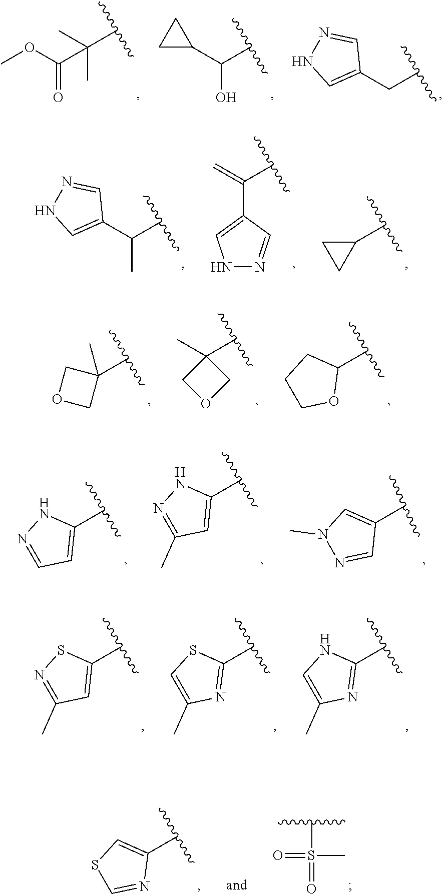

R^{7d} is selected from H, halogen, C₁-C₂ alkyl, —CN,

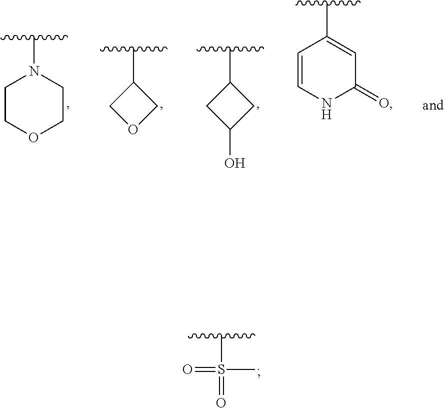

R^{8a}, R^{8b}, R^{8c}, R^{8d}, and R^{8e} are each independently selected from H, halogen, —CN, C₁-C₂ alkyl, —O(C₁-C₂ alkyl), ═O, and —OCF₃; and
all other variables not specifically defined herein are as defined in any one of the suitable preceding embodiments.

In a 18^{th} embodiment, a compound of the disclosure is a compound of the following structural Formula 6-2a, 6-2b, 6-2c, 6-2d, 6-2e, or 6-2f:

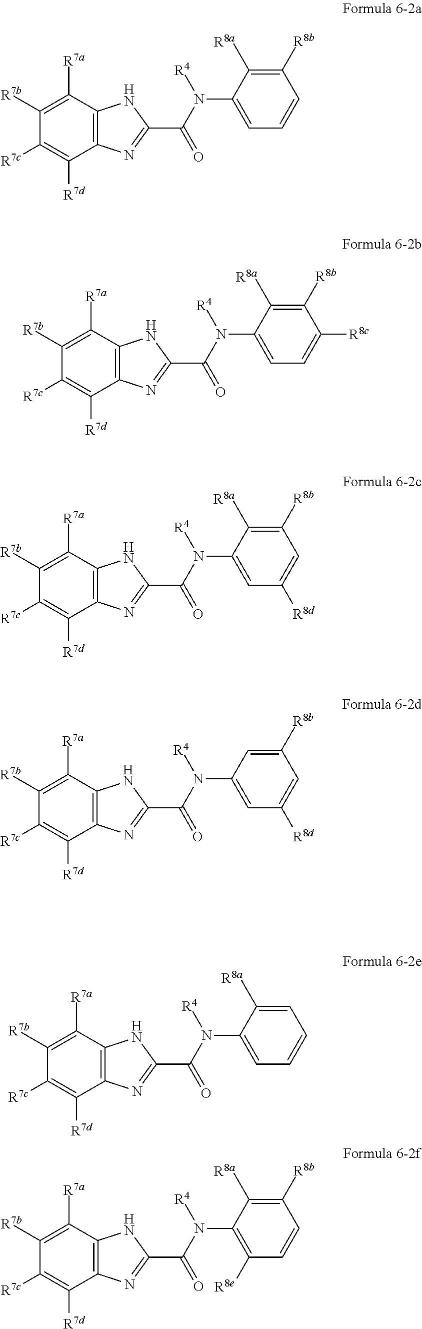

a tautomer thereof, a solvate or stereoisomer of the compound or the tautomer, or a pharmaceutically acceptable salt of the foregoing, wherein R^{8a}, R^{8b}, R^{8c}, R^{8d}, and R^{8e} are independently selected from F, Cl, CN, CH₃, ═O, —OCH₃, and —OCF₃; and all other variables not specifically defined herein are as defined in any one of the suitable preceding embodiments.

In a 19^{th} embodiment, in a compound, tautomer, a solvate or stereoisomer of the compound or the tautomer, or pharmaceutically acceptable salt of this disclosure,

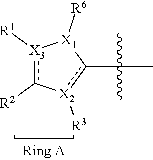

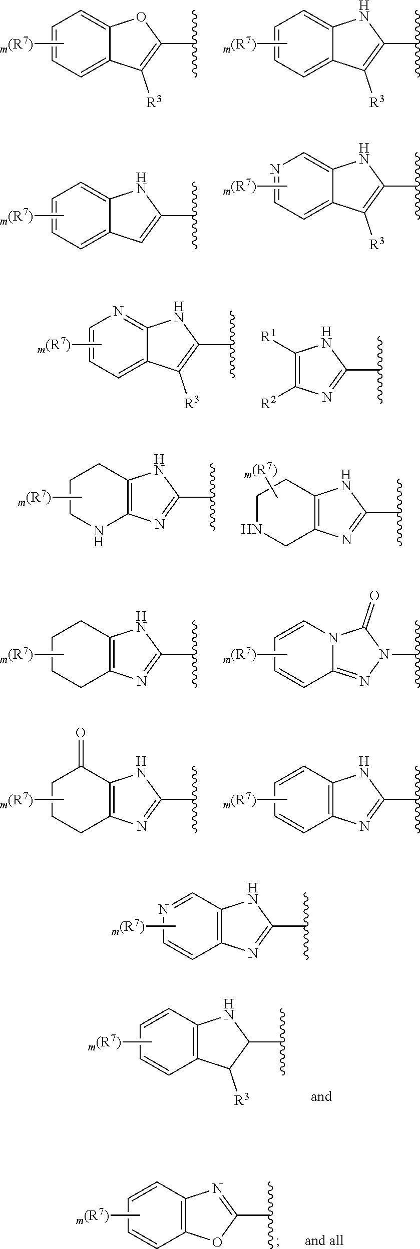

other variables not specifically defined herein are as defined in any one of the suitable preceding embodiments.

In a 20^{th} embodiment, in a compound, tautomer, a solvate or stereoisomer of the compound or the tautomer, or pharmaceutically acceptable salt of this disclosure,

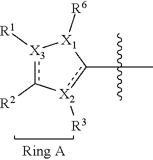

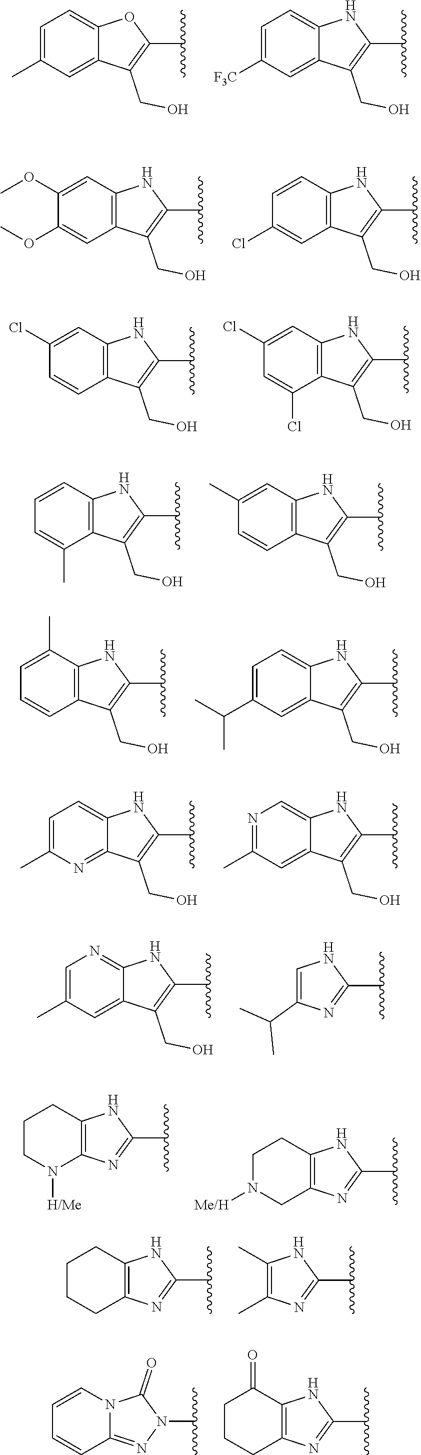

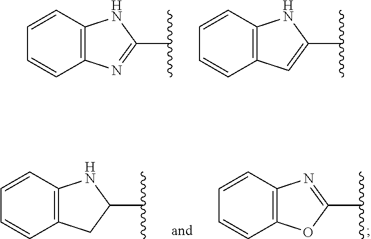

and all other variables not specifically defined herein are as defined in any one of the suitable preceding embodiments.

In a 21^{st} embodiment, in a compound, tautomer, a solvate or stereoisomer of the compound or the tautomer, or pharmaceutically acceptable salt of this disclosure,

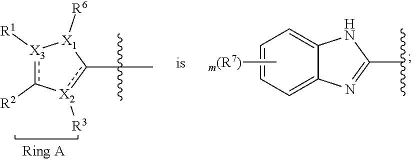

herein are as defined in any one of the suitable preceding embodiments.

In a 22^{nd} embodiment, in a compound, tautomer, a solvate or stereoisomer of the compound or the tautomer, or pharmaceutically acceptable salt of this disclosure,

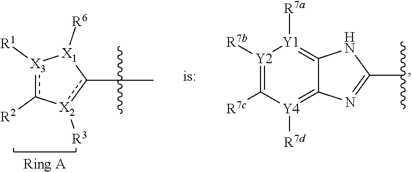

wherein:
Y₁, Y₂, and Y₃ are each independently selected from C and N;
R^{7a} and R^{7b} are each independently selected from H, CH₃, Cl, F, Br, and —OCH₃;
R^{7c} is selected from H, Me, Cl, F, Br, —OMe, CF₃, CHF₂, CH₂CF₃, CH₂CHF₂, —OCF₃, —OCHF₂, —SO₂Me, —CN, OH, CH₂OH, —COOH, —CONH₂, —CO₂Me,

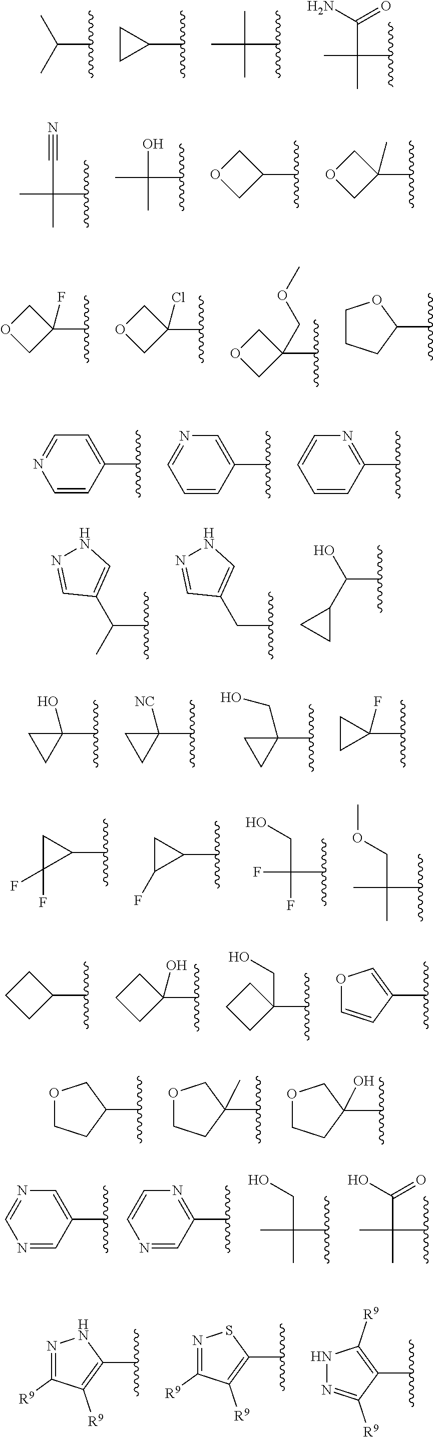

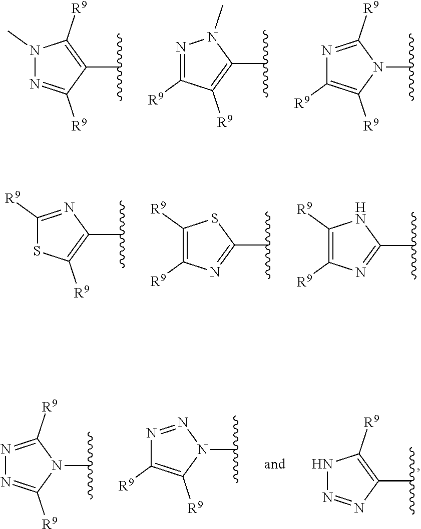

wherein R⁹, for each occurrence, is independently from H, Me, Cl, F, and —CN; and
R^{7d} is selected from H, Me, Cl, F, Br, —OMe, —CN, —CONH₂, —SO₂Me,

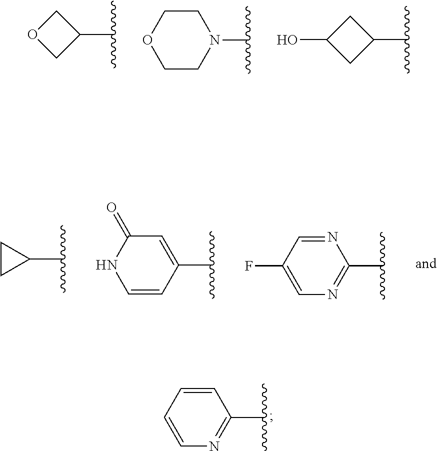

and all other variables not specifically defined herein are as defined in any one of the suitable preceding embodiments.

In a 23^{rd} embodiment, in a compound, tautomer, a solvate or stereoisomer of the compound or the tautomer, or pharmaceutically acceptable salt of this disclosure,

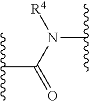

of Formula 1 is selected from:

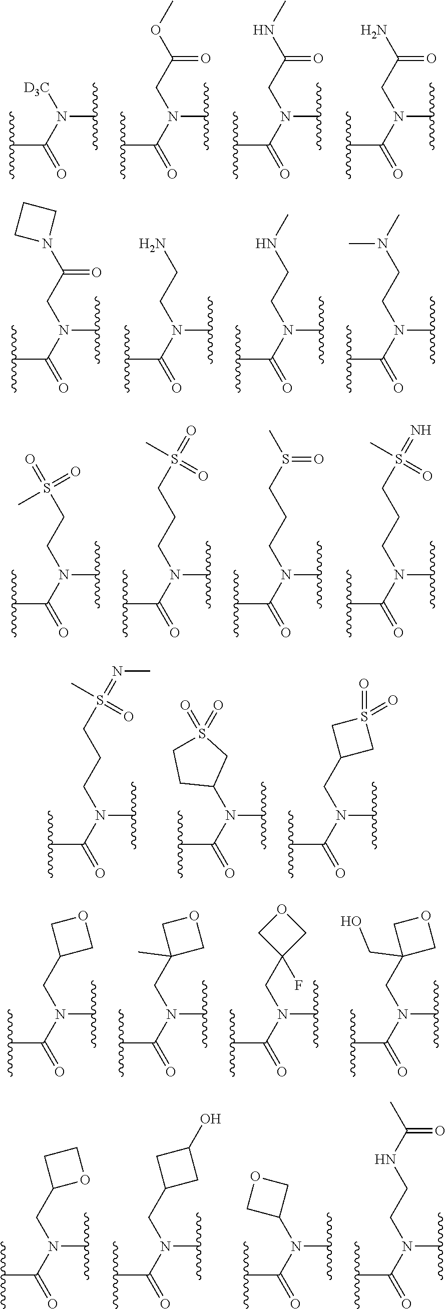

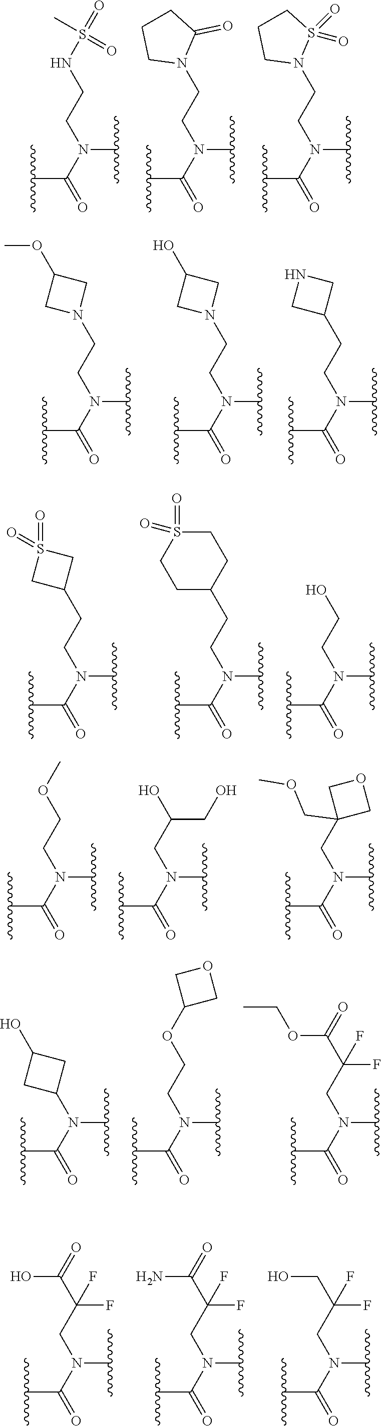

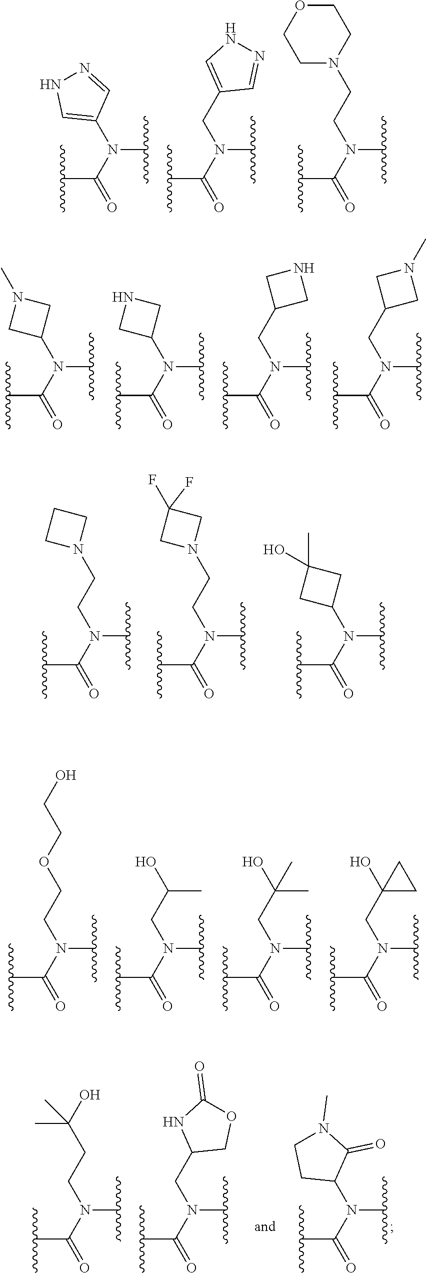

and all other variables not specifically defined herein are as defined in any one of the suitable preceding embodiments.

In a 24^{th} embodiment, in a compound, tautomer, a solvate or stereoisomer of the compound or the tautomer, or pharmaceutically acceptable salt of this disclosure, R³ is selected from absent, H, CN, —C(═O)NH₂, and C₁-C₄ alkyl, wherein
the C₁-C₄ alkyl of R³ is optionally substituted with 1 to 2 groups selected from OH, —O(C₁-C₃ alkyl), CN, —C(═O)NH₂, —NH₂, and —NHR^{p1}, wherein
R^{p1} is selected from —C(═O)(C₁-C₃ alkyl) and 5- to 6-membered heteroaryl, wherein the 5- to 6-membered heteroaryl of R^{p1} is optionally substituted with 1-3 groups selected from C₁-C₃ alkyl, —C(═O)OH, —C(═O)NH₂, and optionally substituted phenyl; and
all other variables not specifically defined herein are as defined in any one of the suitable preceding embodiments.

In a 25^{th} embodiment, in a compound, tautomer, a solvate or stereoisomer of the compound or the tautomer, or pharmaceutically acceptable salt of this disclosure, R³ is selected from absent, H, CN, —C(═O)NH₂, and C₁-C₃ alkyl, wherein the C₁-C₃ alkyl of R³ is optionally substituted with a group selected from OH, —O(C₁-C₂ alkyl), CN, —C(═O)NH₂, —NH₂, and —NHR^{p1}, wherein
R^{p1} is selected from —C(═O)(C₁-C₂ alkyl) and 5- to 6-membered heteroaryl, wherein the 5- to 6-membered heteroaryl of R^{p1} is optionally substituted with 1-2 groups selected from C₁-C₃ alkyl, —C(═O)OH, —C(═O)NH₂, and phenyl optionally substituted with 1 to 2 groups selected from halogen; and
all other variables not specifically defined herein are as defined in any one of the suitable preceding embodiments.

In a 26^{th} embodiment, in a compound, tautomer, a solvate or stereoisomer of the compound or the tautomer, or pharmaceutically acceptable salt of this disclosure, R³ is selected from absent, H, CN, —C(═O)NH₂, CH₂OH, and

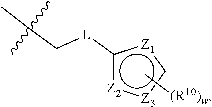

wherein L is N or O, Z1, Z2, and Z3 are independent selected from C, N, and O, R¹⁰ is selected from C₁-C₃ alkyl, —C(═O)NH₂, —C(═O)OH, and phenyl optionally substituted with 1 to 3 groups selected from halogen, and w is an integer selected from 0, 1, and 2; and all other variables not specifically defined herein are as defined in any one of the suitable preceding embodiments.

In a 27^{th} embodiment, in a compound, tautomer, a solvate or stereoisomer of the compound or the tautomer, or pharmaceutically acceptable salt of this disclosure, R³ is selected from absent, H, CH₂CH₂CH₃, CN, —C(═O)NH₂, CH₂OH, CH₂CH₂OH, CH₂OCH₃, CH₂CH₂OCH₃, CH₂NH₂, CH₂CH₂NH₂, CH₂CN, CH₂C(═O)NH₂,

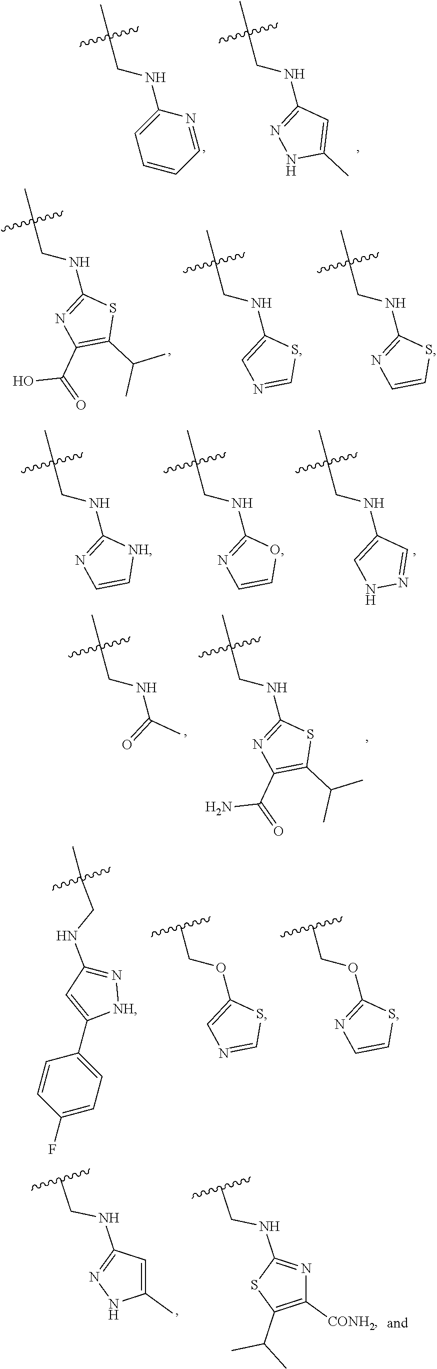

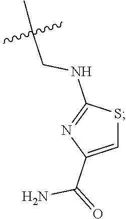 [SMILES: *CNc1nc(C(N)=O)cs1]

and all other variables not specifically defined herein are as defined in any one of the suitable preceding embodiments.

In a 28^{th} embodiment, in a compound, tautomer, a solvate or stereoisomer of the compound or the tautomer, or pharmaceutically acceptable salt of this disclosure, R⁴ is selected from H, C₁-C₃ alkyl, and 3- to 4-membered heterocyclyl,
wherein the C₁-C₃ alkyl of R⁴ is optionally substituted with a group selected from OH, O(C₁-C₃ alkyl), 3- to 4-membered heterocyclyl, and —NR^{p}R^{q}, and wherein
the 3- to 4-membered heterocyclyl of the C₁-C₃ alkyl of R⁴ is optionally substituted with a group selected from C₁-C₃ alkyl, and
wherein R^{p} and R^{q} are each independently selected from H and C₁-C₂ alkyl; and all other variables not specifically defined herein are as defined in any one of the suitable preceding embodiments.

In a 29^{th} embodiment, in a compound, tautomer, a solvate or stereoisomer of the compound or the tautomer, or pharmaceutically acceptable salt of this disclosure, R⁴ is selected from is H, methyl, CH₂CH₂OH, CH₂CH₂OCH₃, CH₂CH₂NH₂, CH₂CH₂NHCH₃, CH₂CH₂N(CH₃)₂,

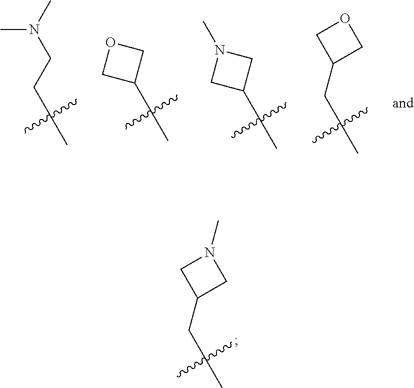

and all other variables not specifically defined herein are as defined in any one of the suitable preceding embodiments.

In a 30^{th} embodiment, in a compound, tautomer, a solvate or stereoisomer of the compound or the tautomer, or pharmaceutically acceptable salt of this disclosure, R⁴ is selected from:
C₁-C₆ alkyl optionally substituted by 1-3 groups selected from D, halogen, —OR^{b}, 3- to 5-membered cycloalkyl (optionally substituted by 1-3 groups selected from —OH, C₁-C₃ alkyl, and halogen), 4- to 6-membered heterocyclyl (optionally substituted by 1-3 groups selected from —OR^{b}, halogen, ═O, and C₁-C₃ alkyl optionally substituted by —OR^{b}), and 5- to 6-membered heteroaryl (optionally substituted by 1-3 groups selected from —OR^{b}, halogen, and C₁-C₃ alkyl optionally substituted by —ORD);
4- to 6-membered heterocyclyl optionally substituted by 1-3 groups selected from —OR^{b}, halogen, ═O, and C₁-C₃ alkyl optionally substituted by —OR^{b};
5- to 6-membered heteroaryl optionally substituted by 1-3 groups selected from —OR^{b}, halogen, and C₁-C₃ alkyl optionally substituted by —OR^{b};
—R^{a}C(═O)R^{b}; —R^{a}NHC(═O)R^{b}; —R^{a}NR^{c}R^{d}; —R^{a}S(═O)R^{b}; —R^{a}S(═O)₂R^{b};

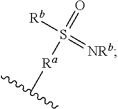

R^{a}NHS(═O)₂R^{b}; and —R^{a}C(═O)NR^{c}R^{d};
wherein:
R^{a}, for each occurrence, is independently selected from C₁-C₆ alkyl optionally substituted by 1-3 groups selected from —OH and halogen;
R^{b}, for each occurrence, is independently selected from H, —OH, —O(C₁-C₃ alkyl), 4- to 5-membered heterocyclyl, and C₁-C₃ alkyl optionally substituted by 1-3 groups selected from —OH and halogen;
R^{c} and R^{d}, for each occurrence, are independently selected from H, C₁-C₃ alkyl, or
R^{c} and R^{d} join to form a 3- to 6-membered cycloalkyl or 4- to 6-membered heterocyclyl optionally substituted by 1-3 groups selected from —OH and halogen;
and all other variables not specifically defined herein are as defined in any one of the suitable preceding embodiments.

In a 31^{st} embodiment, in a compound, tautomer, a solvate or stereoisomer of the compound or the tautomer, or pharmaceutically acceptable salt of this disclosure, R⁴ is selected from:
—CH₃,

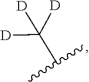 [SMILES: *C([2H])([2H])[2H]]

—CH₂CH₂OH,

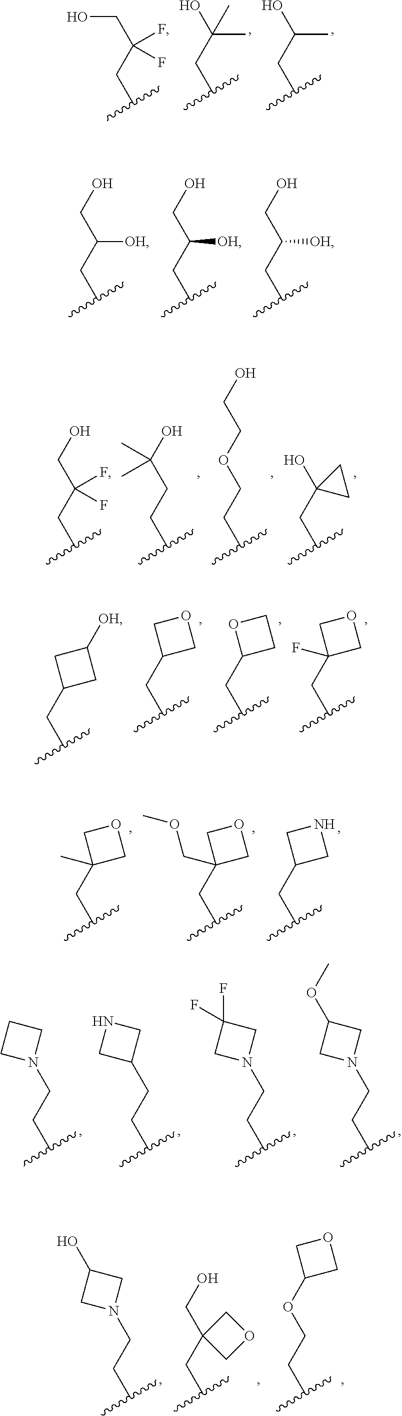

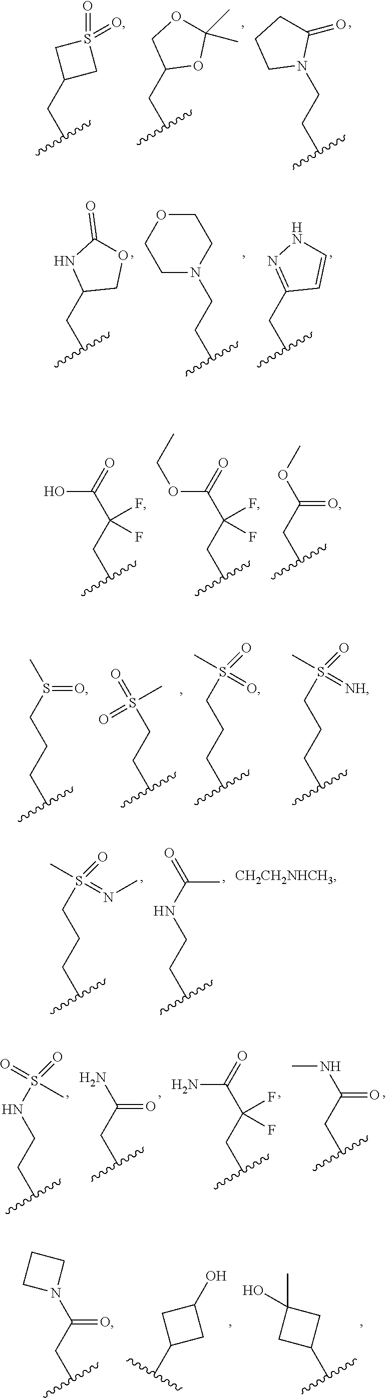

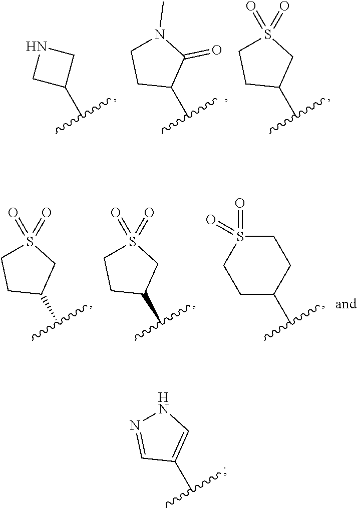

and all other variables not specifically defined herein are as defined in any one of the suitable preceding embodiments.

In a 32^{nd} embodiment, in a compound, tautomer, a solvate or stereoisomer of the compound or the tautomer, or pharmaceutically acceptable salt of this disclosure, R⁵ is selected from: C₁-C₆ alkyl, phenyl, 5- to 6-membered heteroaryl, 5- to 6-membered cycloalkyl, and 5- to 6-membered heterocyclyl,
wherein:
the C₁-C₆ alkyl of R⁵ is optionally substituted with —C(═O)NHR^{p2}, wherein R^{p2} is selected from C₁-C₃ alkyl and phenyl; and
the phenyl, 5- to 6-membered heteroaryl, 5- to 6-membered cycloalkyl, and 5- to 6-membered heterocyclyl of R⁵ is each optionally substituted with 1-3 groups selected from C₁-C₂ alkyl (optionally substituted with 1-3 groups selected from halogen), halogen, —CN, —O(C₁-C₂ alkyl), and —C(═O)O(C₁-C₄ alkyl); and
all other variables not specifically defined herein are as defined in any one of the suitable preceding embodiments.

In a 33^{rd} embodiment, in a compound, tautomer, a solvate or stereoisomer of the compound or the tautomer, or pharmaceutically acceptable salt of this disclosure, R⁵ is selected from: methyl, —CH(CH₃)₂,

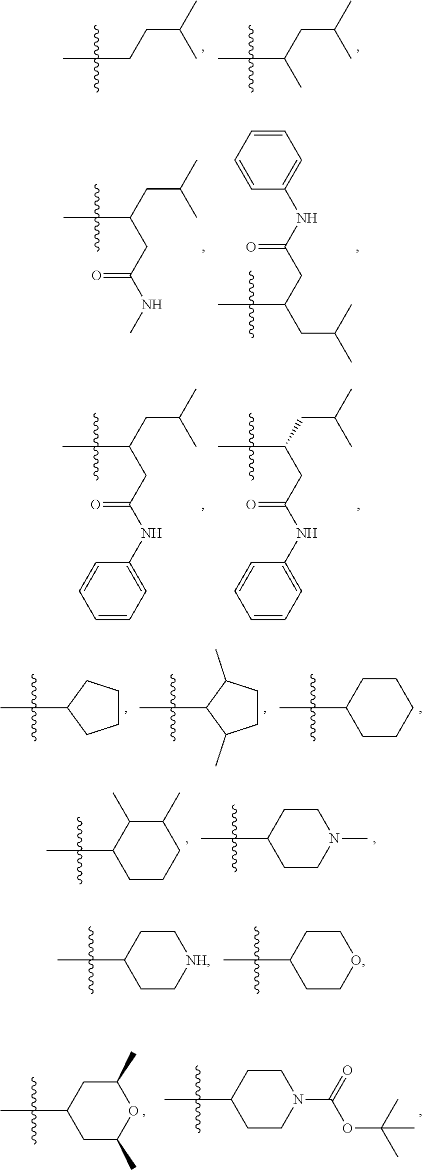

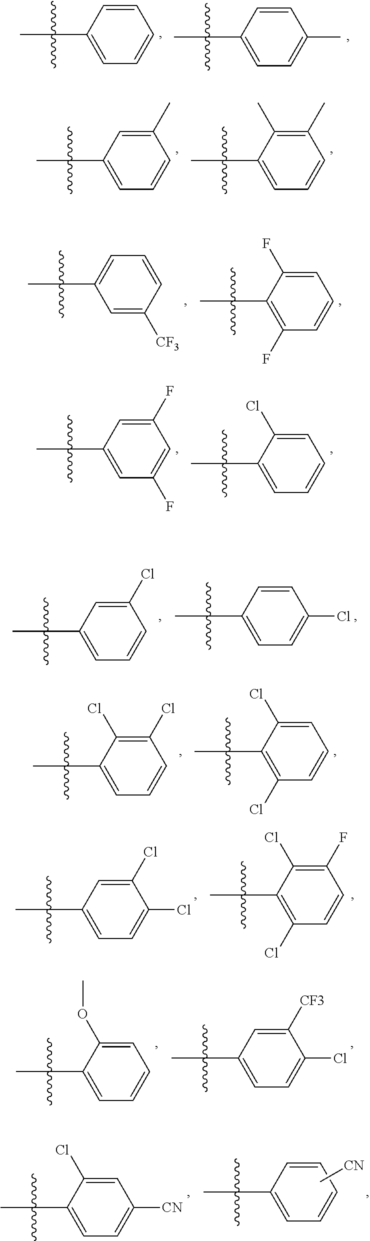

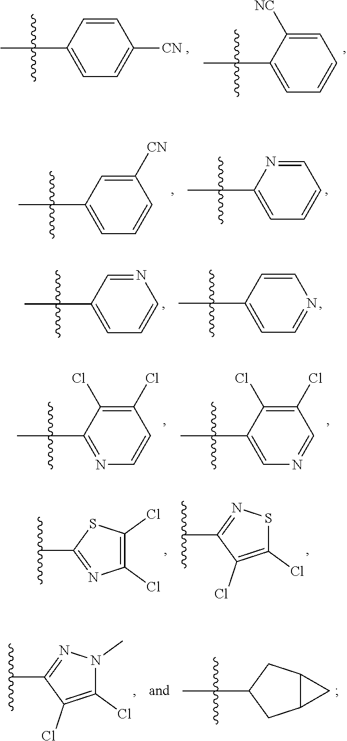

and all other variables not specifically defined herein are as defined in any one of the suitable preceding embodiments.

In a 34^{th} embodiment, in a compound, tautomer, a solvate or stereoisomer of the compound or the tautomer, or pharmaceutically acceptable salt of this disclosure, R⁵ is selected from phenyl, 5- to 6-membered heteroaryl, 5- to 6-membered cycloalkyl, and 5- to 6-membered heterocyclyl, wherein the phenyl, heteroaryl, cycloalkyl, and heterocyclyl are optionally substituted with 1-3 groups selected from halogen, —CN, C₁-C₂ alkyl (optionally substituted with 1-3 groups selected from halogen), ═O, and —O(C₁-C₂ alkyl), wherein the C₁-C₂ alkyl of the —O(C₁-C₂ alkyl), is optionally substituted with 1-3 groups selected from halogen; and all other variables not specifically defined herein are as defined in any one of the suitable preceding embodiments.

In a 35^{th} embodiment, in a compound, tautomer, a solvate or stereoisomer of the compound or the tautomer, or pharmaceutically acceptable salt of this disclosure, R⁵ is selected from:

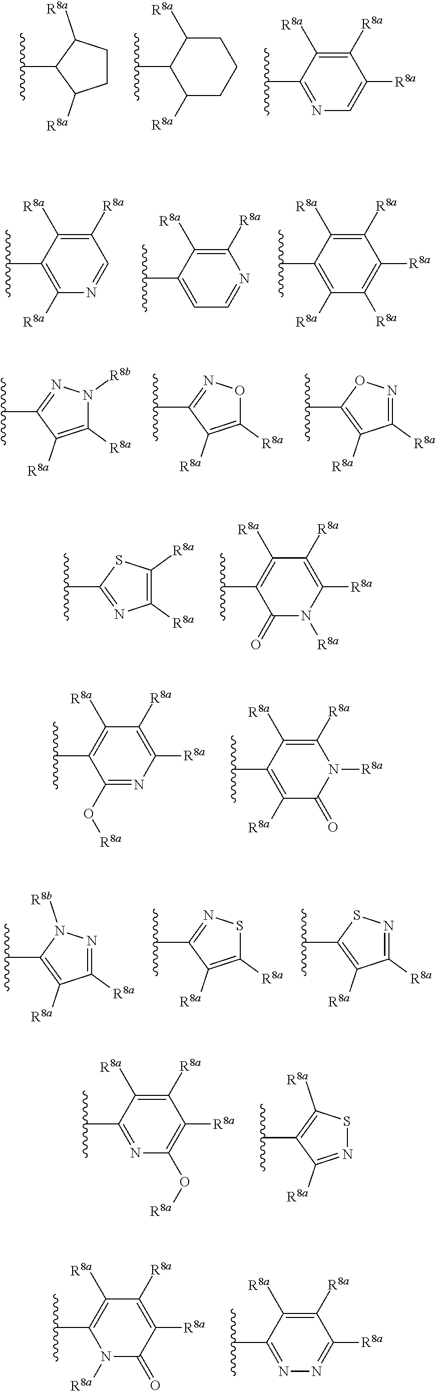

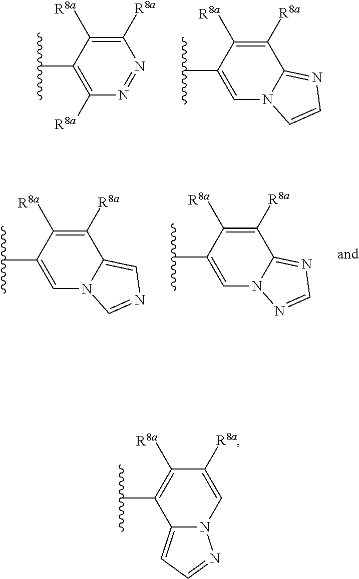

wherein: R^{8a}, for each occurrence, is independently selected from H, F, Cl, Me, OMe, —OCF₃, —OCHF₂, CHF₂, CF₃, —CN, —SO₂Me, —SMe, CH₂CF₃, CH₂SO₂Me, —CONH₂, CH₂OH,

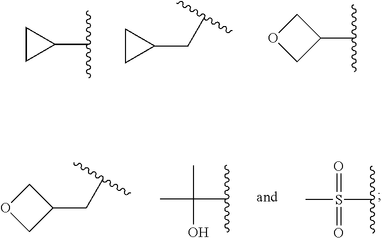

R^{8b}, for each occurrence, is independently selected from:
Me, CF₃, CHF₂, CH₂CF₃, Acetyl, SO₂Me,

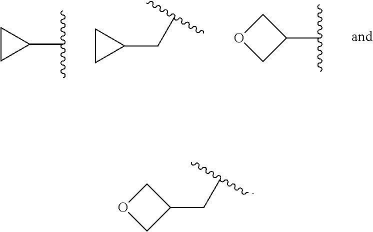

In a 36^{th} embodiment, in a compound, tautomer, a solvate or stereoisomer of the compound or the tautomer, or pharmaceutically acceptable salt of this disclosure, R⁵ is selected from:

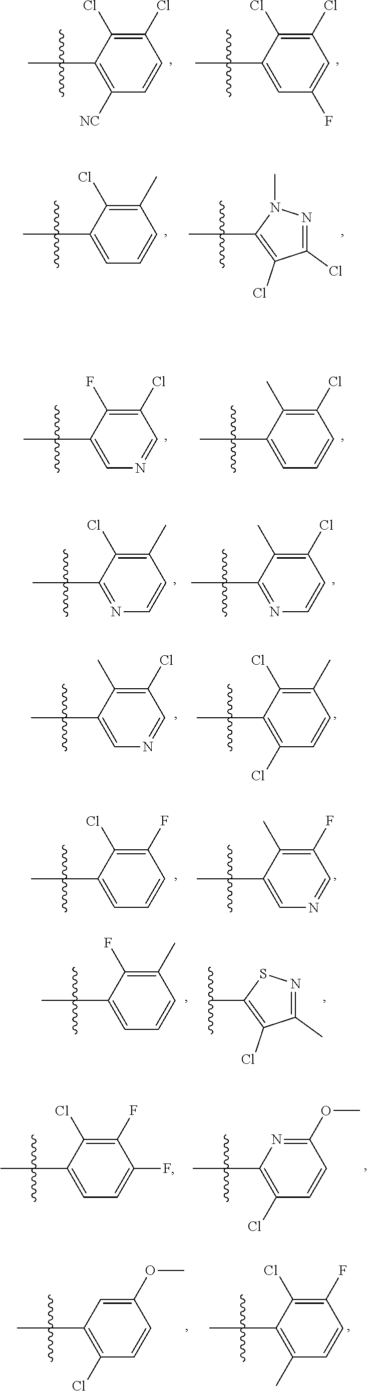

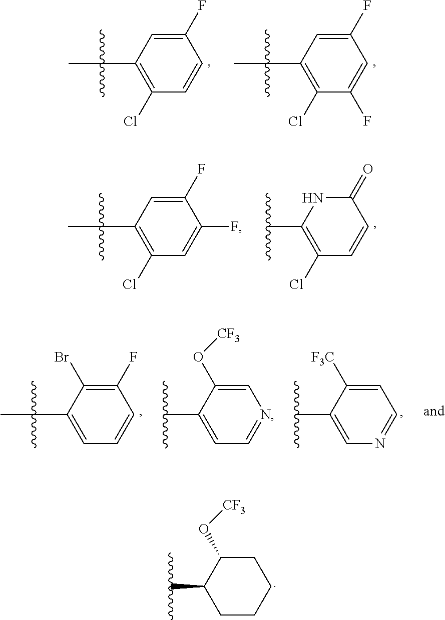

In a 37^{th} embodiment, in a compound, tautomer, a solvate or stereoisomer of the compound or the tautomer, or pharmaceutically acceptable salt of this disclosure, R⁷, for each occurrence, is independently selected from halogen, —CN, —CONH₂, 4- to 6-membered heterocyclyl, 5- to 6-membered heteroaryl, phenyl, 3-5 membered cycloalkyl, C₁-C₄ alkyl (optionally substituted by 1-3 groups selected from halogen and OH), ═O (provided that Fused Ring A is not Fused Ring A-1), O(C₁-C₄ alkyl), and m is an integer selected from 0, 1, and 2; and all other variables not specifically defined herein are as defined in any one of the suitable preceding embodiments.

In a 38^{th} embodiment, in a compound, tautomer, a solvate or stereoisomer of the compound or the tautomer, or pharmaceutically acceptable salt of this disclosure, R⁷, for each occurrence, is independently selected from methyl, ethyl, —CH(CH₃)₂, OCH₃, F, Cl, Br, —CN, —CONH₂, —C(CH₃)₂OH, CF₃, and

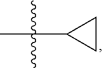 [SMILES: *C1CC1]

and m is an integer selected from 0, 1, and 2; and all other variables not specifically defined herein are as defined in any one of the suitable preceding embodiments.

In a 39^{th} embodiment, in a compound, tautomer, a solvate or stereoisomer of the compound or the tautomer, or pharmaceutically acceptable salt of this disclosure, R⁷, for each occurrence, is independently selected from:
C₁-C₆ alkyl (optionally substituted by 1-3 groups selected from halogen, —OH, —CN, —C(═O)OCH₃, 3- to 4-membered cycloalkyl, and 5- to 6-membered heteroaryl),
C₁-C₆ alkenyl (optionally substituted by 1-3 groups selected from halogen, —OH, —CN, —C(═O)OCH₃, 3- to 4-membered cycloalkyl, and 5- to 6-membered heteroaryl),
3- to 5-membered cycloalkyl (optionally substituted by 1-3 groups selected from halogen, —OH, and C₁-C₂ alkyl),
4- to 6-membered heterocyclyl (optionally substituted by 1-3 groups selected from halogen, —OH, C₁-C₄ alkyl, and ═O),
5- to 6-membered heteroaryl (optionally substituted by 1-3 groups selected from halogen and C₁-C₂ alkyl), and
—S(═O)₂(C₁-C₂ alkyl), and
m is an integer selected from 0, 1, and 2;
and all other variables not specifically defined herein are as defined in any one of the suitable preceding embodiments.

In a 40^{th} embodiment, in a compound, tautomer, a solvate or stereoisomer of the compound or the tautomer, or pharmaceutically acceptable salt of this disclosure, R⁷, for each occurrence, is independently selected from

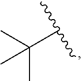 [SMILES: *C(C)(C)C]

### CH₂OH,

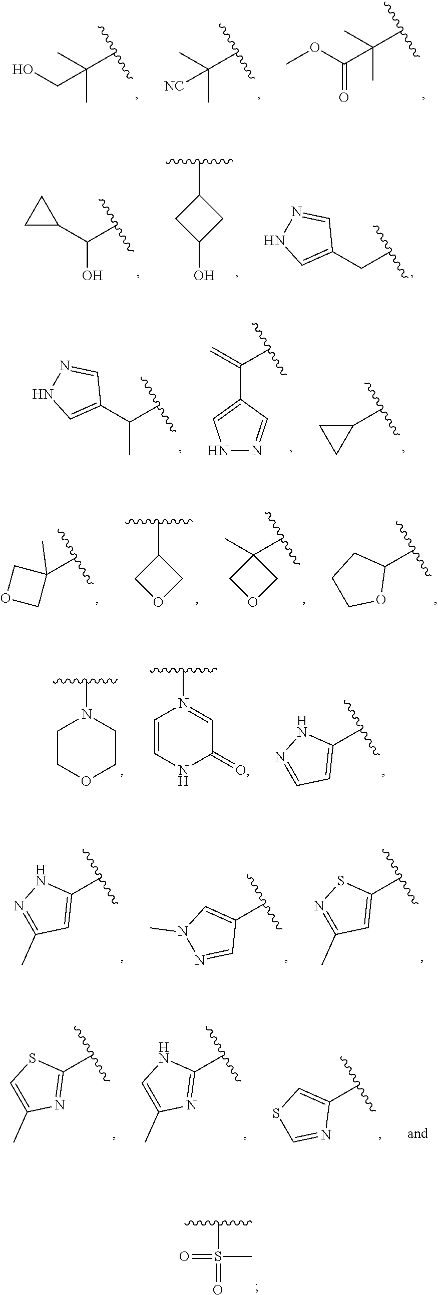

and all other variables not specifically defined herein are as defined in any one of the suitable preceding embodiments.

In certain embodiments, a compound of the disclosure is selected from Compounds 1 to 240 depicted in Table 1, a tautomer thereof, a solvate or stereoisomer of the compound or the tautomer, or a pharmaceutically acceptable salt of the foregoing.

TABLE 1
Compounds 1 to 240

| | |
|---|---|
| 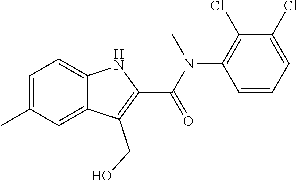 [SMILES: Cc1ccc2[nH]c(C(=O)N(C)c3cccc(Cl)c3Cl)c(CO)c2c1] | 1 |
| 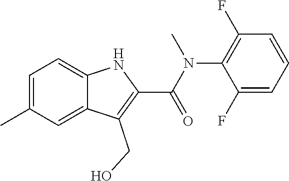 [SMILES: Cc1ccc2[nH]c(C(=O)N(C)c3c(F)cccc3F)c(CO)c2c1] | 2 |
| 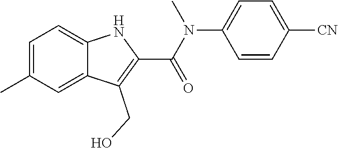 [SMILES: Cc1ccc2[nH]c(C(=O)N(C)c3ccc(C#N)cc3)c(CO)c2c1] | 3 |
| 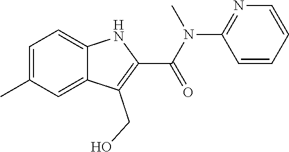 [SMILES: Cc1ccc2[nH]c(C(=O)N(C)c3ccccn3)c(CO)c2c1] | 4 |
| 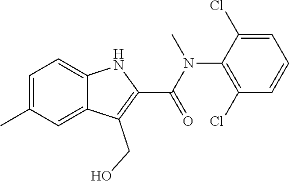 [SMILES: Cc1ccc2[nH]c(C(=O)N(C)c3c(Cl)cccc3Cl)c(CO)c2c1] | 5 |
| 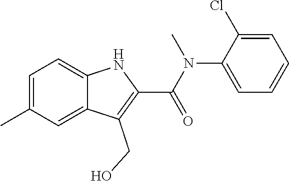 [SMILES: Cc1ccc2[nH]c(C(=O)N(C)c3ccccc3Cl)c(CO)c2c1] | 6 |
| 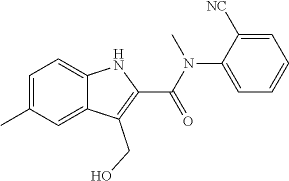 [SMILES: Cc1ccc2[nH]c(C(=O)N(C)c3ccccc3C#N)c(CO)c2c1] | 7 |
| 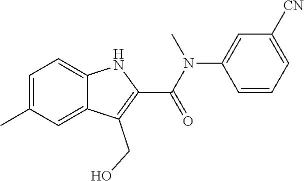 [SMILES: Cc1ccc2[nH]c(C(=O)N(C)c3cccc(C#N)c3)c(CO)c2c1] | 8 |
| 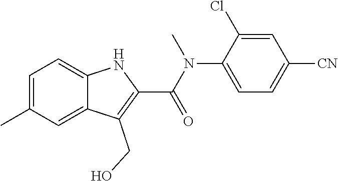 [SMILES: Cc1ccc2[nH]c(C(=O)N(C)c3ccc(C#N)cc3Cl)c(CO)c2c1] | 9 |
| 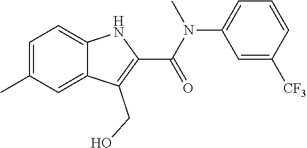 [SMILES: Cc1ccc2[nH]c(C(=O)N(C)c3cccc(C(F)(F)F)c3)c(CO)c2c1] | 10 |
| 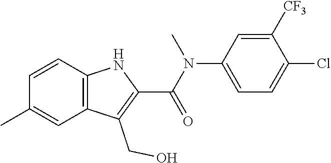 [SMILES: Cc1ccc2[nH]c(C(=O)N(C)c3ccc(Cl)c(C(F)(F)F)c3)c(CO)c2c1] | 11 |
| 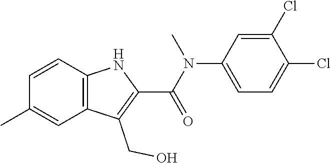 [SMILES: Cc1ccc2[nH]c(C(=O)N(C)c3ccc(Cl)c(Cl)c3)c(CO)c2c1] | 12 |
| 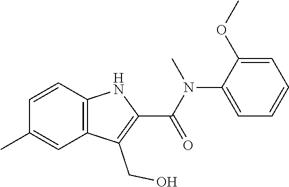 [SMILES: COc1ccccc1N(C)C(=O)c1[nH]c2ccc(C)cc2c1CO] | 13 |
| 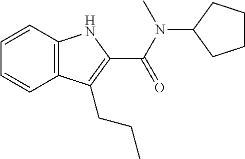 [SMILES: CCCc1c(C(=O)N(C)C2CCCC2)[nH]c2ccccc12] | 14 |
| 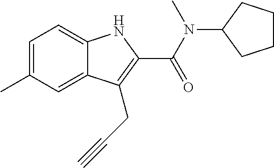 [SMILES: C#CCc1c(C(=O)N(C)C2CCCC2)[nH]c2ccc(C)cc12] | 15 |
| 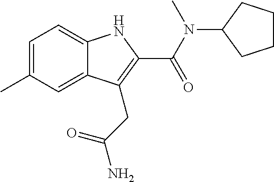 [SMILES: Cc1ccc2[nH]c(C(=O)N(C)C3CCCC3)c(CC(N)=O)c2c1] | 16 |
| 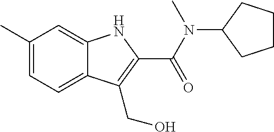 [SMILES: Cc1ccc2c(CO)c(C(=O)N(C)C3CCCC3)[nH]c2c1] | 17 |
| 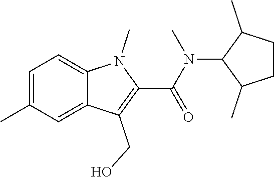 [SMILES: Cc1ccc2c(c1)c(CO)c(C(=O)N(C)C1C(C)CCC1C)n2C] | 18 |
| 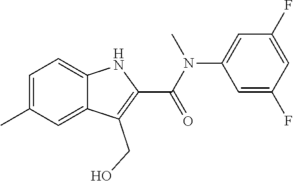 [SMILES: Cc1ccc2[nH]c(C(=O)N(C)c3cc(F)cc(F)c3)c(CO)c2c1] | 19 |
| 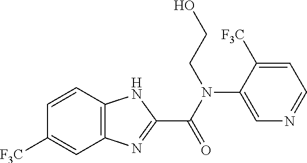 [SMILES: O=C(c1nc2cc(C(F)(F)F)ccc2[nH]1)N(CCO)c1cnccc1C(F)(F)F] | 20 |
| 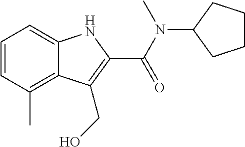 [SMILES: Cc1cccc2[nH]c(C(=O)N(C)C3CCCC3)c(CO)c12] | 21 |
| 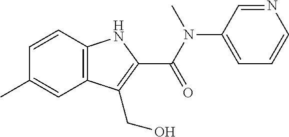 [SMILES: Cc1ccc2[nH]c(C(=O)N(C)c3cccnc3)c(CO)c2c1] | 22 |
| 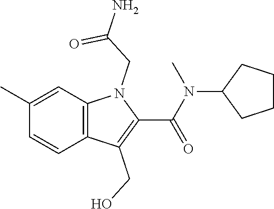 [SMILES: Cc1ccc2c(CO)c(C(=O)N(C)C3CCCC3)n(CC(N)=O)c2c1] | 23 |
| 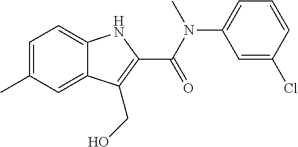 [SMILES: Cc1ccc2[nH]c(C(=O)N(C)c3cccc(Cl)c3)c(CO)c2c1] | 24 |
| 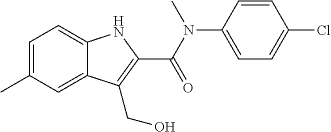 [SMILES: Cc1ccc2[nH]c(C(=O)N(C)c3ccc(Cl)cc3)c(CO)c2c1] | 25 |
| 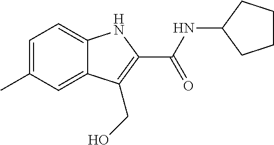 [SMILES: Cc1ccc2[nH]c(C(=O)NC3CCCC3)c(CO)c2c1] | 26 |
| 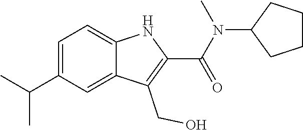 [SMILES: CC(C)c1ccc2[nH]c(C(=O)N(C)C3CCCC3)c(CO)c2c1] | 27 |
| 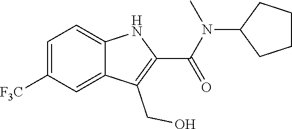 [SMILES: CN(C(=O)c1[nH]c2ccc(C(F)(F)F)cc2c1CO)C1CCCC1] | 28 |
| 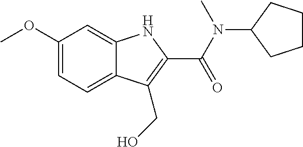 [SMILES: COc1ccc2c(CO)c(C(=O)N(C)C3CCCC3)[nH]c2c1] | 29 |
| 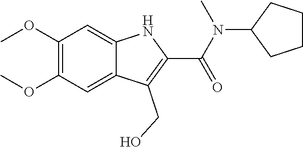 [SMILES: COc1cc2[nH]c(C(=O)N(C)C3CCCC3)c(CO)c2cc1OC] | 30 |
| 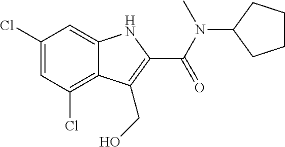 [SMILES: CN(C(=O)c1[nH]c2cc(Cl)cc(Cl)c2c1CO)C1CCCC1] | 31 |
| 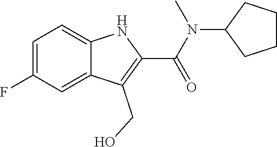 [SMILES: CN(C(=O)c1[nH]c2ccc(F)cc2c1CO)C1CCCC1] | 32 |
| 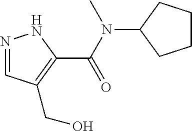 [SMILES: CN(C(=O)c1[nH]ncc1CO)C1CCCC1] | 33 |
| 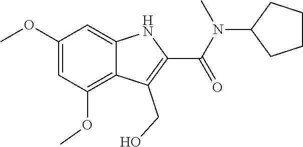 [SMILES: COc1cc(OC)c2c(CO)c(C(=O)N(C)C3CCCC3)[nH]c2c1] | 34 |
| 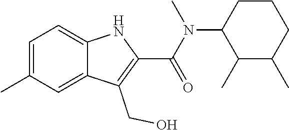 | 35 |
| 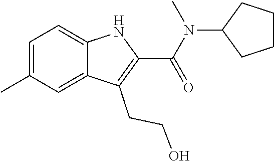 [SMILES: Cc1ccc2[nH]c(C(=O)N(C)C3CCCC3)c(CCO)c2c1] | 36 |
| 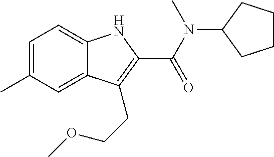 [SMILES: COCCc1c(C(=O)N(C)C2CCCC2)[nH]c2ccc(C)cc12] | 37 |
| 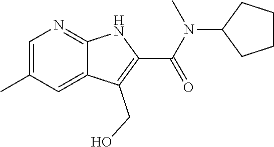 [SMILES: Cc1cnc2[nH]c(C(=O)N(C)C3CCCC3)c(CO)c2c1] | 38 |
| 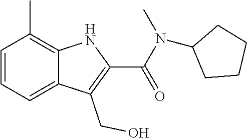 [SMILES: Cc1cccc2c(CO)c(C(=O)N(C)C3CCCC3)[nH]c12] | 39 |
| 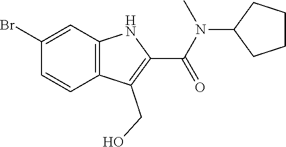 [SMILES: CN(C(=O)c1[nH]c2cc(Br)ccc2c1CO)C1CCCC1] | 40 |
| 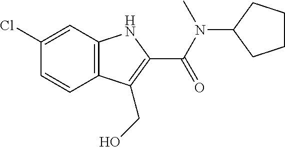 [SMILES: CN(C(=O)c1[nH]c2cc(Cl)ccc2c1CO)C1CCCC1] | 41 |
| 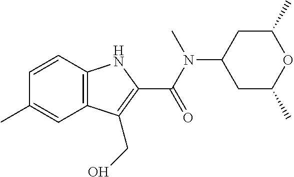 | 42 |
| 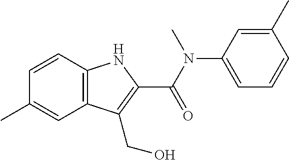 | 43 |
| 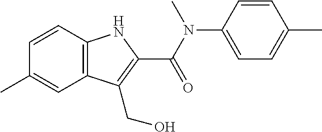 | 44 |
| 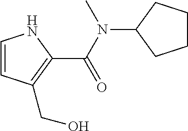 [SMILES: CN(C(=O)c1[nH]ccc1CO)C1CCCC1] | 45 |
| 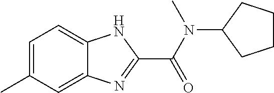 [SMILES: Cc1ccc2[nH]c(C(=O)N(C)C3CCCC3)nc2c1] | 46 |
| 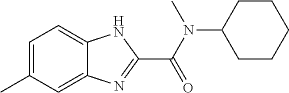 [SMILES: Cc1ccc2[nH]c(C(=O)N(C)C3CCCCC3)nc2c1] | 47 |
| 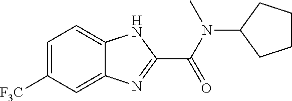 [SMILES: CN(C(=O)c1nc2cc(C(F)(F)F)ccc2[nH]1)C1CCCC1] | 48 |
| 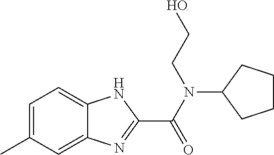 [SMILES: Cc1ccc2[nH]c(C(=O)N(CCO)C3CCCC3)nc2c1] | 49 |
| 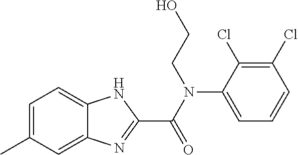 [SMILES: Cc1ccc2[nH]c(C(=O)N(CCO)c3cccc(Cl)c3Cl)nc2c1] | 50 |
| 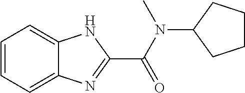 [SMILES: CN(C(=O)c1nc2ccccc2[nH]1)C1CCCC1] | 51 |
| 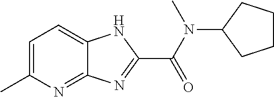 [SMILES: Cc1ccc2[nH]c(C(=O)N(C)C3CCCC3)nc2n1] | 52 |
| 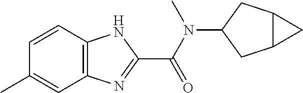 [SMILES: Cc1ccc2[nH]c(C(=O)N(C)C3CC4CC4C3)nc2c1] | 53 |
| 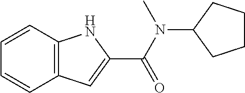 [SMILES: CN(C(=O)c1cc2ccccc2[nH]1)C1CCCC1] | 54 |
| 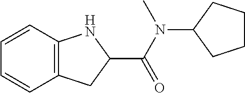 [SMILES: CN(C(=O)C1Cc2ccccc2N1)C1CCCC1] | 55 |
| 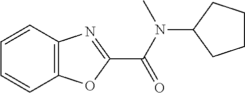 [SMILES: CN(C(=O)c1nc2ccccc2o1)C1CCCC1] | 56 |
| 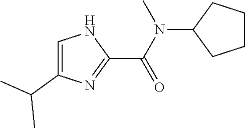 [SMILES: CC(C)c1c[nH]c(C(=O)N(C)C2CCCC2)n1] | 57 |
| 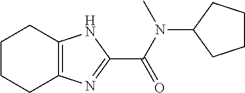 [SMILES: CN(C(=O)c1nc2c([nH]1)CCCC2)C1CCCC1] | 58 |
| 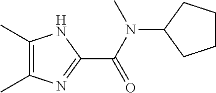 [SMILES: Cc1nc(C(=O)N(C)C2CCCC2)[nH]c1C] | 59 |
| 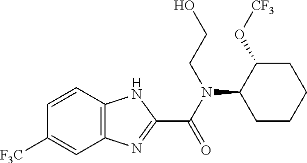 [SMILES: O=C(c1nc2cc(C(F)(F)F)ccc2[nH]1)N(CCO)[C@@H]1CCCC[C@H]1OC(F)(F)F] | 60 |
| 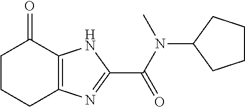 [SMILES: CN(C(=O)c1nc2c([nH]1)C(=O)CCC2)C1CCCC1] | 61 |
| 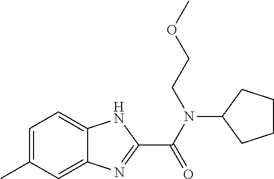 [SMILES: COCCN(C(=O)c1nc2cc(C)ccc2[nH]1)C1CCCC1] | 62 |
| 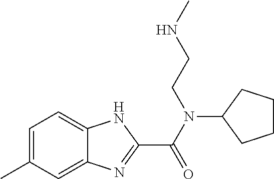 [SMILES: CNCCN(C(=O)c1nc2cc(C)ccc2[nH]1)C1CCCC1] | 63 |
| 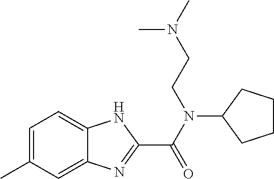 [SMILES: Cc1ccc2[nH]c(C(=O)N(CCN(C)C)C3CCCC3)nc2c1] | 64 |
| 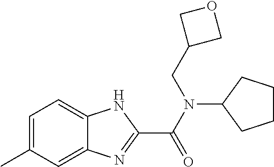 [SMILES: Cc1ccc2[nH]c(C(=O)N(CC3COC3)C3CCCC3)nc2c1] | 65 |
| 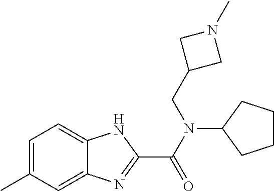 [SMILES: Cc1ccc2[nH]c(C(=O)N(CC3CN(C)C3)C3CCCC3)nc2c1] | 66 |
| 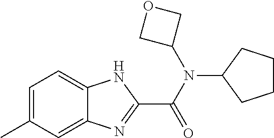 [SMILES: Cc1ccc2[nH]c(C(=O)N(C3CCCC3)C3COC3)nc2c1] | 67 |
| 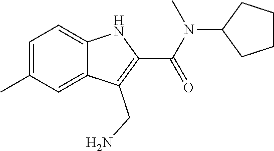 [SMILES: Cc1ccc2[nH]c(C(=O)N(C)C3CCCC3)c(CN)c2c1] | 68 |
| 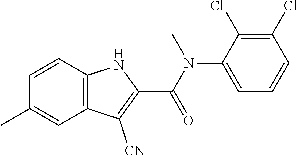 [SMILES: Cc1ccc2[nH]c(C(=O)N(C)c3cccc(Cl)c3Cl)c(C#N)c2c1] | 69 |
| 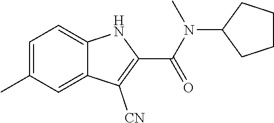 [SMILES: Cc1ccc2[nH]c(C(=O)N(C)C3CCCC3)c(C#N)c2c1] | 70 |
| 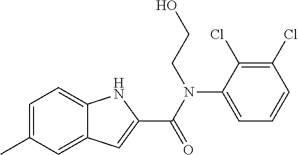 [SMILES: Cc1ccc2[nH]c(C(=O)N(CCO)c3cccc(Cl)c3Cl)cc2c1] | 71 |
| 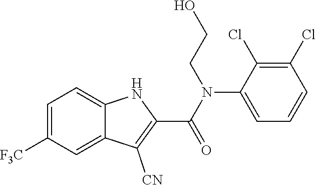 [SMILES: N#Cc1c(C(=O)N(CCO)c2cccc(Cl)c2Cl)[nH]c2ccc(C(F)(F)F)cc12] | 72 |
| 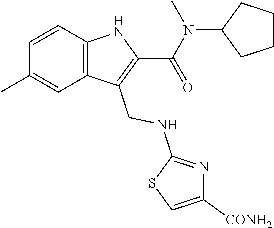 [SMILES: Cc1ccc2[nH]c(C(=O)N(C)C3CCCC3)c(CNc3nc(C(N)=O)cs3)c2c1] | 73 |
| 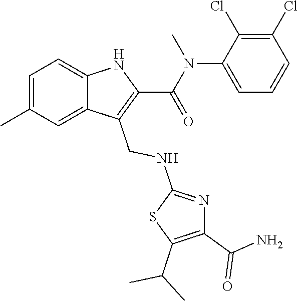 [SMILES: Cc1ccc2[nH]c(C(=O)N(C)c3cccc(Cl)c3Cl)c(CNc3nc(C(N)=O)c(C(C)C)s3)c2c1] | 74 |
| 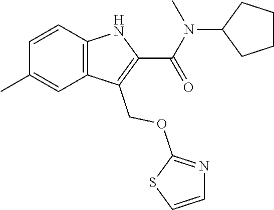 [SMILES: Cc1ccc2[nH]c(C(=O)N(C)C3CCCC3)c(COc3nccs3)c2c1] | 75 |
| 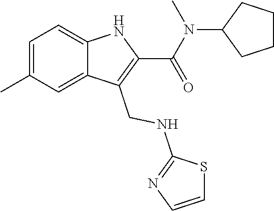 [SMILES: Cc1ccc2[nH]c(C(=O)N(C)C3CCCC3)c(CNc3nccs3)c2c1] | 76 |
| 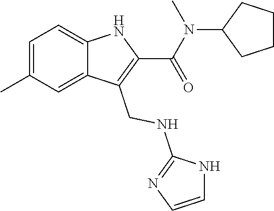 [SMILES: Cc1ccc2[nH]c(C(=O)N(C)C3CCCC3)c(CNc3ncc[nH]3)c2c1] | 77 |
| 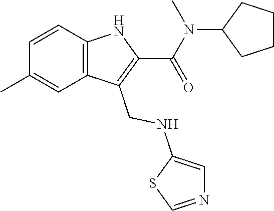 [SMILES: Cc1ccc2[nH]c(C(=O)N(C)C3CCCC3)c(CNc3cncs3)c2c1] | 78 |
| 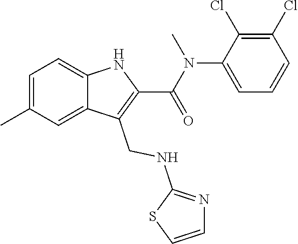 [SMILES: Cc1ccc2[nH]c(C(=O)N(C)c3cccc(Cl)c3Cl)c(CNc3nccs3)c2c1] | 79 |
| 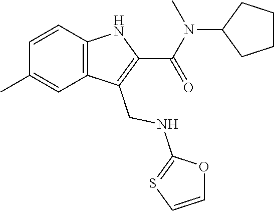 | 80 |
| 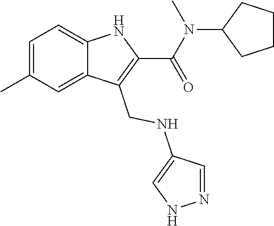 [SMILES: Cc1ccc2[nH]c(C(=O)N(C)C3CCCC3)c(CNc3cn[nH]c3)c2c1] | 81 |
| 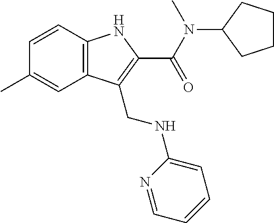 [SMILES: Cc1ccc2[nH]c(C(=O)N(C)C3CCCC3)c(CNc3ccccn3)c2c1] | 82 |
| 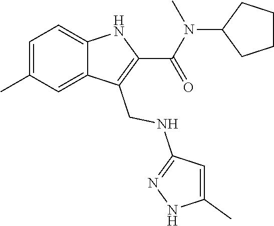 [SMILES: Cc1ccc2[nH]c(C(=O)N(C)C3CCCC3)c(CNc3cc(C)[nH]n3)c2c1] | 83 |
| 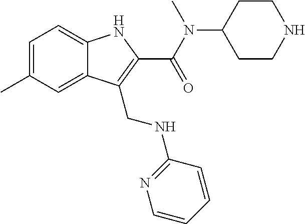 [SMILES: Cc1ccc2[nH]c(C(=O)N(C)C3CCNCC3)c(CNc3ccccn3)c2c1] | 84 |
| 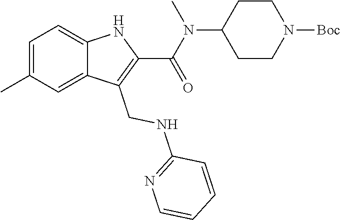 [SMILES: Cc1ccc2[nH]c(C(=O)N(C)C3CCN(C(=O)OC(C)(C)C)CC3)c(CNc3ccccn3)c2c1] | 85 |
| 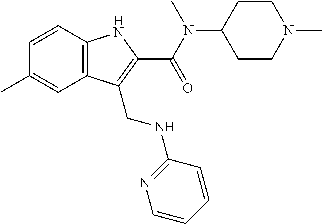 | 86 |
| 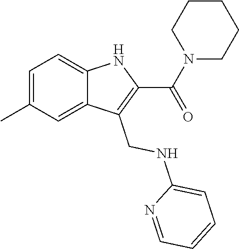 [SMILES: Cc1ccc2[nH]c(C(=O)N3CCCCC3)c(CNc3ccccn3)c2c1] | 87 |
| 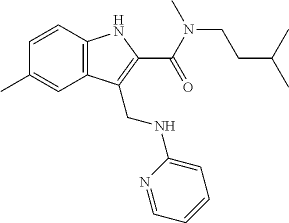 [SMILES: Cc1ccc2[nH]c(C(=O)N(C)CCC(C)C)c(CNc3ccccn3)c2c1] | 88 |
| 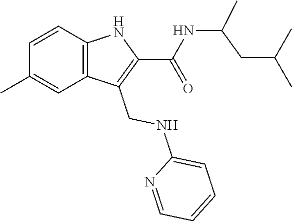 [SMILES: Cc1ccc2[nH]c(C(=O)NC(C)CC(C)C)c(CNc3ccccn3)c2c1] | 89 |
| 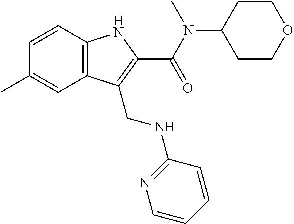 [SMILES: Cc1ccc2[nH]c(C(=O)N(C)C3CCOCC3)c(CNc3ccccn3)c2c1] | 90 |
| 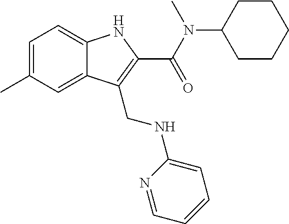 [SMILES: Cc1ccc2[nH]c(C(=O)N(C)C3CCCCC3)c(CNc3ccccn3)c2c1] | 91 |
| 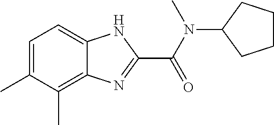 [SMILES: Cc1ccc2[nH]c(C(=O)N(C)C3CCCC3)nc2c1C] | 92 |
| 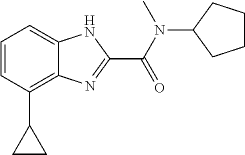 [SMILES: CN(C(=O)c1nc2c(C3CC3)cccc2[nH]1)C1CCCC1] | 93 |
| 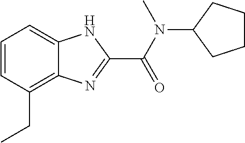 [SMILES: CCc1cccc2[nH]c(C(=O)N(C)C3CCCC3)nc12] | 94 |
| 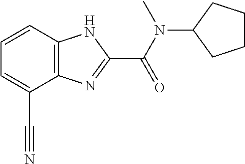 [SMILES: CN(C(=O)c1nc2c(C#N)cccc2[nH]1)C1CCCC1] | 95 |
| 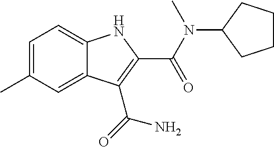 [SMILES: Cc1ccc2[nH]c(C(=O)N(C)C3CCCC3)c(C(N)=O)c2c1] | 96 |
| 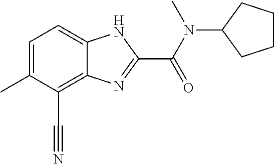 [SMILES: Cc1ccc2[nH]c(C(=O)N(C)C3CCCC3)nc2c1C#N] | 97 |
| 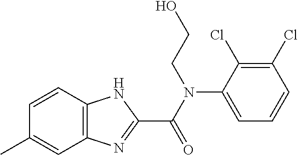 [SMILES: Cc1ccc2[nH]c(C(=O)N(CCO)c3cccc(Cl)c3Cl)nc2c1] | 98 |
| 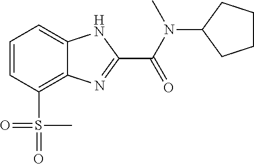 [SMILES: CN(C(=O)c1nc2c(S(C)(=O)=O)cccc2[nH]1)C1CCCC1] | 99 |
| 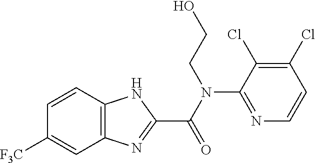 [SMILES: O=C(c1nc2cc(C(F)(F)F)ccc2[nH]1)N(CCO)c1nccc(Cl)c1Cl] | 100 |
| 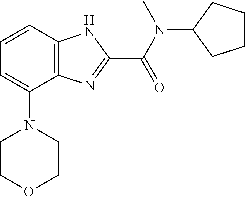 [SMILES: CN(C(=O)c1nc2c(N3CCOCC3)cccc2[nH]1)C1CCCC1] | 101 |
| 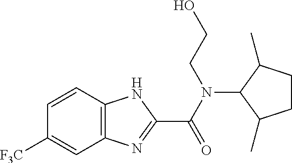 [SMILES: CC1CCC(C)C1N(CCO)C(=O)c1nc2cc(C(F)(F)F)ccc2[nH]1] | 102 |
| 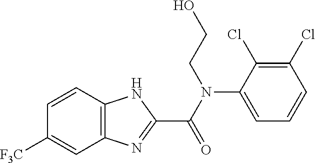 [SMILES: O=C(c1nc2cc(C(F)(F)F)ccc2[nH]1)N(CCO)c1cccc(Cl)c1Cl] | 103 |
| 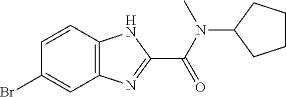 [SMILES: CN(C(=O)c1nc2cc(Br)ccc2[nH]1)C1CCCC1] | 104 |
| 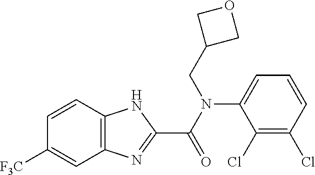 [SMILES: O=C(c1nc2cc(C(F)(F)F)ccc2[nH]1)N(CC1COC1)c1cccc(Cl)c1Cl] | 105 |
| 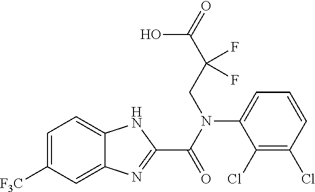 [SMILES: O=C(c1nc2cc(C(F)(F)F)ccc2[nH]1)N(CC(F)(F)C(=O)O)c1cccc(Cl)c1Cl] | 106 |
| 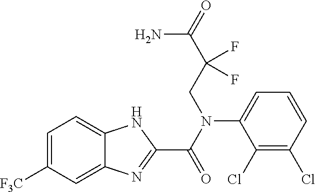 [SMILES: NC(=O)C(F)(F)CN(C(=O)c1nc2cc(C(F)(F)F)ccc2[nH]1)c1cccc(Cl)c1Cl] | 107 |
| 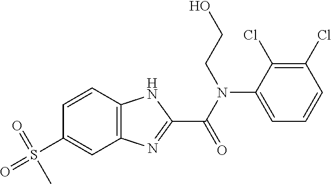 [SMILES: CS(=O)(=O)c1ccc2[nH]c(C(=O)N(CCO)c3cccc(Cl)c3Cl)nc2c1] | 108 |
| 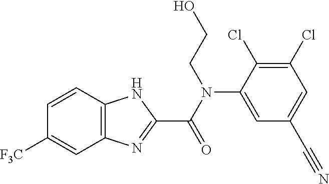 [SMILES: N#Cc1cc(Cl)c(Cl)c(N(CCO)C(=O)c2nc3cc(C(F)(F)F)ccc3[nH]2)c1] | 109 |
| 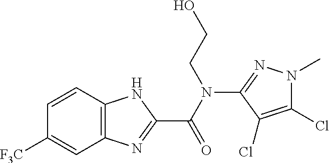 [SMILES: Cn1nc(N(CCO)C(=O)c2nc3cc(C(F)(F)F)ccc3[nH]2)c(Cl)c1Cl] | 110 |
| 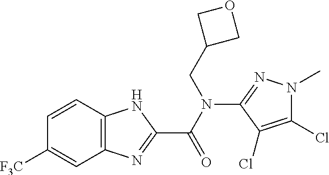 [SMILES: Cn1nc(N(CC2COC2)C(=O)c2nc3cc(C(F)(F)F)ccc3[nH]2)c(Cl)c1Cl] | 111 |
| 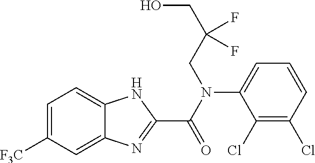 [SMILES: O=C(c1nc2cc(C(F)(F)F)ccc2[nH]1)N(CC(F)(F)CO)c1cccc(Cl)c1Cl] | 112 |
| 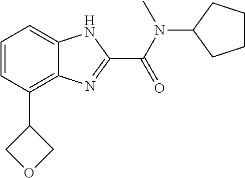 [SMILES: CN(C(=O)c1nc2c(C3COC3)cccc2[nH]1)C1CCCC1] | 113 |
| 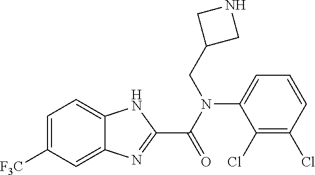 [SMILES: O=C(c1nc2cc(C(F)(F)F)ccc2[nH]1)N(CC1CNC1)c1cccc(Cl)c1Cl] | 114 |
| 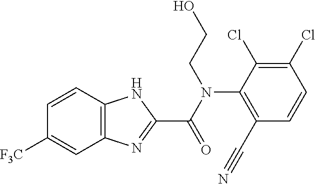 [SMILES: N#Cc1ccc(Cl)c(Cl)c1N(CCO)C(=O)c1nc2cc(C(F)(F)F)ccc2[nH]1] | 115 |
| 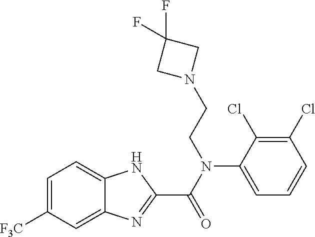 [SMILES: O=C(c1nc2cc(C(F)(F)F)ccc2[nH]1)N(CCN1CC(F)(F)C1)c1cccc(Cl)c1Cl] | 116 |
| 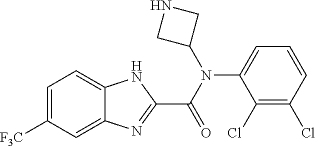 [SMILES: O=C(c1nc2cc(C(F)(F)F)ccc2[nH]1)N(c1cccc(Cl)c1Cl)C1CNC1] | 117 |
| 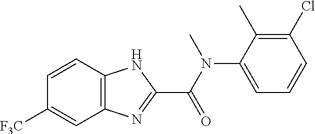 [SMILES: Cc1c(Cl)cccc1N(C)C(=O)c1nc2cc(C(F)(F)F)ccc2[nH]1] | 118 |
| 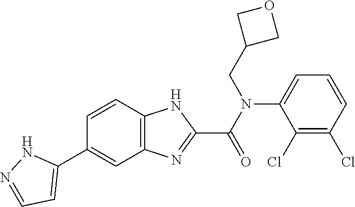 [SMILES: O=C(c1nc2cc(-c3ccn[nH]3)ccc2[nH]1)N(CC1COC1)c1cccc(Cl)c1Cl] | 119 |
| 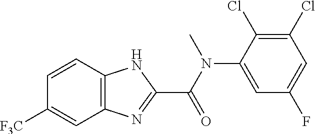 [SMILES: CN(C(=O)c1nc2cc(C(F)(F)F)ccc2[nH]1)c1cc(F)cc(Cl)c1Cl] | 120 |
| 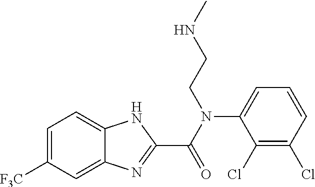 [SMILES: CNCCN(C(=O)c1nc2cc(C(F)(F)F)ccc2[nH]1)c1cccc(Cl)c1Cl] | 121 |
| 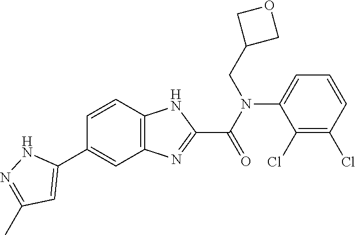 [SMILES: Cc1cc(-c2ccc3[nH]c(C(=O)N(CC4COC4)c4cccc(Cl)c4Cl)nc3c2)[nH]n1] | 122 |
| 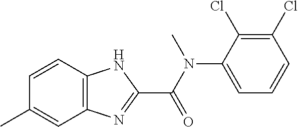 [SMILES: Cc1ccc2[nH]c(C(=O)N(C)c3cccc(Cl)c3Cl)nc2c1] | 123 |
| 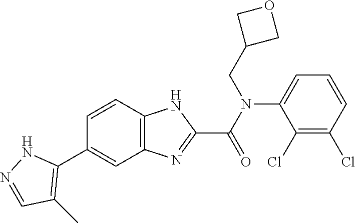 [SMILES: Cc1cn[nH]c1-c1ccc2[nH]c(C(=O)N(CC3COC3)c3cccc(Cl)c3Cl)nc2c1] | 124 |
| 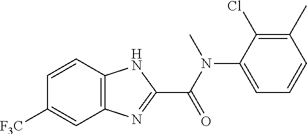 [SMILES: Cc1cccc(N(C)C(=O)c2nc3cc(C(F)(F)F)ccc3[nH]2)c1Cl] | 125 |
| 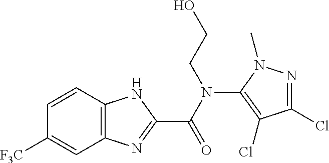 [SMILES: Cn1nc(Cl)c(Cl)c1N(CCO)C(=O)c1nc2cc(C(F)(F)F)ccc2[nH]1] | 126 |
| 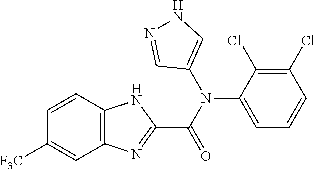 [SMILES: O=C(c1nc2cc(C(F)(F)F)ccc2[nH]1)N(c1cn[nH]c1)c1cccc(Cl)c1Cl] | 127 |
| 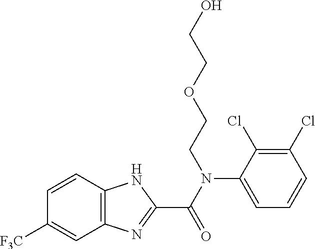 [SMILES: O=C(c1nc2cc(C(F)(F)F)ccc2[nH]1)N(CCOCCO)c1cccc(Cl)c1Cl] | 128 |
| 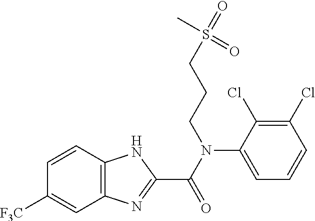 [SMILES: CS(=O)(=O)CCCN(C(=O)c1nc2cc(C(F)(F)F)ccc2[nH]1)c1cccc(Cl)c1Cl] | 129 |
| 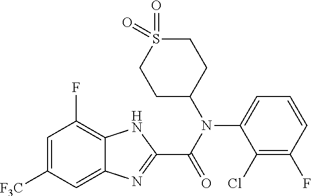 [SMILES: O=C(c1nc2cc(C(F)(F)F)cc(F)c2[nH]1)N(c1cccc(F)c1Cl)C1CCS(=O)(=O)CC1] | 130 |
| 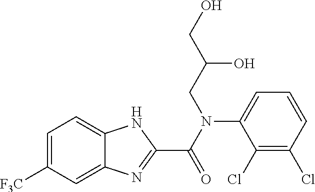 [SMILES: O=C(c1nc2cc(C(F)(F)F)ccc2[nH]1)N(CC(O)CO)c1cccc(Cl)c1Cl] | 131 |
| 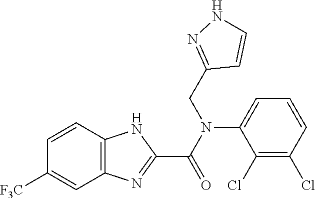 [SMILES: O=C(c1nc2cc(C(F)(F)F)ccc2[nH]1)N(Cc1cc[nH]n1)c1cccc(Cl)c1Cl] | 132 |
| 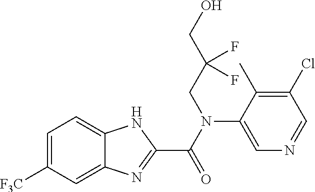 [SMILES: Cc1c(Cl)cncc1N(CC(F)(F)CO)C(=O)c1nc2cc(C(F)(F)F)ccc2[nH]1] | 133 |
| 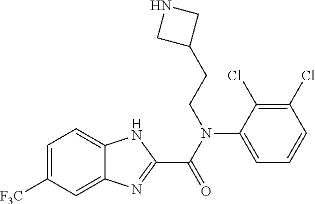 [SMILES: O=C(c1nc2cc(C(F)(F)F)ccc2[nH]1)N(CCC1CNC1)c1cccc(Cl)c1Cl] | 134 |
| 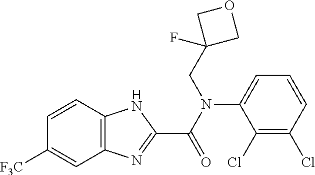 [SMILES: O=C(c1nc2cc(C(F)(F)F)ccc2[nH]1)N(CC1(F)COC1)c1cccc(Cl)c1Cl] | 135 |
| 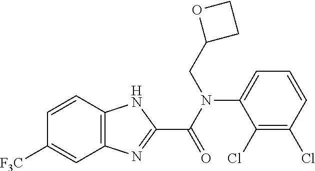 [SMILES: O=C(c1nc2cc(C(F)(F)F)ccc2[nH]1)N(CC1CCO1)c1cccc(Cl)c1Cl] | 136 |
| 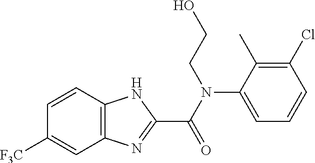 [SMILES: Cc1c(Cl)cccc1N(CCO)C(=O)c1nc2cc(C(F)(F)F)ccc2[nH]1] | 137 |
| 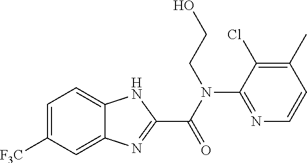 [SMILES: Cc1ccnc(N(CCO)C(=O)c2nc3cc(C(F)(F)F)ccc3[nH]2)c1Cl] | 138 |
| 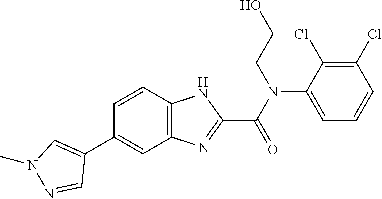 [SMILES: Cn1cc(-c2ccc3[nH]c(C(=O)N(CCO)c4cccc(Cl)c4Cl)nc3c2)cn1] | 139 |
| 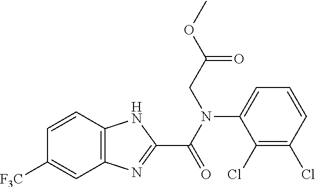 [SMILES: COC(=O)CN(C(=O)c1nc2cc(C(F)(F)F)ccc2[nH]1)c1cccc(Cl)c1Cl] | 140 |
| 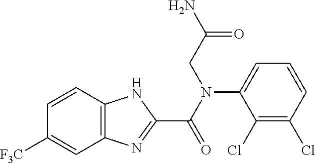 [SMILES: NC(=O)CN(C(=O)c1nc2cc(C(F)(F)F)ccc2[nH]1)c1cccc(Cl)c1Cl] | 141 |
| 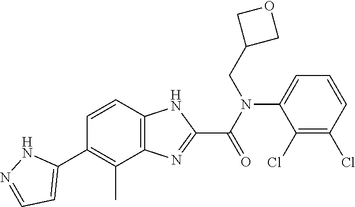 [SMILES: Cc1c(-c2ccn[nH]2)ccc2[nH]c(C(=O)N(CC3COC3)c3cccc(Cl)c3Cl)nc12] | 142 |
| 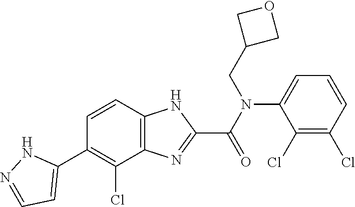 [SMILES: O=C(c1nc2c(Cl)c(-c3ccn[nH]3)ccc2[nH]1)N(CC1COC1)c1cccc(Cl)c1Cl] | 143 |
| 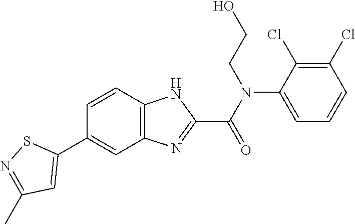 [SMILES: Cc1cc(-c2ccc3[nH]c(C(=O)N(CCO)c4cccc(Cl)c4Cl)nc3c2)sn1] | 144 |
| 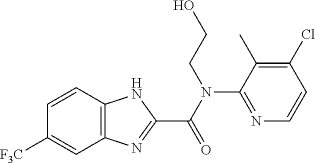 [SMILES: Cc1c(Cl)ccnc1N(CCO)C(=O)c1nc2cc(C(F)(F)F)ccc2[nH]1] | 145 |
| 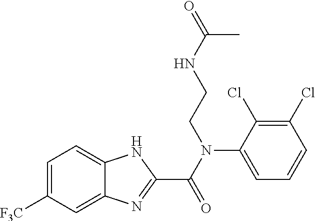 [SMILES: CC(=O)NCCN(C(=O)c1nc2cc(C(F)(F)F)ccc2[nH]1)c1cccc(Cl)c1Cl] | 146 |
| 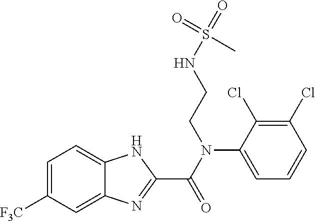 [SMILES: CS(=O)(=O)NCCN(C(=O)c1nc2cc(C(F)(F)F)ccc2[nH]1)c1cccc(Cl)c1Cl] | 147 |
| 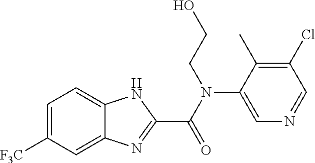 [SMILES: Cc1c(Cl)cncc1N(CCO)C(=O)c1nc2cc(C(F)(F)F)ccc2[nH]1] | 148 |
| 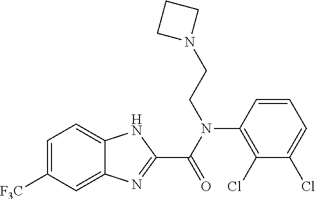 [SMILES: O=C(c1nc2cc(C(F)(F)F)ccc2[nH]1)N(CCN1CCC1)c1cccc(Cl)c1Cl] | 149 |
| 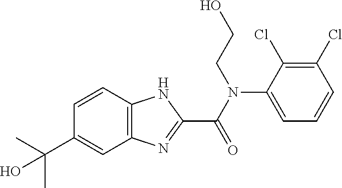 [SMILES: CC(C)(O)c1ccc2[nH]c(C(=O)N(CCO)c3cccc(Cl)c3Cl)nc2c1] | 150 |
| 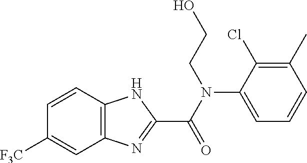 [SMILES: Cc1cccc(N(CCO)C(=O)c2nc3cc(C(F)(F)F)ccc3[nH]2)c1Cl] | 151 |
| 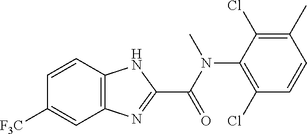 [SMILES: Cc1ccc(Cl)c(N(C)C(=O)c2nc3cc(C(F)(F)F)ccc3[nH]2)c1Cl] | 152 |
| 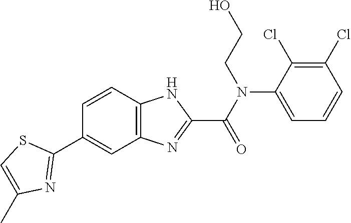 [SMILES: Cc1csc(-c2ccc3[nH]c(C(=O)N(CCO)c4cccc(Cl)c4Cl)nc3c2)n1] | 153 |
| 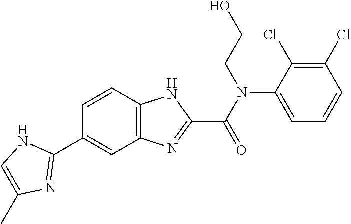 [SMILES: Cc1c[nH]c(-c2ccc3[nH]c(C(=O)N(CCO)c4cccc(Cl)c4Cl)nc3c2)n1] | 154 |
| 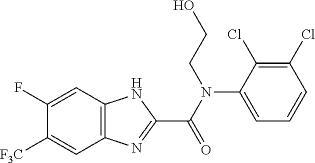 [SMILES: O=C(c1nc2cc(C(F)(F)F)c(F)cc2[nH]1)N(CCO)c1cccc(Cl)c1Cl] | 155 |
| 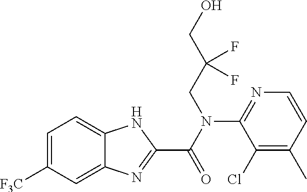 [SMILES: Cc1ccnc(N(CC(F)(F)CO)C(=O)c2nc3cc(C(F)(F)F)ccc3[nH]2)c1Cl] | 156 |
| 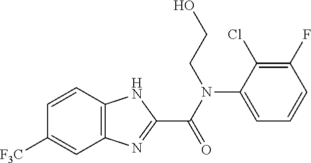 [SMILES: O=C(c1nc2cc(C(F)(F)F)ccc2[nH]1)N(CCO)c1cccc(F)c1Cl] | 157 |
| 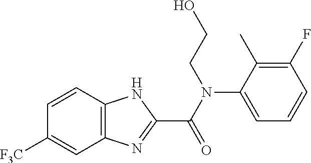 [SMILES: Cc1c(F)cccc1N(CCO)C(=O)c1nc2cc(C(F)(F)F)ccc2[nH]1] | 158 |
| 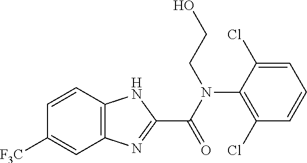 [SMILES: O=C(c1nc2cc(C(F)(F)F)ccc2[nH]1)N(CCO)c1c(Cl)cccc1Cl] | 159 |
| 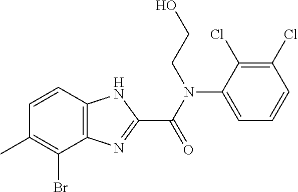 [SMILES: Cc1ccc2[nH]c(C(=O)N(CCO)c3cccc(Cl)c3Cl)nc2c1Br] | 160 |
| 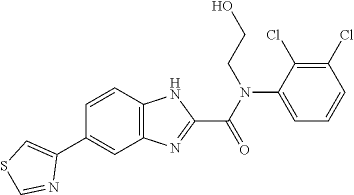 [SMILES: O=C(c1nc2cc(-c3cscn3)ccc2[nH]1)N(CCO)c1cccc(Cl)c1Cl] | 161 |
| 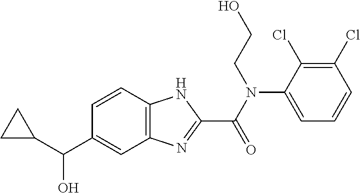 [SMILES: O=C(c1nc2cc(C(O)C3CC3)ccc2[nH]1)N(CCO)c1cccc(Cl)c1Cl] | 162 |
| 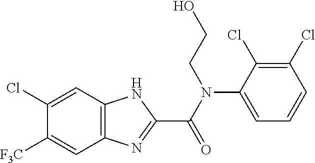 [SMILES: O=C(c1nc2cc(C(F)(F)F)c(Cl)cc2[nH]1)N(CCO)c1cccc(Cl)c1Cl] | 163 |
| 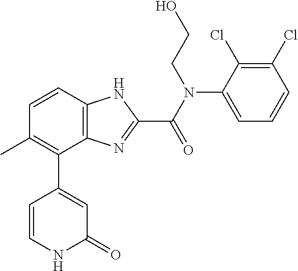 [SMILES: Cc1ccc2[nH]c(C(=O)N(CCO)c3cccc(Cl)c3Cl)nc2c1-c1cc[nH]c(=O)c1] | 164 |
| 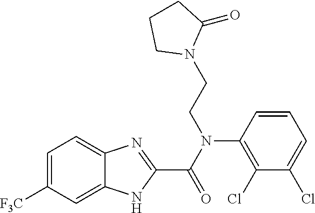 [SMILES: O=C1CCCN1CCN(C(=O)c1nc2ccc(C(F)(F)F)cc2[nH]1)c1cccc(Cl)c1Cl] | 165 |
| 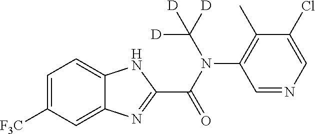 [SMILES: [2H]C([2H])([2H])N(C(=O)c1nc2cc(C(F)(F)F)ccc2[nH]1)c1cncc(Cl)c1C] | 166 |
| 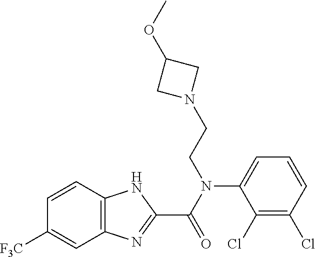 [SMILES: COC1CN(CCN(C(=O)c2nc3cc(C(F)(F)F)ccc3[nH]2)c2cccc(Cl)c2Cl)C1] | 167 |
| 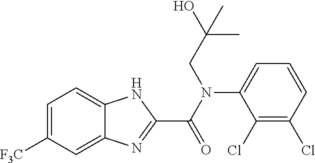 [SMILES: CC(C)(O)CN(C(=O)c1nc2cc(C(F)(F)F)ccc2[nH]1)c1cccc(Cl)c1Cl] | 168 |
| 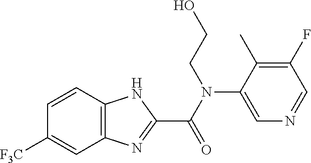 [SMILES: Cc1c(F)cncc1N(CCO)C(=O)c1nc2cc(C(F)(F)F)ccc2[nH]1] | 169 |
| 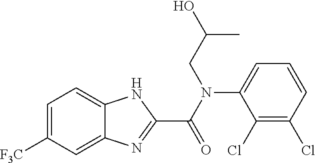 [SMILES: CC(O)CN(C(=O)c1nc2cc(C(F)(F)F)ccc2[nH]1)c1cccc(Cl)c1Cl] | 170 |
| 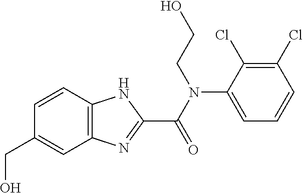 [SMILES: O=C(c1nc2cc(CO)ccc2[nH]1)N(CCO)c1cccc(Cl)c1Cl] | 171 |
| 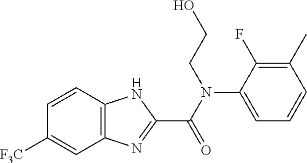 [SMILES: Cc1cccc(N(CCO)C(=O)c2nc3cc(C(F)(F)F)ccc3[nH]2)c1F] | 172 |
| 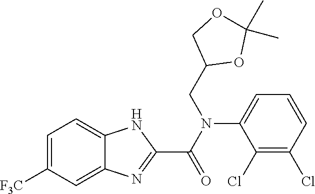 [SMILES: CC1(C)OCC(CN(C(=O)c2nc3cc(C(F)(F)F)ccc3[nH]2)c2cccc(Cl)c2Cl)O1] | 173 |
| 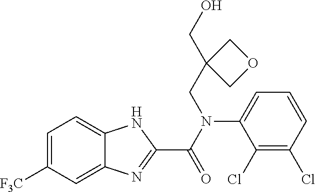 [SMILES: O=C(c1nc2cc(C(F)(F)F)ccc2[nH]1)N(CC1(CO)COC1)c1cccc(Cl)c1Cl] | 174 |
| 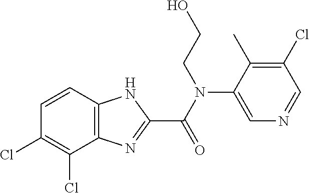 [SMILES: Cc1c(Cl)cncc1N(CCO)C(=O)c1nc2c(Cl)c(Cl)ccc2[nH]1] | 175 |
| 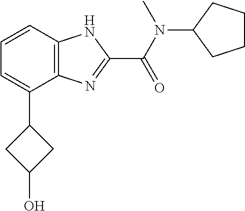 [SMILES: CN(C(=O)c1nc2c(C3CC(O)C3)cccc2[nH]1)C1CCCC1] | 176 |
| 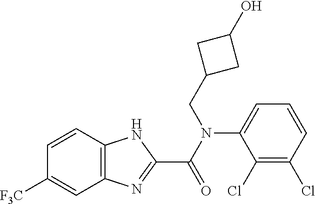 [SMILES: O=C(c1nc2cc(C(F)(F)F)ccc2[nH]1)N(CC1CC(O)C1)c1cccc(Cl)c1Cl] | 177 |
| 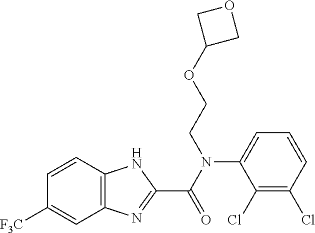 [SMILES: O=C(c1nc2cc(C(F)(F)F)ccc2[nH]1)N(CCOC1COC1)c1cccc(Cl)c1Cl] | 178 |
| 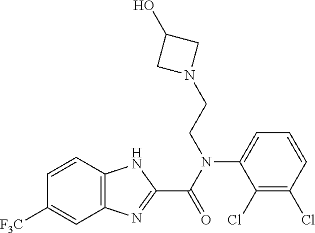 [SMILES: O=C(c1nc2cc(C(F)(F)F)ccc2[nH]1)N(CCN1CC(O)C1)c1cccc(Cl)c1Cl] | 179 |
| 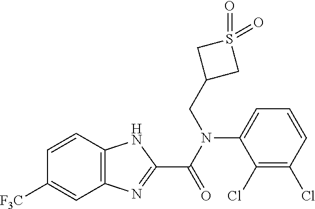 [SMILES: O=C(c1nc2cc(C(F)(F)F)ccc2[nH]1)N(CC1CS(=O)(=O)C1)c1cccc(Cl)c1Cl] | 180 |
| 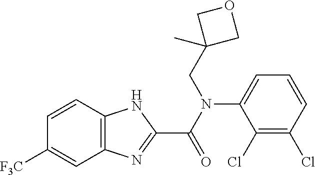 [SMILES: CC1(CN(C(=O)c2nc3cc(C(F)(F)F)ccc3[nH]2)c2cccc(Cl)c2Cl)COC1] | 181 |
| 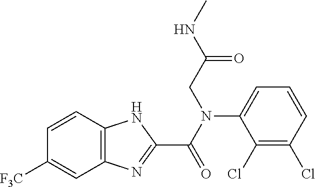 [SMILES: CNC(=O)CN(C(=O)c1nc2cc(C(F)(F)F)ccc2[nH]1)c1cccc(Cl)c1Cl] | 182 |
| 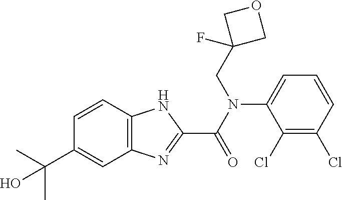 [SMILES: CC(C)(O)c1ccc2[nH]c(C(=O)N(CC3(F)COC3)c3cccc(Cl)c3Cl)nc2c1] | 183 |
| 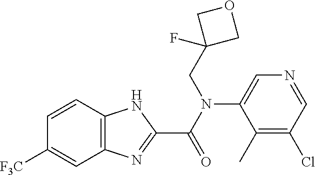 [SMILES: Cc1c(Cl)cncc1N(CC1(F)COC1)C(=O)c1nc2cc(C(F)(F)F)ccc2[nH]1] | 184 |
| 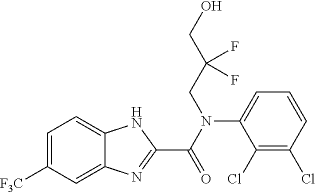 [SMILES: O=C(c1nc2cc(C(F)(F)F)ccc2[nH]1)N(CC(F)(F)CO)c1cccc(Cl)c1Cl] | 185 |
| 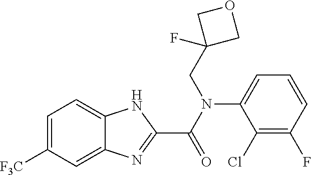 [SMILES: O=C(c1nc2cc(C(F)(F)F)ccc2[nH]1)N(CC1(F)COC1)c1cccc(F)c1Cl] | 186 |
| 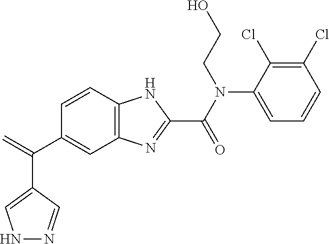 [SMILES: C=C(c1cn[nH]c1)c1ccc2[nH]c(C(=O)N(CCO)c3cccc(Cl)c3Cl)nc2c1] | 187 |
| 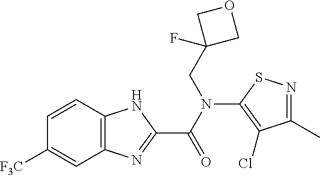 [SMILES: Cc1nsc(N(CC2(F)COC2)C(=O)c2nc3cc(C(F)(F)F)ccc3[nH]2)c1Cl] | 188 |
| 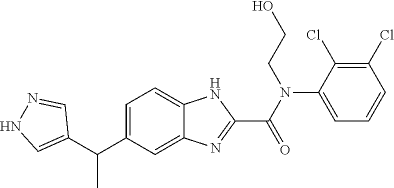 [SMILES: CC(c1cn[nH]c1)c1ccc2[nH]c(C(=O)N(CCO)c3cccc(Cl)c3Cl)nc2c1] | 189 |
| 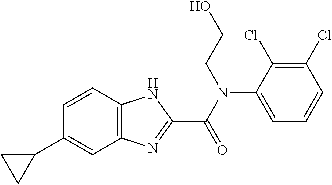 [SMILES: O=C(c1nc2cc(C3CC3)ccc2[nH]1)N(CCO)c1cccc(Cl)c1Cl] | 190 |
| 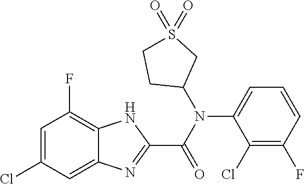 [SMILES: O=C(c1nc2cc(Cl)cc(F)c2[nH]1)N(c1cccc(F)c1Cl)C1CCS(=O)(=O)C1] | 191 |
| 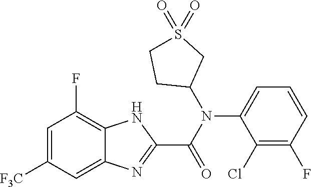 [SMILES: O=C(c1nc2cc(C(F)(F)F)cc(F)c2[nH]1)N(c1cccc(F)c1Cl)C1CCS(=O)(=O)C1] | 192 |
| 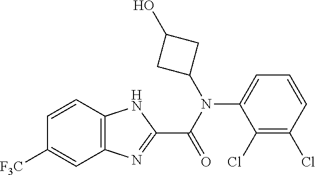 [SMILES: O=C(c1nc2cc(C(F)(F)F)ccc2[nH]1)N(c1cccc(Cl)c1Cl)C1CC(O)C1] | 193 |
| 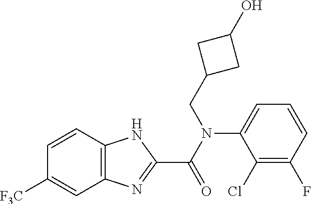 [SMILES: O=C(c1nc2cc(C(F)(F)F)ccc2[nH]1)N(CC1CC(O)C1)c1cccc(F)c1Cl] | 194 |
| 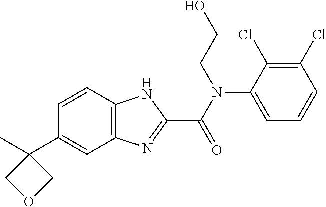 [SMILES: CC1(c2ccc3[nH]c(C(=O)N(CCO)c4cccc(Cl)c4Cl)nc3c2)COC1] | 195 |
| 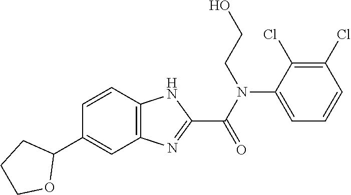 [SMILES: O=C(c1nc2cc(C3CCCO3)ccc2[nH]1)N(CCO)c1cccc(Cl)c1Cl] | 196 |
| 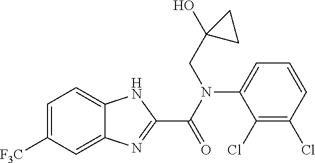 [SMILES: O=C(c1nc2cc(C(F)(F)F)ccc2[nH]1)N(CC1(O)CC1)c1cccc(Cl)c1Cl] | 197 |
| 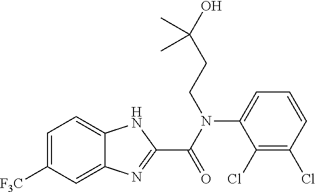 [SMILES: CC(C)(O)CCN(C(=O)c1nc2cc(C(F)(F)F)ccc2[nH]1)c1cccc(Cl)c1Cl] | 198 |
| 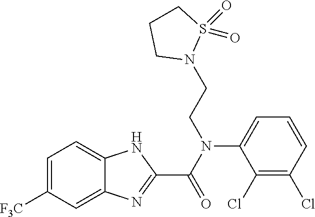 [SMILES: O=C(c1nc2cc(C(F)(F)F)ccc2[nH]1)N(CCN1CCCS1(=O)=O)c1cccc(Cl)c1Cl] | 199 |
| 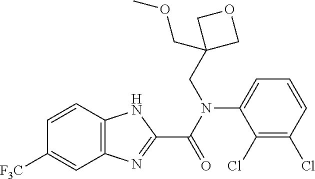 [SMILES: COCC1(CN(C(=O)c2nc3cc(C(F)(F)F)ccc3[nH]2)c2cccc(Cl)c2Cl)COC1] | 200 |
| 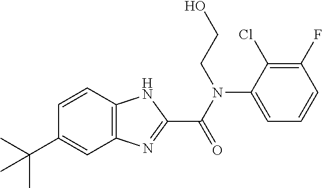 [SMILES: CC(C)(C)c1ccc2[nH]c(C(=O)N(CCO)c3cccc(F)c3Cl)nc2c1] | 201 |
| 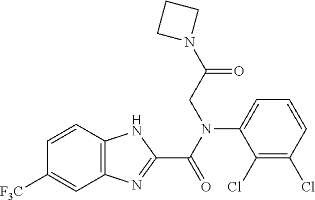 [SMILES: O=C(CN(C(=O)c1nc2cc(C(F)(F)F)ccc2[nH]1)c1cccc(Cl)c1Cl)N1CCC1] | 202 |
| 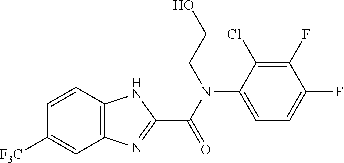 [SMILES: O=C(c1nc2cc(C(F)(F)F)ccc2[nH]1)N(CCO)c1ccc(F)c(F)c1Cl] | 203 |
| 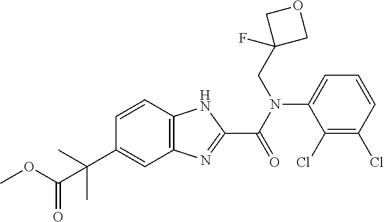 [SMILES: COC(=O)C(C)(C)c1ccc2[nH]c(C(=O)N(CC3(F)COC3)c3cccc(Cl)c3Cl)nc2c1] | 204 |
| 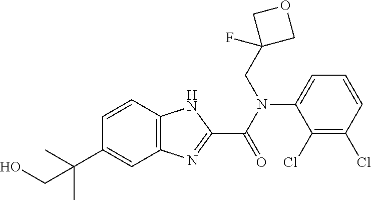 [SMILES: CC(C)(CO)c1ccc2[nH]c(C(=O)N(CC3(F)COC3)c3cccc(Cl)c3Cl)nc2c1] | 205 |
| 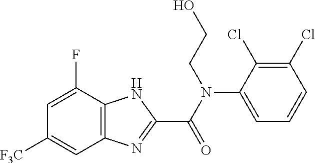 [SMILES: O=C(c1nc2cc(C(F)(F)F)cc(F)c2[nH]1)N(CCO)c1cccc(Cl)c1Cl] | 206 |
| 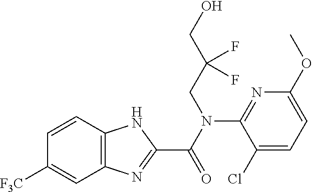 [SMILES: COc1ccc(Cl)c(N(CC(F)(F)CO)C(=O)c2nc3cc(C(F)(F)F)ccc3[nH]2)n1] | 207 |
| 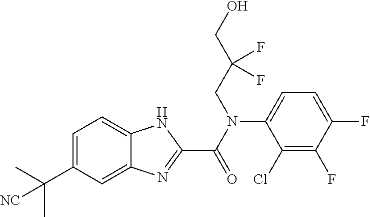 [SMILES: CC(C)(C#N)c1ccc2[nH]c(C(=O)N(CC(F)(F)CO)c3ccc(F)c(F)c3Cl)nc2c1] | 208 |
| 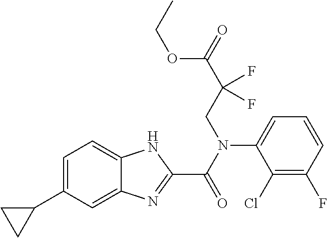 [SMILES: CCOC(=O)C(F)(F)CN(C(=O)c1nc2cc(C3CC3)ccc2[nH]1)c1cccc(F)c1Cl] | 209 |
| 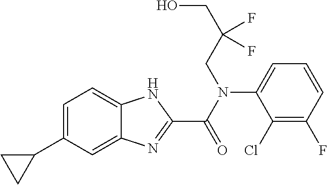 [SMILES: O=C(c1nc2cc(C3CC3)ccc2[nH]1)N(CC(F)(F)CO)c1cccc(F)c1Cl] | 210 |
| 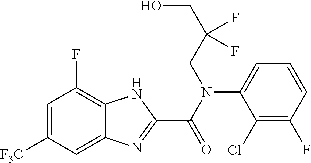 [SMILES: O=C(c1nc2cc(C(F)(F)F)cc(F)c2[nH]1)N(CC(F)(F)CO)c1cccc(F)c1Cl] | 211 |
| 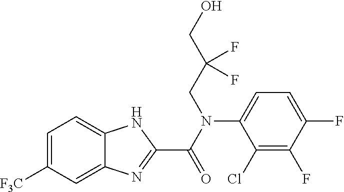 [SMILES: O=C(c1nc2cc(C(F)(F)F)ccc2[nH]1)N(CC(F)(F)CO)c1ccc(F)c(F)c1Cl] | 212 |
| 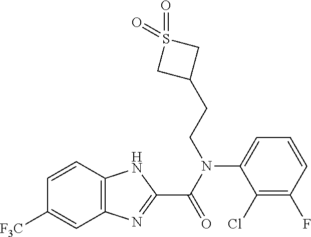 [SMILES: O=C(c1nc2cc(C(F)(F)F)ccc2[nH]1)N(CCC1CS(=O)(=O)C1)c1cccc(F)c1Cl] | 213 |
| 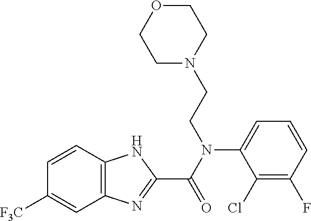 [SMILES: O=C(c1nc2cc(C(F)(F)F)ccc2[nH]1)N(CCN1CCOCC1)c1cccc(F)c1Cl] | 214 |
| 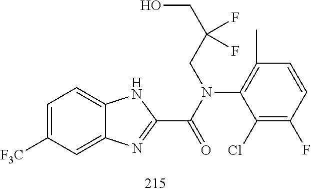 [SMILES: Cc1ccc(F)c(Cl)c1N(CC(F)(F)CO)C(=O)c1nc2cc(C(F)(F)F)ccc2[nH]1] | 215 |
| 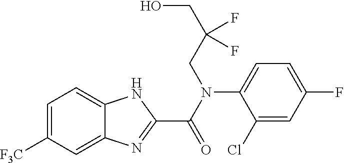 [SMILES: O=C(c1nc2cc(C(F)(F)F)ccc2[nH]1)N(CC(F)(F)CO)c1ccc(F)cc1Cl] | 216 |
| 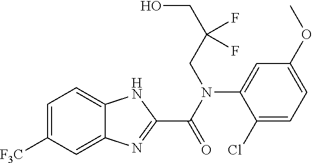 [SMILES: COc1ccc(Cl)c(N(CC(F)(F)CO)C(=O)c2nc3cc(C(F)(F)F)ccc3[nH]2)c1] | 217 |
| 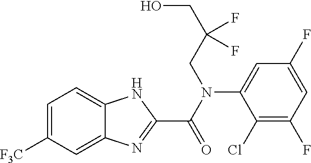 [SMILES: O=C(c1nc2cc(C(F)(F)F)ccc2[nH]1)N(CC(F)(F)CO)c1cc(F)cc(F)c1Cl] | 218 |
| 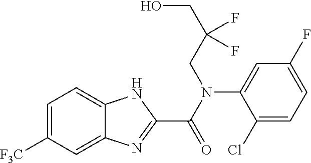 [SMILES: O=C(c1nc2cc(C(F)(F)F)ccc2[nH]1)N(CC(F)(F)CO)c1cc(F)ccc1Cl] | 219 |
| 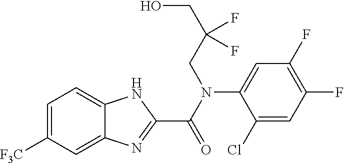 [SMILES: O=C(c1nc2cc(C(F)(F)F)ccc2[nH]1)N(CC(F)(F)CO)c1cc(F)c(F)cc1Cl] | 220 |
| 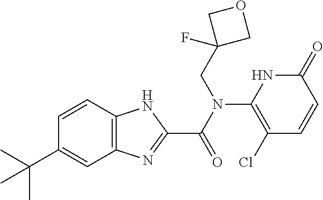 [SMILES: CC(C)(C)c1ccc2[nH]c(C(=O)N(CC3(F)COC3)c3[nH]c(=O)ccc3Cl)nc2c1] | 221 |
| 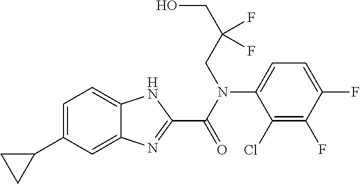 [SMILES: O=C(c1nc2cc(C3CC3)ccc2[nH]1)N(CC(F)(F)CO)c1ccc(F)c(F)c1Cl] | 222 |
| 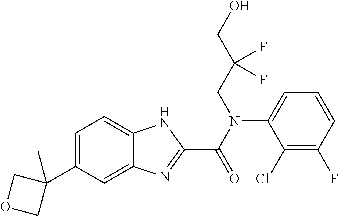 [SMILES: CC1(c2ccc3[nH]c(C(=O)N(CC(F)(F)CO)c4cccc(F)c4Cl)nc3c2)COC1] | 223 |
| 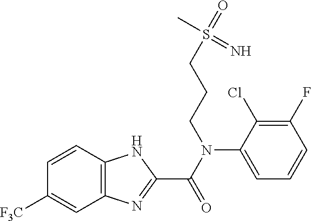 [SMILES: CS(=N)(=O)CCCN(C(=O)c1nc2cc(C(F)(F)F)ccc2[nH]1)c1cccc(F)c1Cl] | 224 |
| 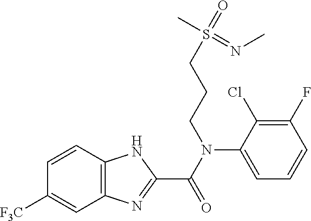 [SMILES: CN=S(C)(=O)CCCN(C(=O)c1nc2cc(C(F)(F)F)ccc2[nH]1)c1cccc(F)c1Cl] | 225 |
| 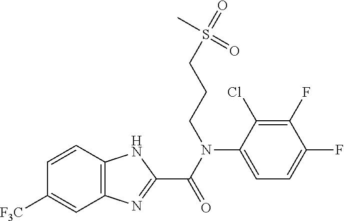 [SMILES: CS(=O)(=O)CCCN(C(=O)c1nc2cc(C(F)(F)F)ccc2[nH]1)c1ccc(F)c(F)c1Cl] | 226 |
| 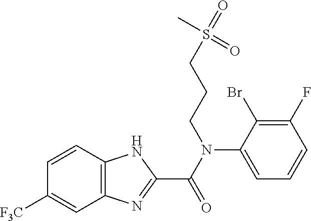 [SMILES: CS(=O)(=O)CCCN(C(=O)c1nc2cc(C(F)(F)F)ccc2[nH]1)c1cccc(F)c1Br] | 227 |
| 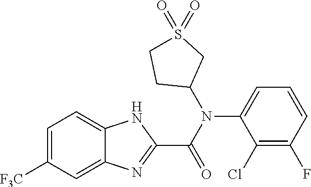 [SMILES: O=C(c1nc2cc(C(F)(F)F)ccc2[nH]1)N(c1cccc(F)c1Cl)C1CCS(=O)(=O)C1] | 228 |
| 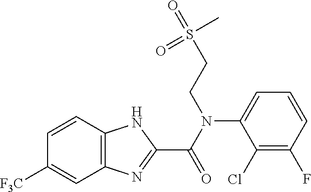 [SMILES: CS(=O)(=O)CCN(C(=O)c1nc2cc(C(F)(F)F)ccc2[nH]1)c1cccc(F)c1Cl] | 229 |
| 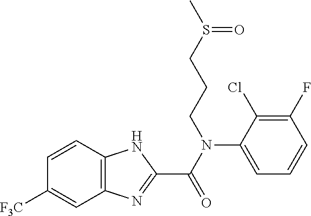 [SMILES: CS(=O)CCCN(C(=O)c1nc2cc(C(F)(F)F)ccc2[nH]1)c1cccc(F)c1Cl] | 230 |
| 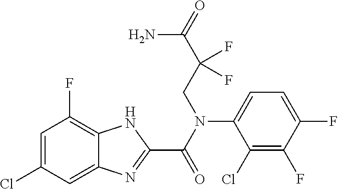 [SMILES: NC(=O)C(F)(F)CN(C(=O)c1nc2cc(Cl)cc(F)c2[nH]1)c1ccc(F)c(F)c1Cl] | 231 |
| 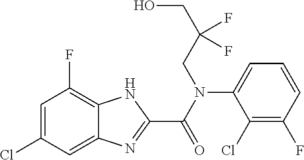 [SMILES: O=C(c1nc2cc(Cl)cc(F)c2[nH]1)N(CC(F)(F)CO)c1cccc(F)c1Cl] | 232 |
| 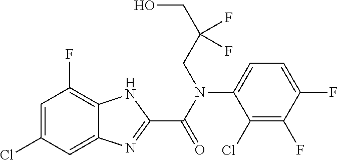 [SMILES: O=C(c1nc2cc(Cl)cc(F)c2[nH]1)N(CC(F)(F)CO)c1ccc(F)c(F)c1Cl] | 233 |
| 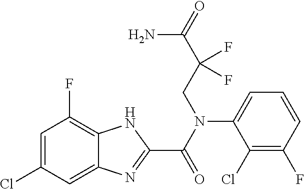 [SMILES: NC(=O)C(F)(F)CN(C(=O)c1nc2cc(Cl)cc(F)c2[nH]1)c1cccc(F)c1Cl] | 234 |
| 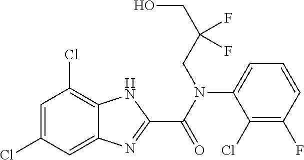 [SMILES: O=C(c1nc2cc(Cl)cc(Cl)c2[nH]1)N(CC(F)(F)CO)c1cccc(F)c1Cl] | 235 |
| 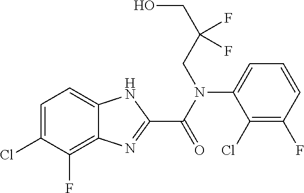 [SMILES: O=C(c1nc2c(F)c(Cl)ccc2[nH]1)N(CC(F)(F)CO)c1cccc(F)c1Cl] | 236 |
| 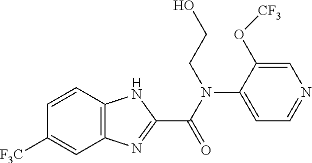 [SMILES: O=C(c1nc2cc(C(F)(F)F)ccc2[nH]1)N(CCO)c1ccncc1OC(F)(F)F] | 237 |
| 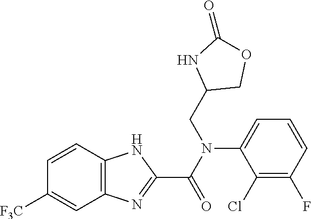 [SMILES: O=C1NC(CN(C(=O)c2nc3cc(C(F)(F)F)ccc3[nH]2)c2cccc(F)c2Cl)CO1] | 238 |
| 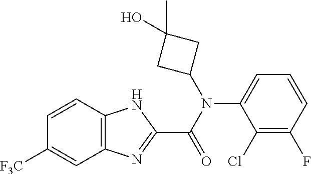 [SMILES: CC1(O)CC(N(C(=O)c2nc3cc(C(F)(F)F)ccc3[nH]2)c2cccc(F)c2Cl)C1] | 239 |
| 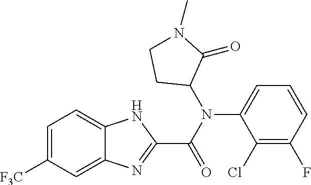 [SMILES: CN1CCC(N(C(=O)c2nc3cc(C(F)(F)F)ccc3[nH]2)c2cccc(F)c2Cl)C1=O] | 240 |

Another aspect of the disclosure provides a pharmaceutical composition comprising at least one compound selected from a compound of Formulae 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, and 6-2f (e.g., Compounds 1 to 240) disclosed herein, a tautomer thereof, a solvate or stereoisomer of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of Formulae 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, and 6-2f (e.g., Compounds 1 to 240), a tautomer thereof, a solvate or stereoisomer of the compound or the tautomer, or a pharmaceutically acceptable salt of the foregoing can be administered as a separate composition concurrently with, prior to, or subsequent to, a composition comprising an additional active pharmaceutical agent.

In some embodiments, the pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams &Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A compound selected from a compound of Formulae 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, and 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, solvate, stereoisomer, or pharmaceutically acceptable salt described herein can also be administered parenterally, in sterile liquid dosage forms, such as dispersions, suspensions or solutions. Other dosages forms that can also be used to administer the compound, tautomer, solvate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing a compound, a tautomer thereof, a solvate or stereoisomer of the compound or the tautomer, and/or a pharmaceutically acceptable salt of the foregoing disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water-soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol.

For administration by inhalation, the compound, tautomer, solvate, stereoisomer, or pharmaceutically acceptable salt described herein may be conveniently delivered in the form of an aerosol spray presentation from pressurized packs or nebulisers. The compound, tautomer, solvate, stereoisomer, or pharmaceutically acceptable salt described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a compound, tautomer, solvate, stereoisomer, or pharmaceutically acceptable salt described herein in at least one suitable propellant, selected, for example, from fluorocarbons and hydrocarbons.

For ocular administration, an ophthalmic preparation may be formulated with an appropriate weight percentage of a solution or suspension of the compound, tautomer, solvate, stereoisomer, or pharmaceutically acceptable salt described herein in an appropriate ophthalmic vehicle, such that the compound, tautomer, solvate, stereoisomer, or pharmaceutically acceptable salt described herein is maintained in contact with the ocular surface for a sufficient time period to allow the compound to penetrate the corneal and internal regions of the eye.

Useful pharmaceutical dosage-forms for administration of the compound, tautomer, solvate, stereoisomer, or pharmaceutically acceptable salt described herein include, but are not limited to, hard and soft gelatin capsules, tablets, parenteral injectables, and oral suspensions. In some embodiments, the pharmaceutical compositions disclosed herein may be in the form of controlled release or sustained release compositions as known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the active material is usually a component ranging from about 0.1 to about 50% by weight or preferably from about 1 to about 40% by weight with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blister pack comprising sheets of at least 6, 9 or 12 unit dosage forms.

In some embodiments, unit capsules can be prepared by filling standard two-piece hard gelatin capsules each with, for example, 100 milligrams of the compound, tautomer, solvate, stereoisomer, or pharmaceutically acceptable salt described herein in powder, 150 milligrams of lactose, 50 milligrams of cellulose, and 6 milligrams magnesium stearate.

In some embodiments, a mixture of the compound, tautomer, solvate, stereoisomer, or pharmaceutically acceptable salt described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of the compound, stereoisomers thereof, or pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereoisomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, or pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, tautomer, solvate, stereoisomer, or pharmaceutically acceptable salt described herein is administered stepwise or in conjunction with at least one other therapeutic agent. When drugs are administered in physical combination, the dosage form and administration route should be selected depending on the compatibility of the combined drugs. Thus, the term coadministration is understood to include the administration of at least two agents concomitantly or sequentially, or alternatively as a fixed dose combination of the at least two active components.

The compound, tautomer, solvate, stereoisomer, or pharmaceutically acceptable salt disclosed herein can be administered as the sole active ingredient or in combination with at least one second active ingredient.

The compound, tautomer, solvate, or stereoisomer described herein may be used in the aforementioned form or in the form of their pharmaceutically acceptable salts, such as hydrochlorides, hydrobromides, acetates, sulfates, citrates, carbonates, trifluoroacetates and the like. When the compound, tautomer, solvate, stereoisomer, or pharmaceutically acceptable salt described herein contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When the compound, tautomer, solvate, or stereoisomer described herein contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts,” Journal of Pharmaceutical Science, 1977, 66, 1-19).

Neutral forms of the pharmaceutically acceptable salt described herein may be regenerated by contacting the salt with a base or acid, and isolating the parent compound in the conventional manner.

This disclosure provides prodrugs. Prodrugs of the compound, tautomer, solvate, stereoisomer, or pharmaceutically acceptable salt described herein that readily undergo chemical changes under physiological conditions to provide the compound, tautomer, solvate, stereoisomer, or pharmaceutically acceptable salt of the present disclosure. Additionally, prodrugs can be converted to the compound, tautomer, solvate, stereoisomer, or a pharmaceutically acceptable salt of the present disclosure by chemical or biochemical methods in an ex vivo environment. For example, prodrugs can be slowly converted to the compound, tautomer, solvate, stereoisomer, or pharmaceutically acceptable salt of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, i.e., the active entity.

Certain compound, tautomer, stereoisomer, or pharmaceutically acceptable salt of the disclosure can exist in unsolvated forms as well as solvated forms, including hydrate forms. Certain compound, tautomer, solvate, stereoisomer, or pharmaceutically acceptable salt of the disclosure may exist in multiple crystalline or amorphous forms.

Certain compound, tautomer, solvate, or pharmaceutically acceptable salt in this disclosure possesses asymmetric carbon atoms (optical centers) or double bonds; the racemates, enantiomers, diastereoisomers, geometric isomers and individual isomers are all intended to be encompassed within the scope of the present disclosure.

### III. Methods of Treatment and Uses

The present disclosure provides methods of treatment and uses utilizing a compound set forth in any one of the various embodiments of Section II (Compounds and Compositions) and Table 1, e.g., a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f, as well as Compounds 1 to 240 in Table 1, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

One aspect of the disclosure provides a method of treating a disease or condition, comprising administering to a subject in need thereof, a therapeutically effective amount of a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, wherein the disease or condition includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but not limited to taxanes, vinca alkaloids and proteasome inhibitors. In some embodiments, the disease or condition is caused by axonal degeneration or neuronal cells damage.

In another aspect, disclosed herein is a compound, tautomer, solvate, stereoisome r, or pharmaceutically acceptable salt as described herein, including a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for use as a medicament.

In another aspect, disclosed herein is use of a compound, tautomer, solvate, stereoisomer, or pharmaceutically acceptable salt as described herein, including a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for the manufacture of a medicament for treating a disease or condition that includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by axonal degeneration or neuronal cell damage.

In a further aspect of this disclosure, a compound, tautomer, solvate, stereoisomer, or pharmaceutically acceptable salt as described herein, including a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in treating a disease or condition that includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by axonal degeneration or neuronal cell damage.

Another aspect of the disclosure provides a method of inhibiting or preventing axonal degeneration, comprising administering to a subject in need thereof, a therapeutically effective amount of a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In another aspect, disclosed herein is use of a compound, tautomer, solvate, stereoisomer, or pharmaceutically acceptable salt as described herein, including a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for the manufacture of a medicament for inhibiting or preventing axonal degeneration or neuronal cells damage.

In a further aspect of this disclosure, a compound, tautomer, solvate, stereoisomer, or pharmaceutically acceptable salt as described herein, including a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in inhibiting or preventing axonal degeneration or neuronal cells damage.

Another aspect of the disclosure provides a method of modulating, e.g., inhibiting, SARM1 in a subject in need thereof, comprising administering to the subject, a therapeutically effective amount of a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In another aspect, disclosed herein is use of a compound, tautomer, solvate, stereoisomer, or pharmaceutically acceptable salt as described herein, including a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for modulating, e.g., inhibiting, SARM1 in a subject in need thereof.

In another aspect of this disclosure, a compound, tautomer, a solvate or stereoisomer of the compound or the tautomer, or pharmaceutically acceptable salt as described herein, including a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in modulating, e.g., inhibiting, SARM1 in a subject in need thereof by contacting the subject with the compound, tautomer, a solvate or stereoisomer of the compound or the tautomer, pharmaceutically acceptable salt, or pharmaceutical composition.

A compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt may be administered once daily, twice daily, or three times daily, for example, for the treatment of a disease or condition, that includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by axonal degeneration or neuronal cells damage.

A compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of one or more compounds, tautomers, solvates, stereoisomers, and pharmaceutically acceptable salt disclosed herein. Generally, administration is adjusted to achieve a therapeutic dosage of about 0.1 to 50 mg/kg, preferably 0.5 to 10 mg/kg, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, 2 mg to 1500 mg or 5 mg to 1000 mg of a compound of Formula 1, 2, 2-1, 2-2, 2-3, 2-4, 3, 3-1, 3-2, 3-3, 4, 4-1, 5, 6-1, 6-2, 6-3, 6-4, 6-1a, 6-2a, 6-2b, 6-2c, 6-2d, 6-2e, or 6-2f (e.g., Compounds 1 to 240), disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt are administered once daily, twice daily, or three times daily. The compound, tautomer, solvate, stereoisomer, or pharmaceutically acceptable salt described herein is administered for morning/daytime dosing, with off period at night.

### A. EXAMPLES

In order that the disclosure described herein may be more fully understood, the following examples are disclosed herein. It should be understood that these examples are for illustrative purposes only and are not to be construed as limiting this disclosure in any way.

### Example I. Synthesis of Exemplary Compounds

The compounds of the disclosure, selected from a compound of the Formulae depicted herein, a tautomer thereof, a solvate or stereoisomer of the compound or the tautomer, or a pharmaceutically acceptable salt of the foregoing, can be made according to standard chemical practices or as illustrated herein, including the following general synthetic procedures and specific synthetic schemes for Compounds 1 to 240.

### Preparation of N-(2,3-dichlorophenyl)-3-(hydroxymethyl)-N,5-dimethyl-1H-indole-2-carboxamide (1) Method 1

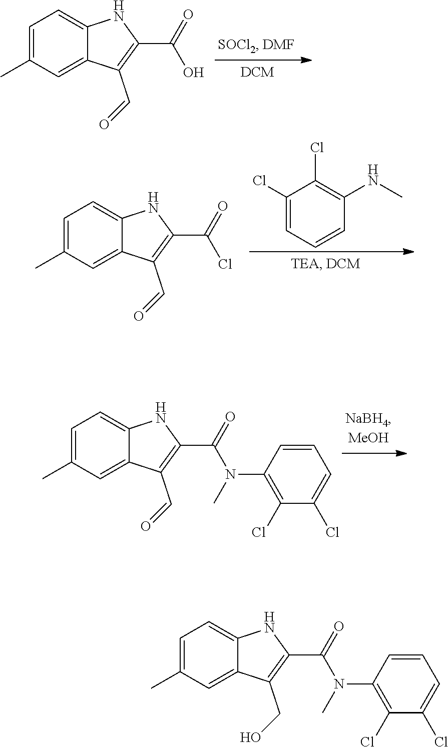

### Step 1. 3-formyl-5-methyl-1H-indole-2-carbonyl chloride

To a solution of 3-formyl-5-methyl-1H-indole-2-carboxylic acid (200 mg, 1.0 mmol), and SOCl₂ (1.28 mL, 15 mmol) in DCM (10 mL) was added 2 drops of DMF at 0° C. The reaction was stirred at room temperature for 2 hours. The mixture was concentrated under vacuum. The residue was used directly for the next step.

### Step 2. N-(2,3-dichlorophenyl)-3-formyl-N,5-dimethyl-1H-indole-2-carboxamide

To a solution of 2,3-dichloro-N-methylaniline (194 mg, 1.1 mmol) and TEA (695 μL, 5.0 mmol) in DCM (10 mL) was added 3-formyl-5-methyl-1H-indole-2-carbonyl chloride (220 mg, 1.0 mmol) at 0° C. and stirred at room temperature for 2 hours. The mixture was concentrated under vacuum, and the mixture was extracted with DCM (20 mL) and H₂O (20 mL). The combined organic layers were dried over Na₂SO₄, filtered and concentrated under vacuum. The residue was purified by FCC (PE:EA=5:1) to give N-(2,3-dichlorophenyl)-3-formyl-N,5-dimethyl-1H-indole-2-carboxamide (75 mg, 21%) as a yellow solid. MS (ESI) m/z 361 [M+H]⁺.

Step 3. N-(2,3-dichlorophenyl)-3-(hydroxymethyl)-N,5-dimethyl-1H-indole-2-carboxamide To a solution of N-(2,3-dichlorophenyl)-3-formyl-N,5-dimethyl-1H-indole-2-carboxamide (75 mg, 0.2 mmol) in MeOH (10 mL) was added NaBH₄ (8 mg, 0.2 mmol) at 0° C. and stirred at room temperature for 1 hour. The mixture was concentrated under vacuum, and the mixture was extracted with EA (20 mL) and H₂O (20 mL). The combined organic layers were dried over Na₂SO₄, filtered and concentrated under vacuum. The residue was purified by prep-HPLC (NH₄HCO₃) to give N-(2,3-dichlorophenyl)-3-(hydroxymethyl)-N,5-dimethyl-1H-indole-2-carboxamide (20 mg, 28%) as a white solid. MS (ESI) m/z 360.9 [M−H]⁻.

¹H NMR (400 MHz, DMSO-d₆) δ 11.09 (d, J=135.5 Hz, 1H), 7.62-7.38 (m, 3H), 7.36-6.79 (m, 3H), 4.74-4.53 (m, 3H), 3.33 (s, 3H), 2.34 (s, 3H).

Examples (Compounds) 2-13 were synthesized using a method similar to that used in Example 1.

| Example | | | | |
|---|---|---|---|---|
| No. | | | | |
| (Compound | | Appearance | | |
| No.) | | and | | MS (m/z) |
| Method | Structure and Name | Yield | ¹H NMR Data | [M − H]⁻ |
| Example 2 Method 1 | 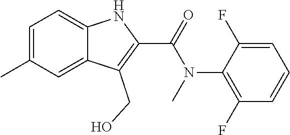 [SMILES: Cc1ccc2[nH]c(C(=O)N(C)c3c(F)cccc3F)c(CO)c2c1] | white solid, yield: 19% | ¹H NMR (400 MHz, DMSO-d₆) δ 11.14 (s, 1H), 7.42-7.21 (m, 3H), 7.17-6.96 (m, 3H), 4,64 (s, 1H), 4.26 (s, 2H), 2.31 (s, 3H). | 329.1 |
| | N-(2,6- | | | |
| | difluorophenyl)-3- | | | |
| | (hydroxymethyl)- | | | |
| | N,5-dimethyl-1H- | | | |
| | indole-2- | | | |
| | carboxamide | | | |
| Example 3 Method 1 | 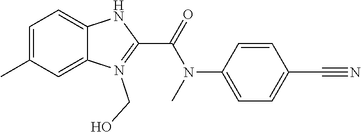 | white solid, yield: 10% | ¹H NMR (400 MHz, DMSO-d₆) δ 11.09 (s, 1H), 7.70 (d, J = 8.8 Hz, 2H), 7.38-7.34 (m, 3H), 7.16 (d, J = 8.3 Hz, 1H), 6.96 (dd, J = 8.4, 1.7 Hz, 1H), 4.73 (t, J = 5.3 Hz, 1H), 4.36 (d, J = 5.3 Hz, 2H), | 318.1 |
| | N-(4-cyanophenyl)- | | 3.45 (s, 3H), 2.33 (s, | |
| | 3-(hydroxymethyl)- | | 3H). | |
| | N,5-dimethyl-1H- | | | |
| | indole-2- | | | |
| | carboxamide | | | |
| Example 4 Method 1 | 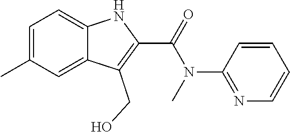 [SMILES: Cc1ccc2[nH]c(C(=O)N(C)c3ccccn3)c(CO)c2c1] | white solid, yield: 18% | ¹H NMR (400 MHz, DMSO-d₆) δ 11.16 (s, 1H), 8.40-8.38 (m, 1H), 7.57-7.55 (m, 1H), 7.39 (s, 1H), 7.18 (d, J = 8.3 Hz, 1H), 7.11 (ddd, J = 7.4, 4.9, 1.0 Hz, 1H), 7.02 (dt, J = 8.2, 1,0 Hz, 1H), 6.96 (dd, J = | 294.1 |
| | 3-(hydroxymethyl)- | | 8.3, 1.7 Hz, 1H), 4.67 | |
| | N,5-dimethyl-N- | | (t, J = 5.3 Hz, 1H), 4.34 | |
| | (pyridin-2-yl)-1H- | | (d, J = 5.4 Hz, 2H), | |
| | indole-2- | | 3.48 (s, 3H), 3.30 (s, | |
| | carboxamide | | 3H). | |
| Example 5 Method 1 | 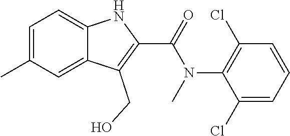 [SMILES: Cc1ccc2[nH]c(C(=O)N(C)c3c(Cl)cccc3Cl)c(CO)c2c1] | white solid, yield: 64.2% | ¹H NMR (400 MHz, CD₃OD-d₄) δ 7.66- 6.89 (m, 6H), 4.97 (s, 1H), 4.75 (s, 1H), 3.40- 3.30 (m, 3H), 2.45-2.03 (m, 3H). | 362.1 |
| | N-(2,6- | | | |
| | dichlorophenyl)-3- | | | |
| | (hydroxymethyl)- | | | |
| | N,5-dimethyl-1H- | | | |
| | indole-2- | | | |
| | carboxamide | | | |
| Example 6 Method 1 | 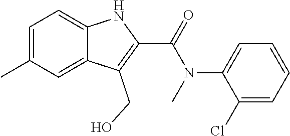 [SMILES: Cc1ccc2[nH]c(C(=O)N(C)c3ccccc3Cl)c(CO)c2c1] | white solid, yield: 44% | ¹H NMR (400 MHz, DMSO-d₆) δ 10.87 (s, 1H), 7.54-7.09 (m, 6H), 6.94 (s, 1H), 4.81-4.32 (m, 3H), 3.31 (s, 3H), 2.32 (s, 3H). | 327.1 |
| | N-(2-chlorophenyl)- | | | |
| | 3-(hydroxymethyl)- | | | |
| | N,5-dimethyl-1H- | | | |
| | indole-2- | | | |
| | carboxamide | | | |
| Example 7 Method 1 | 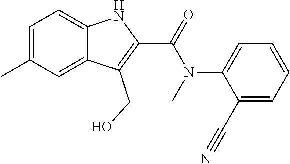 [SMILES: Cc1ccc2[nH]c(C(=O)N(C)c3ccccc3C#N)c(CO)c2c1] | white solid, yield: 44% | ¹H NMR (300 MHz, DMSO-d₆) δ 8.64-8.04 (m, 1H), 7,90-7.66 (m, 3H), 7.55-7.45 (m, 1H), 7.23-7.12 (m, 1H), 7.01-6.87 (m, 1H), 5.76-4.55 (m, 3H), 3.48 (s, 1H), 2.78 (d, J = 6.0 Hz, 2H), 2.46-2.11 (m, 3H). | 318.1 |
| | N-(2-cyanophenyl)- | | | |
| | 3-(hydroxymethyl)- | | | |
| | N,5-dimethyl-1H- | | | |
| | indole-2- | | | |
| | carboxamide | | | |
| Example 8 Method 1 | 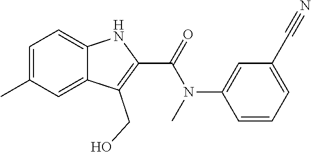 [SMILES: Cc1ccc2[nH]c(C(=O)N(C)c3cccc(C#N)c3)c(CO)c2c1] | white solid, yield: 6% | ¹H NMR (400 MHz, DMSO-d₆) δ 10.98 (s, 1H), 7.81 (t, J = 1.6 Hz, 1H), 7.60-7.57 (m, 1H), 7.42-7.38 (m, 3H), 7.15 (d, J = 8.4 Hz, 1H), 6.94 (dd, J = 8.4, 1.6 Hz, 1H), 4.75 (t, J = 5.2 Hz, 1H), 4,41 (d, J = 5.6 Hz, 2H), 3.43 (s, | 302.0 [M −OH]⁺ |
| | N-(3-cyanophenyl)- | | 3H), 2.33 (s, 3H). | |
| | 3-(hydroxymethyl)- | | | |
| | N,5-dimethyl-1//- | | | |
| | indole-2- | | | |
| | carboxamide | | | |
| Example 9 Method 1 | 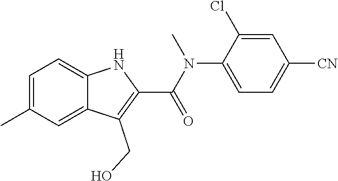 [SMILES: Cc1ccc2[nH]c(C(=O)N(C)c3ccc(C#N)cc3Cl)c(CO)c2c1] | white solid, yield: 19% | ¹H NMR (400 MHz, CD₃OD-d₄) δ 7.87 (s, 1H), 7.66-7.62 (m, 2H), 7.47 (s, 1H), 7.16 (d, J = 8.0 Hz, 1H), 7.02 (d, J = 8.0 Hz, 1H), 4.77 (s, 2H), 3.43 (s, 3H), 2.38 (s, 3H). | 352.1 |
| | N-(2-chloro-4- | | | |
| | cyanophenyl)-3- | | | |
| | (hydroxymethyl)- | | | |
| | N,5-dimethyl-1H- | | | |
| | indole-2- | | | |
| | carboxamide | | | |
| Example 10 Method 1 | 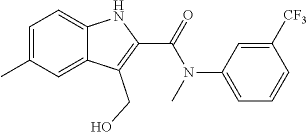 [SMILES: Cc1ccc2[nH]c(C(=O)N(C)c3cccc(C(F)(F)F)c3)c(CO)c2c1] | white solid, yield: 45% | ¹H NMR (400 MHz, DMSO-d₆) δ 11.05 (s, 1H), 7.64 (s, 1H), 7.48- 7.43 (m, 3H), 7.36 (s, 1H), 7.15 (d, J = 8.4 Hz, 1H), 6.94 (dd, J = 8.0, 1.2 Hz, 1H), 4.74 d, J = 5.2 Hz, 1H), 4.39 (d, J = 5.2 Hz, 2H), 3.44 (s, | 345.1 [M − OH]⁺ |
| | 3-(hydroxymethyl)- | | 3H), 2.32 (m, 3H). | |
| | N,5-dimethyl-N-(3- | | | |
| | (trifluoromethyl)phen | | | |
| | yl)-1H-indole-2- | | | |
| | carboxamide | | | |
| Example 11 Method 1 | 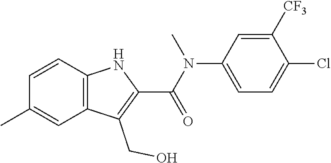 [SMILES: Cc1ccc2[nH]c(C(=O)N(C)c3ccc(Cl)c(C(F)(F)F)c3)c(CO)c2c1] | white solid, yield: 52% | ¹H NMR (400 MHz, CD₃OD-d₄) δ 7.69 (d, J = 2.4 Hz, 1H), 7.44- 7.39 (m, 3H), 7.12 (d, J = 8.4 Hz, 1H), 7.02 (d, J = 1.2. Hz, 1H), 4,72 (s, 2H), 3.51 (s, 3H), 2.39 (s, 3H). | 395.1 |
| | N-(4-chloro-3- | | | |
| | (trifluoromethyl)phenyl)- | | | |
| | 3- | | | |
| | (hydroxymethyl)- | | | |
| | N,5-dimethyl-1H- | | | |
| | indole-2- | | | |
| | carboxamide | | | |
| Example 12 Method 1 | 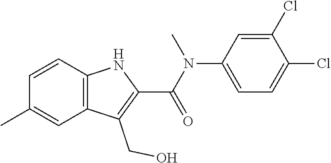 [SMILES: Cc1ccc2[nH]c(C(=O)N(C)c3ccc(Cl)c(Cl)c3)c(CO)c2c1] | white solid, yield: 27% | ¹H NMR (400 MHz, DMSO-d6) δ 11.02 (s, 1H), 7.61 (d, J = 6.4 Hz, 1H), 7.46 (d, J = 8.0 Hz, 1H), 7.39 (s, 1H), 7.18-7.10 (m, 2H), 6.95 (dd, J = 8.4, 1.6 Hz, 1H), 4.75 (t, J = 5.2 Hz, 1H), 4.44 (d, J = 5.2 Hz, 2H), 3.40 (s, | 345.1 [M − OH]⁺ |
| | dichlorophenyl)-3- | | 3H), 2.33 (s, 3H). | |
| | (hydroxymethyl)- | | | |
| | N,5-dimethyl-1H- | | | |
| | indole-2- | | | |
| | carboxamide | | | |
| Example 13 Method 1 | 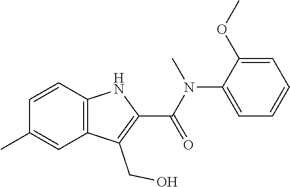 [SMILES: COc1ccccc1N(C)C(=O)c1[nH]c2ccc(C)cc2c1CO] | white solid, yield: 31% | ¹H NMR (400 MHz, DMSO-d₆) δ 10.91 (s, 1H), 7.33 (s, 1H), 7.19- 7.12 (m, 3H), 6.94-6.88 (m, 2H), 6.82 (t, J = 7.2 Hz, 1H), 4.49 (s, 1H), 4.34 (s, 2H), 3.67 (s, 3H), 3.25 (s, 3H), 2.30 (s, 3H). | 323.2 |
| | 3-(hydroxymethyl)- | | | |
| | N-(2- | | | |
| | methoxyphenyl)-N,5- | | | |
| | dimethyl-1H-indole- | | | |
| | 2-carboxamide | | | |

### Preparation of N-cyclopentyl-N,5-dimethyl-3-propyl-1H-indole-2-carboxamide (14)

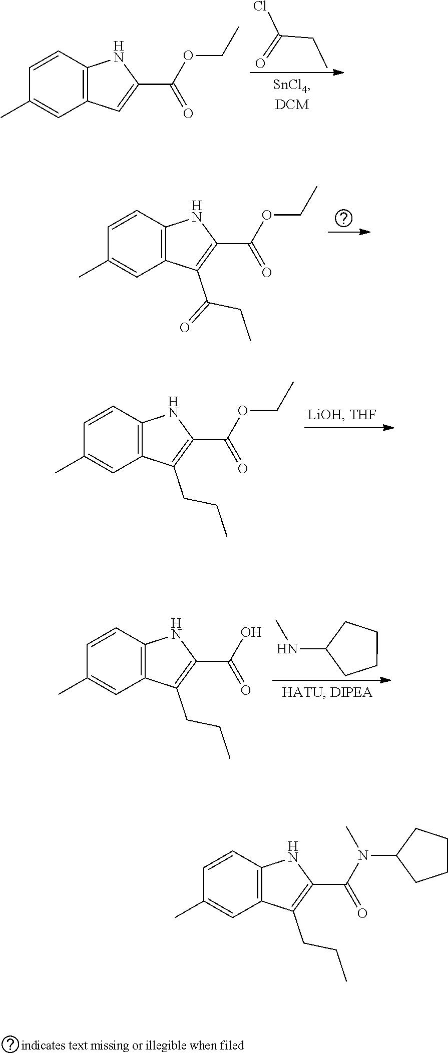

### Step 1. ethyl 5-methyl-3-propionyl-1H-indole-2-carboxylate

To a solution of ethyl 5-methyl-1H-indole-2-carboxylate (3 g, 14.7 mmol) in DCM (30 mL) was added SnCl₄ (4.58 g, 17.6 mmol); the mixture was stirred at 0° C. for 1 h, then propionyl chloride (1.35 g, 14.7 mmol) was added; the mixture was stirred at rt for another 16 h. The mixture was poured into ice-water (30 mL), and was extracted with DCM (30 mL*2). The combined organic layers were washed with water (20 mL*2) and brine (20 mL), dried over Na₂SO₄, filtered and concentrated under vacuum. The residue was purified by FCC (PE:EA=5:1) to give ethyl 5-methyl-3-propionyl-1H-indole-2-carboxylate (1.1 g, 28.9%) as a yellow solid. MS (ESI) m/z 260 [M+H]⁺.

### Step 2. ethyl 5-methyl-3-propyl-1H-indole-2-carboxylate

To a solution of ethyl 5-methyl-3-propionyl-1H-indole-2-carboxylate (1 g, 3.8 mmol) in TFA (15 mL) was added triethylsilane (1.8 g, 15.4 mmol), the mixture was stirred at 70° C. for 3 h. The mixture was concentrated and the pH of the mixture was adjusted to 9 with sat. NaHCO₃; and then the mixture was extracted with EA (30 mL*2). The combined organic layers were washed with water (20 mL*2) and brine (20 mL), dried over Na₂SO₄, filtered and concentrated under vacuum. The residue was purified by FCC (PE:EA=3:1) to give ethyl 5-methyl-3-propyl-1H-indole-2-carboxylate (600 mg, 63%) as a yellow solid. MS (ESI) m/z 240 [M+H]⁺.

### Step 3. 5-methyl-3-propyl-1H-indole-2-carboxylic acid

A solution of ethyl 5-methyl-3-propyl-1H-indole-2-carboxylate (600 mg, 2.45 mmol) and LiOH·H₂O (411 mg, 9.8 mmol) in THF/H₂O (20 mL/5 mL) was stirred at 50° C. for 4 hours. After THE was removed, the pH of the residue was adjusted to 6 with 1M HCl, after filtration the cake was dried to give 5-methyl-3-propyl-1H-indole-2-carboxylic acid (320 mg, 69%) as a yellow solid. MS (ESI) m/z 218 ([M+H]⁺).

### Step 4. N-cyclopentyl-N,5-dimethyl-3-propyl-1H-indole-2-carboxamide

To a solution of 5-methyl-3-propyl-1H-indole-2-carboxylic acid (150 mg, 0.69 mmol) in DMF (10 mL) was added N-methylcyclopentanamine (68 mg, 0.69 mmol), DIPEA (267 mg, 2.07 mmol) and HATU (314 mg, 0.82 mmol) at rt. The mixture was stirred at rt for 16 hours. To the mixture was added water (20 mL), and the mixture was extracted with EA (30 mL*2). The combined organic layers were washed with water (20 mL*2) and brine (20 mL), dried over anhydrous Na₂SO₄, filtered and concentrated. The residue was purified by prep-HPLC (NH₄HCO₃) to give N-cyclopentyl-N, 5-dimethyl-3-propyl-1H-indole-2-carboxamide (32.6 mg, 15.8%) as a white solid. MS (ESI) m/z 299 ([M+H]⁺).

¹H NMR (400 MHz, CD₃OD-d₄) δ 7.34 (s, 1H), 7.22 (d, J=8.4 Hz, 1H), 7.00 (dd, J=8.4, 1.6 Hz, 1H), 4.47 (brs, 1H), 2.96 (s, 3H), 2.72 (t, J=7.6 Hz, 2H), 2.41 (s, 3H), 1.92-1.53 (m, 10H), 0.93 (t, J=7.6 Hz, 3H).

### Preparation of 3-(cyanomethyl)-N-cyclopentyl-N,5-dimethyl-1H-indole-2-carboxamide (15) and 3-(2-amino-2-oxoethyl)-N-cyclopentyl-N,5-dimethyl-1H-indole-2-carboxamide (16)

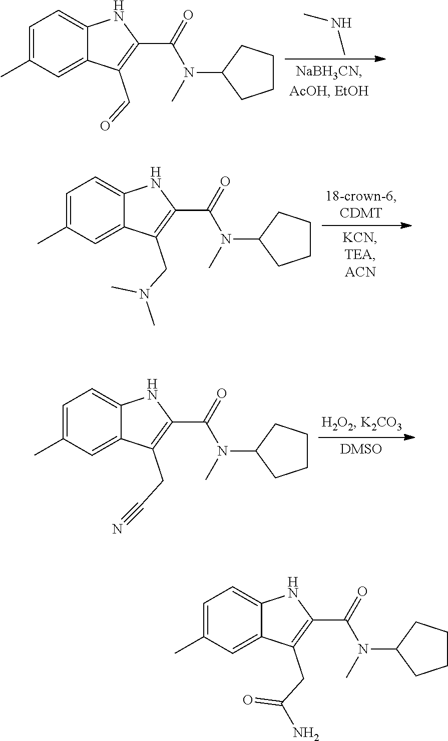

Step 1. N-cyclopentyl-3-((dimethylamino)methyl)-N,5-dimethyl-1H-indole-2-carboxamide To a solution of N-cyclopentyl-3-formyl-N,5-dimethyl-1H-indole-2-carboxamide (1.0 g, 3.52 mmol) in EtOH (30 mL) was added dimethylamine (8.8 mL, 17.6 mmol), AcOH (5 drop) and NaBH₃CN (436 mg, 7.04 mmol). The mixture was stirred at 90° C. for 5 hours. The mixture was poured into ice-water (30 mL), and was extracted with EA (30 mL*2). The combined organic layers were washed with water (30 mL*2) and brine (30 mL), dried over Na₂SO₄, filtered and concentrated under vacuum. The residue was purified by FCC (DCM:MeOH=10:1) to give N-cyclopentyl-3-((dimethylamino)methyl)-N,5-dimethyl-1H-indole-2-carboxamide (500 mg, 45%) as a yellow oil. MS (ESI) m/z 314.1 [M+H]⁺.

### Step 2. 3-(cyanomethyl)-N-cyclopentyl-N,5-dimethyl-1H-indole-2-carboxamide

CMDT (245 mg, 1.4 mmol) was added to a solution of N-cyclopentyl-3-((dimethylamino)methyl)-N,5-dimethyl-1H-indole-2-carboxamide (400 mg, 1.3 mmol), 18-crown-6 (370 mg, 1.4 mmol), KCN (91 mg, 1.4 mmol) and TEA (131 mg, 1.3 mmol) in ACN (5 mL). The mixture was stirred at rt for 2 hours. To the mixture was added water (20 mL), and extracted with EA (20 mL*2). The combined organic layers were washed with brine (30 mL), dried over Na₂SO₄, filtered and concentrated under vacuum. The residue was purified by FCC (PE:EA=2:1) to give 3-(cyanomethyl)-N-cyclopentyl-N,5-dimethyl-1H-indole-2-carboxamide (160 mg, 43%) as a yellow solid. MS (ESI) m/z 296.2 [M+H]⁺. ¹H NMR (400 MHz, DMSO-d₆) δ11.46 (s, 1H), 7.44 (s, 1H), 7.29 (d, J=7.6 Hz, 1H), 7.06 (dd, J=8.4, 1.2 Hz, 1H), 4.45 (brs, 1H), 3.96 (s, 2H), 2.88 (s, 3H), 2.41 (s, 3H), 1.79-1.47 (m, 8H).

### Step 3. 3-(2-amino-2-oxoethyl)-N-cyclopentyl-N,5-dimethyl-1H-indole-2-carboxamide

H₂O₂ (0.1 mL, 0.6 mmol) was added to a mixture of 3-(cyanomethyl)-N-cyclopentyl-N, 5-dimethyl-1H-indole-2-carboxamide (80 mg, 0.3 mmol) and K₂CO 3 (8.3 mg, 0.06 mmol) in DMSO (5 mL). The mixture was stirred at rt for 12 hours. To the mixture was added water (20 mL), and the mixture was extracted with EA (20 mL*2). The combined organic layers were washed with brine (30 mL), dried over Na₂SO₄, filtered and concentrated under vacuum. The residue was purified by prep-HPLC (NH₄HCO₃) to give 3-(2-amino-2-oxoethyl)-N-cyclopentyl-N,5-dimethyl-1H-indole-2-carboxamide (36.1 mg, 42%) as a white solid. MS (ESI) m/z 314.2 [M+H]⁺. ¹H NMR (400 MHz, DMSO-d₆) δ11.12 (s, 1H), 7.37-7.31 (m, 2H), 7.23 (d, J=8.4 Hz, 1H), 6.98 (dd, J=8.4, 1.2 Hz, 1H), 6.82 (s, 1H), 4.50 (brs, 1H), 3.43 (s, 2H), 2.87 (s, 3H), 2.37 (s, 3H), 1.78-1.46 (m, 8H).

### Preparation of N-cyclopentyl-3-(hydroxymethyl)-N,6-dimethyl-1H-indole-2-carboxamide (17) Method 2

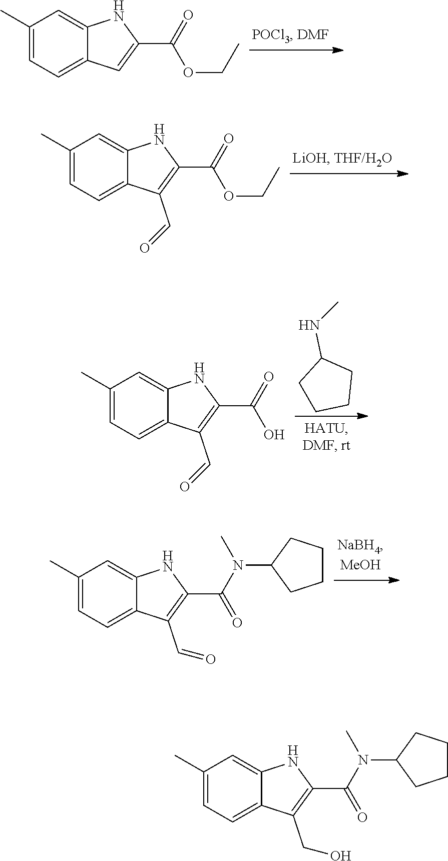

### Step 1. ethyl 3-formyl-6-methyl-1H-indole-2-carboxylate

To a solution of ethyl 6-methyl-1H-indole-2-carboxylate (500 mg, 2.46 mmol) in DMF (10 mL) was added POCl₃ (0.46 mL, 4.92 mmol) at 0° C. The reaction was stirred at 90° C. for 3 hours. The mixture was poured into ice-water (30 mL), and the mixture was extracted with EA (30 mL*2). The combined organic layers were washed with water (100 mL*2) and brine (30 mL), dried over Na₂SO₄, filtered and concentrated under vacuum. The residue was purified by FCC (PE:EA=24:1) to give ethyl 3-formyl-6-methyl-1H-indole-2-carboxylate (310 mg, 54%) as a yellow solid. MS (ESI) m/z 232 [M+H]⁺.

### Step 2. 3-formyl-6-methyl-1H-indole-2-carboxylic acid

A solution of ethyl 3-formyl-6-methyl-1H-indole-2-carboxylate (300 mg, 1.29 mmol) and LiOH·H₂O (163 mg, 3.89 mmol) in THF/H₂O (10 mL/2 mL) was stirred at 50° C. for 4 hours. After concentration the pH of the residue was adjusted to 6 with 1M HCl, filtered to give 3-formyl-6-methyl-1H-indole-2-carboxylic acid (230 mg, 90%) as a yellow solid. MS (ESI) m/z 204 [M+H]⁺.

### Step 3. N-cyclopentyl-3-formyl-N,6-dimethyl-1H-indole-2-carboxamide

To a solution of 3-formyl-6-methyl-1H-indole-2-carboxylic acid (200 mg, 0.98 mmol) in DMF (10 mL) was added N-methylcyclopentanamine (87 mg, 0.89 mmol), DIPEA (379 mg, 2.94 mmol) and HATU (447 mg, 1.18 mmol) at rt. The mixture was stirred at rt for 2 hours. To the mixture was added water (20 mL), and the mixture was extracted with EA (30 mL*2). The combined organic layers were washed with water (20 mL*2) and brine (20 mL), dried over anhydrous Na₂SO₄, filtered and concentrated. The residue was purified by FCC (PE:EA=1:1) to give N-cyclopentyl-3-formyl-N,6-dimethyl-1H-indole-2-carboxamide (80 mg, 28.7%) as a yellow solid. MS (ESI) m/z 285 [M+H]⁺.

### Step 4. N-cyclopentyl-3-(hydroxymethyl)-N,6-dimethyl-1H-indole-2-carboxamide

To a solution of N-cyclopentyl-3-formyl-N,6-dimethyl-1H-indole-2-carboxamide (90 mg, 0.21 mmol) in MeOH (8 mL) was added NaBH₄ (10 mg, 0.25 mmol) at rt. The reaction was stirred at rt for 1 hour. To the mixture was added water (20 mL) at ice bath, and the mixture was extracted with EA (20 mL*2), dried over Na₂SO₄, filtered and concentrated. The residue was purified by prep-HPLC (NH₄HCO₃) to give N-cyclopentyl-3-(hydroxymethyl)-N,6-dimethyl-1H-indole-2-carboxamide (57.7 mg, 63%) as a white solid. MS (ESI) m/z 285 [M−H]⁻.

¹H NMR (400 MHz, DMSO-d₆) δ 11.11 (s, 1H), 7.57 (d, J=8.0 Hz, 1H), 7.12 (s, 1H), 6.87 (dd, J=8.0, 1.6 Hz, 1H), 4.78 (t, J=5.2 Hz, 1H), 4.56 (d, J=5.2 Hz, 3H), 2.87 (s, 3H), 2.39 (s, 3H), 1.82-1.45 (m, 8H).

Examples (Compounds) 18, 19, 21-45 were synthesized using a method similar to that used in Example 17.

| Example | | | | |
|---|---|---|---|---|
| No. | | | | |
| (Compound | | Appearance | | |
| No.) | | and | | MS (m/z) |
| Method | Structure and Name | Yield | ¹H NMR Data | [M − H]⁻ |
| Example 18 Method 2 | 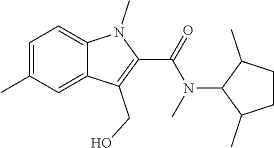 [SMILES: Cc1ccc2c(c1)c(CO)c(C(=O)N(C)C1C(C)CCC1C)n2C] | white solid, yield: 58% | ¹H NMR (400 MHz, DMSO-d₆) δ 7.49 (s, 1H), 7.33 (t, J = 7.7 Hz, 1H), 7.04 (dd, J = 8.5, 1.8 Hz, 1H), 4.92-4.73 (m, 1H), 4.66-4.44 (m, 2H), 4.31-4.13 (m, 1H), 3.65-3.53 (m, 3H), 3.05-2.69 (m, 3H), 2.40 | 311.2 [M − OH]⁻ |
| | N-(2,5- | | (s, 3H), 2.31-1.70 (m, | |
| | dimethylcyclopentyl)- | | 4H), 1.34-0.86 (m, 8H). | |
| | 3-(hydroxymethyl)- | | | |
| | N,1,5-trimethyl-1H- | | | |
| | indole-2- | | | |
| | carboxamide | | | |
| Example 19 Method 2 | 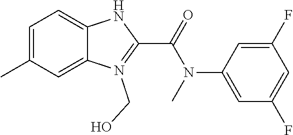 | white solid, yield: 45% | ¹H NMR (400 MHz, DMSO-d₆) δ 10.97 (s, 1H), 7.41 (d, J = 1.6 Hz, 1H), 7.17 (d, J = 8.4 Hz, 1H), 7.07-6.90 (m, 4H), 4.77 (d, J = 4.8 Hz, 1H), 4.49 (d, J = 3.2 Hz, 2H), 3.41 (s, 3H), 2.34 (s, 3H). | 329.1 |
| | N-(3,5- | | | |
| | difluorophenyl)-3- | | | |
| | (hydroxymethyl)- | | | |
| | N,5-dimethyl-1H- | | | |
| | indole-2- | | | |
| | carboxamide | | | |
| Example 21 Method 2 | 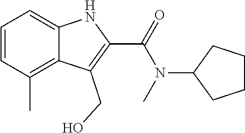 [SMILES: Cc1cccc2[nH]c(C(=O)N(C)C3CCCC3)c(CO)c12] | white solid, yield: 55% | ¹H NMR (400 MHz, DMSO-d₆) δ 11.27 (s, 1H), 7.15 (d, J = 8.0 Hz, 1H), 7.01 (t, J = 7.6 Hz, 1H), 6.78 (d, J = 7.2 Hz, 1H), 4.73 (t, J = 4.0 Hz, 1H), 4.59 (d, J = 4.4 Hz, 3H), 2.87 (s, 3H), 2.69 (s, 3H), 1.88-1.41 (m, | 285.2 |
| | N-cyclopenty1-3- | | 8H). | |
| | (hydroxymethyl)- | | | |
| | N,4-dimethyl-1H- | | | |
| | indole-2- | | | |
| | carboxamide | | | |
| Example 22 Method 2 | 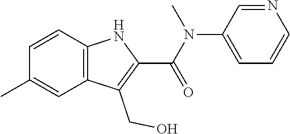 [SMILES: Cc1ccc2[nH]c(C(=O)N(C)c3cccnc3)c(CO)c2c1] | yellow solid, yield: 17% | ¹H NMR (400 MHz, DMSO-d₆) δ 11.02 (s, 1H), 8.36 (d, J = 2.4 Hz, 1H), 8.29 (dd, J = 4.8, 1.6 Hz, 1H), 7.66 (ddd, J = 8.4, 2.8, 1.6 Hz, 1H), 7.36 (s, 1H), 7.32-7.26 (m, 1H), 7.13 (d, J = 8.4 Hz, 1H), 6.93 (dd, J = | 294.1 |
| | 3-(hydroxymethyl)- | | 8.4, 1.6 Hz, 1H), 4,74 (t, | |
| | N,5-dimethyl-N- | | J = 5.2 Hz, 1H), 4.38 (d, | |
| | (pyridin-3-yl)-1H- | | J = 5.6 Hz, 2H), 3.43 (s, | |
| | indole-2- | | 3H), 2.32 (s, 3H). | |
| | carboxamide | | | |
| Example 23 Method 2 | 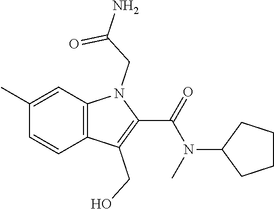 [SMILES: Cc1ccc2c(CO)c(C(=O)N(C)C3CCCC3)n(CC(N)=O)c2c1] | yellow solid, yield: 54% | ¹H NMR (400 MHz, CD₃OD-d₄) δ 7.64 (d, J = 8.0 Hz, 1H), 7.15 (s, 1H), 7.00 (d, J = 8.0 Hz, 1H), 4.84-4.91(m, 0.5H), 4.79-4.66 (m, 4H), 4.42-4.44 (m, 0.5H), 3.01 (s, 3H), 2.46 (s, 3H), 2.03-1,40 (m, 8H). | 342.2 |
| | 1-(2-amino-2- | | | |
| | oxoethyl)-N- | | | |
| | cyclopentyl-3- | | | |
| | (hydroxymethyl)- | | | |
| | N,6-dimethyl-1H- | | | |
| | indole-2- | | | |
| | carboxamide | | | |
| Example 24 Method 2 | 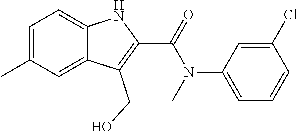 [SMILES: Cc1ccc2[nH]c(C(=O)N(C)c3cccc(Cl)c3)c(CO)c2c1] | white solid, yield: 27% | ¹H NMR (400 MHz, DMSO-d₆) δ 11.00 (s, 1H), 7.38 (dd, J = 4.3, 2.1 Hz, 2H), 7.24-7.13 (m, 3H), 7.09 (dt, J = 7.6, 1.9 Hz, 1H), 6.94 (dd, J= 8.3, 1,7 Hz, 1H), 4.71 (t, J = 5.2 Hz, 1H), 4.41 (d, J = 4.7 Hz, 2H), | 327.1 |
| | N-(3-chlorophenyl)- | | 3.40 (s, 3H), 2.33 (s, | |
| | 3-(hydroxymethyl)- | | 3H). | |
| | N,5-dimethyl-1H- | | | |
| | indole-2- | | | |
| | carboxamide | | | |
| Example 25 Method 2 | 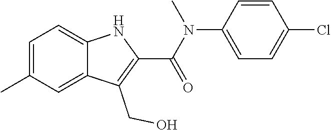 [SMILES: Cc1ccc2[nH]c(C(=O)N(C)c3ccc(Cl)cc3)c(CO)c2c1] | white solid, yield: 46% | ¹H NMR (400 MHz, DMSO-d₆) δ 10.99 (s, 1H), 7.36 (s, 1H), 7.29 (d, J = 8.7 Hz, 2H), 7.20 (d, J = 8.7 Hz, 2H), 7.14 (d, J = 8.2 Hz, 1H), 6.93 (d, J = 8.2 Hz, 1H), 4.68 (t, J = 5,4 Hz, 1H), 4.37 | 311.1 [M − OH]⁺ |
| | N-(4-chlorophenyl)- | | (d, J = 5.5 Hz, 2H), 3.39 | |
| | 3-(hydroxymethyl)- | | (s, 3H), 2.32 (s, 3H). | |
| | N,5-dimethyl-1H- | | | |
| | indole-2- | | | |
| | carboxamide | | | |
| Example 26 Method 2 | 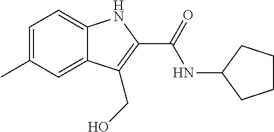 [SMILES: Cc1ccc2[nH]c(C(=O)NC3CCCC3)c(CO)c2c1] | white solid, yield: 42% | ¹H NMR (400 MHz, DMSO-d₆) δ 11.37 (s, 1H), 8.84 (d, J = 6.8 Hz, 1H), 7.45 (s, 1H), 7.29 (d, J = 8.0 Hz, 1H), 7.01 (dd, J = 8.0, 1.2 Hz, 1H), 5.84 (s, 1H), 4.80 (d, J = 4.0 Hz, 2H), 4.26 (t, J = 6.4 Hz, 1H), 2.38 (s, | 271.2 |
| | N-cyclopenty1-3- | | 3H), 1.95-1.90 (m, 2H), | |
| | (hydroxymethyl)-5- | | 1.73-1.49 (m, 6H). | |
| | methyl-1H-indole-2- | | | |
| | carboxamide | | | |
| Example 27 Method 2 | 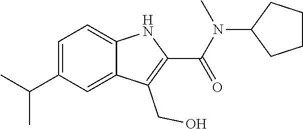 [SMILES: CC(C)c1ccc2[nH]c(C(=O)N(C)C3CCCC3)c(CO)c2c1] | white solid, yield: 60% | ¹H NMR (300 MHz, DMSO-d₆) δ 11.18 (s, 1H), 7.55 (s, 1H), 7.53 (d, J = 11.2 Hz, 1H), 7.28 (dd, J = 11.2, 1.2 Hz, 1H), 4.82 (t, J = 4.4 Hz, 1H), 4.60 (d, J = 4.4 Hz, 2H), 4.50 (brs, 1H), | 313.2 |
| | N-cyclopenty1-3- | | 2.99-2.90 (m, 1H), 1.79- | |
| | (hydroxymethyl)-5- | | 1.50 (m, 8H), 1.28 (d, J = | |
| | isopropyl-N-methyl- | | 9.2 Hz, 6H). | |
| | 1H-indole-2- | | | |
| | carboxamide | | | |
| Example 28 Method 2 | 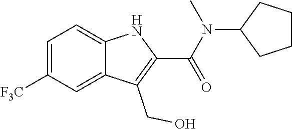 [SMILES: CN(C(=O)c1[nH]c2ccc(C(F)(F)F)cc2c1CO)C1CCCC1] | white solid, yield: 59% | ¹H NMR (400 MHz, DMSO-d₆) δ 11.79 (s, 1H), 8.10 (s, 1H), 7.58- 7.37 (m, 2H), 5.04 (t, J = 5.2 Hz, 1H), 4.79-4.10 (m, 3H), 2.88 (s, 3H), 1.83-1.40 (m, 8H). | 339.1 |
| | N-cyclopentyl-3- | | | |
| | (hydroxymethyl)-N- | | | |
| | methyl-5- | | | |
| | (trifluoromethyl)-1H- | | | |
| | indole-2- | | | |
| | carboxamide | | | |
| Example 29 Method 2 | 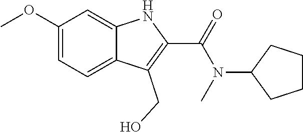 [SMILES: COc1ccc2c(CO)c(C(=O)N(C)C3CCCC3)[nH]c2c1] | white solid, yield: 66% | ¹H NMR (400 MHz, DMSO-d₆) δ 11.08 (s, 1H), 7.57 (d, J = 8.7 Hz, 1H), 6.80 (d, J = 2.3 Hz, 1H), 6.69 (dd, J = 8.7, 2.3 Hz, 1H), 4.80 (s, 1H), 4.62-4.43 (m, 3H), 3.76 (s, 3H), 2,88 (s, 3H), 1.81-1.43 (m, | 301.2 |
| | N-cyclopentyl-3- | | 8H). | |
| | (hydroxymethyl)-6- | | | |
| | methoxy-N-methyl- | | | |
| | 1H-indole-2- | | | |
| | carboxamide | | | |
| Example 30 Method 2 | 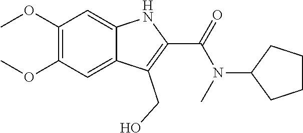 [SMILES: COc1cc2[nH]c(C(=O)N(C)C3CCCC3)c(CO)c2cc1OC] | white solid, yield: 27% | ¹H NMR (400 MHz, DMSO-d₆) δ 10.96 (s, 1H), 7.18 (s, 1H), 6.83 (s, 1H), 4.80 (t, J = 5.3 Hz, 1H), 4.60-4.42 (m, 3H), 3.77 (d, J = 1.7 Hz, 6H), 2.87 (s, 3H), 1.84- 1.41 (m, 8H). | 315.2 [M − OH]⁺ |
| | N-cyclopentyl-3- | | | |
| | (hydroxymethyl)-5,6- | | | |
| | dimethoxy-N-methyl- | | | |
| | 1H-indole-2- | | | |
| | carboxamide | | | |
| Example 31 Method 2 | 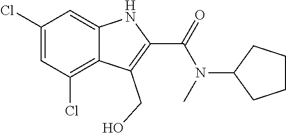 [SMILES: CN(C(=O)c1[nH]c2cc(Cl)cc(Cl)c2c1CO)C1CCCC1] | white solid, yield: 35% | ¹H NMR (400 MHz, DMSO-d₆) δ 11.89 (s, 1H), 7.36 (d, J = 1.8 Hz, 1H), 7.15 (d, J = 1.8 Hz, 1H), 4.78-4.57 (m, 3H), 4.11 (brs, 1H), 2.86 (s, 3H), 1.86-1.36 (m, 8H). | 339.1 |
| | 4,6-dichloro-N- | | | |
| | cyclopentyl-3- | | | |
| | (hydroxymethyl)-N- | | | |
| | methyl-1H-indole-2- | | | |
| | carboxamide | | | |
| Example 32 Method 2 | 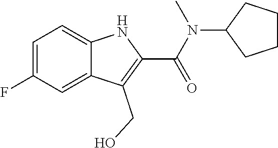 [SMILES: CN(C(=O)c1[nH]c2ccc(F)cc2c1CO)C1CCCC1] | white solid, yield: 55% | ¹H NMR (400 MHz, DMSO-d₆) δ 11.41 (s, 1H), 7.42 (dd, J = 10.0, 2.6 Hz, 1H), 7.32 (dd, J = 8.9, 4.6 Hz, 1H), 6.99 (td, J = 9.2, 2.6 Hz, 1H), 4.96-4.39 (m, 4H), 2.87 (s, 3H), 1.81-1.71 (m, 2H), 1.69-1.42 (m, 6H). | 273.1 [M − OH]⁺ |
| | N-cyclopentyl-5- | | | |
| | fluoro-3- | | | |
| | (hydroxymethyl)-N- | | | |
| | methyl-1H-indole-2- | | | |
| | carboxamide | | | |
| Example 33 Method 2 | 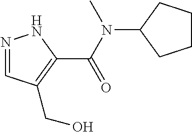 [SMILES: CN(C(=O)c1[nH]ncc1CO)C1CCCC1] | white solid, yield: 30% | ¹H NMR (400 MHz, DMSO-d₆) δ 7.66 (s, 1H), 4.87-4.67 (m, 2H), 4.38 (s, 2H), 3.00-2.78 (m, 3H), 1.80-1.40 (m, 8H). | 224.1 [M + H]⁺ |
| | N-cyclopentyl-4- | | | |
| | (hydroxymethyl)-N- | | | |
| | methyl-1H-pyrazole- | | | |
| | 5-carboxamide | | | |
| Example 34 Method 2 | 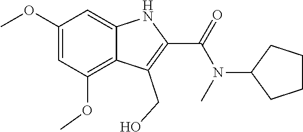 [SMILES: COc1cc(OC)c2c(CO)c(C(=O)N(C)C3CCCC3)[nH]c2c1] | white solid, yield: 66% | ¹H NMR (400 MHz, DMSO-d₆) δ 10.68 (s, 1H), 6.72 (s, 1H), 6.40 (s, 1H), 4.93-4.81 (m, 2H), 4.73 (d, J = 5.8 Hz, 2H), 3.90 (s, 3H), 3.83 (s, 3H), 3.03 (s, 3H), 1.88-1.54 (m, 8H). | 333.2 [M + H]⁺ |
| | N-cyclopentyl-3- | | | |
| | (hydroxymethyl)-4,6- | | | |
| | dimethoxy-N-methyl- | | | |
| | 1H-indole-2- | | | |
| | carboxamide | | | |
| Example 35 Method 2 | 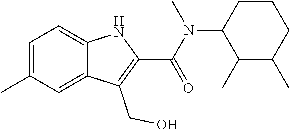 | white solid, yield: 20% | ¹H NMR (400 MHz, DMSO-d₆) δ 11.15- 10.98 (m, 1H), 7.23- 6.95 (m, 3H), 4.74-3.67 (m, 4H), 2.92-2.79 (m, 3H), 2.37-2.24 (m, 3H), 1.79-0.75 (m, 14H). | 329.2 [M + H]⁺ |
| | N-(2,3- | | | |
| | dimethylcyclohexyl)- | | | |
| | 3-(hydroxymethyl)- | | | |
| | N,5-dimethyl-1H- | | | |
| | indole-2- | | | |
| | carboxamide | | | |
| Example 36 Method 2 | 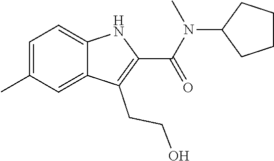 [SMILES: Cc1ccc2[nH]c(C(=O)N(C)C3CCCC3)c(CCO)c2c1] | white solid, yield: 8.1% | ¹H NMR (400 MHz, CD₃OD-d₄) δ 7.38 (s, 1H), 7.24 (d, J = 8.4 Hz, 1H), 7.02 (dd, J = 8.2, 1.7 Hz, 1H), 4.49 (brs, 1H), 3.74 (t, J = 7.2 Hz, 2H), 3.03-2.93 (m, 5H), 2.42 (s, 3H), 1.92-1.53 (m, 8H). | 301.2 [M + H]⁺ |
| | N-cyclopentyl-3-(2- | | | |
| | hydroxyethyl)-N,5- | | | |
| | dimethyl-1H-indole- | | | |
| | 2-carboxamide | | | |
| Example 37 Method 2 | 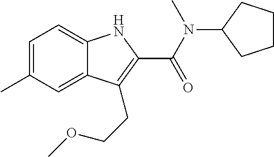 [SMILES: COCCc1c(C(=O)N(C)C2CCCC2)[nH]c2ccc(C)cc12] | white solid, yield: 13% | ¹H NMR (400 MHz, CD₃OD-d₄) δ 7.37 (s, 1H), 7.23 (d, J = 8.3 Hz, 1H), 7.01 (dd, J = 8.3, 1.6 Hz, 1H), 4.62-4.39 (m, 1H), 3.60 (t, J = 7.2 Hz, 2H), 3.30 (s, 3H), 3.01 (t, J = 7.2 Hz, 2H), 2.95 (s, 3H), 2.42 (s, 3H), 1.90-1.55 (m, | 315.2 [M + H]⁺ |
| | N-cyclopenty1-3-(2- | | 8H). | |
| | methoxyethyl)-N,5- | | | |
| | dimethyl-1H-indole- | | | |
| | 2-carboxamide | | | |
| Example 38 Method 2 | 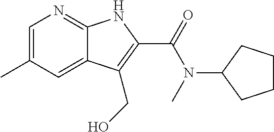 [SMILES: Cc1cnc2[nH]c(C(=O)N(C)C3CCCC3)c(CO)c2c1] | white solid, yield: 28% | ¹H NMR (400 MHz, DMSO-d₆) δ 11.66 (s, 1H), 8,11 (d, J = 2.0 Hz, 1H), 7.88 (d, J = 2.1 Hz, 1H), 4.88 (s, 1H), 4.60- 3.94 (m, 3H), 2.86 (s, 3H), 2.38 (s, 3H), 1.85- 1.34 (m, 8H). | 286.2 |
| | N-cyclopentyl-3- | | | |
| | (hydroxymethyl)- | | | |
| | N,5-dimethyl-1H- | | | |
| | pyrrolo[2,3- | | | |
| | b]pyridine-2- | | | |
| | carboxamide | | | |
| Example 39 Method 2 | 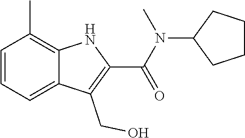 [SMILES: Cc1cccc2c(CO)c(C(=O)N(C)C3CCCC3)[nH]c12] | white solid, yield: 53% | ¹H NMR (400 MHz, DMSO-d₆) δ 11.18 (s, 1H), 7.52-7.50 (m, 1H), 7.96-7.93 (m, 2H), 4.78 (t, J = 4.8 Hz, 1H), 4.57-4.56 (m, 3H), 2.88 (s, 3H), 2.44 (s, 3H), 1.76-1.46 (m, 8H) | 309.2 [M + Na]⁺ |
| | N-cyclopentyl-3- | | | |
| | (hydroxymethyl)- | | | |
| | N,7-dimethyl-1H- | | | |
| | indole-2- | | | |
| | carboxamide | | | |
| Example 40 Method 2 | 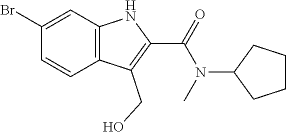 [SMILES: CN(C(=O)c1[nH]c2cc(Br)ccc2c1CO)C1CCCC1] | white solid, yield: 58% | ¹H NMR (400 MHz, DMSO-d₆) δ 11.48 (s, 1H), 7.65 (d, J = 8.4 Hz, 1H), 7.49 (d, J = 1.6 Hz, 1H), 7.19-7.16 (m, 1H), 4.91 (t, J = 5.2 Hz, 1H), 4.58-4.56 (m, 3H), 2.87 (s, 3H), 1.78- 1.45 (m, 8H). | 373.1 [M + Na]⁺ |
| | 6-bromo-N- | | | |
| | cyclopentyl-3- | | | |
| | (hydroxymethyl)-N- | | | |
| | methyl-1H-indole-2- | | | |
| | carboxamidc | | | |
| Example 41 Method 2 | 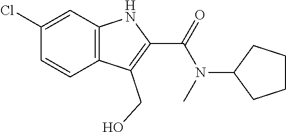 [SMILES: CN(C(=O)c1[nH]c2cc(Cl)ccc2c1CO)C1CCCC1] | white solid, yield: 45% | ¹H NMR (400 MHz, DMSO--d₆) δ 11.47 (s, 1H), 7.70 (d, J = 8.4 Hz, 1H), 7.35 (d, J = 1.6 Hz, 1H), 7.07-7.04 (m, 1H), 4.90 (t, J = 4.8 Hz, 1H), 4.58-4.57 (m, 3H), 2.87 (s, 3H),1.78- 1.48 (m, 8H) | 329.1 [M + Na]⁺ |
| | 6-chloro-N- | | | |
| | cyclopentyl-3- | | | |
| | (hydroxymethyl)-N- | | | |
| | methyl-1H-indole-2- | | | |
| | carboxamide | | | |
| Example 42 Method 2 | 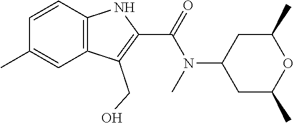 | white solid, yield: 57% | ¹H NMR (400 MHz, DMSO-d₆) δ 11.14 (s, 1H), 7.47 (s, 1H), 7.23 (d, J = 8.4 Hz, 1H), 6.99 (dd, J = 8.4, 1,2 Hz, 1H), 4.76 (d, J = 5.2 Hz, 1H), 4.60-4.56 (m, 2H), 3.85- 3.80 (m, 1H), 3.45-3.38 | 353.2 [M + Na]⁺ |
| | N-((2R,6S)-2,6- | | (m, 2H), 3.07-3.56 (m, | |
| | dimethyltetrahydro- | | 3H), 2.38 (s, 3H), 1.66- | |
| | 2H-pyran-4-yl)-3- | | 1.62 (m, 2H), 1.44-1.36 | |
| | (hydroxymethyl)- | | (m, 2H), 1.12-1.07 (m, | |
| | N,5-dimethyl-1H- | | 6H). | |
| | indole-2- | | | |
| | carboxamide | | | |
| Example 43 Method 2 | 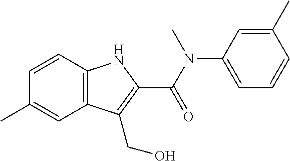 | white solid, yield: 71% | ¹H NMR (400 MHz, CD₃OD-d₄) δ 7.40 (s, 1H), 7.10-7.07 (m, 3H), 7.00-6.95 (m, 3H), 4.68 (s, 2H), 3.31-3.30 (m, 3H), 2.36 (s, 3H), 2.23 (s, 3H). | 307.1 |
| | 3-(hydroxymethyl)- | | | |
| | N,5-dimethyl-N-(m- | | | |
| | tolyl)-1H-indole-2- | | | |
| | carboxamidc | | | |
| Example 44 Method 2 | 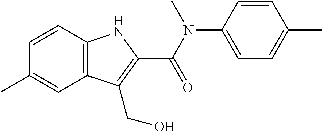 | white solid, yield: 52% | ¹H NMR (400 MHz, DMSO-d₆) δ 10.96 (s, 1H), 7.34 (s, 1H), 7.13 (d, J = 8.4 Hz, 1H), 7.07-7.01 (m, 4H), 6.90 (dd, J = 8.4, 1.2 Hz, 1H), 4.58 (t, J = 5.6 Hz, 1H), 4.34 (d, J = 5.6 Hz, 2H), 3.38 (s, 3H), 2.31 (s, | 291.2 [M − OH]⁺ |
| | 3-(hydroxymethyl)- | | 3H), 2.19 (s, 3H). | |
| | N,5-dimethyl-N-(p- | | | |
| | tolyl)-1H-indole-2- | | | |
| | carboxamide | | | |
| Example 45 Method 1 | 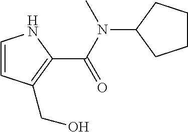 [SMILES: CN(C(=O)c1[nH]ccc1CO)C1CCCC1] | white solid, yield: 7% | ¹H NMR (400 MHz, DMSO-d₆) δ 10.98 (s, 1H), 6.74 (s, 1H), 6.08 (s, 1H), 4.50 (t, J = 8.0 Hz, 1H), 4.32 (s, 2H), 2.83 (s, 3H), 1.78-1.47 (m, 8H). | 221.1 |
| | N-cyclopentyl-3- | | | |
| | (hydroxymethyl)-N- | | | |
| | mcthyl-1H-pyrrole-2- | | | |
| | carboxamide | | | |

### Preparation of N-cyclopentyl-N,5-dimethyl-1H-benzo[d]imidazole-2-carboxamide (46) Method 3

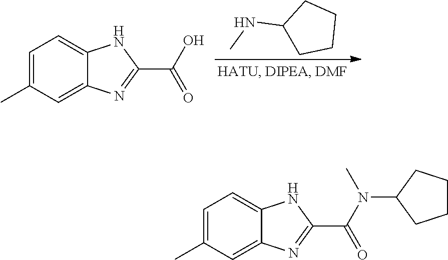

### Step 1. N-cyclopentyl-N,5-dimethyl-1H-benzo[d]imidazole-2-carboxamide

To a solution of 5-methyl-1H-benzo[d]imidazole-2-carboxylic acid (150 mg, 0.85 mmol) in DMF (10 mL) was added N-methylcyclopentanamine (76 mg, 0.76 mmol), DIPEA (0.47 mL, 2.55 mmol) and HATU (387 mg, 1.02 mmol) at rt. The mixture was stirred at rt for 3 hours. To the mixture was added water (20 mL), and the mixture was extracted with EA (30 mL*2). The combined organic layers were washed with water (20 mL*2) and brine (20 mL), dried over anhydrous Na₂SO₄, filtered and concentrated. The residue was purified by prep-HPLC to give N-cyclopentyl-N,5-dimethyl-1H-benzo[d]imidazole-2-carboxamide (33.5 mg, 15.2%) as a white solid. MS (ESI) m/z 258 [M+H]⁺.

¹H NMR (400 MHz, DMSO-d₆) δ 12.88 (s, 1H), 7.63-7.48 (m, 1H), 7.42-7.27 (m, 1H), 7.16-7.03 (m, 1H), 5.79-4.91 (m, 1H), 3.41-2.93 (m, 3H), 2.42 (s, 3H), 1.98-1.41 (m, 8H).

Examples (Compounds) 47-59, 61-65 were synthesized using a method similar to that used in Example 46.

| Example No. | | Appearance | | |
|---|---|---|---|---|
| (Compound No.) | | and | | MS (m/z) |
| Method | Structure and Name | Yield | ¹H NMR Data | [M + H]⁺ |
| Example 47 Method 3 | 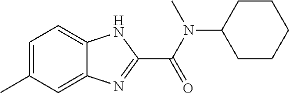 [SMILES: Cc1ccc2[nH]c(C(=O)N(C)C3CCCCC3)nc2c1] | white solid, yield: 16% | ¹H NMR (400 MHz, DMSO-d₆) δ 12.86 (s, 1H), 7.60-7.50 (m, 1H), 7.40-7.05 (m, 2H), 4.42-4.36 (m, 1H), 3.40 (s, 1.5H), 2.94 (s, 1.5H), 2.42 (s, 3H), 1.83-1.20 (m, 10H). | 272.2 |
| | N-cyclohexyl-N,5- | | | |
| | dimethyl-1H- | | | |
| | benzo[d]imidazole-2- | | | |
| | carboxamide | | | |
| Example 48 Method 3 | 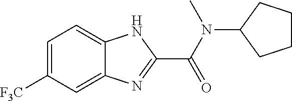 [SMILES: CN(C(=O)c1nc2cc(C(F)(F)F)ccc2[nH]1)C1CCCC1] | white solid, yield: 16% | ¹H NMR (400 MHz, CDCl₃) δ 11.49 (bs, 1H), 8.04 (s, 1H), 7.80-7.70 (m, 1H), 7.62- 7.56 (m, 1H), 6.35-5.05 (m, 1H), 3.65 (s, 1.5H), 3.10 (s, 1.5H), 2.15-1.62 (m, 8H). | 312.1 |
| | N-cyclopentyl-N- | | | |
| | methyl-5- | | | |
| | (trifluoromethyl)-1H- | | | |
| | benzo[d]imidazole-2- | | | |
| | carboxamide | | | |
| Example 49 Method 3 | 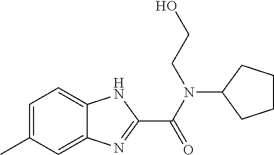 [SMILES: Cc1ccc2[nH]c(C(=O)N(CCO)C3CCCC3)nc2c1] | white solid, yield: 14% | ¹H NMR (400 MHz, DMSO-d₆) δ 12.90 (s, 1H), 7.70-6.97 (m, 3H), 5.82-4.32 (m, 0.5H), 4.99-4.82 (m, 1H), 4.45-4.41 (m, 1H), 4.04 (s, 1H), 3.60 (q, J = 8.0, 7.4 Hz, 2H), 3.41 (t, J = 7.0 Hz, 1H), 2.42 (s, 3H), 2.09-1.35 (m, 8H). | 288.2 |
| | N-cyclopentyl-N-(2- | | | |
| | hydroxyethyl)-5- | | | |
| | methyl-1H- | | | |
| | benzo[d]imidazole-2- | | | |
| | carboxamide | | | |
| | benzo[d]imidazole-2- | | | |
| | carboxamide | | | |
| Example 50 Method 3 | 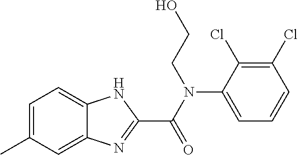 [SMILES: Cc1ccc2[nH]c(C(=O)N(CCO)c3cccc(Cl)c3Cl)nc2c1] | white solid, yield: 14% | 1H NMR (400 MHz, DMSO-d₆) δ 7.43-7.26 (m, 1H), 7.22-7.02 (m, 3H), 6.93-6.89 (m, 1H), 6.84-6.79 (m, 1H), 5.89 (t, J = 5.9 Hz, 1H), 4.52 (q, J = 5.7 Hz, 2H), 3.75-3.56 (m, 2H), 2.37-2.33 (m, 3H). | 364.1 |
| | N-(2,3- | | | |
| | dichlorophenyl)-N- | | | |
| | (2-hydroxyethyl)-5- | | | |
| | methyl-1H- | | | |
| | benzo[d]imidazole-2- | | | |
| | carboxamide | | | |
| Example 51 Method 3 | 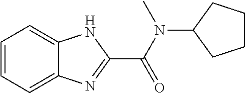 [SMILES: CN(C(=O)c1nc2ccccc2[nH]1)C1CCCC1] | white solid, yield: 30% | ¹H NMR (400 MHz, DMSO-d₆) δ 10.70 (brs, 1H), 7.63 (dd, J = 6.1, 3.3 Hz, 2H), 7.27 (dd, J = 6.1, 3.2 Hz, 2H), 5.72-4.92 (m, 1H), 2.94- 3.41 (m, 3H), 1.95-1.49 (m, 8H). | 244.1 |
| | N-cyclopentyl-N- | | | |
| | methyl-1H- | | | |
| | benzo[d]imidazole-2- | | | |
| | carboxamide | | | |
| Example 52 Method 3 | 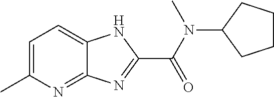 [SMILES: Cc1ccc2[nH]c(C(=O)N(C)C3CCCC3)nc2n1] | white solid, yield: 1% | ¹H NMR (400 MHz, MeOH-d₄) δ 8.40 (s, 1H), 7.86 (d, J = 8.2 Hz, 1H), 7.17 (d, J = 8.2 Hz, 1H), 4.97-4.75 (m, 1H), 3.33-2.92 (m, 2H), 2.54 (s, 3H), 1.95-1.45 (m, 8H). | 259.2 |
| | N-cyclopentyl-N,5- | | | |
| | dimethyl-1H- | | | |
| | imidazo[4,5-b] | | | |
| | pyridine-2-carboxamide | | | |
| Example 53 Method 3 Mixture of Enantiomers | 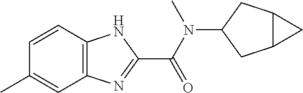 [SMILES: Cc1ccc2[nH]c(C(=O)N(C)C3CC4CC4C3)nc2c1] | white solid, yield: 32% | ¹H NMR (400 MHz, DMSO-d₆) δ 12.41 (s, 1H), 7.59-7.27 (m, 2H), 7.10 (d, J = 7.2 Hz, 1H), 6.19-5.26 (m, 1H), 2.83 (s, 2H), 2.51 (s, 1H), 2.42 (s, 3H), 2.23-2.17 (m, 2H), 1.49- 1.43 (m, 2H), 1.29-1.24 (m, 2H), 0.92-0.86 (m, 1H), 0.30 (s, 1H). | 270.1 |
| | N- | | | |
| | (bicyclo[3.1.0]hexan- | | | |
| | 1H- | | | |
| | benzo[d]imidazole-2- | | | |
| | carboxamide | | | |
| Example 54 Method 3 | 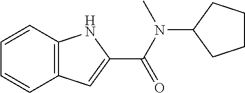 [SMILES: CN(C(=O)c1cc2ccccc2[nH]1)C1CCCC1] | white solid, yield: 11.7% | ¹H NMR (400 MHz, MeOD-d₄) δ 7.61 (d, J = 8.0 Hz, 1H), 7.42 (dd, J = 8.0 Hz, 0.8 Hz, 1H), 7.22-7.18 (m, 1H), 7.07-7.03 (m, 1H), 6.83 (s, 1H), 4.97-4.86 (m, 1H), 3.12 (s, 3H), 1.93-1.91 (m, 2H), 1.82-1.63 (m, 6H). | 243.1 |
| | N-cyclopentyl-N- | | | |
| | methyl-1H-indole-2- | | | |
| | carboxamide | | | |
| Example 55 Method 3 | 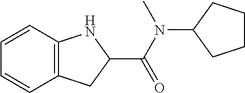 [SMILES: CN(C(=O)C1Cc2ccccc2N1)C1CCCC1] | off-red solid, yield: 17% | ¹H NMR (400 MHz, MeOD-d₄) δ 7.26-7.22 (m, 2H), 7.10-7.06 (m, 2H), 5.07-4.89 (m, 1H), 4.85 (s, 0.4H), 4.37-4.35 (m, 0.6), 3.67- 3.64 (m, 1H), 3.33-3.20 (m, 1H), 3.16 (s, 1.3H), 2.89 (s, 1.7H), 1.74-1.64 (m, 8H). | 245.1 |
| | N-cyclopentyl-N- | | | |
| | methylindoline-2- | | | |
| | carboxamide | | | |
| Example 56 Method 3 | 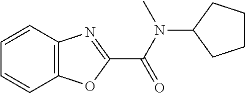 [SMILES: CN(C(=O)c1nc2ccccc2o1)C1CCCC1] | colorless oil, yield: 20.6% | ¹H NMR (400 MHz, DMSO-d₆) δ 7.93-7.81 (m, 2H), 7.58-7.44 (m, 2H), 4.94-4.55 (m, 1H), 3.14 (s, 1.2H), 2.95 (s, 1.8H), 1.95-1.40 (m, 8H). | 245.1 |
| | N-cyclopentyl-N- | | | |
| | methylbenzo[d]oxazole-2- | | | |
| | carboxamide | | | |
| Example 57 Method 3 | 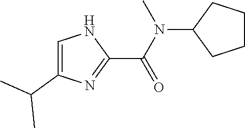 [SMILES: CC(C)c1c[nH]c(C(=O)N(C)C2CCCC2)n1] | yellow solid, yield: 0.9% | ¹H NMR (400 MHz, CDCl₃) δ 7.05-6.87 (m, 1H), 6.00-4.80 (m, 1H), 3.50-2.75 (m, 4H), 2.05-1.52 (m, 8H), 1.27 (d, J = 6.4 Hz, 6H). | 236.2 |
| | N-cyclopentyl-4- | | | |
| | isopropyl-N-methyl- | | | |
| | 1H-imidazole-2- | | | |
| | carboxamide | | | |
| Example 58 Method 3 | 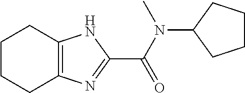 [SMILES: CN(C(=O)c1nc2c([nH]1)CCCC2)C1CCCC1] | white solid, yield: 8.89% | ¹H NMR (400 MHz, CDCl₃) δ 10.66 (bs, 1H), 6.40-6.15 (m, 0.4H), 5.20-4.90 (m, 0.6H), 3.60-2.80 (m, 3H), 2.70-2.50 (m, 4H), 2.10-1.50 (m, 12H). | 248.2 |
| | N-cyclopentyl-N- | | | |
| | methyl-4,5,6,7- | | | |
| | tetrahydro-1H- | | | |
| | benzo[d]imidazole-2- | | | |
| | carboxamide | | | |
| Example 59 Method 3 | 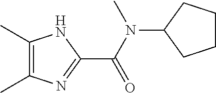 [SMILES: Cc1nc(C(=O)N(C)C2CCCC2)[nH]c1C] | white solid, yield: 5.89% | ¹H NMR (400 MHz, CDCl₃) δ 10.95 (bs, 1H), 6.23-5.02 (m, 1H), 3.52-2.95 (m, 2H), 2.20 (s, 6H), 2.05-1.81 (m, 2H), 1.77-1.55 (m, 6H). | 222.2 |
| | N-cyclopentyl-N,4,5- | | | |
| | trimethyl-1H- | | | |
| | imidazole-2- | | | |
| | carboxamide | | | |
| Example 61 Method 3 | 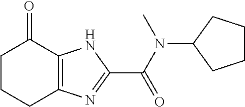 [SMILES: CN(C(=O)c1nc2c([nH]1)C(=O)CCC2)C1CCCC1] | white solid, yield: 34% | ¹H NMR (400 MHz, CDCl₃) δ 12.1-10.6 (m, 1H), 6.40-4.90 (m, 1H), 3.70-2.95 (m, 3H), 2.95-2.85 (m, 2H), 2.64-2.54 (m, 2H), 2.26-2.15 (m, 2H), 2.12-1.54 (m, 8H). | 262.1 |
| | N-cyclopentyl-N- | | | |
| | methyl-7-oxo- | | | |
| | 4,5,6,7-tetrahydro- | | | |
| | 1H- | | | |
| | benzo[d]imidazole-2- | | | |
| | carboxamide | | | |
| Example 62 Method 3 | 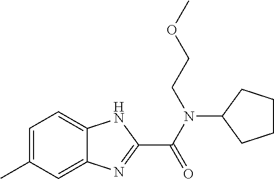 [SMILES: COCCN(C(=O)c1nc2cc(C)ccc2[nH]1)C1CCCC1] | white solid, yield: 55% | ¹H NMR (400 MHz, CDCl₃) δ 11.5-11.0 (m, 1H), 7.77-7.52 (m, 1H), 7.46-7.28 (m, 1H), 7.22-7.06 (m, 1H), 6.50-4.60 (m, 1H), 4.48-4.36 (m, 1H), 3.78-3.62 (m, 3H), 3.43- 3.33 (m, 3H), 2.48 (s, 3H), 2.15-1.57 (m, 8H). | 302.2 |
| | N-cyclopentyl-N-(2- | | | |
| | methoxyethyl)-5- | | | |
| | methyl-1H- | | | |
| | benzo[d]imidazole-2- | | | |
| | carboxamide | | | |
| Example 63 Method 3 | 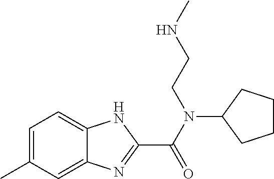 [SMILES: CNCCN(C(=O)c1nc2cc(C)ccc2[nH]1)C1CCCC1] | white solid, yield: 17% | ¹H NMR (400 MHz, CDCl₃) δ 8.71 (s, 1H), 7.49 (d, J = 8.0 Hz, 1H), 7.37 (s, 1H), 7.11 (d, J = 8.0 Hz, 1H), 4.45-3.35 (m, 5H), 3.30-2.67 (m, 3H), 2.46 (s, 3H), 2.17-1.50 (m, 8H). | 301.2 |
| | N-cyclopentyl-5- | | | |
| | methyl-N-(2- | | | |
| | (methylamino)ethyl)-1H- | | | |
| | benzo[d]imidazol-2- | | | |
| | carboxamide | | | |
| Example 64 Method 3 | 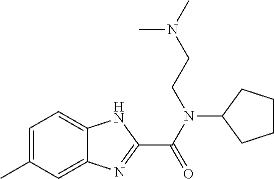 [SMILES: Cc1ccc2[nH]c(C(=O)N(CCN(C)C)C3CCCC3)nc2c1] | white solid, yield: 27% | ¹H NMR (400 MHz, CDCl₃) δ 10.68 (bs, 1H), 7.65-7.30 (m, 2H), 7.15-7.13 (m, 1H), 6.31-4.70 (m, 1H), 4.42-4.20 (m, 1H), 3.70- 3.50 (m, 1H), 2.75-2.70 (m, 2H), 2.49 (s, 3H), 2.39 (s, 6H), 2.10-1.75 (m, 8H). | 315.1 |
| | N-cyclopentyl-N-(2- | | | |
| | (dimethylamino)ethyl)- | | | |
| | 5-methyl-1H-benzo[d] | | | |
| | imidazole-2-carboxamide | | | |
| Example 65 Method 3 | 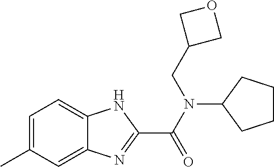 [SMILES: Cc1ccc2[nH]c(C(=O)N(CC3COC3)C3CCCC3)nc2c1] | white solid, yield: 27% | ¹H NMR (400 MHz, CDCl₃) δ 7.65-7.41 (m, 1H), 7.39-7.16 (m, 1H), 7.15-7.01 (m, 1H), 4.97-4.80 (m, 1H), 4.53-4.28 (m, 1H), 4.26-4.08 (m, 1H), 3.62-3.30 (m, 3H), 2.92- 2.52 (m, 2H), 2.42 (d, J = 7.2 Hz, 3H), 2.10- 1.30 (m, 8H). | 314.1 |
| | N-cyclopentyl-5- | | | |
| | methyl-N-(oxetan-3- | | | |
| | ylmethyl)-1H- | | | |
| | benzo[d]imidazole-2- | | | |
| | carboxamide | | | |

### Preparation of 3-(aminomethyl)-N-cyclopentyl-N,5-dimethyl-1H-indole-2-carboxamide (68)

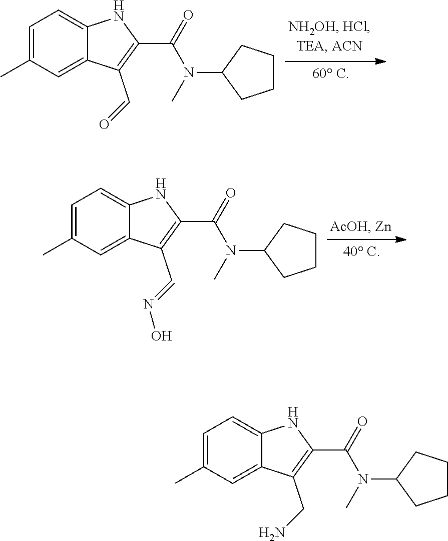 [SMILES: Cc1ccc2[nH]c(C(=O)N(C)C3CCCC3)c(C=O)c2c1 ; Cc1ccc2[nH]c(C(=O)N(C)C3CCCC3)c(/C=N/O)c2c1 ; Cc1ccc2[nH]c(C(=O)N(C)C3CCCC3)c(CN)c2c1]

Step 1. N-cyclopentyl-3-((hydroxyimino)methyl)-N,5-dimethyl-1H-indole-2-carboxamide To a solution of N-cyclopentyl-3-formyl-N,5-dimethyl-1H-indole-2-carboxamide (1.0 g, 3.20 mmol) in ACN (15 mL) was added NH₂OH·HCl (986 mg, 17.6 mmol) and TEA (1.8 g, 17.6 mmol) at 0° C. and stirred at 60° C. overnight. To the mixture was added water (50 mL), and the mixture was extracted with EA (80 mL*2). The combined organic layers were washed with water (50 mL*2) and brine (100 mL), dried over Na₂SO₄, filtered and concentrated under vacuum. The residue was purified by FCC (PE:EA=1:1) to give N-cyclopentyl-3-((hydroxyimino)methyl)-N,5-dimethyl-1H-indole-2-carboxamide (900 mg, 86%) as a white solid. MS (ESI) m/z 300 [M+H]⁺.

### Step 2. 3-(aminomethyl)-N-cyclopentyl-N,5-dimethyl-1H-indole-2-carboxamide

To a solution of N-cyclopentyl-3-((hydroxyimino)methyl)-N,5-dimethyl-1H-indole-2-carboxamide (300 mg, 1.00 mmol) in AcOH (15 mL) was added Zn powder (325 mg, 5.00 mmol) at rt and stirred at 40° C. for 16 hours. The mixture was added water (20 mL), and the mixture was extracted with EA (30 mL*2). The combined organic layers were washed with water (20 mL*2) and brine (20 mL), dried over Na₂SO₄, filtered and concentrated under vacuum. The residue was purified by prep-HPLC to give 3-(aminomethyl)-N-cyclopentyl-N,5-dimethyl-1H-indole-2-carboxamide (formate, 14.8 mg, 6%) as a white solid. MS (ESI) m/z 269 [M−NH₂]⁺.

¹H NMR (400 MHz, DMSO-d₆) δ 11.40 (s, 1H), 8.40 (s, 1H), 7.56 (s, 1H), 7.28 (d, J=8.4 Hz, 1H), 7.03 (d, J=8.4 Hz, 1H), 4.49 (brs, 2H), 3.99 (d, J=3.6 Hz, 2H), 3.87-3.56 (m, 1H), 2.90 (s, 3H), 2.39 (s, 3H), 1.89-1.35 (m, 8H).

### Preparation of 3-cyano-N-(2,3-dichlorophenyl)-N,5-dimethyl-1H-indole-2-carboxamide (69) Method 4

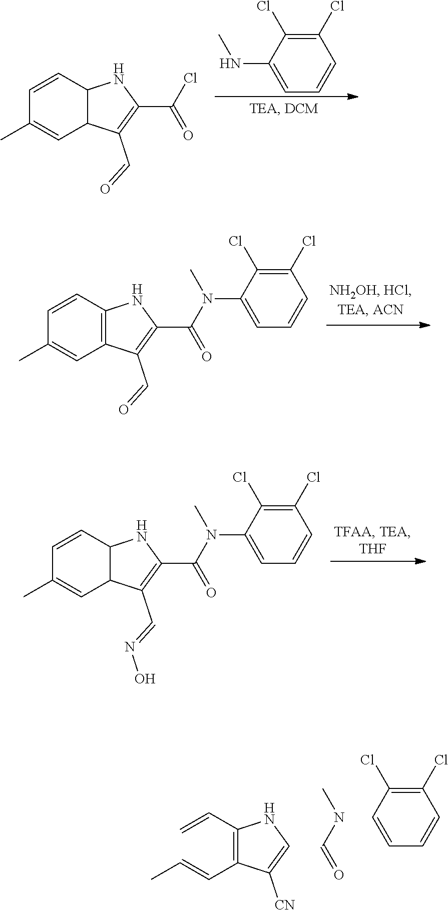

### Step 1. N-(2,3-dichlorophenyl)-3-formyl-N,5-dimethyl-1H-indole-2-carboxamide

To a solution of 2,3-dichloro-N-methylaniline (794 mg, 4.51 mmol) in DCM (10 mL) was added TEA (1.37 g, 13.5 mmol) and a solution of 3-formyl-5-methyl-1H-indole-2-carbonyl chloride (1 g, 4.52 mmol) in DCM (10 mL) slowly at 0° C. and the mixture was stirred at rt for 2 hours. To the mixture was added water (10 mL), and the mixture was extracted with EA (30 mL*2) and concentrated under vacuum. The residue was purified by FCC (PE/EA=1/1) to give N-(2,3-dichlorophenyl)-3-formyl-N,5-dimethyl-1H-indole-2-carboxamide (200 mg, 12.3%) as a yellow solid. MS (ESI) m/z 361 [M+H]⁺.

### Step 2. N-(2,3-dichlorophenyl)-3-((hydroxyimino)methyl)-N,5-dimethyl-1H-indole-2-carboxamide

The title compound was prepared in 57% yield from N-(2,3-dichlorophenyl)-3-formyl-N,5-dimethyl-1H-indole-2-carboxamide using general procedure of N-cyclopentyl-3-((hydroxyimino)methyl)-N,5-dimethyl-1H-indole-2-carboxamide. MS (ESI) m/z 376 [M+H]⁺.

### Step 3. 3-cyano-N-(2,3-dichlorophenyl)-N,5-dimethyl-1H-indole-2-carboxamide

The title compound was prepared in 35.2% yield from N-(2,3-dichlorophenyl)-3-((hydroxyimino)methyl)-N,5-dimethyl-1H-indole-2-carboxamide using the general procedure for preparing 3-cyano-N-cyclopentyl-N,5-dimethyl-1H-indole-2-carb oxamide. MS (ESI) m/z 358.0 [M+H]⁺.

¹H NMR (400 MHz, DMSO-d₆) δ 10.35 (brs, 1H), 7.58 (d, J=7.6 Hz, 2H), 7.38-7.29 (m, 3H), 7.12 (s, 1H), 3.39 (s, 3H), 2.37 (s, 3H).

Examples (Compounds) 70-72 were synthesized using a method similar to that used in Example 69.

| Example No. | | Appearance | | |
|---|---|---|---|---|
| (Compound No.) | | and | | MS (m/z) |
| Method | Structure and Name | Yield | ¹H NMR Data | [M + H]⁺ |
| Example 70 Method 4 | 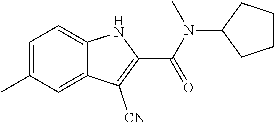 [SMILES: Cc1ccc2[nH]c(C(=O)N(C)C3CCCC3)c(C#N)c2c1] | white solid, yield: 54% | ¹H NMR (400 MHz, DMSO-d₆) δ 12.86 (s, 1H), 7.60-7.50 (m, 1H), 7.40-7.05 (m, 2H), 4.42-4.36 (m, 1H), 3.40 (s, 1.5H), 2.94 (s, 1.5H), 2.42 (s, 3H), 1.83-1.20 (m, 10H). | 282.2 |
| | 3-cyano-N- | | | |
| | cyclopentyl-N,5- | | | |
| | dimethyl-1H-indole- | | | |
| | 2-carboxamide | | | |
| Example 71 Method 4 | 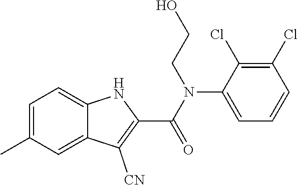 [SMILES: Cc1ccc2[nH]c(C(=O)N(CCO)c3cccc(Cl)c3Cl)c(C#N)c2c1] | white solid, yield: 54% | 1H NMR (400 MHz, DMSO-d₆) δ 9.40 (bs, 1H), 7.75-7.65 (m, 1H), 7.59-7.24 (m, 4H), 7.14-7.07 (m, 1H), 4.85 (bs, 1H), 4.25-4.10 (m, 1H), 3.80-3.41 (m, 3H), 2.45-2.30 (m, 3H). | 388.1 |
| | 3-cyano-N-(2,3- | | | |
| | dichlorophenyl)-N- | | | |
| | (2-hydroxyethyl)-5- | | | |
| | methyl-1H-indole-2- | | | |
| | carboxamide | | | |
| Example 72 Method 4 | 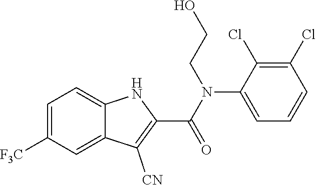 [SMILES: N#Cc1c(C(=O)N(CCO)c2cccc(Cl)c2Cl)[nH]c2ccc(C(F)(F)F)cc12] | white solid, yield: 46% | ¹H NMR (400 MHz, DMSO-d₆) δ 8.08-7.88 (m, 1H), 7.84-7.38 (m, 5H), 4.90-4.88 (m, 1H), 4.27-3.73 (m, 2H), 3.69-3.46 (m, 2H). | 441.9 |
| | 3-cyano-N-(2,3- | | | |
| | dichlorophenyl)-N- | | | |
| | (2-hydroxyethyl)-5- | | | |
| | (trifluoromethyl)-1H- | | | |
| | indole-2-carboxamide | | | |

### Preparation of 2-(((2-(cyclopentyl(methyl)carbamoyl)-5-methyl-1H-indol-3-yl)methyl)amino)thiazole-4-carboxamide (73)

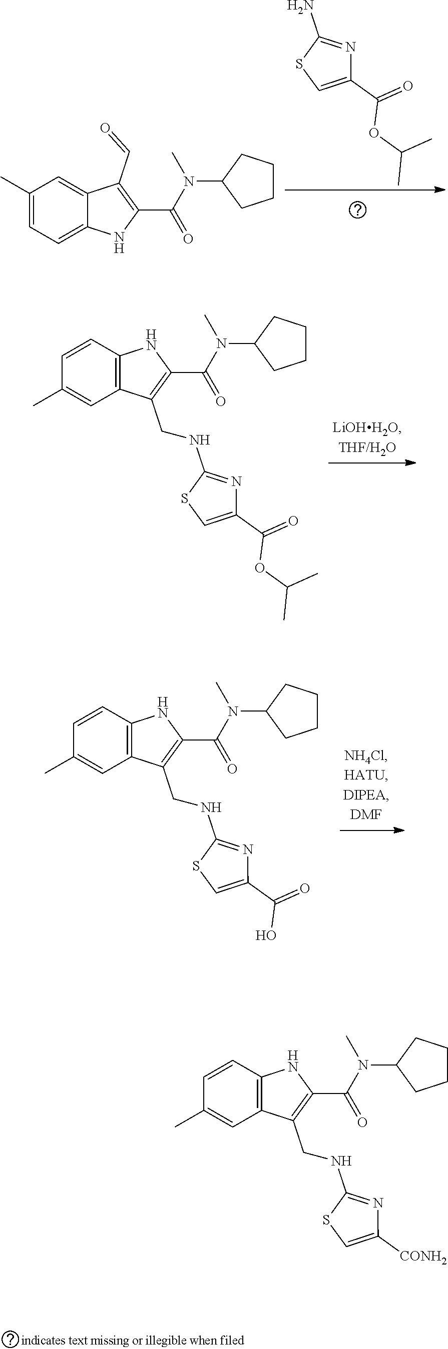

### Step 1. isopropyl 2-(((2-(cyclopentyl(methyl)carbamoyl)-5-methyl-1H-indol-3-yl)methyl)amino)thiazole-4-carboxylate

To a solution of N-cyclopentyl-3-formyl-N,5-dimethyl-1H-indole-2-carboxamide (1.0 g, 3.52 mmol) and isopropyl 2-aminothiazole-4-carboxylate (611 mg, 3.87 mmol) in THF (15 mL) was added Ti(OPr-i)₄ (4.0 g, 14.08 mmol) at room temperature and the resulted mixture was stirred at 85° C. overnight in sealed tube. NaBH₃CN (436 mg, 7.04 mmol) was added to the solution at room temperature and the reaction was stirred for 8 hours at 85° C. The mixture was concentrated under vacuum, H₂O (20 mL) was added and extracted with EA (20 mL). The combined organic layers were dried over Na₂SO₄, filtered and concentrated under vacuum. The residue was purified by FCC (PE/EA=1/1) to give isopropyl 2-(((2-(cyclopentyl(methyl)carbamoyl)-5-methyl-1H-indol-3-yl)methyl)amino)thiazole-4-carboxylate (500 mg, 31%) as a yellow solid. MS (ESI) m/z 455 [M+H]⁺.

### Step 2. 2-(((2-(cyclopentyl(methyl)carbamoyl)-5-methyl-1H-indol-3-yl)methyl)amino)thiazole-4-carboxylic Acid

A solution of isopropyl 2-(((2-(cyclopentyl(methyl)carbamoyl)-5-methyl-1H-indol-3-yl)methyl)amino)thiazole-4-carboxylate (500 mg, 1.1 mmol) and LiOH·H₂O (118 mg, 2.75 mmol) in THF/H₂O (10 mL/5 mL) was stirred at 55° C. for 5 hours. The pH of the mixture was adjusted to 5 with 1N HCl; after filtration, the cake was dried to give 2-(((2-(cyclopentyl(methyl)carbamoyl)-5-methyl-1H-indol-3-yl)methyl)amino)thiazole-4-carboxylic acid (350 mg, 77%) as a yellow solid. MS (ESI) m/z 413 [M+H]⁺.

### Step 3. 2-(((2-(cyclopentyl(methyl)carbamoyl)-5-methyl-1H-indol-3-yl)methyl)amino)thiazole-4-carboxamide

To a solution of 2-(((2-(cyclopentyl(methyl)carbamoyl)-5-methyl-1H-indol-3-yl)methyl)amino)thiazole-4-carboxylic acid (150 mg, 0.36 mmol), NH₄Cl (99 mg, 1.8 mmol) and DIPEA (140 mg, 1.08 mmol) in DMF (10 mL) was added HATU (163 mg, 0.43 mmol).

The reaction was stirred at room temperature overnight. To the mixture was added H₂O (50 mL), and the mixture was extracted with EA (30 mL*3). The combined organic layers were dried over Na₂SO₄, filtered and concentrated under vacuum. The residue was purified by prep-HPLC (NH₄HCO₃) to give 2-(((2-(cyclopentyl(methyl) carbamoyl)-5-methyl-1H-indol-3-yl)methyl)amino)thiazole-4-carboxamide (56.7 mg, 38%) as a white solid. MS (ESI) m/z 412.2 [M+H]⁺.

¹H NMR (400 MHz, DMSO-d₆) δ 11.25 (s, 1H), 8.02 (t, J=5.2 Hz, 1H), 7.47 (s, 1H), 7.36-7.23 (m, 3H), 7.16 (s, 1H), 6.99 (dd, J=8.0, 1.2 Hz, 1H), 5.00-4.40 (brs, 1H), 4.53 (d, J=5.2 Hz, 2H), 2.87 (s, 3H), 2.37 (s, 3H), 1.72-1.45 (m, 8H).

### Preparation of 2-(((2-((2,3-dichlorophenyl)(methyl)carbamoyl)-5-methyl-1H-indol-3-yl)methyl)amino)-5-isopropylthiazole-4-carboxamide (74)

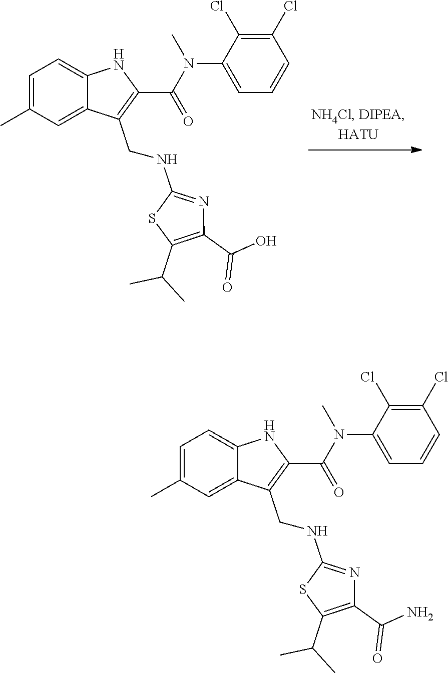 [SMILES: Cc1ccc2[nH]c(C(=O)N(C)c3cccc(Cl)c3Cl)c(CNc3nc(C(=O)O)c(C(C)C)s3)c2c1 ; Cc1ccc2[nH]c(C(=O)N(C)c3cccc(Cl)c3Cl)c(CNc3nc(C(N)=O)c(C(C)C)s3)c2c1]

### Step 1. 2-(((2-((2,3-dichlorophenyl)(methyl)carbamoyl)-5-methyl-1H-indol-3-yl)methyl)amino)-5-isopropylthiazole-4-carboxamide

To a solution of 2-(((2-((2,3-dichlorophenyl)(methyl)carbamoyl)-5-methyl-1H-indol-3-yl)methyl)amino)-5-isopropylthiazole-4-carboxylic acid (90 mg, 0.16 mmol) in DMF (10 mL) was added NH₄Cl (47 mg, 0.85 mmol), DIPEA (62 mg, 0.48 mmol) and HATU (73 mg, 0.19 mmol) at rt. The mixture was stirred at rt for 16 hours. To the mixture was added water (20 mL), and the mixture was extracted with EA (50 mL*2), dried over anhydrous Na₂SO₄, filtered and concentrated. The residue was purified by prep-HPLC to give 2-(((2-((2,3-dichlorophenyl)(methyl)carbamoyl)-5-methyl-1H-indol-3-yl)methyl)amino)-5-isopropylthiazole-4-carboxamide (44.5 mg, 49.5%) as a white solid. MS (ESI) m/z 530.1 [M+H]⁺.

¹H NMR (400 MHz, CD₃OD-d₄) δ 7.58-6.91 (m, 6H), 4.58 (brs, 2H), 4.21 (m, J=6.8 Hz, 1H), 3.40 (s, 3H), 2.37 (s, 3H), 1.22 (d, J=6.8 Hz, 6H).

### Preparation of N-cyclopentyl-N,5-dimethyl-3-((thiazol-2-yloxy)methyl)-1H-indole-2-carboxamide (75)

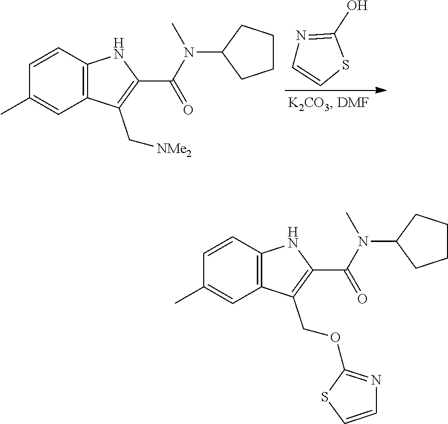

Step 1. N-cyclopentyl-N,5-dimethyl-3-((thiazol-2-yloxy)methyl)-1H-indole-2-carboxamide A mixture of N-cyclopentyl-3-((dimethylamino)methyl)-N,5-dimethyl-1H-indole-2-carboxamide (20 mg, 0.064 mmol), thiazol-2-ol (13 mg, 0.128 mmol) and K₂CO₃ (17.7 mg, 0.128 mmol) in DMF (3 mL) was stirred at 100° C. for 12 hours. To the mixture was added EA (30 mL) and the mixture was extracted with brine (20 mL*3), dried over Na₂SO₄, filtered and concentrated under vacuum. The residue was purified by prep-HPLC (NH₄HCO₃) to give N-cyclopentyl-N,5-dimethyl-3-((thiazol-2-yloxy)methyl)-1H-indole-2-carboxamide (1.4 mg, 6%) as a white solid. MS (ESI) m/z 370.2 [M+H]⁺.

¹H NMR (400 MHz, DMSO-d₆) δ 11.44 (s, 1H), 7.39 (s, 1H), 7.28 (d, J=8.0 Hz, 1H), 7.03-6.98 (m, 2H), 6.36 (d, J=6.0 Hz, 1H), 4.88 (s, 3H), 2.86 (s, 3H), 2.36 (s, 3H), 1.77-1.46 (m, 8H).

### Preparation of N-cyclopentyl-N,5-dimethyl-3-((thiazol-2-ylamino)methyl)-1H-indole-2-carboxamide (76) Method 5

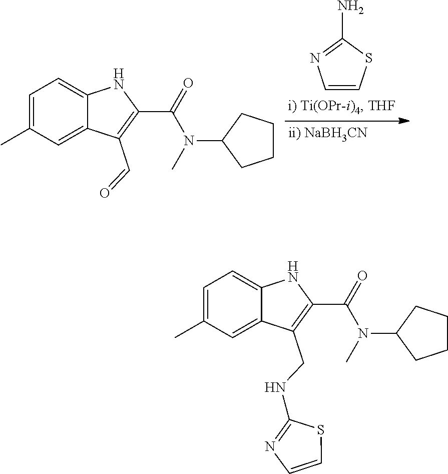 [SMILES: Cc1ccc2[nH]c(C(=O)N(C)C3CCCC3)c(C=O)c2c1 ; Nc1nccs1 ; Cc1ccc2[nH]c(C(=O)N(C)C3CCCC3)c(CNc3nccs3)c2c1]

Step 1. N-cyclopentyl-N,5-dimethyl-3-((thiazol-2-ylamino)methyl)-1H-indole-2-carboxamide To a solution of N-cyclopentyl-3-formyl-N,5-dimethyl-1H-indole-2-carboxamide (100 mg, 0.35 mmol) and thiazol-2-amine (35 mg, 0.35 mmol) in THF (10 mL) was added Ti(OPr-i)₄ (298 mg, 1.05 mmol) at room temperature and the mixture was stirred at 80° C. for 5 hours. NaBH₃CN (43.4 mg, 0.7 mmol) was added to the solution at room temperature and the resulted solution was stirred for 5 hours. The mixture was concentrated under vacuum, and was extracted with EA (20 mL) and H₂O (20 mL). The combined organic layers were dried over Na₂SO₄, filtered and concentrated under vacuum. The residue was purified by prep-HPLC to give N-cyclopentyl-N, 5-dimethyl-3-((thiazol-2-ylamino)methyl)-1H-indole-2-carboxamide (11.4 mg, 9%) as a white solid. MS (ESI) m/z 369.2 ([M+H]⁺).

¹H NMR (400 MHz, DMSO-d₆) δ 11.24 (s, 1H), 7.73 (t, J=4.8 Hz, 1H), 7.42 (s, 1H), 7.25 (d, J=8.4 Hz, 1H), 7.05-6.95 (m, 2H), 6.57 (d, J=3.6 Hz, 1H), 4.65-4.31 (m, 3H), 2.85 (s, 3H), 2.36 (s, 3H), 1.79-1.43 (m, 8H).

Examples (Compounds) 77-91 were synthesized using a similar method to that used in Example 76.

| Example No. | | | | |
|---|---|---|---|---|
| (Compound | | Appearance | | |
| No.) | | and | | MS (m/z) |
| Method | Structure and Name | Yield | ¹H NMR Data | [M + H]⁺ |
| Example 77 Method 5 | 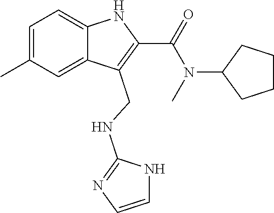 [SMILES: Cc1ccc2[nH]c(C(=O)N(C)C3CCCC3)c(CNc3ncc[nH]3)c2c1] | white solid, yield: 19.6% | ¹H NMR (400 MHz, DMSO-d₆) δ 11.18 (s, 1H), 10.57 (s, 1H), 7.51 (s, 1H), 7.24 (d, J = 8.3 Hz, 1H), 6.99 (dd, J = 8.3, 1.7 Hz, 1H), 6.45 (s, 2H), 5.86 (s, 1H), 4.81-4.19 (m, 3H), 2.88 (s, 3H), 2.36 (s, 3H), 1.88-1.39 (m, 8H). | 352.2 |
| | 3-(((1H-imidazol-2- | | | |
| | yl)amino)methyl)-N- | | | |
| | cyclopentyl-N,5- | | | |
| | dimethyl-1H-indole- | | | |
| | 2-carboxamide | | | |
| Example 78 Method 5 | 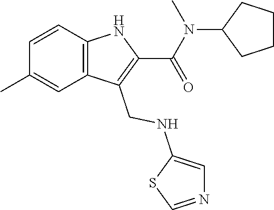 [SMILES: Cc1ccc2[nH]c(C(=O)N(C)C3CCCC3)c(CNc3cncs3)c2c1] | white solid, yield: 0.51% | ¹H NMR (300 MHz, DMSO-d₆) δ 11.22 (s, 1H), 7.71 (s, 1H), 7.34 (s, 1H), 7.23 (d, J = 8.3 Hz, 1H), 7.10-6.74 (m, 2H), 4.42 (brs, 1H), 4.16 (d, J = 6.3 Hz, 2H), 2.84 (s, 3H), 2.39 (d, J = 4.0 Hz, 3H), 1.83-1.49 (m, 8H). | 369.2 |
| | N-cyclopentyl-N,5- | | | |
| | dimethyl-3-((thiazol- | | | |
| | 5-ylamino)methyl)- | | | |
| | 1H-indole-2- | | | |
| | carboxamide | | | |
| Example 79 Method 5 | 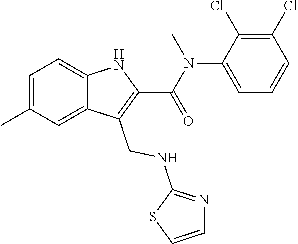 [SMILES: Cc1ccc2[nH]c(C(=O)N(C)c3cccc(Cl)c3Cl)c(CNc3nccs3)c2c1] | white solid, yield: 30% | ¹H NMR (400 MHz, CD₃OD-d₄) δ 7.22-7.44 (m, 5H), 7.01-7.09 (m, 2H), 6.57 (d, J = 3.6 Hz, 1H), 4.53 (s, 2H), 3.31 (s, 3H), 2.35 (s, 3H). | 445.1 |
| | N-(2,3- | | | |
| | dichlorophenyl)-N,5- | | | |
| | dimethyl-3-((thiazol- | | | |
| | 2-ylamino)methyl)- | | | |
| | 1H-indole-2- | | | |
| | carboxamide | | | |
| Example 80 Method 5 | 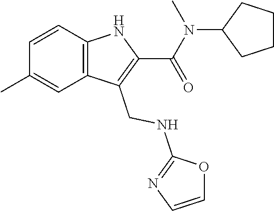 [SMILES: Cc1ccc2[nH]c(C(=O)N(C)C3CCCC3)c(CNc3ncco3)c2c1] | off-white solid, yield: 7% | ¹H NMR (400 MHz, DMSO-d₆) δ 11.20 (s, 1H), 7.42-7.38 (m, 2H), 7.31-7.22 (m, 2H), 6.98 (dd, J = 8.3, 1.2 Hz, 1H), 6.76 (d, J = 1.2 Hz, 1H), 4.45-4.40 (brs, 1H), 4.43 (d, J = 5.2 Hz, 2H), 2.84 (s, 3H), 2.36 (s, 3H), 1.73-1.46 (m, 8H). | 353.2 |
| | N-cyclopentyl-N,5- | | | |
| | dimethyl-3-((oxazol- | | | |
| | 2-ylamino)methyl)-1H- | | | |
| | indole-2-carboxamide | | | |
| Example 81 Method 5 | 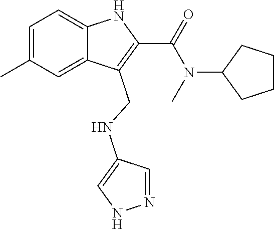 [SMILES: Cc1ccc2[nH]c(C(=O)N(C)C3CCCC3)c(CNc3cn[nH]c3)c2c1] | white solid, yield: 21% | ¹H NMR (400 MHz, DMSO-d₆) δ 11.98 (s, 1H), 11.14 (s, 1H), 7.49 (s, 1H), 7.22 (d, J = 8.2 Hz, 1H), 7.05 (s, 2H), 6.97 (dd, J = 8.4, 1.7 Hz, 1H), 4.60-4.00 (m, 4H), 2.83 (s, 3H), 2.37 (s, 3H), 1.76-1.44 (m, 8H). | 352.2 |
| | 3-(((1H-pyrazol-4- | | | |
| | yl)amino)methyl)-N- | | | |
| | cyclopentyl-N,5- | | | |
| | dimethyl-1H-indole- | | | |
| | 2-carboxamide | | | |
| Example 82 Method 5 | 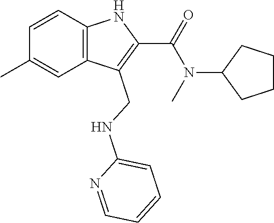 [SMILES: Cc1ccc2[nH]c(C(=O)N(C)C3CCCC3)c(CNc3ccccn3)c2c1] | white solid, yield: 15.7% | ¹H NMR (300 MHz, DMSO-d₆) δ 11.24 (s, 1H), 8.19 (s, 1H), 8.01 (dd, J = 5.2, 2.0 Hz, 1H), 7.44 (s, 1H), 7.35 (ddd, J = 8.8, 7.2, 2.0 Hz, 1H), 7.27 (d, J = 8.4 Hz, 1H), 7.01 (dd, J = 8.4, 1.6 Hz, 1H), 6.64 (t, J = 4.8 Hz, 1H), 6.58-6.44 (m, 2H), 4.50 (d, J = 4.8 Hz, 3H), 2.87 (s, 3H), 2.38 (s, 3H), 1.80-1.44 (m, 8H). | 363.2 |
| | N-cyclopentyl-N,5- | | | |
| | dimethyl-3-((pyridin- | | | |
| | 2-ylamino)methyl)- | | | |
| | 1H-indole-2- | | | |
| | carboxamide | | | |
| Example 83 Method 5 | 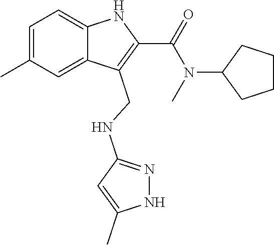 [SMILES: Cc1ccc2[nH]c(C(=O)N(C)C3CCCC3)c(CNc3cc(C)[nH]n3)c2c1] | white solid, yield: 2% | ¹H NMR (400 MHz, DMSO-d₆) δ 11.14 (s, 1H), 8.24 (s, 0.3H), 7.46 (s, 1H), 7.22 (d, J = 8.4 Hz, 1H), 6.97 (dd, J = 8.0, 1.2 Hz, 1H), 5.22 (s, 1H), 5.02 (s, 1H), 4.22 (s, 2H), 3.39 (s, 1H), 2.85 (s, 3H), 2.36 (s, 3H), 2.05 (s, 3H), 1.73-1.46 (m, 8H). | 366.2 |
| | N-cyclopentyl-N,5- | | | |
| | dimethyl-3-(((5- | | | |
| | methyl-1H-pyrazol- | | | |
| | 3-yl)amino)methyl)- | | | |
| | 1H-indole-2- | | | |
| | carboxamide | | | |
| Example 84 Method 5 | 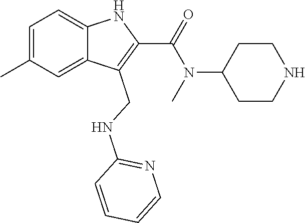 [SMILES: Cc1ccc2[nH]c(C(=O)N(C)C3CCNCC3)c(CNc3ccccn3)c2c1] | white solid, yield: 25% | ¹H NMR (400 MHz, DMSO-d₆) δ 11.20 (s, 1H), 7.97 (dd, J = 5.2, 2.0 Hz, 1H), 7.43 (s, 1H), 7.31 (ddd, J = 8.6, 7.2, 2.0 Hz, 1H), 7.24 (d, J = 8.4 Hz, 1H), 6.98 (dd, J = 8.4, 1.6 Hz, 1H), 6.59 (t, J = 4.6 Hz, 1H), 6.53-6.41 (m, 2H), 4.47 (d, J = 4.8 Hz, 2H), 3.32 (br, 6H), 2.83 (s, 3H), 2.36 (s, 3H), 1.70-1.41 (m, 4H). | 378.2 |
| | N,5-dimethyl-N- | | | |
| | (piperidin-4-yl)-3- | | | |
| | ((pyridin-2- | | | |
| | ylamino)methyl)-1H- | | | |
| | indole-2- | | | |
| | carboxamide | | | |
| Example 85 Method 5 | 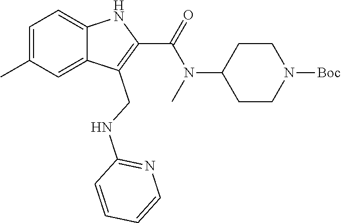 [SMILES: Cc1ccc2[nH]c(C(=O)N(C)C3CCN(C(=O)OC(C)(C)C)CC3)c(CNc3ccccn3)c2c1] | white solid, yield: 6% | ¹H NMR (400 MHz, DMSO-d₆) δ 11.21 (s, 1H), 7.98 (dd, J = 5.2, 1.6 Hz, 1H), 7.43 (s, 1H), 7.32 (ddd, J = 8.8, 7.2, 2.0 Hz, 1H), 7.25 (d, J = 8.4 Hz, 1H), 6.99 (dd, J = 8.4, 1.6 Hz, 1H), 6.60 (t, J = 5.2 Hz, 1H), 6.53-6.42 (m, 2H), 4.47 (d, J = 4.8 Hz, 2H), 3.96 (brs, 2H), 2.81 (s, 3H), 2.49 (s, 3H), 2.36 (s, 3H), 1.69-1.51 (m, 4H), 1.39 (s, 9H). | 478.3 |
| | tert-butyl 4-(N,5- | | | |
| | dimethyl-3-((pyridin- | | | |
| | 2-ylamino)methyl)- | | | |
| | 1H-indole-2- | | | |
| | carboxamido)piperidine-1- | | | |
| | carboxylate | | | |
| Example 86 Method 5 | 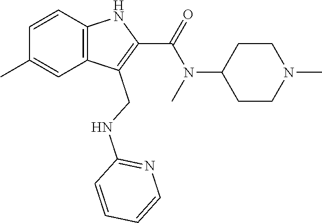 | white solid, yield: 12% | ¹H NMR (400 MHz, DMSO-d₆) δ 11.18 (s, 1H), 7.97 (d, J = 3.6 Hz, 1H), 7.43 (s, 1H), 7.34-7.28 (m, 1H), 7.24 (d, J = 8.4 Hz, 1H), 6.98 (d, J = 7.2 Hz, 1H), 6.58 (d, J = 4.8 Hz, 1H), 6.50 (d, J = 8.4 Hz, 1H), 6.47-6.42 (m, 1H), 4.47 (d, J = 4.4 Hz, 2H), 2.83 (s, 3H), 2.75-2.66 (m, 2H), 2.36 (s, 3H), 2.11 (s, 3H), 1.81-1.75 (m, 4H), 1.54 (s, 2H). | 392.2 |
| | N,5-dimethyl-N-(1- | | | |
| | methylpiperidin-4- | | | |
| | yl)-3-((pyridin-2- | | | |
| | ylamino)methyl)-1H- | | | |
| | indole-2- | | | |
| | carboxamide | | | |
| Example 87 Method 5 | 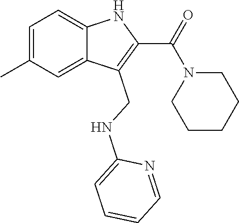 [SMILES: Cc1ccc2[nH]c(C(=O)N3CCCCC3)c(CNc3ccccn3)c2c1] | white solid, yield: 21% | ¹H NMR (400 MHz, DMSO-d₆) δ 11.20 (s, 1H), 7.99 (dd, J = 5.3, 1.9 Hz, 1H), 7.41 (s, 1H), 7.32 (ddd, J = 8.7, 7.0, 2.0 Hz, 1H), 7.24 (d, J = 8.2 Hz, 1H), 6.98 (dd, J = 8.3, 1.6 Hz, 1H), 6.57 (t, J = 5.1 Hz, 1H), 6.54-6.49 (m, 1H), 6.46 (ddd, J = 7.1, 5.0, 1.0 Hz, 1H), 4.51 (d, J = 5.0 Hz, 2H), 3.31 (s, 4H), 2.34 (s, 3H), 1.64-1.54 (m, 2H), 1.52-1.36 (m, 4H). | 349.2 |
| | (5-methyl-3- | | | |
| | ((pyridin-2- | | | |
| | ylamino)methyl)-1H- | | | |
| | indol-2-yl)(piperidin- | | | |
| | 1-yl)methanone | | | |
| Example 88 Method 5 | 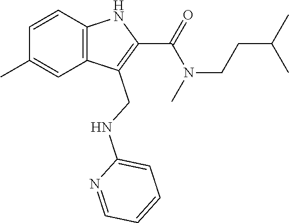 [SMILES: Cc1ccc2[nH]c(C(=O)N(C)CCC(C)C)c(CNc3ccccn3)c2c1] | yellow solid, yield: 49% | ¹H NMR (400 MHz, DMSO-d₆) δ 11.19 (s, 1H), 7.98 (dd, J = 5.2, 2.0 Hz, 1H), 7.43 (s, 1H), 7.31 (ddd, J = 8.6, 7.2, 2.0 Hz, 1H), 7.24 (d, J = 8.4 Hz, 1H), 6.97 (dd, J = 8.4, 1.6 Hz, 1H), 6.61- 6.39 (m, 3H), 4.50 (d, J = 5.2 Hz, 2H), 3.31 (s, 2H), 2.93 (s, 3H), 2.35 (s, 3H), 1.42 (s, 3H), 0.86 (s, 6H). | 365.2 |
| | N-isopentyl-N,5- | | | |
| | dimethyl-3-((pyridin- | | | |
| | 2-ylamino)methyl)- | | | |
| | 1H-indole-2- | | | |
| | carboxamide | | | |
| Example 89 Method 5 | 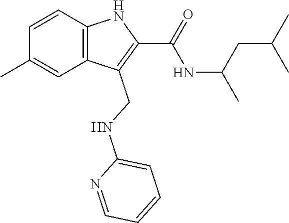 [SMILES: Cc1ccc2[nH]c(C(=O)NC(C)CC(C)C)c(CNc3ccccn3)c2c1] | white solid, yield: 48% | ¹H NMR (400 MHz, DMSO-d₆) δ 11.19 (s, 1H), 9.89 (d, J = 8.4 Hz, 1H), 7.91 (d, J = 4.0 Hz, 1H), 7.66 (s, 1H), 7.47 (t, J = 6.0 Hz, 1H), 7.39- 7.34 (m, 1H), 7.23 (d, J = 8.4 Hz, 1H), 6.98 (d, J = 8.4 Hz, 1H), 6.58-6.48 (m, 2H), 4.70-4.58 (m, 2H), 4.21-4.13 (m, 1H), 2.38 (s, 3H), 1.76- 1.68 (m, 1H), 1.53-1.46 (m, 1H), 1.34-1.30 (m, 1H), 1.23 (d, J = 6.4 Hz, 3H), 0.91-0.88 (m, 6H). | 365.1 |
| | 5-methyl-N-(4- | | | |
| | methylpentan-2-yl)- | | | |
| | 3-((pyridin-2- | | | |
| | ylamino)methyl)-1H- | | | |
| | indole-2- | | | |
| | carboxamide | | | |
| Example 90 Method 5 | 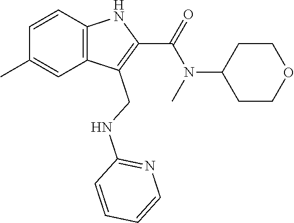 [SMILES: Cc1ccc2[nH]c(C(=O)N(C)C3CCOCC3)c(CNc3ccccn3)c2c1] | white solid, yield: 54% | ¹H NMR (400 MHz, Methanol-d₄) δ 7.95 (ddd, J = 5.2, 2.0, 0.8 Hz, 1H), 7.48-7.37 (m, 2H), 7.27 (d, J = 8.4 Hz, 1H), 7.05 (dd, J = 8.4, 1.6 Hz, 1H), 6.59-6.50 (m, 2H), 4.58 (s, 2H), 3.92 (s, 2H), 3.31 (s, 3H), 2.94 (s, 3H), 2.40 (s, 3H), 1.91-1.87 (m, 2H), 1.62 (s, 2H). | 379.2 |
| | N,5-dimethyl-3- | | | |
| | ((pyridin-2- | | | |
| | ylamino)methyl)-N- | | | |
| | (tetrahydro-2H- | | | |
| | pyran-4-yl)-1H- | | | |
| | indole-2- | | | |
| | carboxamide | | | |
| Example 91 Method 5 | 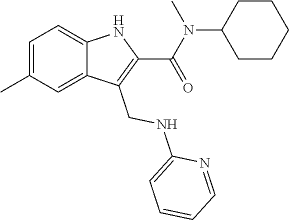 [SMILES: Cc1ccc2[nH]c(C(=O)N(C)C3CCCCC3)c(CNc3ccccn3)c2c1] | white solid, yield: 19% | ¹H NMR (400 MHz, DMSO-d₆) δ 11.17 (s, 1H), 8.16 (s, 0.35H) 7.97 (d, J = 3.6 Hz, 1H), 7.43 (s, 1H), 7.31 (t, J = 7.6 Hz, 1H), 7.24 (d, J = 8.4 Hz, 1H), 6.98 (d, J = 8.4 Hz, 1H), 6.57 (s, 1H), 6.51 (d, J = 8.4 Hz, 1H), 6.45 (t, J = 6 Hz, 1H), 4.47 (d, J = 3.6 Hz, 2H), 2.83 (s, 3H), 2.36 (s, 3H), 1.61-1.47 (m, 8H), 1.07 (m, 3H). | 377.2 |
| | N-cyclohexyl-N,5- | | | |
| | dimethyl-3-((pyridin- | | | |
| | 2-ylamino)methyl)- | | | |
| | 1H-indole-2- | | | |
| | carboxamide | | | |

### Preparation of N-cyclopentyl-N,6,7-trimethyl-1H-benzo[d]imidazole-2-carboxamide (92) Method 6

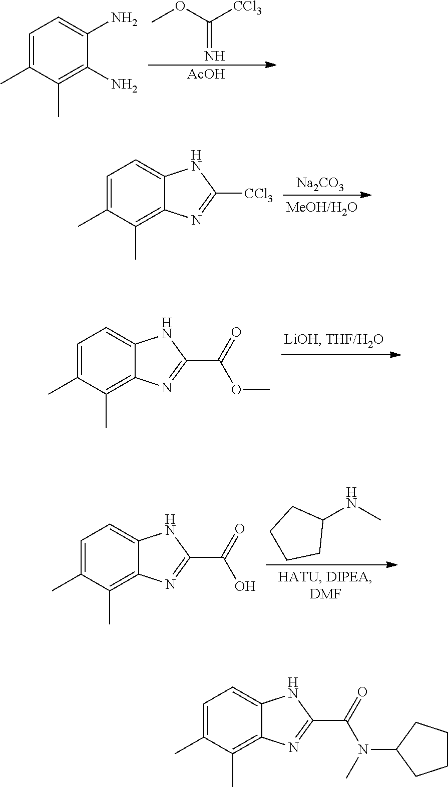

### Step 1. 6,7-dimethyl-2-(trichloromethyl)-1H-benzo[d]imidazole

To a cooled solution of 3,4-dimethylbenzene-1,2-diamine (900 mg, 6.6 mmol) in AcOH (40 mL) was added dropwise methyl 2,2,2-trichloroacetimidate (1.3 g, 7.3 mmol). The mixture was stirred at room temperature for 1 hour. The mixture was concentrated under vacuum, extracted with EA (50 mL), and washed with NaHCO₃ (50 mL*3). The combined organic layers were dried over Na₂SO₄, filtered and concentrated under vacuum. The residue was purified by FCC (PE:EA=5:1) to give 6,7-dimethyl-2-(trichloromethyl)-1H-benzo[d]imidazole (360 mg, 21%) as a yellow solid. MS Calcd.: 262; MS Found: 263 ([M+H]⁺).

### Step 2. methyl 6,7-dimethyl-1H-benzo[d]imidazole-2-carboxylate

To a solution of Na₂CO₃ (145 mg, 1.4 mmol) in water (10 mL) was added a solution of 6,7-dimethyl-2-(trichloromethyl)-1H-benzo[d]imidazole (360 mg, 1.4 mmol) in MeOH (145 mL). The mixture was refluxed for 24 hours. The mixture was concentrated under vacuum, and extracted with EA (50 mL) and H₂O (50 mL). The combined organic layers were dried over Na₂SO₄, filtered and concentrated under vacuum. The residue was purified by FCC (PE:EA=1:2) to give methyl 6,7-dimethyl-1H-benzo[d]imidazole-2-carboxylate (242 mg, 85%) as a yellow solid. MS Calcd.: 204; MS Found: 205 ([M+H]⁺).

### Step 3. 6,7-dimethyl-1H-benzo[d]imidazole-2-carboxylic acid

To a solution of 6,7-dimethyl-1H-benzo[d]imidazole-2-carboxylate (242 mg, 1.7 mmol) in THF (10 mL) and H₂O (3 mL) was added LiOH (214 mg, 5.1 mmol) at room temperature. The mixture was stirred at 50° C. for 4 hours. To the mixture was added EA (20 mL) and the mixture was extracted with H₂O (20 mL). The pH of the combined water layers were adjusted to 7 by 1 M HCl and then filtered the solid to give 6,7-dimethyl-1H-benzo[d]imidazole-2-carboxylic acid (228 mg, 70%) as a yellow solid. MS Calcd.: 190; MS Found: 191 ([M+H]⁺).

### Step 4. N-cyclopentyl-N,6,7-trimethyl-1H-benzo[d]imidazole-2-carboxamide

To a solution of 6,7-dimethyl-1H-benzo[d]imidazole-2-carboxylic acid (228 mg, 1.2 mmol), N-methylcyclopentanamine (107 mg, 1.1 mmol) and DIPEA (594 μL, 3.6 mmol) in DMF (5 mL) was added HATU (547 mg, 1, 44 mmol) at room temperature. The reaction was stirred at room temperature for 2 hours. To the mixture was added EA (50 mL), and the mixture was washed with brine (50 mL*3). The combined organic layers were dried over Na₂SO₄, filtered and concentrated under vacuum. The residue was purified by prep-HPLC (FA) to give N-cyclopentyl-N,6,7-trimethyl-1H-benzo[d]imidazole-2-carboxamide (67.4 mg, 21%) as a white solid. MS Found: 272.2 ([M+H]⁺).

¹H NMR (400 MHz, DMSO-d₆) δ 7.33 (d, J=8.2 Hz, 1H), 7.10 (d, J=8.0 Hz, 1H), 5.51-4.86 (m, 1H), 3.35-2.94 (m, 3H), 2.46 (s, 3H), 2.33 (s, 3H), 1.97-1.44 (m, 8H).

Examples (Compounds) 93-95, 97, 99, 101, 104, 113, 176 were synthesized using a similar method to that used in Example 92.

| Example No. | | | | |
|---|---|---|---|---|
| (Compound No.) | | Appearance and | | MS (m/z) |
| Method | Structure and Name | Yield | ¹H NMR Data | [M + H]⁺ |
| Example 93 Method 6 | 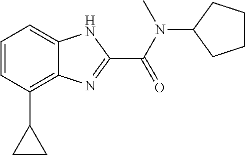 [SMILES: CN(C(=O)c1nc2c(C3CC3)cccc2[nH]1)C1CCCC1] | white solid, yield: 2% | ¹H NMR (400 MHz, Methanol-d₄) δ 7.39 (s, 1H), 7.20 (t, J = 7.8 Hz, 1H), 6.90-6.88 (m, 1H), 4.99-4.56 (m, 1H), 3.30 (s, 1H), 3.05 (s, 2H), 2.34-1.10 (m, 13H). | 284.2 |
| | N-cyclopentyl-4- | | | |
| | cyclopropyl-N- | | | |
| | methyl-1H- | | | |
| | benzo[d]imidazole-2- | | | |
| | carboxamide | | | |
| Example 94 Method 6 | 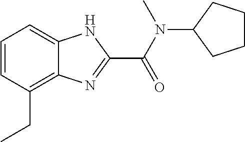 [SMILES: CCc1cccc2[nH]c(C(=O)N(C)C3CCCC3)nc12] | colorless oil, yield: 13% | ¹H NMR (400 MHz, Methanol-d₄) δ 7.59- 7.33 (m, 1H), 7.24 (t, J = 7.6 Hz, 1H), 7.13 (d, J = 20.0 Hz, 1H), 5.38-4.93 (m, 1H), 3.31 (s, 1H), 3.25-2.87 (m, 4H), 2.01-1.54 (m, 8H), 1.40-1.30 (m, 3H). | 272.2 |
| | N-cyclopentyl-4- | | | |
| | ethyl-N-methyl-1H- | | | |
| | benzo[d]imidazole-2- | | | |
| | carboxamide | | | |
| Example 95 Method 6 | 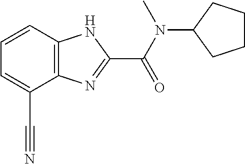 [SMILES: CN(C(=O)c1nc2c(C#N)cccc2[nH]1)C1CCCC1] | colorless oil, yield: 15% | 1H NMR (400 MHz, Methanol-d₄) δ 7.90 (dd, J = 8.2, 1.0 Hz, 1H), 7.71 (dd, J = 7.6, 1.0 Hz, 1H), 7.45 (t, J = 7.9 Hz, 1H), 5.37-4.95 (m, 1H), 3.40 (s, 1.3H), 3.06 (s, 1.7H), 2.07-1.92 (m, 2H), 1.88-1.54 (m, 6H). | 269.1 |
| | 4-cyano-N- | | | |
| | cyclopentyl-N- | | | |
| | methyl-1H- | | | |
| | benzo[d]imidazole-2- | | | |
| | carboxamide | | | |
| Example 97 Method 6 | 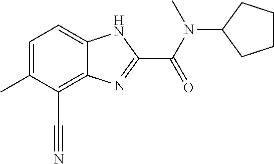 [SMILES: Cc1ccc2[nH]c(C(=O)N(C)C3CCCC3)nc2c1C#N] | white solid, yield: 19% | ¹H NMR (400 MHz, DMSO-d₆) δ 13.53 (s, 1H), 7.74 (d, J = 8.0 Hz, 1H), 7.36 (d, J = 8.0 Hz, 1H), 5.35 (s, 0.5H), 4.95-4.90 (m, 0.5H), 3.31 (s, 1.5H), 2.95 (s, 1.5H), 2.60 (s, 3H), 1.91-1.51 (m, 8H). | 283.1 |
| | 4-cyano-N- | | | |
| | cyclopentyl-N,5- | | | |
| | dimethyl-1H- | | | |
| | benzo[d]imidazole-2- | | | |
| | carboxamide | | | |
| Example 99 Method 6 | 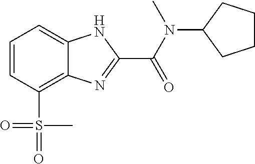 [SMILES: CN(C(=O)c1nc2c(S(C)(=O)=O)cccc2[nH]1)C1CCCC1] | white solid, yield: 14% | ¹H NMR (400 MHz, Methanol-d₄) δ 7.96 (d, J = 7.2 Hz, 1H), 7.87 (d, J = 8.0 Hz, 1H), 7.51 (t, J = 7.2 Hz, 1H) 1H), 5.31-5.03 (m, 1H), 3.39 (d, J = 6.4 Hz, 4H), 3.06 (s, 2H), 2.00-1.98 (m, 2H), 1.79-1.60 (m, 6H). | 322.1 |
| | N-cyclopentyl-N- | | | |
| | methyl-4- | | | |
| | (methylsulfonyl)-1H- | | | |
| | benzo[d]imidazole-2- | | | |
| | carboxamide | | | |
| Example 101 Method 6 | 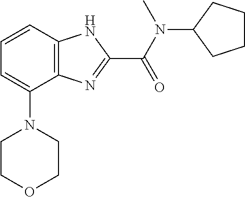 [SMILES: CN(C(=O)c1nc2c(N3CCOCC3)cccc2[nH]1)C1CCCC1] | yellow solid, yield: 9% | ¹H NMR (400 MHz, DMSO-d₆) δ 12.98 (s, 1H), 7.15 (t, J = 7.6 Hz, 1H), 7.02 (d, J = 8.0 Hz, 1H), 6.54 (d, J = 8.0 Hz, 1H), 5.73-5.71 (m, 0.6H), 4.95-4.90 (m, 0.4H), 3.82-3.80 (m, 4H), 3.52- 3.50 (m, 4H), 3.40 (s, 1H), 2.93 (s, 2H), 1.90- 1.50 (m, 8H). | 329.2 |
| | N-cyclopentyl-N- | | | |
| | methyl-4- | | | |
| | morpholino-1H- | | | |
| | benzo[d]imidazole-2- | | | |
| | carboxamide | | | |
| Example 104 Method 6 | 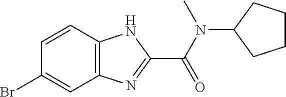 [SMILES: CN(C(=O)c1nc2cc(Br)ccc2[nH]1)C1CCCC1] | white solid, yield: 45% | ¹HNMR (400 MHz, DMSO-d₆) δ 13.22 (s, 1H), 7.93-7.41 (m, 3H), 5.60-5.59 (m, 0.5H), 4.95- 4.91 (m, 0.5H), 3.39 (s, 1.5H), 2.94 (s, 1.5H), 1.87-1.52 (m, 8H). | 322.1 |
| | 5-bromo-N- | | | |
| | cyclopentyl-N- | | | |
| | methyl-1H- | | | |
| | benzo[d]imidazole-2- | | | |
| | carboxamide | | | |
| Example 113 Method 6 | 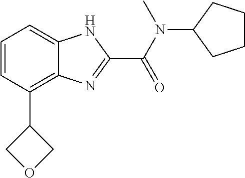 [SMILES: CN(C(=O)c1nc2c(C3COC3)cccc2[nH]1)C1CCCC1] | white solid, yield: 36% | ¹H NMR (400 MHz, Methanol-d₄) δ 7.54-7.50 (m, 1H), 7.37-7.29 (m, 2H), 5.48-5.42 (m, 1H), 5.18-4.97 (m, 4H), 4.61-4.59 (m, 1H), 4.78 (s, 1H), 3.04 (s, 2H), 2.02-1.60 (m, 8H) | 300.2 |
| | N-cyclopentyl-N- | | | |
| | methyl-4-(oxetan-3- | | | |
| | yl)-1H-benzo[d] | | | |
| | imidazole-2-carboxamide | | | |
| Example 176 Method 6 Mixture of Enantiomers | 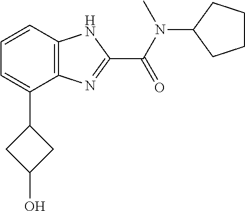 [SMILES: CN(C(=O)c1nc2c(C3CC(O)C3)cccc2[nH]1)C1CCCC1] | white solid, yield: 60% | ¹H NMR (400 MHz, DMSO-d₆) δ 7.40-7.08 (m, 3H), 6.06-5.32 (m, 2H), 4.96-3.95 (m, 2H), 3.39 (s, 1.4H), 2.94 (s, 1.6H), 2.72-2.66 (m, 1H), 2.35-2.32 (m, 1H), 2.09-1.52 (m, 10H) | 314.2 |
| | N-cyclopentyl-4- | | | |
| | ((1s,3s)-3- | | | |
| | hydroxycyclobutyl)- | | | |
| | N-methyl-1H- | | | |
| | benzo[d]imidazole-2- | | | |
| | carboxamide | | | |

### Preparation of N2-cyclopentyl-N2,5-dimethyl-1H-indole-2,3-dicarboxamide (96)

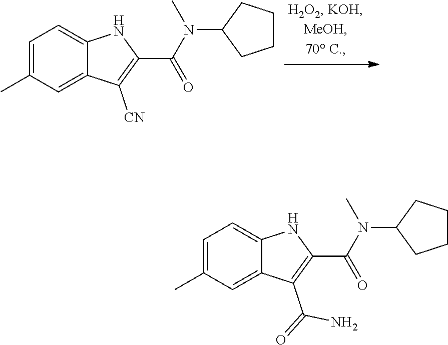 [SMILES: Cc1ccc2[nH]c(C(=O)N(C)C3CCCC3)c(C#N)c2c1 ; Cc1ccc2[nH]c(C(=O)N(C)C3CCCC3)c(C(N)=O)c2c1]

### Step 1. N2-cyclopentyl-N2,5-dimethyl-1H-indole-2,3-dicarboxamide

To a solution of 3-cyano-N-cyclopentyl-N,5-dimethyl-1H-indole-2-carboxamide (100 mg, 0.34 mmol) in MeOH (10 mL) was added H₂O₂ (10 mL) and KOH (5 mL, 2 mol/L in H₂O) and stirred at 70° C. for 1 hour. To the mixture was added water (10 mL), adjusted to pH at 4 with sat. HCl, and extracted with EA (30 mL*2). The combined organic layers were concentrated under vacuum. The residue was purified by prep-HPLC to give N2-cyclopentyl-N2,5-dimethyl-1H-indole-2,3-dicarboxamide (15.7 mg, 14.8%) as a white solid. MS (ESI) m/z 300.2 [M+H]⁺. ¹H NMR (300 MHz, DMSO-d₆) δ 11.89 (s, 1H), 7.82 (s, 1H) 7.31 (d, J=8.4 Hz, 1H), 7.07 (brs, 2H), 7.06 (d, J=7.8 Hz, 1H), 4.93-4.88 (m, 0.5H), 3.87-3.81 (m, 0.5H), 2.92-2.71 (m, 3H), 2.42 (s, 3H), 1.84-1.36 (m, 8H).

### Preparation of N-(5-chloro-4-methylpyridin-3-yl)-N-(2-hydroxyethyl)-5-(trifluoromethyl)-1H-benzo[d]imidazole-2-carboxamide (148) Method 7

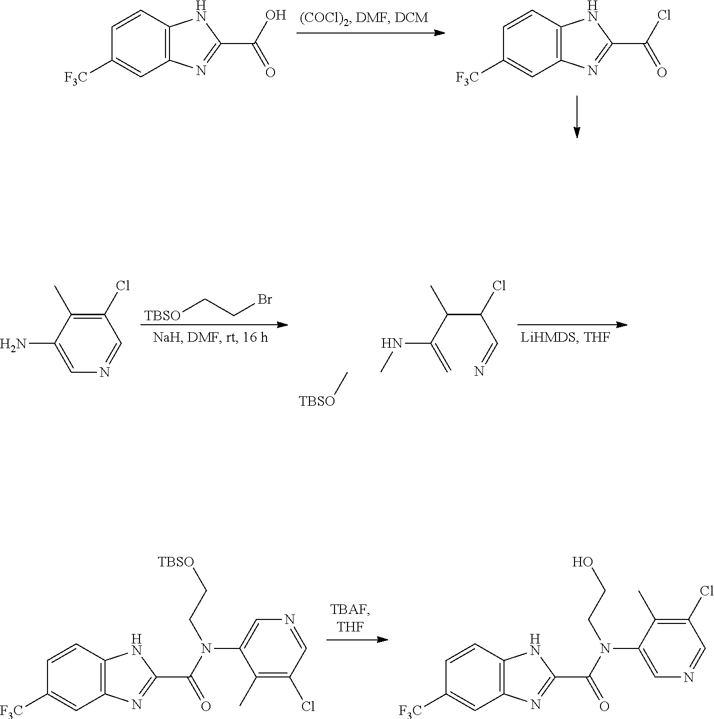

### Step 1. 5-(trifluoromethyl)-1H-benzo[d]imidazole-2-carbonyl chloride

To a solution of 5-(trifluoromethyl)-1H-benzo[d]imidazole-2-carboxylic acid (300 mg, 1.31 mmol) in DCM (15 mL) was added (COCl) 2 (0.21 mL, 2.60 mmol) and DMF (2 drop) at 0° C. The reaction was stirred at RT for 1 hour. The mixture was diluted with DCM (20 mL) and concentrated to give 5-(trifluoromethyl)-1H-benzo[d]imidazole-2-carbonyl chloride (320 mg, crude) as a yellow solid.

### Step 2. N-(2-((tert-butyldimethylsilyl)oxy)ethyl)-5-chloro-4-methylpyridin-3-amine

To a solution of 5-chloro-4-methylpyridin-3-amine (1.0 g, 7.04 mmol) in DMF (15 mL) was added NaH (310 mg, 7.7 mmol, 60% in mineral oil) at 0° C. and stirred for 30 mins. (2-bromoethoxy) (tert-butyl)dimethylsilane (1.85 g, 7.7 mmol) was added to the mixture and the mixture was stirred at RT for 16 hours. The mixture was quenched with ice-water (50 mL), and extracted with EA (50 mL*3). The combined organic layers were washed with brine (80 mL), dried over Na₂SO₄, filtered and concentrated. The residue was purified by FCC (PE/EA=3/1) to give N-(2-((tert-butyldimethylsilyl)oxy)ethyl)-5-chloro-4-methylpyridin-3-amine (140 mg, 7%) as a yellow liquid. MS (ESI) m/z 301 [M+H]⁺.

### Step 3. N-(2-((tert-butyldimethylsilyl)oxy)ethyl)-N-(5-chloro-4-methylpyridin-3-yl)-5-(trifluoromethyl)-1H-benzo[d]imidazole-2-carboxamide

To a solution of N-(2-((tert-butyldimethylsilyl)oxy)ethyl)-5-chloro-4-methylpyridin-3-amine (140 mg, 0.46 mmol) in THF (15 mL) was added LiHMDS (0.92 mL, 0.92 mmol, 1M in THF) at 0° C. and stirred for 20 mins. 5-(trifluoromethyl)-1H-benzo[d]imidazole-2-carbonyl chloride (173 mg, 0.69 mmol) was added to the mixture and the mixture was stirred at RT for 2 hours. The mixture was added to ice-water (20 mL), and extracted with EA (20 mL*3). The combined organic layers were washed with brine (30 mL), dried over Na₂SO₄, filtered and concentrated. The residue was purified by FCC (PE/EA=1/1) to give N-(2-((tert-butyldimethylsilyl)oxy)ethyl)-N-(5-chloro-4-methylpyridin-3-yl)-5-(trifluoromethyl)-1H-benzo[d]imidazole-2-carboxamide (90 mg, 38%) as a yellow solid. MS (ESI) m/z 514 [M+H]⁺.

### Step 4. N-(5-chloro-4-methylpyridin-3-yl)-N-(2-hydroxyethyl)-5-(trifluoromethyl)-1H-benzo[d]imidazole-2-carboxamide

To a solution of N-(2-((tert-butyldimethylsilyl)oxy)ethyl)-N-(5-chloro-4-methylpyridin-3-yl)-5-(trifluoromethyl)-1H-benzo[d]imidazole-2-carboxamide (90 mg, 0.17 mmol) in THF (5 mL) was added TBAF (92 mg, 0.35 mmol). The reaction was stirred at RT for 2 hours. The mixture was quenched with H₂O (20 mL), and extracted with EA (20 mL*2). The combined organic layers were washed with brine (30 mL), dried over Na₂SO₄, filtered and concentrated. The residue was purified by prep-HPLC (HCOOH) to give N-(5-chloro-4-methylpyridin-3-yl)-N-(2-hydroxyethyl)-5-(trifluoromethyl)-1H-benzo[d]imidazole-2-carboxamide (46.8 mg, 67%) as a white solid. MS (ESI) m/z 399 [M+H]⁺.

¹H NMR (400 MHz, DMSO-d₆) δ 8.63-7.47 (m, 5H), 4.86-4.19 (m, 2H), 3.70-3.54 (m, 2H), 2.32-2.26 (m, 3H).

Examples (Compounds) 20, 60, 98, 100, 102, 103, 109, 110, 115, 126, 137, 138, 145, 151, 157, 158, 159, 169, 172, 203, 237 were synthesized using a similar method to that used in Example 148.

| Example No. | | | | |
|---|---|---|---|---|
| (Compound | | Appearance | | |
| No.) | | and | | MS (m/z) |
| Method | Structure and Name | Yield | ¹H NMR Data | [M + H]⁺ |
| Example 20 Method 7 | 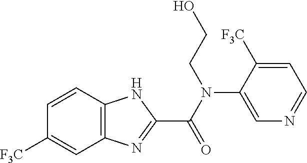 [SMILES: O=C(c1nc2cc(C(F)(F)F)ccc2[nH]1)N(CCO)c1cnccc1C(F)(F)F] | white solid, yield: 17% | ¹H NMR (400 MHz, DMSO-d₆) δ 13.72 (s, 1H), 8.96-8.87 (m, 2H), 7.95-7.47 (m, 4H), 4.99-4.96 (m, 1H), 4.51-3.62 (m, 4H) | 419.1 |
| | N-(2-hydroxyethyl)- | | | |
| | 5-(trifluoromethyl)- | | | |
| | N-(4- | | | |
| | (trifluoromethyl)pyridin- | | | |
| | 3-yl)-1H- | | | |
| | benzo[d]imidazole-2- | | | |
| | carboxamide | | | |
| Example 60 Method 7 Mixture of Enantiomers | 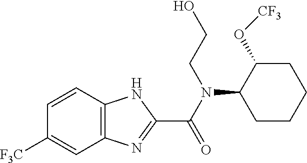 [SMILES: O=C(c1nc2cc(C(F)(F)F)ccc2[nH]1)N(CCO)[C@@H]1CCCC[C@H]1OC(F)(F)F] | white solid, yield: 19% | ¹H NMR (400 MHz, DMSO-d₆) δ 13.61- 13.45 (m, 1H), 8.13-7.62 (m, 3H), 5.66-5.62 (m, 0.5H), 5.08 (brs, 0.5H), 4.84 (t, J = 5.6 Hz, 1H), 3.83-3.50 (m, 5H), 2.19-1.28 (m, 8H) | 440.1 |
| | N-(2-hydroxyethyl)- | | | |
| | N-((1R,2R)-2- | | | |
| | (trifluoromethoxy) | | | |
| | cyclohexyl)-5- | | | |
| | (trifluoromethyl)-1H- | | | |
| | benzo[d]imidazole-2- | | | |
| | carboxamide | | | |
| Example 98 Method 7 | 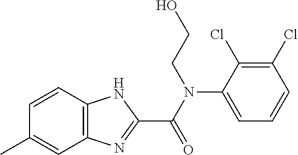 [SMILES: Cc1ccc2[nH]c(C(=O)N(CCO)c3cccc(Cl)c3Cl)nc2c1] | yellow solid, yield: 37.5% | ¹H NMR (400 MHz, Chloroform-d) δ 7.56 (d, J = 8.0 Hz, 2H), 7.44 (d, J = 7.6 Hz, 1H), 7.40-7.32 (m, 2H), 7.22 (d, J = 8.4 Hz, 1H), 4.02-3.66 (m, 4H), 2.50 (s, 3H). | 364.0 |
| | N-(2,3- | | | |
| | dichlorophenyl)-N- | | | |
| | (2-hydroxyethyl)-5- | | | |
| | methyl-1H- | | | |
| | benzo[d]imidazole-2- | | | |
| | carboxamide | | | |
| Example 100 Method 7 | 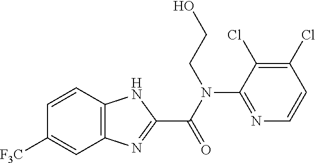 [SMILES: O=C(c1nc2cc(C(F)(F)F)ccc2[nH]1)N(CCO)c1nccc(Cl)c1Cl] | white solid, yield: 32.0% | ¹H NMR (400 MHz, DMSO-d₆) δ 13.81 (s, 1H), 8.43 (d, J = 5.1 Hz, 1H), 7.77 (d, J = 5.3 Hz, 2H), 7.62 (d, J = 41.1 Hz, 2H), 4.76 (s, 1H), 4.04 (d, J = 57.2 Hz, 2H), 3.71 (d, J = 7.3 Hz, 2H). | 419.0 |
| | N-(3,4- | | | |
| | dichloropyridin-2- | | | |
| | yl)-N-(2- | | | |
| | hydroxyethyl)-5- | | | |
| | (trifluoromethyl)-1H- | | | |
| | benzo[d]imidazole-2- | | | |
| | carboxamide | | | |
| Example 102 Method 7 Mixture of Enantiomers | 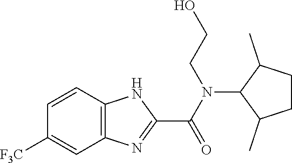 [SMILES: CC1CCC(C)C1N(CCO)C(=O)c1nc2cc(C(F)(F)F)ccc2[nH]1] | white solid, yield: 39% | ¹H NMR (400 MHz, Methanol-d₄) δ 8.02 (brs, 1H), 7.80 (brs, 1H), 7.61-7.60 (m, 1H), 4.20-3.68 (m, 4H), 2.80-2.32 (m, 2H), 1.97- 1.92 (m, 2H), 1.63-1.00 (m, 9H). | 370.1 |
| | N-(2,5- | | | |
| | dimethylcyclopentyl)- | | | |
| | N-(2-hydroxyethyl)- | | | |
| | 5-(trifluoromethyl)- | | | |
| | 1H- | | | |
| | benzo[d]imidazole-2- | | | |
| | carboxamide | | | |
| Example 103 Method 7 | 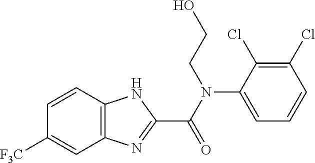 [SMILES: O=C(c1nc2cc(C(F)(F)F)ccc2[nH]1)N(CCO)c1cccc(Cl)c1Cl] | white solid, yield: 28% | ¹HNMR (400 MHz, DMSO-d₆) δ 13.68 (s, 1H), 8.34-7.66 (m, 2H), 7.66-7.59 (m, 2H), 7.59-7.38 (m, 2H), 4.98-4.83 (m, 1H), 4.32-3.95 (m, 1H), 3.78-3.44 (m, 3H). | 418.0 |
| | N-(2,3- | | | |
| | dichlorophenyl)- | | | |
| | (2-hydroxyethyl)-5- | | | |
| | (trifluoromethyl)-1H- | | | |
| | benzo[d]imidazole-2- | | | |
| | carboxamide | | | |
| Example 109 Method 7 | 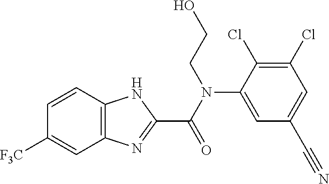 [SMILES: N#Cc1cc(Cl)c(Cl)c(N(CCO)C(=O)c2nc3cc(C(F)(F)F)ccc3[nH]2)c1] | white solid, yield: 24% | ¹H NMR (400 MHz, DMSO-d₆) δ 13.77 (s, 1H), 8.39-8.32 (m, 1H), 8.25-8.17 (m, 1H), 7.92-7.71 (m, 1H), 7.68-7.45 (m, 2H), 4.88 (t, J = 5.4 Hz, 1H), 4.27-4.14 (m, 1H), 3.79-3.68 (m, 1H), 3.67-3.55 (m, 2H). | 442.0 |
| | N-(2,3-dichloro-5- | | | |
| | cyanophenyl)-N-(2- | | | |
| | hydroxyethyl)-5- | | | |
| | (trifluoromethyl)-1H- | | | |
| | benzo[d]imidazole-2- | | | |
| | carboxamide | | | |
| Example 110 Method 7 | 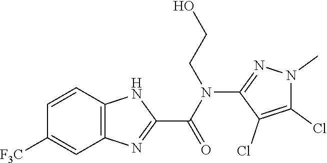 [SMILES: Cn1nc(N(CCO)C(=O)c2nc3cc(C(F)(F)F)ccc3[nH]2)c(Cl)c1Cl] | white solid, yield: 60% | ¹H NMR (400 MHz, DMSO-d₆) δ 13.74 (d, J = 13.2 Hz, 1H), 7.92-7.77 (m, 1H), 7.75-7.66 (m, 1H), 7.65-7.46 (m, 1H), 4.84-4.75 (m, 1H), 4.08-3.77 (m, 5H), 3.70-3.60 (m, 2H). | 422.0 |
| | N-(4,5-dichloro-1- | | | |
| | methyl-1H-pyrazol- | | | |
| | 3-yl)-N-(2- | | | |
| | hydroxyethyl)-5- | | | |
| | (trifluoromethyl)-1H- | | | |
| | benzo[d]imidazole-2- | | | |
| | carboxamide | | | |
| Example 115 Method 7 | 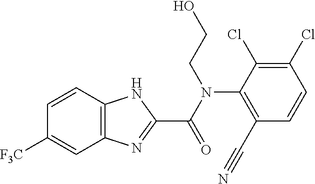 [SMILES: N#Cc1ccc(Cl)c(Cl)c1N(CCO)C(=O)c1nc2cc(C(F)(F)F)ccc2[nH]1] | white solid, yield: 38% | ₁H NMR (400 MHz, Methanol-d₄) δ 7.87- 7.81 (m, 1H), 7.78 (d, J = 8.5 Hz, 1H), 7.72 (d, J = 8.4 Hz, 1H), 7.69-7.64 (m, 1H), 7.59- 7.50 (m, 1H), 4.32-4.24 (m, 1H), 3.97- 3.86 (m, 3H) | 444.2 |
| | N-(2,3-dichloro-6- | | | |
| | cyanophenyl)-N-(2- | | | |
| | hydroxyethyl)-5- | | | |
| | (trifluoromethyl)-1H- | | | |
| | benzo[d]imidazole-2- | | | |
| | carboxamide | | | |
| Example 126 Method 7 | 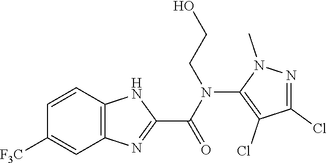 [SMILES: Cn1nc(Cl)c(Cl)c1N(CCO)C(=O)c1nc2cc(C(F)(F)F)ccc2[nH]1] | white solid, yield: 52% | ¹H NMR (400 MHz, Methanol-d₄) δ 8.03 (s, 1H), 7.89-7.81 (m, 1H), 7.72-7.63 (m, 1H), 4.54 (t, J = 5.2 Hz, 1H), 4.30-4.19 (m, 0.5H), 3.96-3.86 (m, 0.5H), 3.80 (s, 1.5H), 3.77- 3.68 (m, 2H), 3.62 (s, 1.5H). | 423.3 |
| | N-(3,4-dichloro-1- | | | |
| | methyl-1H-pyrazol- | | | |
| | 5-yl)-N-(2- | | | |
| | hydroxyethyl)-5- | | | |
| | (trifluoromethyl)-1H- | | | |
| | benzo[d]imidazole-2- | | | |
| | carboxamide | | | |
| Example 137 Method 7 | 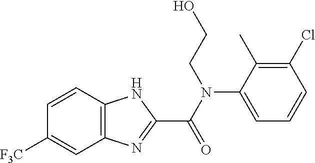 [SMILES: Cc1c(Cl)cccc1N(CCO)C(=O)c1nc2cc(C(F)(F)F)ccc2[nH]1] | white solid, yield: 56% | ¹H NMR (400 MHz, Methanol-d₄) δ 8.49- 7.12 (m, 6H), 4.85-4.29 (m, 1.5H), 3.87- 4.72 (m, 2H), 3.64-3.57 (m, 0.5H), 2.36- 2.30 (m, 3H). | 398.1 |
| | N-(3-chloro-2- | | | |
| | methylphenyl)-N-(2- | | | |
| | hydroxyethyl)-5- | | | |
| | (trifluoromethyl)-1H- | | | |
| | benzo[d]imidazole-2- | | | |
| | carboxamide | | | |
| Example 138 Method 7 | 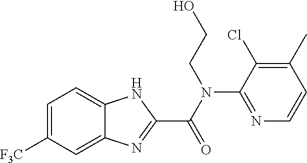 [SMILES: Cc1ccnc(N(CCO)C(=O)c2nc3cc(C(F)(F)F)ccc3[nH]2)c1Cl] | white solid, yield: 55% | ¹H NMR (400 MHz, DMSO-d₆) δ 13.72 (s, 1H), 8.29 (s, 1H), 7.78-7.42 (m, 4H), 4.77 (s, 1H), 4.12 (s, 1H), 3.85 (s, 1H), 3.68 (s, 2H), 2.34 (s, 3H). | 399.1 |
| | N-(3-chloro-4- | | | |
| | methylpyridin-2-yl)- | | | |
| | N-(2-hydroxyethyl)- | | | |
| | 5-(trifluoromethyl)- | | | |
| | 1H- | | | |
| | benzo[d]imidazole-2- | | | |
| | carboxamide | | | |
| Example 145 Method 7 | 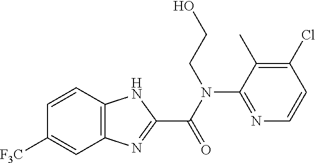 [SMILES: Cc1c(Cl)ccnc1N(CCO)C(=O)c1nc2cc(C(F)(F)F)ccc2[nH]1] | white solid, yield: 40% | ¹H NMR (400 MHz, DMSO-d₆) δ 13.72 (d, J = 10.8 Hz, 1H), 8.21 (d, J = 5.4 Hz, 1H), 7.80-7.63 (m, 2H), 7.62-7.40 (m, 2H), 4.76 (t, J = 5.6 Hz, 1H), 4.08-3.97 (m, 1H), 3.90-3.80 (m, 1H), 3.77-3.67 (m, 1H), 3.66-3.55 (m, 1H), 2.23 (d, 3H). | 399.1 |
| | N-(4-chloro-3- | | | |
| | methylpyridin-2-yl)- | | | |
| | N-(2-hydroxyethyl)- | | | |
| | 5-(trifluoromethyl)- | | | |
| | 1H- | | | |
| | benzo[d]imidazole-2- | | | |
| | carboxamide | | | |
| Example 151 Method 7 | 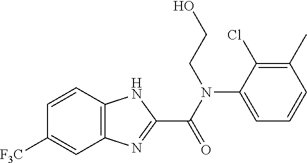 [SMILES: Cc1cccc(N(CCO)C(=O)c2nc3cc(C(F)(F)F)ccc3[nH]2)c1Cl] | white solid, yield: 64% | ¹H NMR (400 MHz, DMSO-d₆) δ 13.58 (s, 1H), 8.08-7.25 (m, 6H), 5.02-4.98 (m, 0.35H), 4.85 (t, J = 5.6 Hz, 1H), 4.29-4.23 (m, 0.65H), 3.89-3.42 (m, 3H), 2.42 (s, 1.2H), 2.29 (s, 1.8H). | 398.1 |
| | N-(2-chloro-3- | | | |
| | methylphenyl)-N-(2- | | | |
| | hydroxyethyl)-5- | | | |
| | (trifluoromethyl)-1H- | | | |
| | benzo[d]imidazole-2- | | | |
| | carboxamide | | | |
| Example 157 Method 7 | 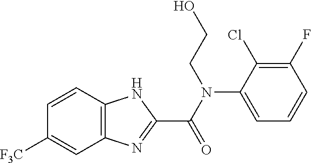 [SMILES: O=C(c1nc2cc(C(F)(F)F)ccc2[nH]1)N(CCO)c1cccc(F)c1Cl] | white solid, yield: 43% | ¹H NMR (400 MHz, Methanol-d₄) δ 8.07 (s, 0.3H), 7.86-7.75 (m, 1H), 7.65-7.60 (m, 1H), 7.51-7.34 (m, 3H), 7.29-7.24 (m, 0.7H), 4.96-4.90 (m, 0.3H), 4.40-4.33 (m, 0.7H), 4.18-3.31 (m, 3H) | 402.1 |
| | N-(2-chloro-3- | | | |
| | fluorophenyl)-N-(2- | | | |
| | hydroxyethyl)-5- | | | |
| | (trifluoromethyl)-1H- | | | |
| | benzo[d]imidazole-2- | | | |
| | carboxamide | | | |
| Example 158 Method 7 | 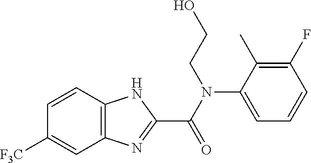 [SMILES: Cc1c(F)cccc1N(CCO)C(=O)c1nc2cc(C(F)(F)F)ccc2[nH]1] | off-white solid, yield: 28% | ¹H NMR (400 MHz, Methanol-d₄) δ 8.65- 6.78 (m, 6H), 4.67-4.20 (m, 1.5H), 3.91- 3.49 (m, 2.5H), 2.37-2.06 (m, 3H) | 382.1 |
| | N-(3-fluoro-2- | | | |
| | methylphenyl)-N-(2- | | | |
| | hydroxyethyl)-5- | | | |
| | (trifluoromethyl)-1H- | | | |
| | benzo[d]imidazole-2- | | | |
| | carboxamide | | | |
| Example 159 Method 7 | 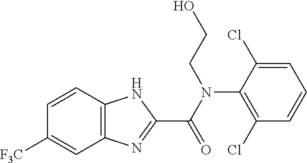 [SMILES: O=C(c1nc2cc(C(F)(F)F)ccc2[nH]1)N(CCO)c1c(Cl)cccc1Cl] | white solid, yield: 37% | ¹H NMR (400 MHz, DMSO-d₆) δ 7.82- 7.22 (m, 6H), 4.81 (t, J = 5.6 Hz, 1H), 3.96- 3.52 (m, 4H) | 418.0 |
| | N-(2,6- | | | |
| | dichlorophenyl)-N- | | | |
| | (2-hydroxyethyl)-5- | | | |
| | (trifluoromethyl)-1H- | | | |
| | benzo[d]imidazole-2- | | | |
| | carboxamide | | | |
| Example 169 Method 7 | 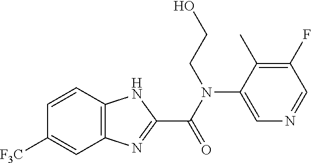 [SMILES: Cc1c(F)cncc1N(CCO)C(=O)c1nc2cc(C(F)(F)F)ccc2[nH]1] | white solid, yield: 59% | ¹H NMR (400 MHz, DMSO-d₆) δ 8.64- 7.39 (m, 5H), 4.94-4.03 (m, 2H), 3.69- 3.57 (m, 3H), 2.22-2.16 (m, 3H) | 383.1 |
| | N-(5-fluoro-4- | | | |
| | methylpyridin-3-yl)- | | | |
| | N-(2-hydroxyethyl)- | | | |
| | 5-(trifluoromethyl)- | | | |
| | 1H- | | | |
| | benzo[d]imidazole-2- | | | |
| | carboxamide | | | |
| Example 172 Method 7 | 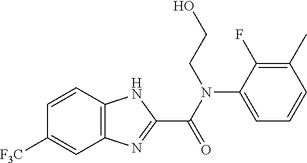 [SMILES: Cc1cccc(N(CCO)C(=O)c2nc3cc(C(F)(F)F)ccc3[nH]2)c1F] | white solid, yield: 69% | ¹H NMR (400 MHz, DMSO-d₆) δ 13.90- 13.18 (m, 1H), 8.17-6.95 (m, 6H), 4.85 (s, 1H), 4.46-3.56 (m, 4H), 2.33-2.15 (m, 3H). | 382.1 |
| | N-(2-fluoro-3- | | | |
| | methylphenyl)-N-(2- | | | |
| | hydroxyethyl)-5- | | | |
| | (trifluoromethyl)-1H- | | | |
| | benzo[d]imidazole-2- | | | |
| | carboxamide | | | |
| Example 203 Method 7 | 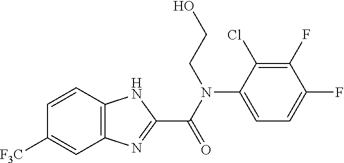 [SMILES: O=C(c1nc2cc(C(F)(F)F)ccc2[nH]1)N(CCO)c1ccc(F)c(F)c1Cl] | white solid, yield: 34% | ¹HNMR (400 MHz, DMSO-d6) δ 13.78- 13.64 (m, 1H), 8.25-7.73 (m, 1H), 7.71- 7.62 (m, 1H), 7.61-7.40 (m, 3H), 4.94- 4.84 (m, 1H), 4.29-4.08 (m, 1H), 3.80- 3.50 (m, 3H). | 420.0 |
| | N-(2-chloro-3,4- | | | |
| | difluorophenyl)-N- | | | |
| | (2-hydroxyethyl)-5- | | | |
| | (trifluoromethyl)-1H- | | | |
| | benzo[d]imidazole-2- | | | |
| | carboxamide | | | |
| Exampel 237 Method 7 | 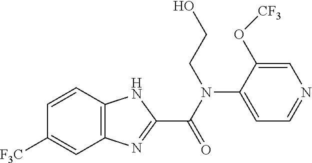 [SMILES: O=C(c1nc2cc(C(F)(F)F)ccc2[nH]1)N(CCO)c1ccncc1OC(F)(F)F] | white solid, yield: 3% | ¹H NMR (400 MHz, Methanol-d₄) δ 8.92- 8.80 (m, 2H), 7.88-7.78 (m, 1H), 7.77- 7.68 (m, 2H), 7.56-7.40 (m, 1H), 2.90 (s, 3H). | 405.0 |
| | N-methyl-N-(3- | | | |
| | (trifluoromethoxy) | | | |
| | pyridin-4-yl)-5- | | | |
| | (trifluoromethyl)-1H- | | | |
| | benzo[d]imidazole-2- | | | |
| | carboxamide | | | |

### Preparation of N-(5-chloro-4-methylpyridin-3-yl)-N-(2,2-difluoro-3-hydroxypropyl)-5-(trifluoromethyl)-1H-benzo[d]imidazole-2-carboxamide (133) Method 8

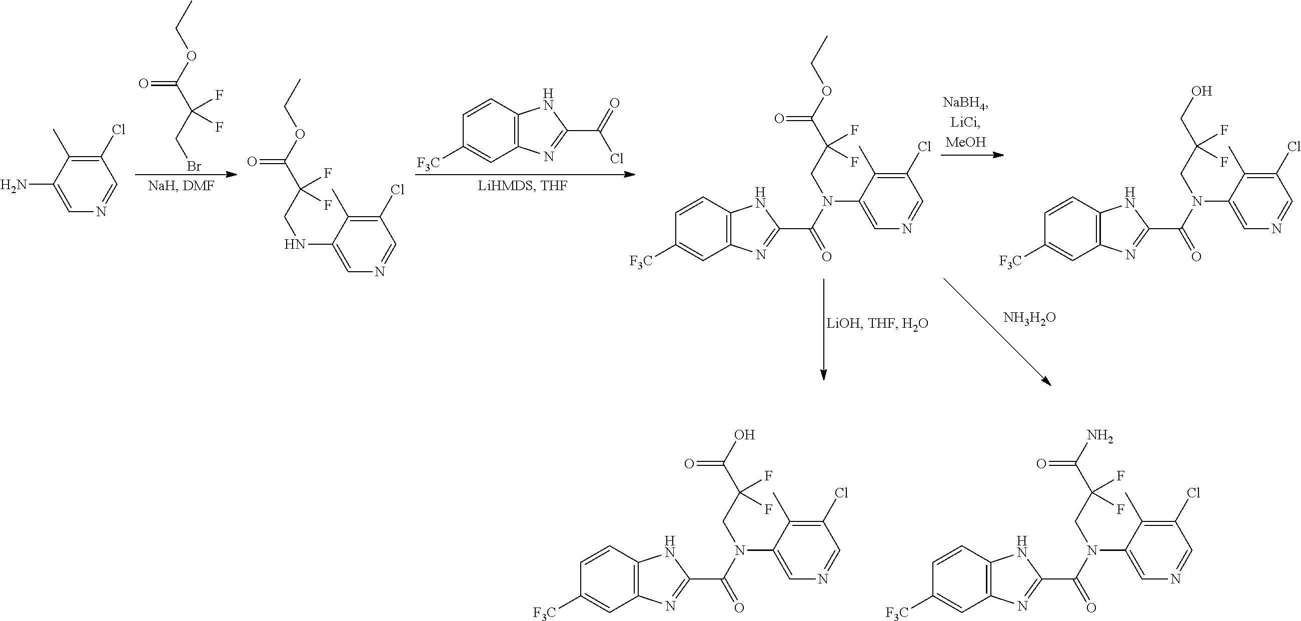

### Step 1. ethyl 3-((5-chloro-4-methylpyridin-3-yl)amino)-2,2-difluoropropanoate

To a solution of 5-chloro-4-methylpyridin-3-amine (100 mg, 0.70 mmol) in DMF (5 mL) was added NaH (31 mg, 0.77 mmol, 60% in mineral oil) at 0° C. The reaction was stirred at 0° C. for 20 mins. Ethyl 3-bromo-2,2-difluoropropanoate (152 mg, 0.70 mmol) added to the mixture and the mixture was stirred at rt for 3 hours. The mixture was quenched with water (10 mL), and extracted with EA (10 mL*2). The combined organic layers were washed with brine (10 mL), dried over Na₂SO₄, filtered and concentrated. The residue was purified by FCC (PE/EA=3/1) to give ethyl 3-((5-chloro-4-methylpyridin-3-yl)amino)-2,2-difluoropropanoate (20 mg, 10%) as a yellow solid. MS (ESI) m/z 279 [M+H]⁺.

### Step 2. ethyl 3-(N-(5-chloro-4-methylpyridin-3-yl)-5-(trifluoromethyl)-1H-benzo[d]imidazole-2-carboxamido)-2,2-difluoropropanoate

To a solution of ethyl 3-((5-chloro-4-methylpyridin-3-yl)amino)-2,2-difluoropropanoate (200 mg, 0.72 mmol) in THF (5 mL) was added LiHMDS (1.08 mL, 1.08 mmol, 1M in THF) and stirred at 0° C. for 30 min. 5-(trifluoromethyl)-1H-benzo[d]imidazole-2-carbonyl chloride (180 mg, 0.72 mmol) was added to the mixture and stirred at rt for 1 hour. To the mixture was added water (10 mL), and the mixture was extracted with EA (10 mL*2). The combined organic layers were washed with brine (20 mL), dried over Na₂SO₄, filtered and concentrated. The residue was purified by FCC (PE/EA=1/1) to give ethyl 3-(N-(5-chloro-4-methylpyridin-3-yl)-5-(trifluoromethyl)-1H-benzo[d]imidazole-2-carboxamido)-2,2-difluoropropanoate (35 mg, 10%) as a yellow solid. MS (ESI) m/z 491 [M+H]⁺.

### Step 3. N-(5-chloro-4-methylpyridin-3-yl)-N-(2,2-difluoro-3-hydroxypropyl)-5-(trifluoromethyl)-1H-benzo[d]imidazole-2-carboxamide

To a solution of ethyl 3-(N-(5-chloro-4-methylpyridin-3-yl)-5-(trifluoromethyl)-1H-benzo[d]imidazole-2-carboxamido)-2,2-difluoropropanoate (35 mg, 0.07 mmol) in MeOH (5 mL) was added NaBH₄ (6 mg, 0.14 mmol) and LiCl (6 mg, 0.14 mmol). The mixture was stirred at rt for 1 hour. To the mixture was added water (5 mL), extracted with DCM (5 mL*2). The combined organic layers were washed with brine (5 mL), dried over Na₂SO₄, filtered and concentrated. The residue was purified by prep-HPLC (NH₄HCO₃) to give N-(5-chloro-4-methylpyridin-3-yl)-N-(2,2-difluoro-3-hydroxypropyl)-5-(trifluoromethyl)-1H-benzo[d]imidazole-2-carboxamide (9.1 mg, 29%) as a white solid. MS (ESI) m/z 449 [M+H]⁺. ¹H NMR (400 MHz, DMSO-d₆) δ 8.61-8.45 (m, 2H), 7.90-7.57 (m, 2H), 7.41-7.40 (m, 1H), 5.07-4.81 (m, 2H), 4.24 (s, 1H), 3.84-3.69 (m, 2H), 2.28-2.23 (m, 3H).

### Step 4. 3-(N-(5-chloro-4-methylpyridin-3-yl)-5-(trifluoromethyl)-1H-benzo[d]imidazole-2-carboxamido)-2,2-difluoropropanoic Acid

To a solution of ethyl 3-(N-(5-chloro-4-methylpyridin-3-yl)-5-(trifluoromethyl)-1H-benzo[d]imidazole-2-carboxamido)-2,2-difluoropropanoate (35 mg, 0.07 mmol) in THF (5 mL) was added LiOH (30 mg) and H₂O (2 mL). The mixture was stirred at rt for 1 hour. To the mixture was added HCl (2 N, to adjust the pH=1), and the mixture was extracted with DCM (5 mL*3). The combined organic layers were washed with brine (5 mL), dried over Na₂SO₄, filtered and concentrated. The residue was purified by prep-HPLC to give 3-(N-(5-chloro-4-methylpyridin-3-yl)-5-(trifluoromethyl)-1H-benzo[d]imidazole-2-carboxamido)-2,2-difluoropropanoic acid (12.1 mg, 33.3%) as a white solid. MS (ESI) m/z 463.1 [M+H]+.

### Step 5. N-(3-amino-2,2-difluoro-3-oxopropyl)-N-(5-chloro-4-methylpyridin-3-yl)-5-(trifluoromethyl)-1H-benzo[d]imidazole-2-carboxamide

To a solution of ethyl 3-(N-(5-chloro-4-methylpyridin-3-yl)-5-(trifluoromethyl)-1H-benzo[d]imidazole-2-carboxamido)-2,2-difluoropropanoate (35 mg, 0.07 mmol) in MeOH (2 mL) was added NH₃H₂O (3 mL). The mixture was stirred at 80° C. for 4 hour. To the mixture was added water (5 mL), and the mixture was extracted with DCM (5 mL*2). The combined organic layers were washed with brine (5 mL), dried over Na₂SO₄, filtered and concentrated. The residue was purified by prep-HPLC (NH₄HCO₃) to give N-(3-amino-2,2-difluoro-3-oxopropyl)-N-(5-chloro-4-methylpyridin-3-yl)-5-(trifluoromethyl)-1H-benzo[d]imidazole-2-carboxamide (9.3 mg, 29%) as a white solid. MS (ESI) m/z 462.1 [M+H]+.

Examples (Compounds) 105, 106, 107, 111, 112, 114, 116, 117, 118, 120, 121, 123, 125, 127, 128, 129, 131, 132, 134, 135, 136, 140, 141, 146, 147, 149, 152, 156, 165-169, 173, 174, 177-182, 184-186, 188, 193, 194, 197-200, 202, 207, 212-220, 224-230, 238-240 were synthesized using a similar method to that used in Example 133.

| Example | | | | |
|---|---|---|---|---|
| No. | | Appear- | | |
| (Compound | | ance | | MS |
| No.) | | and | | (m/z) |
| Method | Structure and Name | Yield | ¹H NMR Data | [M + H]⁺ |
| Example 105 Method 8 | 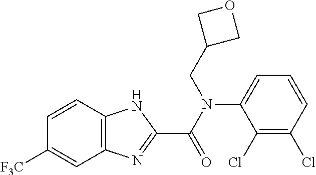 [SMILES: O=C(c1nc2cc(C(F)(F)F)ccc2[nH]1)N(CC1COC1)c1cccc(Cl)c1Cl] | white solid, yield: 23% | ¹H NMR (400 MHz, DMSO-d6) δ 13.72 (s, 1H), 8.25-7.64 (m, 3H), 7.62-7.49 (m, 2H), 7.43 (t, J = 8,0 Hz, 1H), 5.05-4.25 (m, 5H), 4.22-4.12 (m, 1H), 3.31-3.19 (m, 1H). | 444.1 |
| | N-(2,3- | | | |
| | dichlorophenyl)-N- | | | |
| | (oxetan-3-ylmethyl)- | | | |
| | 5-(trifluoromethyl)- | | | |
| | 1H- | | | |
| | benzo[d]imidazole-2- | | | |
| | carboxamide | | | |
| Example 106 Method 8 | 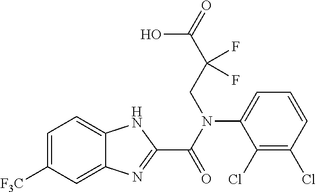 [SMILES: O=C(c1nc2cc(C(F)(F)F)ccc2[nH]1)N(CC(F)(F)C(=O)O)c1cccc(Cl)c1Cl] | yellow solid, yield: 59% | ¹H NMR (400 MHz, Methanol-d₄) δ 7.75- 7.40 (m, 5H), 7.30 (m, 1H), 5.06 (m, 1H), 4.05 (m, 1H) | 482.2 |
| | 3-(N-(2,3- | | | |
| | dichlorophenyl)-6- | | | |
| | (trifluoromethyl)-1H- | | | |
| | benzo[d]imidazole-2- | | | |
| | carboxamido)-2,2- | | | |
| | difluoropropanoic | | | |
| | acid | | | |
| Example 107 Method 8 | 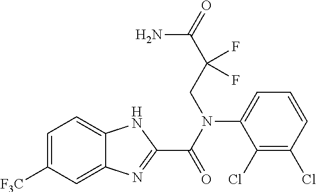 [SMILES: NC(=O)C(F)(F)CN(C(=O)c1nc2cc(C(F)(F)F)ccc2[nH]1)c1cccc(Cl)c1Cl] | white solid, yield: 38% | ¹H NMR (400 MHz, Methanol-d₄) δ 7.81- 7.47 (m, 5H), 7.34 (t, J = 8.1 Hz, 1H), 5.06 (m, 1H), 4.12 (m, 1H). | 481.2 |
| | N-(3-amino-2,2- | | | |
| | difluoro-3- | | | |
| | oxopropyl)-N-(2,3- | | | |
| | dichlorophenyl)-5- | | | |
| | (trifluoromethyl)-1H- | | | |
| | benzo[d]imidazole-2- | | | |
| | carboxamide | | | |
| Example 111 Method 8 | 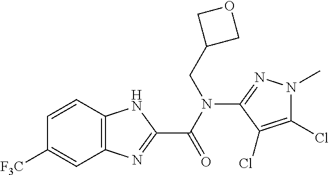 [SMILES: Cn1nc(N(CC2COC2)C(=O)c2nc3cc(C(F)(F)F)ccc3[nH]2)c(Cl)c1Cl] | white solid, yield: 18% | ¹H NMR (400 MHz, DMSO-d₆) δ 13.78 (d, J = 13.5 Hz, 1H), 7.92- 7.77 (m, 1H), 7.74- 7.66 (m, 1H), 7.65- 7.44 (m, 1H), 4.61 (t, J = 7.1 Hz, 2H), 4,39- 4.31 (m, 2H), 4,24- 4.10 (m, 2H), 3.83 (s, 3H). | 448.0 |
| | N-(4,5-dichloro-1- | | | |
| | methyl-1H-pyrazol- | | | |
| | 3-yl)-N-(oxetan-3- | | | |
| | ylmethyl)-5- | | | |
| | (trifluoromethyl)-1H- | | | |
| | benzo[d]imidazole-2- | | | |
| | carboxamide | | | |
| Example 112 Method 8 | 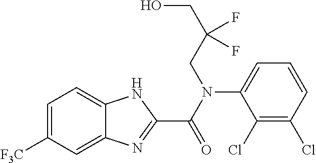 [SMILES: O=C(c1nc2cc(C(F)(F)F)ccc2[nH]1)N(CC(F)(F)CO)c1cccc(Cl)c1Cl] | white solid, yield: 35% | ¹H NMR (400 MHz, DMSO-d₆) δ 13.77 (s, 1H), 7.80-7.65 (m, 2H), 7.65-7.58 (m, 2H), 7.52 (1, J = 8.0 Hz, 1H), 7.42 (t, J = 8.0 Hz, 1H), 5.61 (t, J = 6.2 Hz, 1H), 4.71 (m, 1H), 4.15 (m, 1H), 3.72 (td, J = 13.8, 6.2 Hz, 2H) | 468.0 |
| | N-(2,3- | | | |
| | dichlorophenyl)-N- | | | |
| | (2,2-difluoro-3- | | | |
| | hydroxypropyl)-5- | | | |
| | (trifluoromethyl)-1H- | | | |
| | benzo[d]imidazole-2- | | | |
| | carboxamide | | | |
| Example 114 Method 8 | 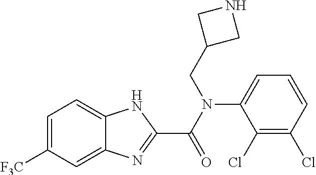 [SMILES: O=C(c1nc2cc(C(F)(F)F)ccc2[nH]1)N(CC1CNC1)c1cccc(Cl)c1Cl] | white solid, yield: 6% | ¹H NMR (400 MHz, Methanol-d₄) δ 8:44 (s, 1H), 8.07-7.61 (m, 1 H), 7.60-7.46 (m, 2 H), 7.44-7.36 (m, 2H), 7.34-7.27 (m, 1H), 5.07-4.10 (m, 2H), 4.10-3.20 (m, 5H) | 443.1 |
| | N-(azetidin-3- | | | |
| | ylmethyl)-N-(2,3- | | | |
| | dichlorophenyl)-5- | | | |
| | (trifluoromethyl)-1H- | | | |
| | benzo[d]imidazole-2- | | | |
| | carboxamide | | | |
| Example 116 Method 8 | 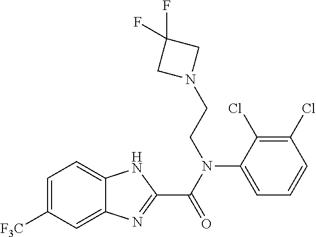 [SMILES: O=C(c1nc2cc(C(F)(F)F)ccc2[nH]1)N(CCN1CC(F)(F)C1)c1cccc(Cl)c1Cl] | off- white solid, yield: 4% | ¹H NMR (400 MHz, Methanol-d₄) δ 7.74- 7.65 (m, 1H), 7.63- 7.57 (m, 2H), 7.54- 7.46 (m, 2H), 7.36 (t, J = 7.6 Hz, 1H), 4.33- 4.27 (m, 1H), 3.90- 3.82 (m, 4H), 3.63- 3.58 (m, 1H), 3.19- 3.04 (m, 2H) | 493.1 |
| | N-(2,3- | | | |
| | dichlorophenyl)-N- | | | |
| | (2-(3,3- | | | |
| | difluoroazetidin-1- | | | |
| | yl)ethyl)-5- | | | |
| | (trifluoromethyl)-1H- | | | |
| | benzo[d]imidazole-2- | | | |
| | carboxamide | | | |
| Example 117 Method 8 | 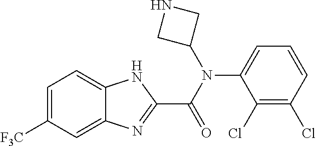 [SMILES: O=C(c1nc2cc(C(F)(F)F)ccc2[nH]1)N(c1cccc(Cl)c1Cl)C1CNC1] | white solid, yield: 22% | ¹H NMR (400 MHz, DMSO-d₆) δ 8.35- 7.97 (m, 3H), 7.78- 7.74 (m, 2H), 7.67- 7.63 (m, 1H), 7.59- 7.51 (m, 1H), 5.16- 5.02 (m, 1H), 4.90- 4.75 (m, 1H), 4.30- 4.07 (m, 1H), 2.94- 2.56 (m, 2H) | 429.0 |
| | N-(azetidin-3-yl)-N- | | | |
| | (2,3-dichlorophenyl)- | | | |
| | 5-(trifluoromethyl)- | | | |
| | 1H- | | | |
| | benzo[d]imidazole-2- | | | |
| | carboxamide | | | |
| Example 118 Method 8 | 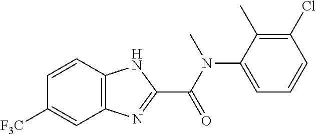 [SMILES: Cc1c(Cl)cccc1N(C)C(=O)c1nc2cc(C(F)(F)F)ccc2[nH]1] | white solid, yield: 17% | ¹HNMR (400 MHz, DMSO-d6) δ 13.60 (s, 1H), 8.16-7.72 (m, 1H), 7.68-7.60 (m, 1H), 7.54-7.46 (m, 1H), 7.44-7.13 (m, 3H), 3.85 (s, 1H), 3.34 (s, 2H), 2.25 (d, J = 7.8 Hz, 3H). | 368.1 |
| | N-(3-chloro-2- | | | |
| | methylphenyl)-N- | | | |
| | methyl-5- | | | |
| | (trifluoromethyl)-1H- | | | |
| | benzo[d]imidazole-2- | | | |
| | carboxamide | | | |
| Example 120 Method 8 | 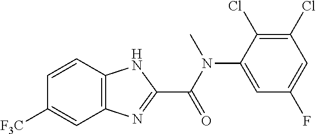 [SMILES: CN(C(=O)c1nc2cc(C(F)(F)F)ccc2[nH]1)c1cc(F)cc(Cl)c1Cl] | white solid, yield: 19% | ¹HNMR (400 MHz, DMSO-d6) δ 13.70 (s, 1H), 7.85-7.55 (m, 5H), 3.92-3.38 (m, 3H). | 406.0 |
| | N-(2,3-dichloro-5- | | | |
| | fluorophenyl)-N- | | | |
| | methyl-5- | | | |
| | (trifluoromethyl)-1H- | | | |
| | benzo[d]imidazole-2- | | | |
| | carboxamido | | | |
| Example 121 Method 8 | 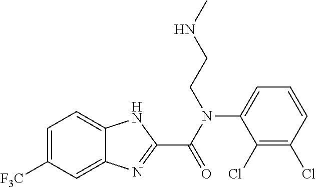 [SMILES: CNCCN(C(=O)c1nc2cc(C(F)(F)F)ccc2[nH]1)c1cccc(Cl)c1Cl] | white solid, yield: 7% | ¹H NMR (400 MHz, DMSO-d₆) δ 8.02- 7.38 (m, 6H), 4.74 4.70 (m, 0.35H), 4.32- 4.00 (m, 1.65H), 3.64- 3.49 (m, 2H), 2.39- 2.28 (m, 3H) | 431.1 |
| | N-(2,3- | | | |
| | dichlorophenyl)-N- | | | |
| | (2- | | | |
| | (methylamino)ethyl)- | | | |
| | 5-(trifluoromethyl)- | | | |
| | 1H- | | | |
| | benzo[d]imidazole-2- | | | |
| | carboxamide | | | |
| Example 123 Method 8 | 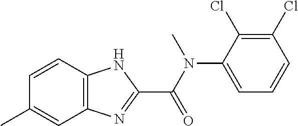 [SMILES: Cc1ccc2[nH]c(C(=O)N(C)c3cccc(Cl)c3Cl)nc2c1] | white solid, yield: 26% | ¹H NMR (400 MHz, DMSO-d₆) δ 13.08 (dd, J = 39.6, 10.4 Hz, 1H), 7.75-6.87 (m, 6H), 3.35 (s, 3H), 2.47- 2.27 (m, 3H) | 334.0 |
| | N-(2,3- | | | |
| | dichlorophenyl)-N,5- | | | |
| | dimethyl-1H- | | | |
| | benzimidazole-2- | | | |
| | carboxamide | | | |
| Example 125 Method 8 | 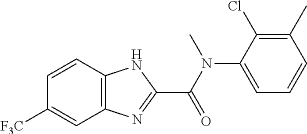 [SMILES: Cc1cccc(N(C)C(=O)c2nc3cc(C(F)(F)F)ccc3[nH]2)c1Cl] | white solid, yield: 3% | ¹H NMR (400 MHz, Methanol-d₄) δ 7.80- 7.30 (m, 3H), 7.30- 7.10 (m, 3H), 3.80- 3.30 (m, 3H), 2.37- 2.21 (m, 3H). | 368.1 |
| | N-(2-chloro-3- | | | |
| | methylphenyl)-N- | | | |
| | methyl-5- | | | |
| | (trifluoromethyl)-1H- | | | |
| | benzo[d]imidazole-2- | | | |
| | carboxamide | | | |
| Example 127 Method 8 | 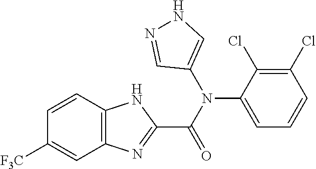 [SMILES: O=C(c1nc2cc(C(F)(F)F)ccc2[nH]1)N(c1cn[nH]c1)c1cccc(Cl)c1Cl] | white solid, yield: 27% | ¹H NMR (400 MHz, DMSO-d₆) δ 13.79 (s, 1H), 12.94 (d, J = 64.2 Hz, 1H), 7.91 (s, 2H), 7.67-7.74 (m, 4H), 7.44-7.60 (m, 2H) | 440.0 |
| | N-(2,3- | | | |
| | dichlorophenyl)-N- | | | |
| | (1H-pyrazol-4-yl)-5- | | | |
| | (trifluoromethyl)-1H- | | | |
| | benzo[d]imidazole-2- | | | |
| | carboxamide | | | |
| Example 128 Method 8 | 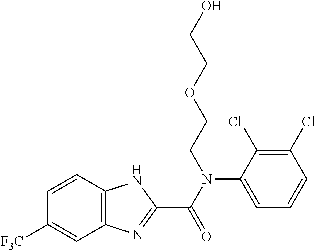 [SMILES: O=C(c1nc2cc(C(F)(F)F)ccc2[nH]1)N(CCOCCO)c1cccc(Cl)c1Cl] | off- white solid, yield: 27% | ¹H NMR (400 MHz, Methanol-d₄) δ 8.56- 7.32 (m, 6H), 5.01- 4.96 (m, 0.3H), 4.48- 4.42 (m, 0.7H), 4.26- 4.23 (m, 0.2H), 3.87- 3.62 (m, 2.8H), 3.58- 3.48 (m, 4H) | 462.1 |
| | N-(2,3- | | | |
| | dichlorophenyl)-N- | | | |
| | (2-(2- | | | |
| | hydroxyethoxy)ethyl)- | | | |
| | 5-(trifluoromethyl)- | | | |
| | 1H- | | | |
| | benzo[d]imidazole-2- | | | |
| | carboxamide | | | |
| Example 129 Method 8 | 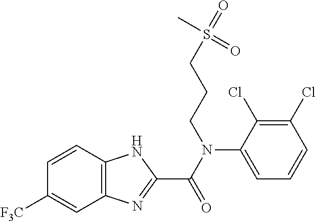 [SMILES: CS(=O)(=O)CCCN(C(=O)c1nc2cc(C(F)(F)F)ccc2[nH]1)c1cccc(Cl)c1Cl] | white solid, yield: 1% | ¹H NMR (400 MHz, DMSO-d₆) δ 8.41- 7.43 (m, 6H), 4.67- 4.61 (m, 0.25H), 4.36- 4.17 (m, 1H), 3.74- 3.65 (m, 0.75H), 3.28- 3.24 (m, 2H), 3.00 (d, J = 10.0 Hz, 3H), 2.22- 1.96 (m, 2H) | 494.1 |
| | N-(2,3- | | | |
| | dichlorophenyl)-N- | | | |
| | (3- | | | |
| | (methylsulfonyl) | | | |
| | propyl)-5- | | | |
| | (trifluoromethyl)-1H- | | | |
| | benzo[d]imidazole-2- | | | |
| | carboxamide | | | |
| Example 131 Method 8 Mixture of Enantiomer | 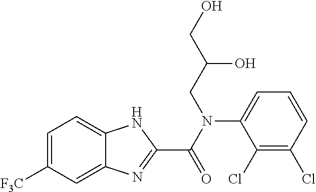 [SMILES: O=C(c1nc2cc(C(F)(F)F)ccc2[nH]1)N(CC(O)CO)c1cccc(Cl)c1Cl] | white solid, yield: 5% | ¹H NMR (400 MHz, DMSO-d₆) δ 13.62 (s, 1H), 8.05-7.51 (m, 6H), 7.44-7.37 (m, 1H), 4.94-4.86 (m, 1H), 4.65 (q, J = 6.0 Hz, 1H), 4.39-4.25 (m, 1H), 4.00-3.71 (m, 1H), 3.63-3.54(m, 1H), 3.42-3.34 (m, 2H) | 448.1 |
| | N-(2,3- | | | |
| | dichlorophenyl)-N- | | | |
| | (2,3- | | | |
| | dihydroxypropyl)-5- | | | |
| | (trifluoromethyl)-1H- | | | |
| | benzo[d]imidazole-2- | | | |
| | carboxamide | | | |
| Example 132 Method 8 | 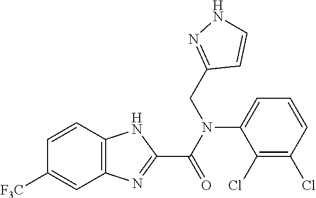 [SMILES: O=C(c1nc2cc(C(F)(F)F)ccc2[nH]1)N(Cc1cc[nH]n1)c1cccc(Cl)c1Cl] | white solid, yield: 66% | ¹H NMR (400 MHz, DMSO-d₆) δ 13.73 (s, 1H), 12.62 (s, 1H), 8.29- 7.22 (m, 7H), 6.34- 6,18 (m, 1.2H), 5,40- 5.36 (m, 1H), 4.69 (d, J = 14.8 Hz, 0.7H) | 454.1 |
| | N-((1H-pyrazol-3- | | | |
| | yl)methyl)-N-(2,3- | | | |
| | dichlorophenyl)-5- | | | |
| | (trifluoromethyl)-1H- | | | |
| | benzo[d]imidazole-2- | | | |
| | carboxamide | | | |
| Example 134 Method 8 | 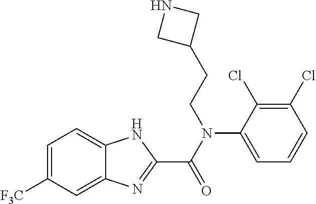 [SMILES: O=C(c1nc2cc(C(F)(F)F)ccc2[nH]1)N(CCC1CNC1)c1cccc(Cl)c1Cl] | white solid, yield: 28% | ¹H NMR (400 MHz, Methanol-d₄) δ 8.54 (s, 1H), 8.15-7.81 (m, 1H), 7.74-7.37 (m, 6H), 4.65-4.36 (m, 1H), 4.21-3.68 (m, 5H), 3.14-3.08 (m, 1H), 2.22-2.05 (m, 2H) | 457.1 |
| | N-(2-(azetidin-3- | | | |
| | yl)ethyl)-N-(2,3- | | | |
| | dichlorophenyl)-5- | | | |
| | (trifluoromethyl)-1H- | | | |
| | benzo[d]imidazole-2- | | | |
| | carboxamide | | | |
| Example 135 Method 8 | 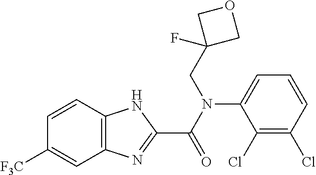 [SMILES: O=C(c1nc2cc(C(F)(F)F)ccc2[nH]1)N(CC1(F)COC1)c1cccc(Cl)c1Cl] | white solid, yield: 13% | ¹H NMR (400 MHz, Methanol-d₄) δ 7.74 (s, 1H), 7.68-7.58 (m, 1H), 7.56-7.42 (m, 3H), 7.35 (t, J = 8.0 Hz, 1H), 5.54-5.37 (m, 0.5H), 4.80-4.64 (m, 4.5H), 4.46-4.37 (m, 1H) | 462.1 |
| | N-(2,3- | | | |
| | dichlorophenyl)-N- | | | |
| | ((3-fluorooxetan-3- | | | |
| | yl)methyl)-5- | | | |
| | (trifluoromethyl)-1H- | | | |
| | benzo[d]imidazole-2- | | | |
| | carboxamido | | | |
| Example 136 Method 8 Mixture of Enantiomer | 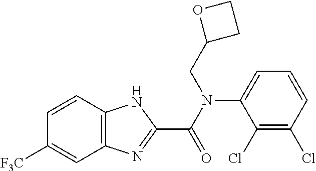 [SMILES: O=C(c1nc2cc(C(F)(F)F)ccc2[nH]1)N(CC1CCO1)c1cccc(Cl)c1Cl] | white solid, yield: 18% | ¹H NMR (400 MHz, DMSO-d₆) δ 13.60 (s, 1H), 8.25-7.39 (m, 6H), 5.21-3.85 (m, 5H), 2.70-2.59 (m, 1H), 2.47-2.40 (m, 1H) | 444.1 |
| | N-(2,3- | | | |
| | dichlorophenyl)-N- | | | |
| | (oxetan-2-ylmethyl)- | | | |
| | 5-(trifluoromethyl)- | | | |
| | 1H- | | | |
| | benzo[d]imidazole-2- | | | |
| | carboxamide | | | |
| Example 140 Method 8 | 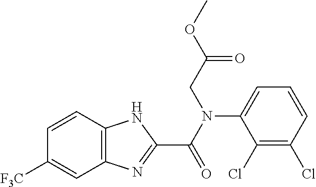 [SMILES: COC(=O)CN(C(=O)c1nc2cc(C(F)(F)F)ccc2[nH]1)c1cccc(Cl)c1Cl] | white solid, yield: 26% | ¹H NMR (400 MHz, Methanol-d₄) δ 8.11- 7.40 (m, 5H), 7.30 (t, J = 8.1 Hz, 1H), 5.02 (d, J = 17.2 Hz, 1H), 4.14 (d, J = 17,2 Hz, 1H), 3.77 (d, J = 13.8 Hz, 3H). | 446.0 |
| | Methyl N-(2,3- | | | |
| | dichlorophenyl)-N- | | | |
| | (5-(trifluoromethyl)- | | | |
| | 1H- | | | |
| | benzo[d]imidazole-2- | | | |
| | carbonyl)glycinate | | | |
| Example 141 Method 8 | 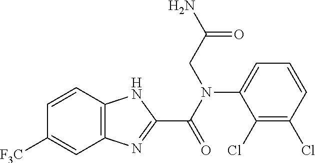 [SMILES: NC(=O)CN(C(=O)c1nc2cc(C(F)(F)F)ccc2[nH]1)c1cccc(Cl)c1Cl] | white solid, yield: 16% | ¹H NMR (400 MHz, Methanol-d₄) δ 7.75 (s, 1H), 7.65-7.39 (m, 6H), 7.30 (t, J = 8.1 Hz, 1H), 5.08 (d, J = 16.4 Hz, 1H), 3,95 (d, J = 16.4 Hz, 1H). | 431.0 |
| | N-(2-amino-2- | | | |
| | oxoethyl)-N-(2,3- | | | |
| | dichlorophenyl)-5- | | | |
| | (trifluoromethyl)-1H- | | | |
| | benzo[d]imidazole-2- | | | |
| | carboxamide | | | |
| Example 146 Method 8 | 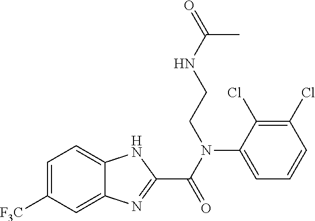 [SMILES: CC(=O)NCCN(C(=O)c1nc2cc(C(F)(F)F)ccc2[nH]1)c1cccc(Cl)c1Cl] | white solid, yield: 22% | ¹H NMR (400 MHz, DMSO-d₆) δ 13.70- 13.68 (m, 1H), 8.21- 7.40 (m, 7H), 4.88- 4.85 (m, 0.3H), 4.25- 3.97 (m, 1H), 3.54- 3.29 (m, 2.7H), 1.76 (s, 2.1H), 1.65 (s, 0.9H) | 459.1 |
| | N-(2- | | | |
| | acctamidoethyl)-N- | | | |
| | (2,3-dichlorophenyl)- | | | |
| | 5-(trifluoromethyl)- | | | |
| | 1H- | | | |
| | benzo[d]imidazole-2- | | | |
| | carboxamide | | | |
| Example 147 Method 8 | 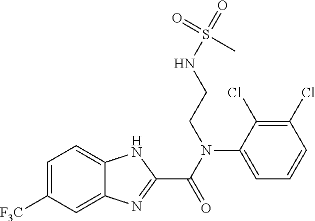 [SMILES: CS(=O)(=O)NCCN(C(=O)c1nc2cc(C(F)(F)F)ccc2[nH]1)c1cccc(Cl)c1Cl] | white solid, yield: 21% | ¹H NMR (400 MHz, DMSO-d₆) δ 13.69 (s, 1H), 8.25-7.24 (m, 7H), 4.89-4,85 (m, 0.3H), 4.33-4.08 (m, 1H), 3.58-3.51 (m, 0.7H), 3.41-3.22 (m, 2H), 2.94 (d, J = 4.0 Hz, 3H) | 495.1 |
| | N-(2,3- | | | |
| | dichlorophenyl)-N- | | | |
| | (2- | | | |
| | (methylsulfonamido) | | | |
| | ethyl)-5- | | | |
| | (trifluoromethyl)-1H- | | | |
| | benzo[d]imidazole-2- | | | |
| | carboxamide | | | |
| Example 149 Method 8 | 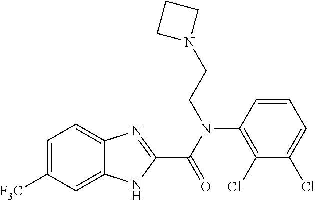 [SMILES: O=C(c1nc2ccc(C(F)(F)F)cc2[nH]1)N(CCN1CCC1)c1cccc(Cl)c1Cl] | white solid, yield: 1% | ¹H NMR (400 MHz, DMSO-d₆) δ 8.31 (s, 1H), 7.73-7.42 (m, 6H), 4.08-4.04 (m, 1H), 3.17-3.03 (m, 5H), 2.63-2.54 (m, 2H), 1.96-1.85 (m, 2H) | 457.1 |
| | N-(2-(azetidin-1- | | | |
| | yl)ethyl)-N-(2,3- | | | |
| | dichlorophenyl)-6- | | | |
| | (trifluoromethyl)-1H- | | | |
| | benzo[d]imidazole-2- | | | |
| | carboxamide | | | |
| Example 152 Method 8 | 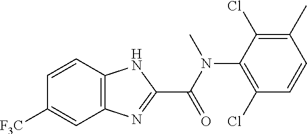 [SMILES: Cc1ccc(Cl)c(N(C)C(=O)c2nc3cc(C(F)(F)F)ccc3[nH]2)c1Cl] | white solid, yield: 95% | ¹HNMR (400 MHz, Methanol-d₄) δ 8.07- 7.34 (m, 5H), 3.39 (s, 3H), 2.44-2.38 (s, 3H) | 402.1 |
| | N-(2,6-dichloro-3- | | | |
| | methylphenyl)-N- | | | |
| | methyl-5- | | | |
| | (trifluoromethyl)-1H- | | | |
| | benzo[d]imidazole-2- | | | |
| | carboxamide | | | |
| Example 156 Method 8 | 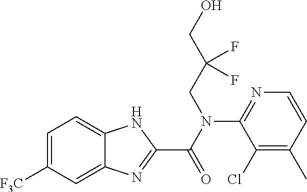 [SMILES: Cc1ccnc(N(CC(F)(F)CO)C(=O)c2nc3cc(C(F)(F)F)ccc3[nH]2)c1Cl] | white solid, yield: 22% | ¹H NMR (400 MHz, DMSO-d₆) δ 8.34 (s, 1H), 7.73-7.63 (m, 2H), 7.52 (d, J = 8.4 Hz, 1H), 7.42 (s, 1H), 5.56 (s, 1H), 4.58 (s, 2H), 3.71 (s, 2H), 2.33- 2.27 (m, 3H) | 449.0 |
| | N-(3-chloro-4- | | | |
| | methylpyridin-2-yl)- | | | |
| | N-(2,2-difluoro-3- | | | |
| | hydroxypropyl)-5- | | | |
| | (trifluoromethyl)-1H- | | | |
| | benzo[d]imidazole-2- | | | |
| | carboxamide | | | |
| Example 165 Method 8 | 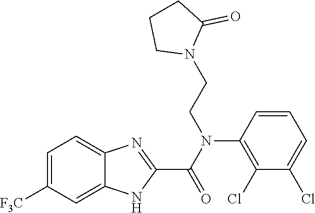 [SMILES: O=C1CCCN1CCN(C(=O)c1nc2ccc(C(F)(F)F)cc2[nH]1)c1cccc(Cl)c1Cl] | yellow solid, yield: 1% | ¹H NMR (400 MHz, DMSO-d₆) δ 8.41 (s, 1H), 7.94 (s, 1H), 7.72 (d, J = 8.4 Hz, 1H), 7.55 (d, J = 8.4 Hz, 1H), 6.72-6.55 (m, 3H), 5.17 (dd, J = 9.2, 5,6 Hz, 1H), 3.80-3.62 (m, 4H), 3.57-3.49 (m, 2H), 2.15-1.40 (m, 2H) | 485.0 |
| | N-(2,3- | | | |
| | dichlorophenyl)-N- | | | |
| | (2-(2-oxopyrrolidin- | | | |
| | 1-yl)ethyl)-6- | | | |
| | (trifluoromethyl)-1H- | | | |
| | benzo[d]imidazole-2- | | | |
| | carboxamide | | | |
| Example 166 Method 8 | 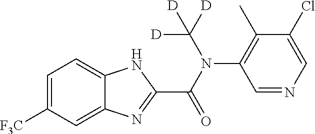 [SMILES: [2H]C([2H])([2H])N(C(=O)c1nc2cc(C(F)(F)F)ccc2[nH]1)c1cncc(Cl)c1C] | white solid, yield: 13% | ¹H NMR (400 MHz, DMSO-d₆) δ 13.68 (s, 1H), 8.65-8.39 (m, 2H), 8.14-7.53 (m, 3H), 2.30 (d, J = 3.6 Hz, 3H) | 372.1 |
| | N-(5-chloro-4- | | | |
| | methylpyridin-3-yl)- | | | |
| | N-(methyl-d3)-5- | | | |
| | (trifluoromethyl)-1H- | | | |
| | benzo[d]imidazole-2- | | | |
| | carboxamido | | | |
| Example 167 Method 8 | 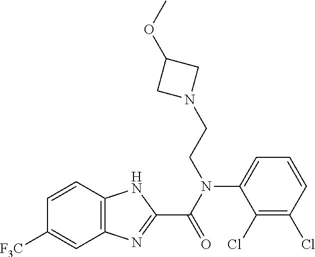 [SMILES: COC1CN(CCN(C(=O)c2nc3cc(C(F)(F)F)ccc3[nH]2)c2cccc(Cl)c2Cl)C1] | white solid, yield: 8% | ¹H NMR (400 MHz, DMSO-d₆) δ 13.69 (s, 1H), 8.17-7.29 (m, 6H), 4.74-3.36 (m, 5H), 3.28-3.03 (m, 3H), 2.87-2.56 (m, 4H) | 487.0 |
| | N-(2,3- | | | |
| | dichlorophenyl)-N- | | | |
| | (2-(3- | | | |
| | methoxyazetidin-1- | | | |
| | yl)ethyl)-5- | | | |
| | (trifluoromethyl)-1H- | | | |
| | benzo[d]imidazole-2- | | | |
| | carboxamide | | | |
| Example 168 Method 8 | 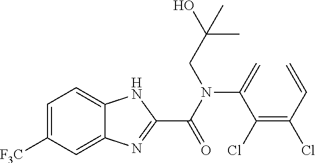 [SMILES: C=C/C(Cl)=C(/Cl)C(=C)N(CC(C)(C)O)C(=O)c1nc2cc(C(F)(F)F)ccc2[nH]1] | white solid, yield: 35% | ¹H NMR (400 MHz, DMSO-d₆) δ 12.76 (s, 1H), 8.10-7.16 (m, 6H), 4.99-3.43 (m, 3H), 1.25-1.03 (m, 6H) | 446.1 |
| | N-(2,3- | | | |
| | dichlorophenyl)-N- | | | |
| | (2-hydroxy-2- | | | |
| | methylpropyl)-5- | | | |
| | (trifluoromethyl)-1H- | | | |
| | benzo[d]imidazole-2- | | | |
| | carboxamide | | | |
| Example 169 Method 8 Mixture of Enantiomers | 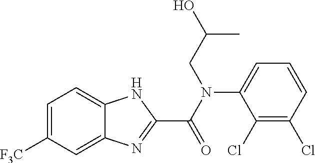 [SMILES: CC(O)CN(C(=O)c1nc2cc(C(F)(F)F)ccc2[nH]1)c1cccc(Cl)c1Cl] | white solid, yield: 51% | ¹H NMR (400 MHz, DMSO-d₆) δ 13.46 (s, 1H), 8.13-7.35 (m, 6H), 4.90-4.80 (m, 1H), 4.30-3.61 (m, 2H), 3.47-3.13 (m, 1H), 1.34-0.92 (m, 3H). | 432.0 |
| | N-(2,3- | | | |
| | dichlorophenyl)-N- | | | |
| | (2-hydroxypropyl)-5- | | | |
| | (trifluoromethyl)-1H- | | | |
| | benzo[d]imidazole-2- | | | |
| | carboxamide | | | |
| Example 173. Method 8 Mixture of Enantiomer | 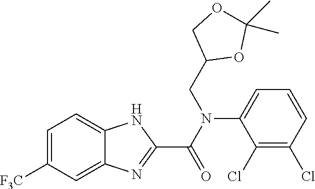 [SMILES: CC1(C)OCC(CN(C(=O)c2nc3cc(C(F)(F)F)ccc3[nH]2)c2cccc(Cl)c2Cl)O1] | white solid, yield: 13% | ¹H NMR (400 MHz, DMSO-d₆) δ 13.72 (s, 1H), 8.44-7.29 (m, 6H), 5.26-3.42 (m, 5H), 1.52-0.74 (m, 6H) | 488.0 |
| | N-(2,3- | | | |
| | dichlorophenyl)-N- | | | |
| | ((2,2-dimethyl-1,3- | | | |
| | dioxolan-4- | | | |
| | yl)methyl)-5- | | | |
| | (trifluoromethyl)-1H- | | | |
| | benzo[d]imidazole-2- | | | |
| | carboxamide | | | |
| Example 174 Method 8 | 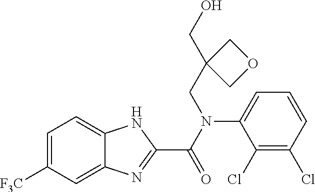 [SMILES: O=C(c1nc2cc(C(F)(F)F)ccc2[nH]1)N(CC1(CO)COC1)c1cccc(Cl)c1Cl] | white solid, yield: 29% | ¹H NMR (400 MHz, DMSO-d₆) δ 8.16- 7.35 (m, 6H), 4.49- 3.64 (m, 8H) | 474.0 |
| | N-(2,3- | | | |
| | dichlorophenyl)-N- | | | |
| | ((3-(hydroxymethyl) | | | |
| | oxetan- | | | |
| | 3-yl)methyl)-5- | | | |
| | (trifluoromethyl)-1H- | | | |
| | benzo[d]imidazole-2- | | | |
| | carboxamido | | | |
| Example 177 Method 8 Mixture of Enantiomers | 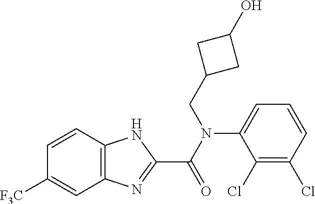 [SMILES: O=C(c1nc2cc(C(F)(F)F)ccc2[nH]1)N(CC1CC(O)C1)c1cccc(Cl)c1Cl] | white solid, yield: 73% | ¹H NMR (400 MHz, DMSO-d₆) δ 7.74- 7.40 (m, 6H), 4.98- 4.78 (m, 1H), 4.53- 3.73 (m, 3H), 2.27- 1.95 (m, 3H), 1.52- 1.47 (m, 2H) | 458.0 |
| | N-(2,3- | | | |
| | dichlorophenyl)-N- | | | |
| | ((3- | | | |
| | hydroxycyclobutyl) | | | |
| | methyl)-5- | | | |
| | (trifluoromethyl)-1H- | | | |
| | benzo[d]imidazole-2- | | | |
| | carboxamide | | | |
| Example 178 Method 8 | 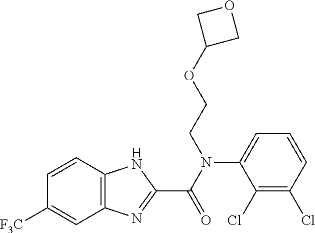 [SMILES: O=C(c1nc2cc(C(F)(F)F)ccc2[nH]1)N(CCOC1COC1)c1cccc(Cl)c1Cl] | white solid, yield: 3% | ¹H NMR (400 MHz, DMSO-d₆) δ 8.08-7.41 (m, 6H), 4.87-4.24 (m, 6H), 3.67-3.57 (m, 3H) | 474.0 |
| | N-(2,3- | | | |
| | dichlorophenyl)-N- | | | |
| | (2-(oxetan-3- | | | |
| | yloxy)ethyl)-5- | | | |
| | (trifluoromethyl)-1H- | | | |
| | benzo[d]imidazole-2- | | | |
| | carboxamide | | | |
| Example 179 Method 8 | 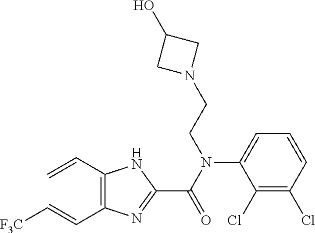 [SMILES: C=Cc1[nH]c(C(=O)N(CCN2CC(O)C2)c2cccc(Cl)c2Cl)nc1/C=C/C(F)(F)F] | white solid, yield: 20% | ¹H NMR (400 MHz, DMSO-d₆) δ 13.68 (brs, 1H), 8.08-7.40 (m, 6H), 5.26-5.20 (m, 1H), 4.68-4.64 (m, 0.4H), 4.14-4.02 (m, 2.6H), 3.53-3.42 (m, 2H), 2.73-2.51 (m, 4H) | 473.0 |
| | N-(2,3- | | | |
| | dichlorophenyl)-N- | | | |
| | (2-(3- | | | |
| | hydroxyazetidin-1- | | | |
| | yl)ethyl)-5- | | | |
| | (trifluoromethyl)-1H- | | | |
| | benzo[d]imidazole-2- | | | |
| | carboxamide | | | |
| Example 180 Method 8 | 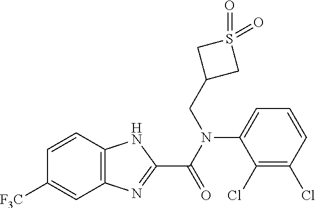 [SMILES: O=C(c1nc2cc(C(F)(F)F)ccc2[nH]1)N(CC1CS(=O)(=O)C1)c1cccc(Cl)c1Cl] | white solid, yield: 14% | ¹H NMR (400 MHz, Methanol-d₄) δ 7.73- 7.60 (m, 3H), 7.55- 7.47 (m, 2H), 7.41 (t, J = 8.0 Hz, 1H), 5.07- 4.86 (m, 1H), 4.41- 4.18 (m, 3H), 4.15- 4.06 (m, 1H), 4.06- 3.94 (m, 1H), 3.06- 2.82 (m, 1H) | 492.1 |
| | N-(2,3- | | | |
| | dichlorophenyl)-N- | | | |
| | ((1,1-dioxidothietan- | | | |
| | 3-yl)methyl)-5- | | | |
| | (trifluoromethyl)-1H- | | | |
| | benzo[d]imidazole-2- | | | |
| | carboxamide | | | |
| Example 181 Method 8 | 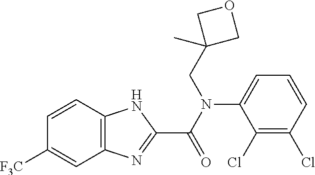 [SMILES: CC1(CN(C(=O)c2nc3cc(C(F)(F)F)ccc3[nH]2)c2cccc(Cl)c2Cl)COC1] | white solid, yield: 27% | ¹H NMR (400 MHz, DMSO-d₆) δ 11.66- 10.40 (m, 1H), 8.09- 7.32 (m, 6H), 5.04- 3.81 (m, 6H), 1.42 (d, J = 12.4 Hz, 3H) | 458.0 |
| | N-(2,3- | | | |
| | dichlorophenyl)-N- | | | |
| | ((3-methyloxetan-3- | | | |
| | yl)methyl)-5- | | | |
| | (trifluoromethyl)-1H- | | | |
| | benzo[d]imidazole-2- | | | |
| | carboxamide | | | |
| Example 182 Method 8 | 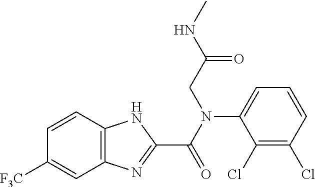 [SMILES: CNC(=O)CN(C(=O)c1nc2cc(C(F)(F)F)ccc2[nH]1)c1cccc(Cl)c1Cl] | white solid, yield: 19% | ¹H NMR (400 MHz, DMSO-d₆) δ 13.57 (s, 1H), 8.15-7.28 (m, 6H), 6.25-3.86 (m, 2H), 2.64-2.54 (m, 3H) | 445.1 |
| | N-(2,3- | | | |
| | dichlorophenyl)-N- | | | |
| | (2-(methylamino)-2- | | | |
| | oxoethyl)-5- | | | |
| | (trifluoromethyl)-1H- | | | |
| | benzo[d]imidazole-2- | | | |
| | carboxamide | | | |
| Example 184 Method 8 | 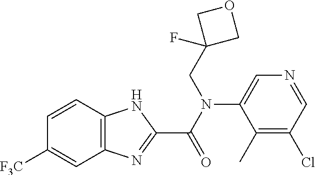 [SMILES: Cc1c(Cl)cncc1N(CC1(F)COC1)C(=O)c1nc2cc(C(F)(F)F)ccc2[nH]1] | white solid, yield: 17% | ¹H NMR (400 MHz, DMSO-d₆) δ 13.81 (s, 1H), 8.76-7.41 (m, 5H), 5.67-4.15 (m, 6H), 2.26 (s, 3H) | 443.1 |
| | N-(5-chloro-4- | | | |
| | methylpyridin-3-yl)- | | | |
| | N-((3-fluorooxetan- | | | |
| | 3-yl)methyl)-5- | | | |
| | (trifluoromethyl)-1H- | | | |
| | benzo[d]imidazole-2- | | | |
| | carboxamide | | | |
| Example 185 Method 8 | 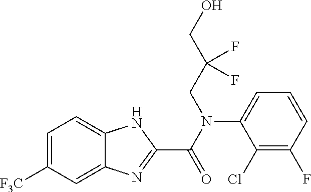 [SMILES: O=C(c1nc2cc(C(F)(F)F)ccc2[nH]1)N(CC(F)(F)CO)c1cccc(F)c1Cl] | white solid, yield: 18% | ¹H NMR (400 MHz, DMSO-d₆) δ 7.74-7.43 (m, 6H), 5.62 (t, J = 6.0 Hz, 1H), 4.38-4.09 (m, 2H), 3.76-3.68 (m, 2H) | 452.1 |
| | N-(2-chloro-3- | | | |
| | fluorophenyl)-N- | | | |
| | (2,2-difluoro-3- | | | |
| | hydroxypropyl)-5- | | | |
| | (trifluoromethyl)-1H- | | | |
| | benzo[d]imidazole-2- | | | |
| | carboxamide | | | |
| Example 186 Method 8 | 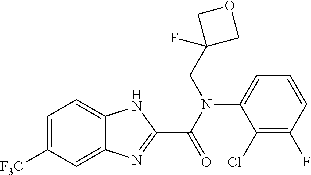 [SMILES: O=C(c1nc2cc(C(F)(F)F)ccc2[nH]1)N(CC1(F)COC1)c1cccc(F)c1Cl] | white solid, yield: 26% | ¹H NMR (400 MHz, Methanol-d₄) δ 8.12- 7.45 (m, 3H), 7.45- 7.20 (m, 3H), 4.80- 4.59 (m, 5H), 4.49- 4.43 (m, 1H) | 446.1 |
| | N-(2-chloro-3- | | | |
| | fluorophenyl)-N-((3- | | | |
| | fluorooxetan-3- | | | |
| | yl)methyl)-5- | | | |
| | (trifluoromethyl)-1H- | | | |
| | benzo[d]imidazole-2- | | | |
| | carboxamide | | | |
| Example 188 Method 8 | 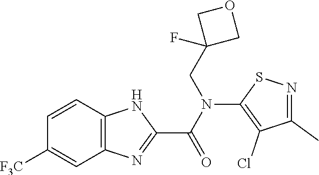 [SMILES: Cc1nsc(N(CC2(F)COC2)C(=O)c2nc3cc(C(F)(F)F)ccc3[nH]2)c1Cl] | white solid, yield: 13% | ¹H NMR (400 MHz, DMSO-d₆) δ 13.93 (s, 1H), 8.12-7.41 (m, 3H), 5.04-4.37 (m, 6H), 2.36 (s, 3H). | 449.2 |
| | N-(4-chloro-3- | | | |
| | methylisothiazol-5- | | | |
| | yl)-N-((3- | | | |
| | fluorooxetan-3- | | | |
| | yl)methyl)-5- | | | |
| | (trifluoromethyl)-1H- | | | |
| | benzo[d]imidazole-2- | | | |
| | carboxamide | | | |
| Example 193 Method 8 | 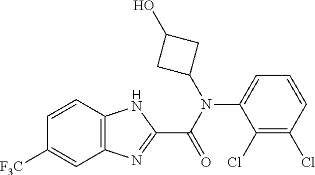 [SMILES: O=C(c1nc2cc(C(F)(F)F)ccc2[nH]1)N(c1cccc(Cl)c1Cl)C1CC(O)C1] | white solid, yield: 6% | ¹H NMR (400 MHz, DMSO-d₆) δ 8.11- 7.45 (m, 6H), 5.13- 5.04 (m, 1H), 4.60- 4.55 (m, 1H), 3.91- 3.75 (m, 1H), 2.62- 2.58 (m, 1H), 2.50- 2.48 (m, 1H), 1.78- 1.71 (m, 1H), 1.59- 1.52 (m, 1H) | 444.0 |
| | N-(2,3- | | | |
| | dichlorophenyl)-N- | | | |
| | (3- | | | |
| | hydroxycyclobutyl)- | | | |
| | 5-(trifluoromethyl)- | | | |
| | 1H- | | | |
| | benzo[d]imidazole-2- | | | |
| | carboxamide | | | |
| Example 194 Method 8 | 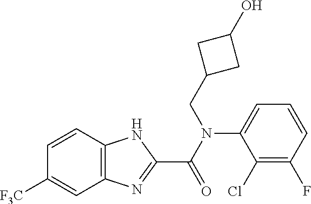 [SMILES: O=C(c1nc2cc(C(F)(F)F)ccc2[nH]1)N(CC1CC(O)C1)c1cccc(F)c1Cl] | white solid, yield: 59% | ¹H NMR (400 MHz, DMSO-d₆) δ 13.69 (s, 1H), 7.91-7.40 (m, 6H), 4.97-4.90 (m, 1H), 4.61-3.80 (m, 3H), 2.28-1.95 (m, 3H), 1.68-1.17 (m, 2H) | 442.0 |
| | N-(2-chloro-3- | | | |
| | fluorophenyl)-N-((3- | | | |
| | hydroxycyclobutyl) | | | |
| | methyl)-5- | | | |
| | (trifluoromethyl)-1H- | | | |
| | benzo[d]imidazole-2- | | | |
| | carboxamide | | | |
| Example 197 Method 8 | 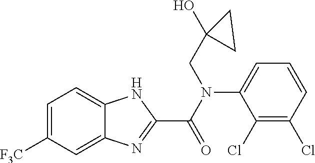 [SMILES: O=C(c1nc2cc(C(F)(F)F)ccc2[nH]1)N(CC1(O)CC1)c1cccc(Cl)c1Cl] | white solid, yield: 36% | ¹H NMR (400 MHz, Methanol-d₄) δ 8.63- 7.27 (m, 6H), 5.47 (d, J = 14.8 Hz, 0.4H), 4.77 (d, J = 14.0 Hz, 0.6H), 3.71-3.36 (m, 1H), 0.86-0.11 (m, 4H) | 444.0 |
| | N-(2,3- | | | |
| | dichlorophenyl)-N- | | | |
| | ((1- | | | |
| | hydroxycyclopropyl) | | | |
| | methyl)-5- | | | |
| | (trifluoromethyl)-1H- | | | |
| | benzo[d]imidazole-2- | | | |
| | carboxamide | | | |
| Example 198 Method 8 | 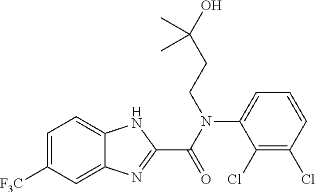 [SMILES: CC(C)(O)CCN(C(=O)c1nc2cc(C(F)(F)F)ccc2[nH]1)c1cccc(Cl)c1Cl] | white solid, yield: 5% | ¹H NMR (400 MHz, DMSO-d₆) δ 13.52 (s, 1H), 8.02-7.40 (m, 6H), 4.35 (s, 1H), 4.14- 4.08 (m, 1H), 3.77- 3.73 (m, 1H), 2.01- 1.99 (m, 1H), 1.74- 1.70 (m, 1H), 1.13- 1.11 (m, 6H) | 460.1 |
| | N-(2,3- | | | |
| | dichlorophenyl)-N- | | | |
| | (3-hydroxy-3- | | | |
| | methylbutyl)-5- | | | |
| | (trifluoromethyl)-1H- | | | |
| | benzo[d]imidazole-2- | | | |
| | carboxamide | | | |
| Example 199 Method 8 | 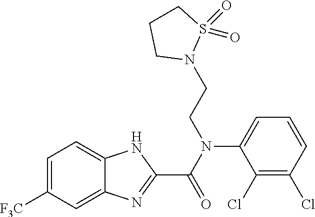 [SMILES: O=C(c1nc2cc(C(F)(F)F)ccc2[nH]1)N(CCN1CCCS1(=O)=O)c1cccc(Cl)c1Cl] | white solid, yield: 1% | ¹H NMR (400 MHz, DMSO-d₆) δ 14.75- 13.04 (m, 1H), 8.19- 7.31 (m, 6H), 5.12- 3.57 (m, 2H), 3.23- 3.07 (m, 5H), 2.55 (s, 1H), 2.23-1.96 (m, 2H) | 521.0 |
| | N-(2,3- | | | |
| | dichlorophenyl)-N- | | | |
| | (2-(1,1- | | | |
| | dioxidoisothiazolidin- | | | |
| | 2-yl)ethyl)-5- | | | |
| | (trifluoromethyl)-1H- | | | |
| | benzo[d]imidazole-2- | | | |
| | carboxamide | | | |
| Example 200 Method 8 | 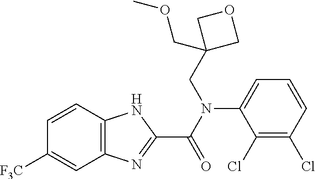 [SMILES: COCC1(CN(C(=O)c2nc3cc(C(F)(F)F)ccc3[nH]2)c2cccc(Cl)c2Cl)COC1] | white solid, yield: 25% | ¹H NMR (400 MHz, DMSO-d₆) δ 13.73 (s, 1H), 7.76-7.39 (m, 6H), 5.09-3.98 (m, 6H), 3.67-3.54 (m, 2H), 3.21-3.03 (m, 3H) | 488.1 |
| | N-(2,3- | | | |
| | dichlorophenyl)-N- | | | |
| | (3-(methoxymethyl) | | | |
| | oxetan- | | | |
| | 3-yl)methyl)-5- | | | |
| | (trifluoromethyl)-1H- | | | |
| | benzo[d]imidazole-2- | | | |
| | carboxamide | | | |
| Example 202 Method 8 | 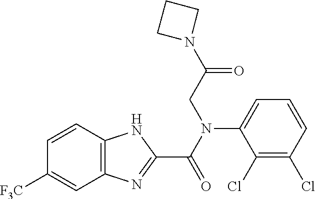 [SMILES: O=C(CN(C(=O)c1nc2cc(C(F)(F)F)ccc2[nH]1)c1cccc(Cl)c1Cl)N1CCC1] | white solid, yield: 14% | ¹H NMR (400 MHz, DMSO-d₆) δ 7.74-7.34 (m, 6H), 6.01-4.87 (m, 1H), 4.27-3.90 (m, 5H), 2.27-2.22 (m, 2H) | 471.1 |
| | N-(2-(azetidin-1-yl)- | | | |
| | 2-oxoethyl)-N-(2,3- | | | |
| | dichlorophenyl)-5- | | | |
| | (trifluoromethyl)-1H- | | | |
| | benzo[d]imidazole-2- | | | |
| | carboxamide | | | |
| Example 207 Method 8 | 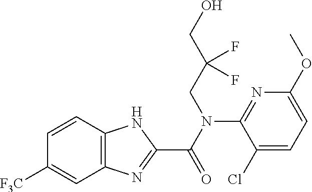 [SMILES: COc1ccc(Cl)c(N(CC(F)(F)CO)C(=O)c2nc3cc(C(F)(F)F)ccc3[nH]2)n1] | white solid, yield: 63% | ¹H NMR (400 MHz, DMSO-d₆) δ 13.87 (s, 1H), 8.12-7.40 (m, 4H), 6.87 (d, J = 8.4 Hz, 1H), 5.58 (s, 1H), 4.62 (s, 2H), 3.85 (s, 3H), 3.74 (s, 2H) | 465.1 |
| | N-(3-chloro-6- | | | |
| | methoxypyridin-2- | | | |
| | yI)-N-(2,2-difluoro- | | | |
| | 3-hydroxypropyl)-5- | | | |
| | (trifluoromethyl)-1H- | | | |
| | benzo[d]imidazole-2- | | | |
| | carboxamide | | | |
| Example 212 Method 8 | 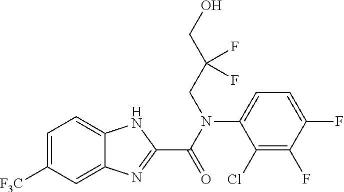 [SMILES: O=C(c1nc2cc(C(F)(F)F)ccc2[nH]1)N(CC(F)(F)CO)c1ccc(F)c(F)c1Cl] | white solid, yield: 52% | ¹H NMR (400 MHz, DMSO-d₆) δ 13.86- 13.80 (m, 1H), 8.55- 7.46 (m, 5H), 5.64- 5.61 (m, 1H), 5.41- 4.16 (m, 2H), 3.76- 3.68 (m, 2H) | 470.1 |
| | N-(2-chloro-3,4- | | | |
| | difluorophenyl)-N- | | | |
| | (2,2-difluoro-3- | | | |
| | hydroxypropyl)-5- | | | |
| | (trifluoromethyl)-1H- | | | |
| | benzo[d]imidazole-2- | | | |
| | carboxamide | | | |
| Example 213 Method 8 | 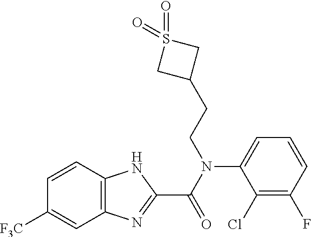 [SMILES: O=C(c1nc2cc(C(F)(F)F)ccc2[nH]1)N(CCC1CS(=O)(=O)C1)c1cccc(F)c1Cl] | white solid, yield: 5% | ¹H NMR (400 MHz, DMSO-d₆) δ 13.70 (brs, 1H), 7.73-7.43 (m, 6H), 4.14-3.33 (m, 6H), 2.67-2.60 (m, 1H), 2.20-1.91 (m, 2H) | 490.1 |
| | N-(2-chloro-3- | | | |
| | fluorophenyl)-N-(2- | | | |
| | (1,1-dioxidothietan- | | | |
| | 3-yl)ethyl)-5- | | | |
| | (trifluoromethyl)-1H- | | | |
| | benzo[d]imidazole-2- | | | |
| | carboxamide | | | |
| Example 214 Method 8 | 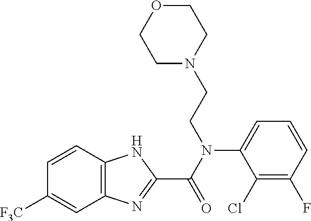 [SMILES: O=C(c1nc2cc(C(F)(F)F)ccc2[nH]1)N(CCN1CCOCC1)c1cccc(F)c1Cl] | white solid, yield: 5% | ¹H NMR (400 MHz, DMSO-d₆) δ 13.68 (s, 1H), 8.29-7.17 (m, 6H), 5.52-3.42 (m, 5H), 3.27-3.18 (m, 1H), 2.71-2.55 (m, 2H), 2.45-2.17 (m, 4H) | 471.1 |
| | N-(2-chloro-3- | | | |
| | fluorophenyl)-N-(2- | | | |
| | morpholinoethyl)-5- | | | |
| | (trifluoromethyl)-1H- | | | |
| | benzo[d]imidazole-2- | | | |
| | carboxamide | | | |
| Example 215 Method 8 | 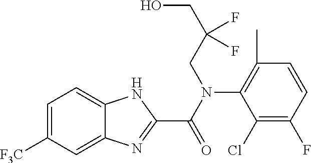 [SMILES: Cc1ccc(F)c(Cl)c1N(CC(F)(F)CO)C(=O)c1nc2cc(C(F)(F)F)ccc2[nH]1] | white solid, yield: 38% | ¹H NMR (400 MHz, DMSO-d₆) δ 13.79 (s, 1H), 8.19-7.13 (m, 5H), 5.95-5.38 (m, 1H), 4.88-3.57 (m, 3H), 2.34-2.16 (m, 3H) | 466.1 |
| | N-(2-chloro-3-fluoro- | | | |
| | 6-methylphenyl)-N- | | | |
| | (2,2-difluoro-3- | | | |
| | hydroxypropyl)-5- | | | |
| | (trifluoromethyl)-1H- | | | |
| | benzo[d]imidazole-2- | | | |
| | carboxamide | | | |
| Example 216 Method 8 | 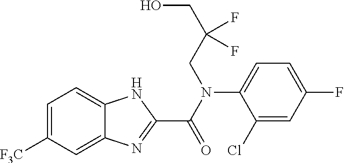 [SMILES: O=C(c1nc2cc(C(F)(F)F)ccc2[nH]1)N(CC(F)(F)CO)c1ccc(F)cc1Cl] | white solid, yield: 57% | ¹H NMR (400 MHz, DMSO-d₆) δ 13.76 (s, 1H), 7.76-7.28 (m, 6H), 5.61 (t, J = 6.0 Hz, 1H), 4.91-4.64 (m, 1H), 4.14-3.32 (m, 3H) | 452.1 |
| | N-(2-chloro-4- | | | |
| | fluorophenyl)-N- | | | |
| | (2,2-difluoro-3- | | | |
| | hydroxypropyl)-5- | | | |
| | (trifluoromethyl)-1H- | | | |
| | benzo[d]imidazole-2- | | | |
| | carboxamide | | | |
| Example 217 Method 8 | 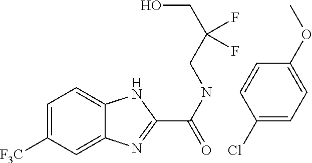 | white solid, yield: 63% | ¹H NMR (400 MHz, DMSO-d₆) δ 13.87 (s, 1H), 8.12-7.40 (m, 4H), 6.87 (d, J = 8.4 Hz, 1H), 5.58 (s, 1H), 4.62 (s, 2H), 3.85 (s, 3H), 3.74 (s, 2H) | 465.1 |
| | N-(3-chloro-6- | | | |
| | methoxypyridin-2- | | | |
| | yl)-N-(2,2-difluoro- | | | |
| | 3-hydroxypropyl)-5- | | | |
| | (trifluoromethyl)-1H- | | | |
| | benzo[d]imidazole-2- | | | |
| | carboxamide | | | |
| Example 218 Method 8 | 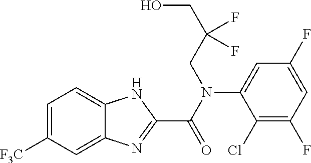 [SMILES: O=C(c1nc2cc(C(F)(F)F)ccc2[nH]1)N(CC(F)(F)CO)c1cc(F)cc(F)c1Cl] | white solid, yield: 49% | ¹H NMR (400 MHz, DMSO-d₆) δ 8.10- 7.54 (m, 5H), 5.63- 4.99 (m, 1H), 4.58- 4.37 (m, 2H), 3.76- 3.69 (m, 2H) | 470.0 |
| | N-(2-chloro-3,5- | | | |
| | difluorophenyl)-N- | | | |
| | (2,2-difluoro-3- | | | |
| | hydroxypropyl)-5- | | | |
| | (trifluoromethyl)-1H- | | | |
| | benzo[d]imidazole-2- | | | |
| | carboxamide | | | |
| Example 219 Method 8 | 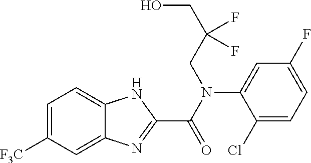 [SMILES: O=C(c1nc2cc(C(F)(F)F)ccc2[nH]1)N(CC(F)(F)CO)c1cc(F)ccc1Cl] | white solid. yield: 58% | ¹H NMR (400 MHz, DMSO-d₆) δ 13.77 (s, 1H), 8.11-7.26 (m, 6H), 5.63-4.94 (m, 1H), 4.68-4.24 (m, 2H), 3.77-3.68 (m, 2H) | 452.1 |
| | N-(2-chloro-5- | | | |
| | fluorophenyl)-N- | | | |
| | (2,2-difluoro-3- | | | |
| | hydroxypropyl)-5- | | | |
| | (trifluoromethyl)-1H- | | | |
| | benzo[d]imidazole-2- | | | |
| | carboxamide | | | |
| Example 220 Method 8 | 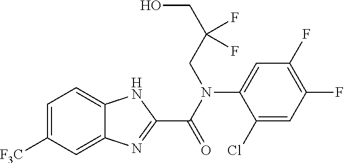 [SMILES: O=C(c1nc2cc(C(F)(F)F)ccc2[nH]1)N(CC(F)(F)CO)c1cc(F)c(F)cc1Cl] | white solid, yield: 24% | ¹H NMR (400 MHz, DMSO-d₆) δ 13.81 (s, 1H), 7.98-7.67 (m, 5H), 5.63 (t, J = 6.4 Hz, 1H), 4.59-4.52 (m, 1H), 4.30-4.25 (m, 1H), 3.38-3.68 (m, 2H) | 470.0 |
| | N-(2-chloro-4,5- | | | |
| | difluorophenyl)-N- | | | |
| | (2,2-difluoro-3- | | | |
| | hydroxypropyl)-5- | | | |
| | (trifluoromethyl)-1H- | | | |
| | benzo[d]imidazole-2- | | | |
| | carboxamide | | | |
| Example 224 Method 8 | 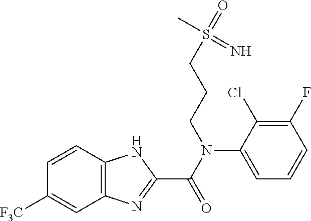 [SMILES: CS(=N)(=O)CCCN(C(=O)c1nc2cc(C(F)(F)F)ccc2[nH]1)c1cccc(F)c1Cl] | white solid, yield: 56% | ¹H NMR (400 MHz, DMSO-d₆) δ 13.70 (s, 1H), 7.73-7.46 (m, 6H), 4.62-3.67 (m, 3H), 3.20-3.17 (m, 2H), 2.91 (d, J = 7.6 Hz, 3H), 2.09-2.01 (m, 2H) | 477.0 |
| | N-(2-chloro-3- | | | |
| | fluorophenyl)-N-(3- | | | |
| | (S- | | | |
| | methylsulfonimidoyl) | | | |
| | propyl)-5- | | | |
| | (trifluoromethyl)-1H- | | | |
| | benzo[d]imidazole-2- | | | |
| | carboxamide | | | |
| Example 225 Method 8 | 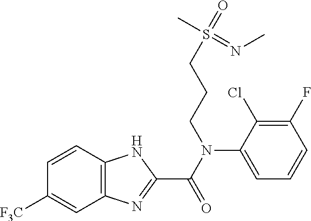 [SMILES: CN=S(C)(=O)CCCN(C(=O)c1nc2cc(C(F)(F)F)ccc2[nH]1)c1cccc(F)c1Cl] | white solid, yield: 8% | ¹H NMR (400 MHz, DMSO-d₆) δ 13.69 (s, 1H), 7.80-7.43 (m, 6H), 4.16-3.52 (m, 2H), 3.24-3.19 (m, 2H), 2.90 (d, J = 7.2 Hz, 3H), 2.60 (s, 3H), 2.02 (s, 2H) | 491.1 |
| | N-(2-chloro-3- | | | |
| | fluorophenyl)-N-(3- | | | |
| | (N,S- | | | |
| | dimethylsulfonimidoyl) | | | |
| | propyl)-5- | | | |
| | (trifluoromethyl)-1H- | | | |
| | benzo[d]imidazole-2- | | | |
| | carboxamide | | | |
| Example 226 Method 8 | 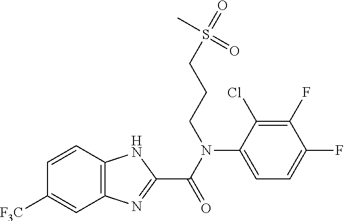 [SMILES: CS(=O)(=O)CCCN(C(=O)c1nc2cc(C(F)(F)F)ccc2[nH]1)c1ccc(F)c(F)c1Cl] | white solid, yield: 11% | ¹H NMR (400 MHz, DMSO-d₆) δ 13.70 (s, 1H), 7.78-7.52 (m, 5H), 4.58-3.70 (m, 2H), 3.30-3.21 (m, 2H), 3.00 (d, J = 11.6 Hz, 3H), 2.19-2.00 (m, 2H) | 496.1 |
| | N-(2-chloro-3,4- | | | |
| | difluorophenyl)-N- | | | |
| | (3- | | | |
| | (methylsulfonyl) | | | |
| | propyl)-5- | | | |
| | (trifluoromethyl)-1H- | | | |
| | benzo[d]imidazole-2- | | | |
| | carboxamide | | | |
| Example 227 Method 8 | 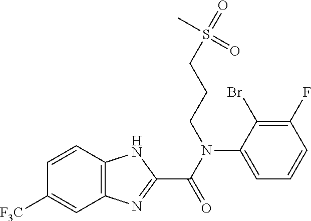 [SMILES: CS(=O)(=O)CCCN(C(=O)c1nc2cc(C(F)(F)F)ccc2[nH]1)c1cccc(F)c1Br] | white solid, yield: 10% | ¹H NMR (400 MHz, DMSO-d₆) δ 13.69 (brs, 1H), 7.72-7.40 (m, 6H), 4.72-3.66 (m, 2H), 3.32-3.23 (m, 2H), 3.02-2.99 (d, J = 11.6 Hz, 3H), 2.23- 2.02 (m, 2H). | 522.1 |
| | N-(2-bromo-3- | | | |
| | fluorophenyl)-N-(3- | | | |
| | (methylsulfonyl) | | | |
| | propyl)-5- | | | |
| | (trifluoromethyl)-1H- | | | |
| | benzo[d]imidazole-2- | | | |
| | carboxamide | | | |
| Example 228 Method 8 Mixture of Enantiomers | 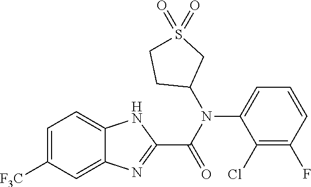 [SMILES: O=C(c1nc2cc(C(F)(F)F)ccc2[nH]1)N(c1cccc(F)c1Cl)C1CCS(=O)(=O)C1] | white solid, yield: 16% | ¹H NMR (400 MHz, Methanol-d₄) δ 7.95- 7.27 (m, 6H), 5.18 (q, J = 8.8 Hz, 1H), 3.81-3.66 (m, 1H), 3.56-3.43 (m, 1H), 3.22 (t, J = 11.2 Hz, 2H), 2.69-2.28 (m, 2H). | 476.1 |
| | N-(2-chloro-3- | | | |
| | fluorophenyl)-N- | | | |
| | (1,1- | | | |
| | dioxidotetrahydro- | | | |
| | thiophen-3-yl)-5- | | | |
| | (trifluoromethyl)-1H- | | | |
| | benzo[d]imidazole-2- | | | |
| | carboxamide | | | |
| Example 229 Method 8 | 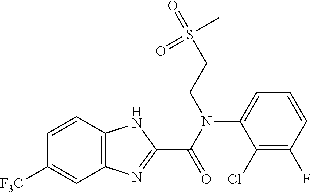 [SMILES: CS(=O)(=O)CCN(C(=O)c1nc2cc(C(F)(F)F)ccc2[nH]1)c1cccc(F)c1Cl] | white solid, yield: 3% | ¹H NMR (400 MHz, DMSO-d₆) δ 13.74 (s, 1H), 8.12-7.46 (m, 6H), 5.05-3.97 (m, 2H), 3.74-3.52 (m, 2H), 3.11 (d, J = 6.0 Hz, 3H) | 464.1 |
| | N-(2-chloro-3- | | | |
| | fluorophenyl)-N-(2- | | | |
| | (methylsulfonyl)ethyl)- | | | |
| | 5-(trifluoromethyl)- | | | |
| | 1H- | | | |
| | benzo[d]imidazole-2- | | | |
| | carboxamide | | | |
| Example 230 Method 8 Mixture of Enantiomers | 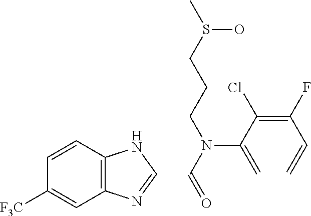 | white solid, yield: 51% | ¹H NMR (400 MHz, DMSO-d₆) δ 13.69 (s, 1H), 7.73-7.42 (m, 6H), 4.62-3.75 (m, 2H), 2.95-2.71 (m, 2H), 2.54 (s, 3H), 2.14- 1.95 (m, 2H) | 462.0 |
| | N-(2-chloro-3- | | | |
| | fluorophenyl)-N-(3- | | | |
| | (methylsulfinyl) | | | |
| | propyl)-5- | | | |
| | (trifluoromethyl)-1H- | | | |
| | benzo[d]imidazole-2- | | | |
| | carboxamide | | | |
| Example 238 Method 8 Mixture of Enantiomers | 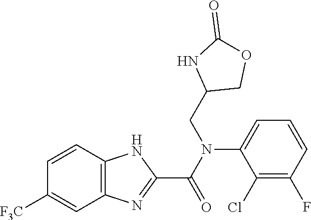 [SMILES: O=C1NC(CN(C(=O)c2nc3cc(C(F)(F)F)ccc3[nH]2)c2cccc(F)c2Cl)CO1] | white solid, yield: 18% | ¹H NMR (400 MHz, DMSO-d₆) δ 13.71 (s, 1H), 8.04-7.44 (m, 6H), 4.47-3.61 (m, 5H) | 457.1 |
| | N-(2-chloro-3- | | | |
| | fluorophenyl)-N-((2- | | | |
| | oxooxazolidin-4- | | | |
| | yl)methyl)-5- | | | |
| | (trifluoromethyl)-1H- | | | |
| | benzo[d]imidazole-2- | | | |
| | carboxamide | | | |
| Example 239 Method 8 Mixture of Enantiomers | 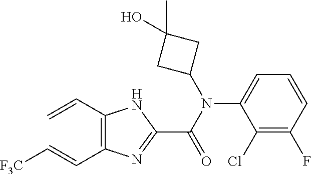 [SMILES: C=Cc1[nH]c(C(=O)N(c2cccc(F)c2Cl)C2CC(C)(O)C2)nc1/C=C/C(F)(F)F] | white solid, yield: 20% | ¹H NMR (400 MHz, DMSO-d₆) δ 13.64 (s, 1H), 7.71-7.31 (m, 6H), 5.01-4.93 (m, 1H), 4.66-4.61 (m, 1H), 2.39-2.33 (m, 1H), 2.26-2.22 (m, 1H), 1.99-1.94 (m, 1H), 1.84-1.78 (m, 1H), 1.30 (s, 3H) | 442.1 |
| | N-(2-chloro-3- | | | |
| | fluorophenyl)-N-(3- | | | |
| | hydroxy-3- | | | |
| | methylcyclobutyl)-5- | | | |
| | (trifluoromethyl)-1H- | | | |
| | benzo[d]imidazole-2- | | | |
| | carboxamide (5.2 mg, | | | |
| | 20%) as a white solid | | | |
| Example 240 Method 8 Mixture of Enantiomers | 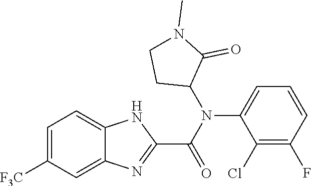 [SMILES: CN1CCC(N(C(=O)c2nc3cc(C(F)(F)F)ccc3[nH]2)c2cccc(F)c2Cl)C1=O] | white solid, yield: 12% | ¹H NMR (400 MHz, DMSO-d₆) δ 13.77 (s, 1H), 7.81-7.63 (m, 2H), 7.60-7,52 (m, 1H), 7.50-7.37 (m, 2H), 7.35-7,23 (m, 1H), 4.33(t, 1H), 3.51 (m, 2H), 2.84 (s, 3H), 2.57 (s, 1H), 2.39- 2.28 (m, 1H) | 455.1 |
| | N-(2-chloro-3- | | | |
| | fluorophenyl)-N-(1- | | | |
| | methyl-2- | | | |
| | oxopyrrolidin-3-yl)- | | | |
| | 5-(trifluoromethyl)- | | | |
| | 1H- | | | |
| | benzo[d]imidazole-2- | | | |
| | carboxamide | | | |

### Preparation of N-(2,3-dichlorophenyl)-N-(2-hydroxyethyl)-5-(methylsulfonyl)-1H-benzo[d]imidazole-2-carboxamide (108)

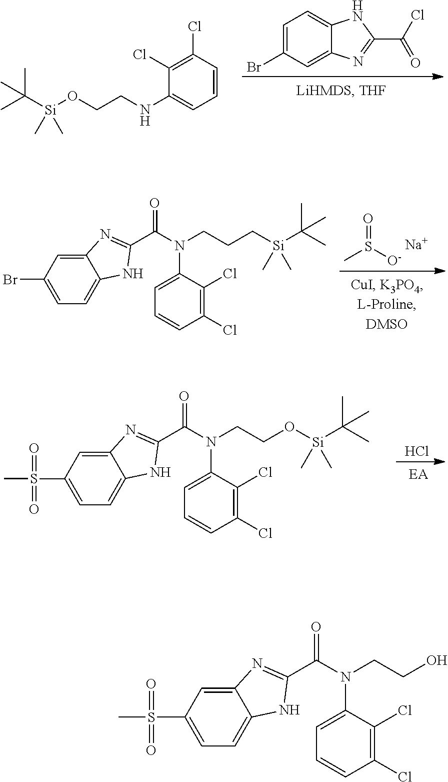

### Step 1: 5-bromo-N-(2-((tert-butyldimethylsilyl)oxy)ethyl)-N-(2,3-dichlorophenyl)-1H-benzo[d]imidazole-2-carboxamide

To a stirred solution of 5-bromo-1H-benzo[d]imidazole-2-carbonyl chloride (1.30 eq, 105 mg, 0.406 mmol) in THF (10 mL) were added LiHMDS (1.50 eq, mg, 0.47 mL) dropwise at 0° C. Then N-(2-((tert-butyldimethylsilyl)oxy)ethyl)-2,3-dichloroaniline (1.00 eq, 100 mg, 0.312 mmol) was added. The resulting mixture was stirred for 4 hours. The reaction was quenched with NH₄Cl (a.q.). The resulting mixture was extracted with EtOAc. The combined organic layers were washed with brine, and dried over anhydrous Na₂SO₄. After filtration, the filtrate was concentrated under reduced pressure. The residue was purified by prep-TLC (MeOH/DCM=1/25) to afford 5-bromo-N-(2-((tert-butyldimethylsilyl)oxy)ethyl)-N-(2,3-dichlorophenyl)-1H-benzo[d]imidazole-2-carboxamide (59 mg, 0.103 mmol, 33.04% yield) as a white solid. MS (ESI) m/z 543 [M+H]⁺

### Step 2: N-(2-((tert-butyldimethylsilyl)oxy)ethyl)-N-(2,3-dichlorophenyl)-5-(methylsulfonyl)-1H-benzo[d]imidazole-2-carboxamide

To a solution of 5-bromo-N-(2-((tert-butyldimethylsilyl)oxy)ethyl)-N-(2,3-dichlorophenyl)-1H-benzo[d]imidazole-2-carboxamide (1.00 eq, 80 mg, 0.147 mmol) in DMSO (5 mL) was added sodium methanesulfinate (1.20 eq, 18 mg, 0.177 mmol), L-Proline (0.200 eq, 3.4 mg, 0.0294 mmol), CuI (0.1000 eq, 2.8 mg, 0.0147 mmol), K₃PO₄ (2.00 eq, 62 mg, 0.294 mmol) and heating at 60° C. for 16 hours under an argon atmosphere. The reaction was quenched with NH₄Cl (a.q.). The resulting mixture was extracted with EtOAc. The combined organic layers were washed with brine, and dried over anhydrous Na₂SO₄. After filtration, the filtrate was concentrated under reduced pressure and purified by Silica gel, eluting with EtOAc in hexane from 0-40%] N-(2-((tert-butyldimethylsilyl)oxy)ethyl)-N-(2,3-dichlorophenyl)-5-(methylsulfonyl)-1H-benzo[d]imidazole-2-carboxamide (42 mg, 0.0735 mmol, 49.95% yield) as a yellow oil. MS (ESI) m/z 542 [M+H]⁺

### Step 3: N-(2,3-dichlorophenyl)-N-(2-hydroxyethyl)-5-(methylsulfonyl)-1H-benzo[d]imidazole-2-carboxamide

To a solution of N-(2-((tert-butyldimethylsilyl)oxy)ethyl)-N-(2,3-dichlorophenyl)-5-(methylsulfonyl)-1H-benzo[d]imidazole-2-carboxamide (1.00 eq, 42 mg, 0.0735 mmol) in Ethyl acetate (5 mL) was added HCl (1.20 eq, mg, 0.3 mL). The solution was stirred at room temperature for 1 hour. The mixture was basified by 1M NaOH to pH=9, then extracted by EA (25 mL×3) and concentrated under vacuum. The combined organic layers were washed with brine (15 mL×3), dried over Na₂SO₄ and concentrated under vacuum, which was purified by prep-HPLC to give the desired product N-(2,3-dichlorophenyl)-N-(2-hydroxyethyl)-5-(methylsulfonyl)-1H-benzo[d]imidazole-2-carboxamide (11 mg, 0.0252 mmol, 34.26% yield) as a white solid. MS (ESI) m/z 428 [M+H]⁺

¹H NMR (400 MHz, DMSO-d₆) δ 13.74 (s, 1H), 8.34-7.81 (m, 2H), 7.77-7.58 (m, 3H), 7.48 (dt, J=43.2, 8.0 Hz, 1H), 4.99-4.82 (m, 1H), 4.26 (dt, J=12.8, 6.0 Hz, 1H), 3.80-3.46 (m, 3H), 3.26 (s, 1H), 3.19 (s, 2H).

### Preparation of N-(2,3-dichlorophenyl)-N-(2-hydroxyethyl)-5-(methylsulfonyl)-1H-benzo[d]imidazole-2-carboxamide (139) Method 9

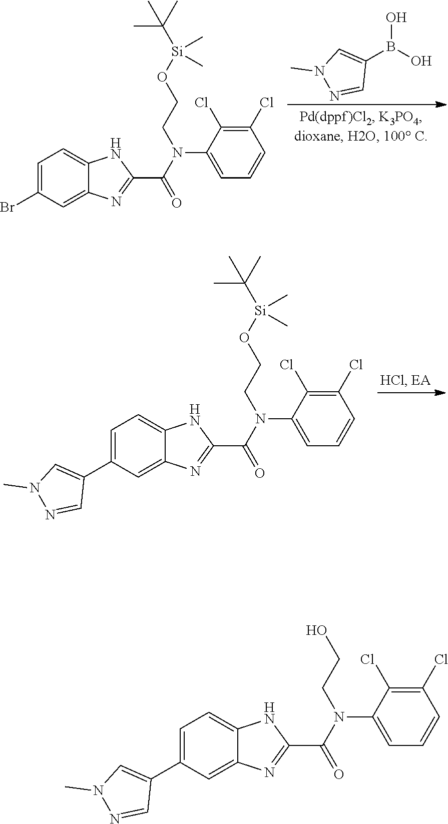

### Step 1. N-(2-((tert-butyldimethylsilyl)oxy)ethyl)-N-(2,3-dichlorophenyl)-5-(1-methyl-1H-pyrazol-4-yl)-1H-benzo[d]imidazole-2-carboxamide

To a solution of 5-bromo-2-[4-(2,3-dichlorophenyl)-1,2,4-triazol-3-yl]-1H-benzimidazole (1.00 eq, 155 mg, 0.379 mmol) in 1,4-Dioxane (5 mL) and water (1 mL), was added (3-methyl-1H-pyrazol-5-yl) boronic acid (2.00 eq, 95 mg, 0.758 mmol), Pd(dppf)Cl₂ (0.300 eq, 92 mg, 0.114 mmol) and K₃PO₄ (3.00 eq, 241 mg, 1.14 mmol). The mixture was stirred at 100° C. for 2 h. The mixture was diluted with EtOAc and washed with water. The organic layers were combined, dried over anhydrous sodium sulfate and purified by prep-HPLC to give 2-[4-(2,3-dichlorophenyl)-1,2,4-triazol-3-yl]-5-(3-methyl-1H-pyrazol-5-yl)-1H-benzimidazole (13 mg, 0.0301 mmol, 7.9% yield).

MS (ESI) m/z 410/412 [M+H]⁺.

### Step 2. N-(2,3-dichlorophenyl)-N-(2-hydroxyethyl)-5-(1-methyl-1H-pyrazol-4-yl)-1H-benzo[d]imidazole-2-carboxamide

To a solution of N-(2-((tert-butyldimethylsilyl)oxy)ethyl)-N-(2,3-dichlorophenyl)-5-(methylsulfonyl)-1H-benzo[d]imidazole-2-carboxamide (1.00 eq, 42 mg, 0.0735 mmol) in ethyl acetate (5 mL) was added HCl (1.20 eq, mg, 0.3 mL). The solution was stirred at room temperature for 1 hour. The mixture was basified by 1M NaOH to pH=9, then extracted by EA (25 mL×3) and concentrated under vacuum. The combined organic layers were washed with brine (15 mL×3), dried over Na₂SO₄ and concentrated under vacuum, which was purified by prep-HPLC to give the desired product N-(2,3-dichlorophenyl)-N-(2-hydroxyethyl)-5-(methylsulfonyl)-1H-benzo[d]imidazole-2-carboxamide (11 mg, 0.0252 mmol, 34.26% yield) as a white solid. MS (ESI) m/z 428 [M+H]⁺

¹H NMR (400 MHz, DMSO) δ 13.56 (s, 1H), 12.52 (s, 1H), 9.03 (s, 1H), 7.95-7.75 (m, 3H), 7.64-7.43 (m, 3H), 6.43 (s, 1H), 2.23 (s, 3H).

Examples (Compounds) 119, 122, 124, 142, 143, 144, 153, 154, 161, 162, 171, 187, 189, 190, 196, 210, 222 were synthesized using a similar method to that used in Example 139.

| Example | | | | |
|---|---|---|---|---|
| No. | | | | |
| (Compound | | Appearance | | MS |
| No.) | | and | | (m/z) |
| Method | Structure and Name | Yield | ¹H NMR Data | [M + H]⁺ |
| Example 119 Method 9 | 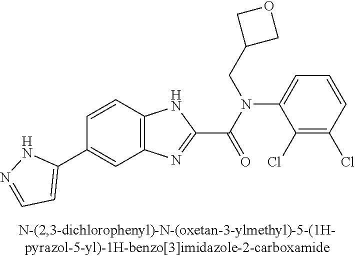 [SMILES: O=C(c1nc2cc(-c3ccn[nH]3)ccc2[nH]1)N(CC1COC1)c1cccc(Cl)c1Cl] | white solid, yield: 29% | ¹H NMR (400 MHz, Methanol-d₄) δ 7.88 (m, 1H), 7.70-7.60 (m, 2H), 7.55 (m, 1H), 7.49-7.31 (m, 3H), 6.68 (m, 1H), 4.78-4.70 (m, 2H), 4.51 (t, J = 6.2 Hz, 1H), 4.43 (t, J = 6.2 Hz, 1H), 4.31 (d, J = 7.4 Hz, 2H), 3.39 (m, 1H). | 442.3 |
| Example 122 Method 9 | 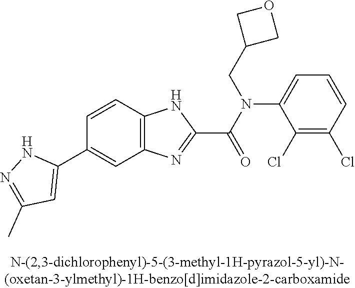 [SMILES: Cc1cc(-c2ccc3[nH]c(C(=O)N(CC4COC4)c4cccc(Cl)c4Cl)nc3c2)[nH]n1] | white solid, yield: 32% | ¹H NMR (400 MHz, DMSO-d₆) δ 13.27 (s, 1H), 12.50 (s, 1H), 7.80-7.64 (m, 2H), 7.52 (d, J = 8.1 Hz, 2H), 7.46-7.22 (m, 2H), 6.40 (s, 1H), 4.94 (m, 1H), 4.67-4.47 (m, 2H), 4.39-4.24 (m, 2H), 4.15 (m, 1H), 3.23 (m, 1H), 2.23 (m, 3H). | 456.2 |
| Example 124 Method 9 | 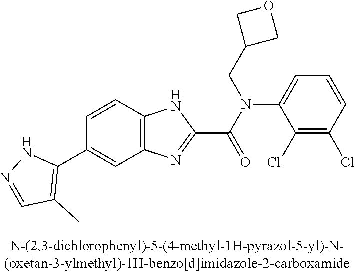 [SMILES: Cc1cn[nH]c1-c1ccc2[nH]c(C(=O)N(CC3COC3)c3cccc(Cl)c3Cl)nc2c1] | white solid, yield: 35% | ¹H NMR (400 MHz, Methanol-d₄) δ 7.68- 7.34 (m, 7H), 4.77- 4.68 (m, 2H), 4.51 (t, J = 6.2 Hz, 1H), 4.43 (t, J = 6.2 Hz, 1H), 4.35-4.26 (m, 2H), 3.39 (m, 1H), 2.31- 2.15 (m, 3H). | 456.2 |
| Example 142 Method 9 | 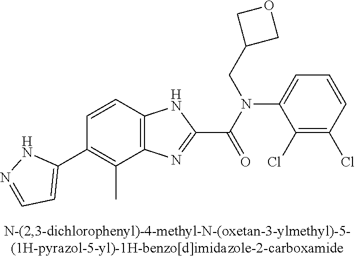 [SMILES: Cc1c(-c2ccn[nH]2)ccc2[nH]c(C(=O)N(CC3COC3)c3cccc(Cl)c3Cl)nc12] | white solid, yield: 32% | ¹H NMR (400 MHz, Methanol-d₄) δ 7.67- 7.24 (m, 6H), 6.43 (m, 1H), 4.81-4.72 (m, 2H), 4.59-4.28 (m, 4H), 3.52-3.37 (m, 1H), 2.21 (s, 3H). | 456.2 |
| Example 143 Method 9 | 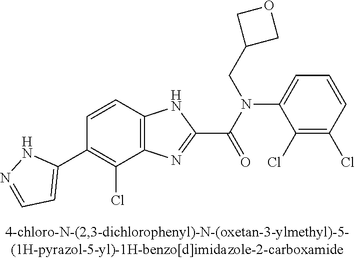 [SMILES: O=C(c1nc2c(Cl)c(-c3ccn[nH]3)ccc2[nH]1)N(CC1COC1)c1cccc(Cl)c1Cl] | white solid, yield: 6% | ¹H NMR (400 MHz, Methanol-d₄) δ 7.76- 7.69 (m, 2H), 7.65 (s, 1H), 7.05 (t, J = 8.1 Hz, 1H), 6.81- 6.75 (m, 2H), 6.72 (dd, J = 7.9, 1.3 Hz, 1H), 4.63-4.54 (m, 2H), 3.82 (d, J = 5.4 Hz, 2H), 3.47 (d, J = 6.7 Hz, 2H), 2.47-2.39 (m, 1H) | 476.0 |
| Example 144 Method 9 | 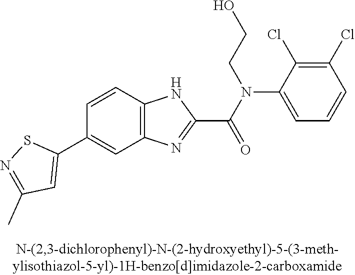 [SMILES: Cc1cc(-c2ccc3[nH]c(C(=O)N(CCO)c4cccc(Cl)c4Cl)nc3c2)sn1] | white solid, yield: 10% | ¹H NMR (400 MHz, Methanol-d₄) δ 7.99- 7.29 (m, 7H), 4.38 (dt, J = 12.8, 6.2 Hz, 1H), 3.99-3.72 (m, 2H), 3.64 (dt, J = 12.6, 6.0 Hz, 1H), 2.50 (d, J = 16.8 Hz, 3H). | 447.0 |
| Example 153 Method 9 | 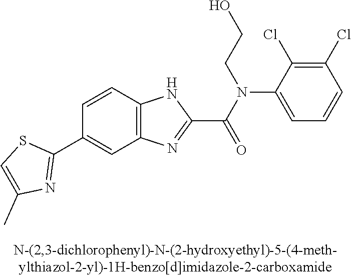 [SMILES: Cc1csc(-c2ccc3[nH]c(C(=O)N(CCO)c4cccc(Cl)c4Cl)nc3c2)n1] | white solid, yield: 39% | ¹H NMR (400 MHz, Methanol-d₄) δ 8.03- 7.93 (m, 1H), 7.80 (dd, J = 8.4, 1.6 Hz, 1H), 7.65-7.49 (m, 3H), 7.36 (t, J = 8.0 Hz, 1H), 7.13 (d, J = 20.4 Hz, 1H), 4.38 (dt, J = 13.6, 6.0 Hz, 1H), 3.91 (tt, J = 12.4, 5.6 Hz, 1H), 3.78 (dq, J = 11.6, 6.0 Hz, 1H), 3.65 (dt, J = 12.8, 6.0 Hz, 1H), 2.48 (d, J = 17.2 Hz, 3H). | 447.0 |
| Example 154 Method 9 | 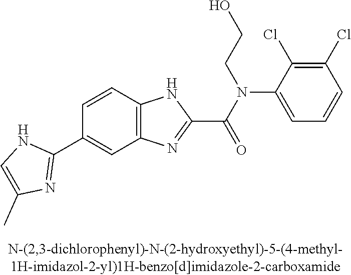 [SMILES: Cc1c[nH]c(-c2ccc3[nH]c(C(=O)N(CCO)c4cccc(Cl)c4Cl)nc3c2)n1] | white solid, yield: 25% | ¹H NMR (400 MHz, Methanol-d₄) δ 8.29 (s, 1H), 7.99-7.80 (m, 1H), 7.77-7.49 (m, 3H), 7.48-4.30 (m, 1H), 7.09 (d, J = 8.8 Hz, 1H), 4.39 (dt, J = 13.6, 6.0 Hz, 1H), 3.97-3.73 (m, 2H), 3.65 (dt, J = 13.6, 6.0 Hz, 1H), 2.36 (d, J = 14.4 Hz, 3H). | 430.1 |
| Example 161 Method 9 | 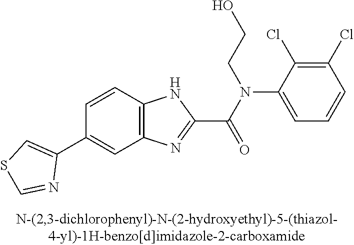 [SMILES: O=C(c1nc2cc(-c3cscn3)ccc2[nH]1)N(CCO)c1cccc(Cl)c1Cl] | white solid, yield: 18% | ¹H NMR (400 MHz, Methanol-d₄) δ 9.12- 8.97 (m, 1H), 8.11- 7.72 (m, 3H), 7.69- 7.32 (m, 4H), 4.38 (dt, J = 12.8, 6.0 Hz, 1H), 3.92 (tt, J = 11.6, 5.6 Hz, 1H), 3.77 (dt, J = 11.6, 6.0 Hz, 1H), 3.65 (dt, J = 12.8, 6.0 Hz, 1H). | 433.0 |
| Example 162 Method 9 Mixture of Enantiomers | 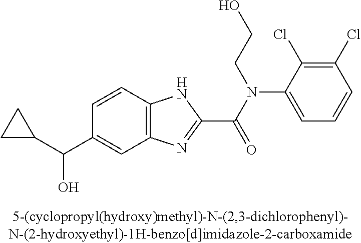 [SMILES: O=C(c1nc2cc(C(O)C3CC3)ccc2[nH]1)N(CCO)c1cccc(Cl)c1Cl] | white solid, yield: 38% | ¹H NMR (400 MHz, Chloroform-d) 7.59- 7.30 (m, 6H), 5.42- 5.28 (m, 1H), 5.19 (m, 1H), 4.18 (m, 1H), 4.00 (m, 1H), 3.93-3.58 (m, 3H), 0.78-0.65 (m, 2H), 0.62-0.50 (m, 2H), 0.43 (m 1H). | 420.2 |
| Example 171 Method 9 | 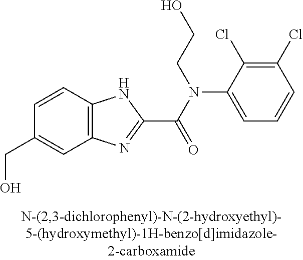 [SMILES: O=C(c1nc2cc(CO)ccc2[nH]1)N(CCO)c1cccc(Cl)c1Cl] | white solid, yield: 78% | ¹H NMR (400 MHz, DMSO-d₆) δ 13.11 (s, 1H), 7.85-7.46 (m, 3H), 7.46-6.97 (m, 3H), 5.32-4.60 (m, 3H), 4.57-3.98 (m, 2H), 3.78-3.43 (m, 3H) | 380.1 |
| Example 187 Method 9 | 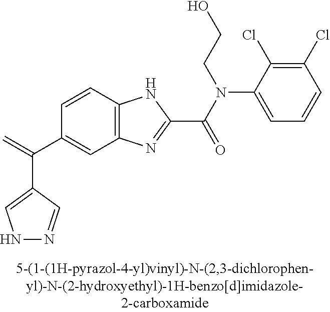 [SMILES: C=C(c1cn[nH]c1)c1ccc2[nH]c(C(=O)N(CCO)c3cccc(Cl)c3Cl)nc2c1] | white solid, yield: 30% | ¹H NMR (400 MHz, Methanol-d₄) δ 7.75 (m, 1H), 7.71-7.54 (m, 3H), 7.52-7.26 (m, 4H), 5.57-5.40 (m, 1H), 5.32-5.17 (m, 1H), 4.37 (m, 1H), 3.98-3.56 (m, 3H). | 442.3 |
| Example 189 Method 9 Mixture of Enantiomers | 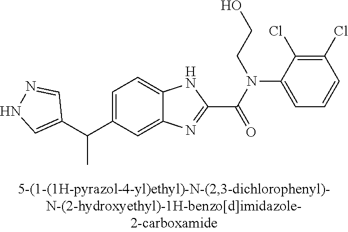 [SMILES: CC(c1cn[nH]c1)c1ccc2[nH]c(C(=O)N(CCO)c3cccc(Cl)c3Cl)nc2c1] | white solid, yield: 26% | ¹H NMR (400 MHz, Methanol-d₄) δ 7.74- 6.88 (m, 8H), 4.29- 4.15 (m, 1H), 4.12- 3.90 (m, 1H), 3.84- 3.48 (m, 3H), 1.61- 1.45 (m, 3H). | 444.3 |
| Example 190 Method 9 | 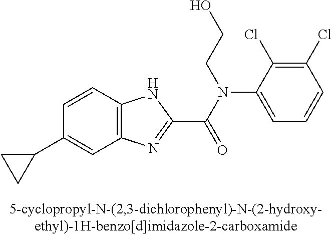 [SMILES: O=C(c1nc2cc(C3CC3)ccc2[nH]1)N(CCO)c1cccc(Cl)c1Cl] | white solid, yield: 31% | ¹H NMR (400 MHz, Methanol-d₄) δ 7.75- 7.67 (m, 1H), 7.62- 7.40 (m, 3H), 7.27- 7.15 (m, 2H), 3.98- 3.50 (m, 4H), 1.90- 1.78 (m, 1H), 1.58- 1.14 (m, 4H). | 390.1 |
| Example 196 Method 9 Mixture of Enantiomers | 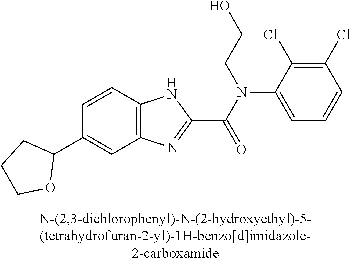 [SMILES: O=C(c1nc2cc(C3CCCO3)ccc2[nH]1)N(CCO)c1cccc(Cl)c1Cl] | white solid, yield: 16% | ¹HNMR (400 MHz, DMSO-d₆) δ 7.72- 7.12 (m, 6H), 4.96- 4.81 (m, 2H), 4.24- 3.47 (m, 5H), 2.41- 2.25 (m, 1H), 1.98- 1.87 (m, 2H), 1.64- 1.59 (m, 1H). | 420.1 |
| Example 210 Method 9 | 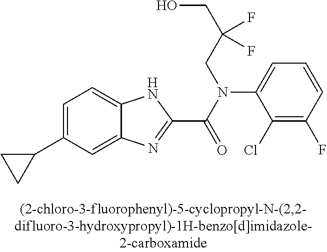 [SMILES: O=C(c1nc2cc(C3CC3)ccc2[nH]1)N(CC(F)(F)CO)c1cccc(F)c1Cl] | white solid, yield: 35% | ¹H NMR (400 MHz, Methanol-d₄) δ 7.67- 6.74 (m, 6H), 4.61 (m, 1H), 4.14-3.61 (m, 3H), 1.90 (m, 1H), 1.02-0.79 (m, 2H), 0.73-0.45 (m, 2H). | 424.2 |
| Example 222 Method 9 | 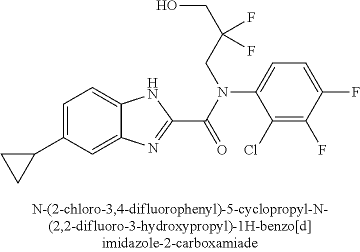 [SMILES: O=C(c1nc2cc(C3CC3)ccc2[nH]1)N(CC(F)(F)CO)c1ccc(F)c(F)c1Cl] | White solid, yield: 22% | ¹H NMR (400 MHz, Methanol-d₄) δ 7.45 (s, 1H), 7.39-7.23 (m, 2H), 7.08 (d, J = 62.7 Hz, 2H), 4.56 (m, 1H), 4.52 (s, 1H), 4.13-3.98 (m, 1H), 3.84 (t, J = 13.4 Hz, 2H), 1.29 (s, 1H), 1.06-0.89 (m, 2H), 0.78-0.62 (m, 2H). | 442.1 |

### Preparation of N-(2,3-dichlorophenyl)-N-(2-hydroxyethyl)-5-(2-hydroxypropan-2-yl)-1H-benzo[d]imidazole-2-carboxamide (150) Method 10

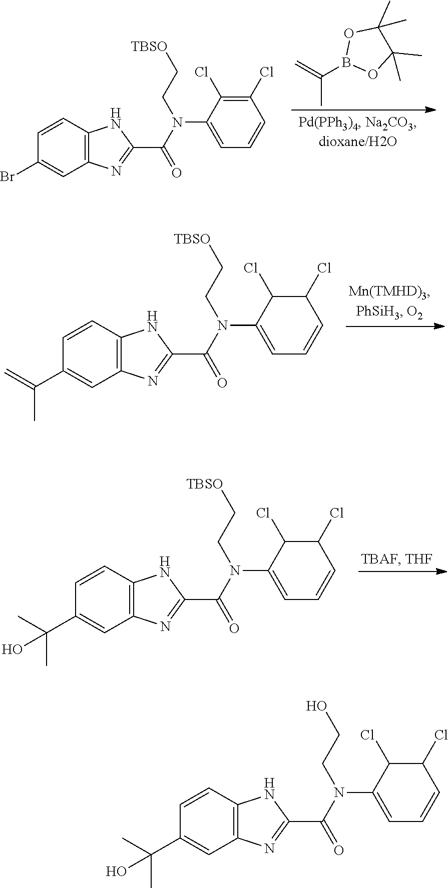

### Step 1. N-(2-((tert-butyldimethylsilyl)oxy)ethyl)-N-(2,3-dichlorophenyl)-5-(prop-1-en-2-yl)-1H-benzo[d]imidazole-2-carboxamide

To a solution of 5-bromo-N-(2-((tert-butyldimethylsilyl)oxy)ethyl)-N-(2,3-dichlorophenyl)-1H-benzo[d]imidazole-2-carboxamide (1.0 g, 1.94 mmol) in dioxane/H₂O (20 mL/5 mL) was added 4,4,5,5-tetramethyl-2-(prop-1-en-2-yl)-1, 3,2-dioxaborolane (613 mg, 3.88 mmol), Pd(PPh₃)₄ (448 mg, 0.39 mmol) and Na₂CO₃ (1.64 g, 15.5 mmol). The mixture was stirred at 95° C. for 16 hours under N₂. The mixture was concentrated, diluted with water (30 mL), and extracted with EA (20 mL*3). The combined organic layers were washed brine (30 mL), dried over anhydrous Na₂SO₄, filtered and concentrated. The residue was purified by FCC (PE/EA=6/1~ 5/1) to give N-(2-((tert-butyldimethylsilyl)oxy)ethyl)-N-(2,3-dichlorophenyl)-5-(prop-1-en-2-yl)-1H-benzo[d]imidazole-2-carboxamide (520 mg, 53%) as a yellow solid. MS (ESI) m/z 504 [M+H]⁺.

### Step 2. N-(2-((tert-butyldimethylsilyl)oxy)ethyl)-N-(2,3-dichlorophenyl)-5-(2-hydroxypropan-2-yl)-1H-benzo[d]imidazole-2-carboxamide

To a solution of N-(2-((tert-butyldimethylsilyl)oxy)ethyl)-N-(2,3-dichlorophenyl)-5-(prop-1-en-2-yl)-1H-benzo[d]imidazole-2-carboxamide (100 mg, 0.2 mmol) in IPA/DCM (10 mL/1 mL) was added Mn(TMHD)₃ (24 mg, 0.04 mmol) and the resulting mixture was stirred at 30° C. for 10 min. Phenylsilane (54 mg, 0.5 mmol) was added to the mixture and stirred at 30° C. for 16 hours under O₂ (1 atm). The mixture was concentrated. The residue was purified by FCC (PE/EA=3/1) to give N-(2-((tert-butyldimethylsilyl)oxy)ethyl)-N-(2,3-dichlorophenyl)-5-(2-hydroxypropan-2-yl)-1H-benzo[d]imidazole-2-carboxamide (50 mg, 48%) as a yellow solid. MS (ESI) m/z 522 [M+H]⁺.

### Step 3. N-(2,3-dichlorophenyl)-N-(2-hydroxyethyl)-5-(2-hydroxypropan-2-yl)-1H-benzo[d]imidazole-2-carboxamide

To a solution of N-(2-((tert-butyldimethylsilyl)oxy)ethyl)-N-(2,3-dichlorophenyl)-5-(2-hydroxypropan-2-yl)-1H-benzo[d]imidazole-2-carboxamide (50 mg, 0.096 mmol) in THF (5 mL) was added TBAF (50.3 mg, 0.19 mmol). The mixture was stirred at rt for 2 hours. The mixture was poured into water, and extracted with EA (5 mL*3). The combined organic layers were washed by brine (5 mL*3) and concentrated. The residue was purified by prep-HPLC (NH₄HCO₃) to give N-(2,3-dichlorophenyl)-N-(2-hydroxyethyl)-5-(2-hydroxypropan-2-yl)-1H-benzo[d]imidazole-2-carboxamide (29.8 mg, 76%) as a white solid. MS (ESI) m/z 408 [M+H]⁺
¹H NMR (400 MHz, DMSO-d₆) δ 13.07 (s, 1H), 7.72-7.20 (m, 6H), 5.04-4.83 (m, 2H), 4.23-4.04 (m, 1H), 3.74-3.47 (m, 3H), 1.49-1.33 (m, 6H).

Examples (Compounds) 183 were synthesized using a similar method to that used in Example 150.

| Example | | | | |
|---|---|---|---|---|
| No. | | | | |
| (Compound | | Appearance | | MS |
| No.) | | and | | (m/z) |
| Method | Structure and Name | Yield | ¹H NMR Data | [M + H]⁺ |
| Example 183 Method 10 | 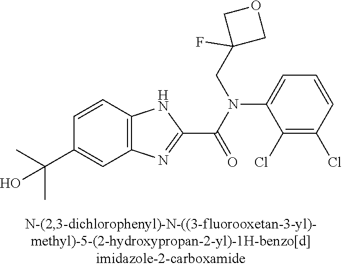 [SMILES: CC(C)(O)c1ccc2[nH]c(C(=O)N(CC3(F)COC3)c3cccc(Cl)c3Cl)nc2c1] | white solid, yield: 9% | ¹H NMR (400 MHz, DMSO-d₆) δ 12.90- 11.71 (m, 1H), 7.75- 7.14 (m, 6H), 5.30- 4.31 (m, 7H), 1.49- 1.42 (m, 6H) | 452.1 |

### Preparation of N-(2-chloro-3-fluorophenyl)-N-(2,2-difluoro-3-hydroxypropyl)-5-(3-methyloxetan-3-yl)-1H-benzo[d]imidazole-2-carboxamide (223) Method 11

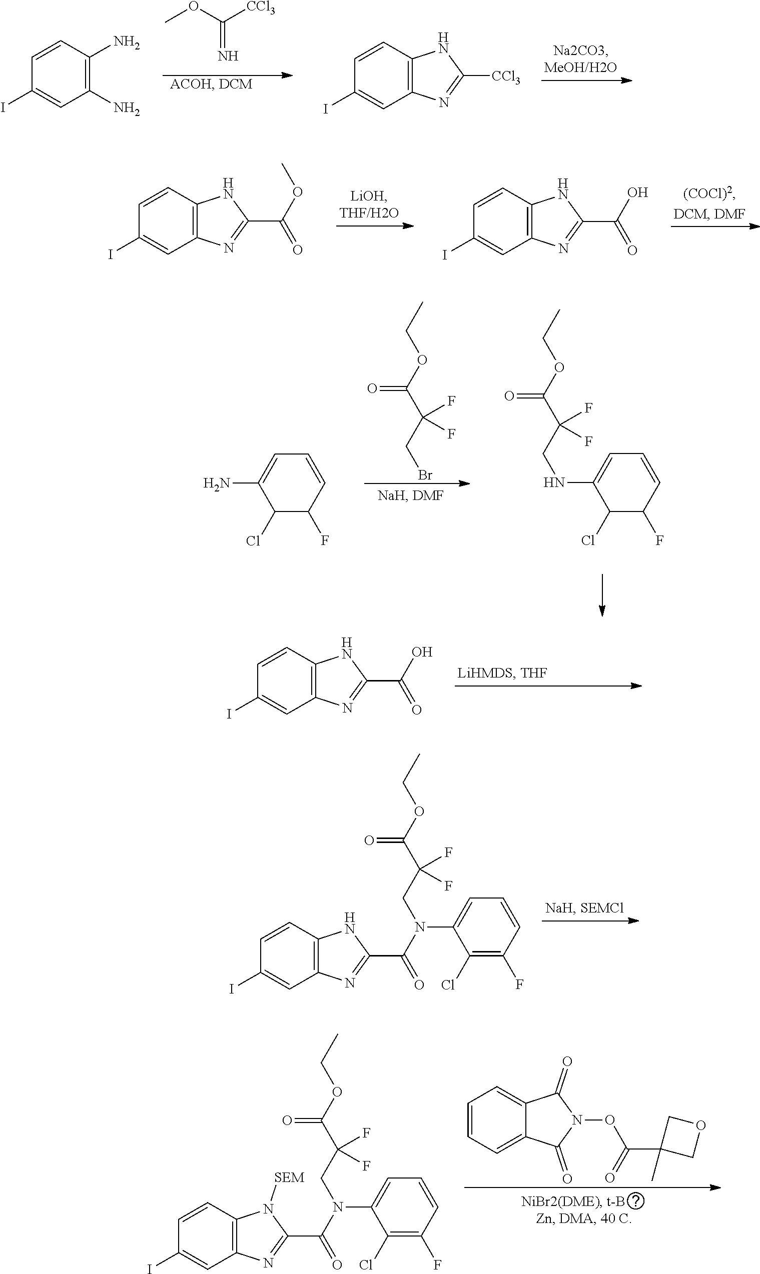

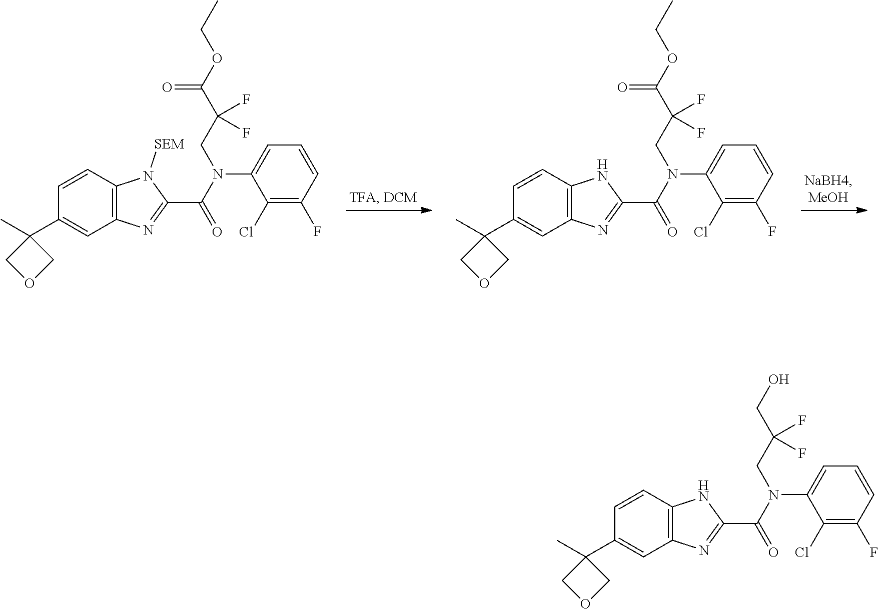 [SMILES: CCOC(=O)C(F)(F)CN(C(=O)c1nc2cc(C3(C)COC3)ccc2n1COCC[Si](C)(C)C)c1cccc(F)c1Cl ; CCOC(=O)C(F)(F)CN(C(=O)c1nc2cc(C3(C)COC3)ccc2[nH]1)c1cccc(F)c1Cl ; CC1(c2ccc3[nH]c(C(=O)N(CC(F)(F)CO)c4cccc(F)c4Cl)nc3c2)COC1]

### Step 1. 5-iodo-2-(trichloromethyl)-1H-benzo[d]imidazole

The solution of 4-iodobenzene-1,2-diamine (15 g, 64.1 mmol) and methyl 2,2,2-trichloroacetimidate (12 g, 70.5 mmol) in DCM (150 mL) and AcOH (75 mL) was stirred at rt for 1 hour. The mixture was concentrated, diluted with water (50 mL) and filtered to give 5-iodo-2-(trichloromethyl)-1H-benzo[d]imidazole (23 g, 99%) as a brown solid. MS (ESI) m/z 361 [M+H]⁺.

### Step 2. methyl 5-iodo-1H-benzo[d]imidazole-2-carboxylate

To a solution of 5-iodo-2-(trichloromethyl)-1H-benzo[d]imidazole (24 g, 66.6 mmol) in MeOH (250 mL) was added Na₂CO₃ (7 g, 66.6 mmol) in water (30 mL) at 0° C. The mixture was stirred at 70° C. for 2 hours. The mixture was poured into water (500 mL) and extracted with EA (250 mL*2). The combined organic layers were dried over Na₂SO₄, filtered and concentrated. The residue was wished with (PE/EA=1/1) 15 mL to give methyl 5-iodo-1H-benzo[d]imidazole-2-carboxylate (14 g, 69%) as a brown solid. MS (ESI) m/z 303 [M+H]⁺.

### Step 3. 5-iodo-1H-benzo[d]imidazole-2-carboxylic Acid

To a solution of methyl 5-iodo-1H-benzo[d]imidazole-2-carboxylate (10 g, 33.1 mmol) in THF (150 mL) and H₂O (50 mL) was added LiOH. H₂O (4 g, 99.3 mmol) at rt. The mixture was stirred at 50° C. for 4 hours. The organic solvent was removed under reduced pressure. The water layer was adjusted to pH 5 with 1M HCl, and filtered to give 5-iodo-1H-benzo[d]imidazole-2-carboxylic acid (7 g, 73%) as a brown solid. MS (ESI) m/z 289 [M+H]⁺.

### Step 4. 5-iodo-1H-benzo[d]imidazole-2-carbonyl chloride

To a solution of 5-iodo-1H-benzo[d]imidazole-2-carboxylic acid (300 mg, 1.04 mmol) in DCM (10 mL) was added DMF (1 drop) and oxalyl dichloride (529 mg, 4.16 mmol) at 0° C. The mixture was stirred at rt for 1 hour. The mixture was concentrated to give 5-iodo-1H-benzo[d]imidazole-2-carbonyl chloride (300 mg, 94%) as a yellow solid.

### Step 5. ethyl 3-((2-chloro-3-fluorophenyl)amino)-2,2-difluoropropanoate

To a solution of 2-chloro-3-fluoroaniline (5.0 g, 34.4 mmol) in DMF (50 mL) was added NaH (1.6 g, 41.3 mmol, 60%) at 0° C. The mixture was stirred at 0° C. for 0.5 hours. Then ethyl 3-bromo-2,2-difluoropropanoate (8.9 g, 41.3 mmol) was slowly added. The reaction mixture was stirred at 0° C. for 1 hour. The mixture was poured into ice water (200 mL) and extracted with EA (100 mL*2). The combined organic layers were washed with water (100 mL*2) and brine (100 mL), dried over Na₂SO₄, filtered and concentrated. The residue was purified by FCC (PE/EA=20/1) to give ethyl 3-((2-chloro-3-fluorophenyl)amino)-2,2-difluoropropanoate (2 g, 20%) as a yellow oil. MS (ESI) m/z 282 [M+H]+.

### Step 6. ethyl 3-(N-(2-chloro-3-fluorophenyl)-5-iodo-1H-benzo[d]imidazole-2-carboxamido)-2,2-difluoropropanoate

To a solution of 5-iodo-1H-benzo[d]imidazole-2-carbonyl chloride (7.0 g, 22.8 mmol) and ethyl 3-((2-chloro-3-fluorophenyl)amino)-2,2-difluoropropanoate (4.2 g, 15.2 mmol) in THF (100 mL) was slowly added LiHMDS (45.7 mL, 45.7 mmol, 1.0 M in THF) at 0° C. The mixture was stirred at rt for 1 hour. The mixture was quenched with NH₄Cl solution (100 mL) and extracted with EA (100 mL*2). The combined organic layers were washed with brine (100 mL), dried over Na₂SO₄, filtered and concentrated. The residue was purified by FCC (PE/EA=3/1) to give ethyl 3-(N-(2-chloro-3-fluorophenyl)-5-iodo-1H-benzo[d]imidazole-2-carboxamido)-2,2-difluoropropanoate (3 g, 23%) as a yellow solid. MS (ESI) m/z 552 [M+H]+.

### Step 7. ethyl 3-(N-(2-chloro-3-fluorophenyl)-5-iodo-1-((2-(trimethylsilyl) ethoxy)methyl)-1H-benzo[d]imidazole-2-carboxamido)-2,2-difluoropropanoate

To a solution of ethyl 3-(N-(2-chloro-3-fluorophenyl)-5-iodo-1H-benzo[d]imidazole-2-carboxamido)-2,2-difluoropropanoate (150 mg, 0.27 mmol) in DMF (5 mL) was added NaH (12 mg, 0.29 mmol, 60%) at 0° C. The mixture was stirred at 0° C. for 0.5 hours. Then SEMC1 (53 mg, 0.32 mmol) was added at 0° C. The reaction mixture was stirred at rt for 1 hour. The mixture was poured into ice water (20 mL) and extracted with EA (10 mL*2). The combined organic layers were washed with water (10 mL*2) and brine (10 mL), dried over Na₂SO₄, filtered and concentrated. The residue was purified by FCC (PE/EA=10/1) to give ethyl 3-(N-(2-chloro-3-fluorophenyl)-5-iodo-1-((2-(trimethylsilyl) ethoxy)methyl)-1H-benzo[d]imidazole-2-carboxamido)-2,2-difluoropropanoate (100 mg, 54%) as a yellow solid. MS (ESI) m/z 682 [M+H]⁺.

Step 8. ethyl 3-(N-(2-chloro-3-fluorophenyl)-5-(3-methyloxetan-3-yl)-1-((2-(trimethylsilyl) ethoxy)methyl)-1H-benzo[d]imidazole-2-carboxamido)-2,2-difluoropropanoate The mixture of ethyl 3-(N-(2-chloro-3-fluorophenyl)-5-iodo-1-((2-(trimethylsilyl) ethoxy)methyl)-1H-benzo[d]imidazole-2-carboxamido)-2,2-difluoropropanoate (100 mg, 0.14 mmol), 1,3-dioxoisoindolin-2-yl 3-methyloxetane-3-carboxylate (42 mg, 0.16 mmol), NiBr₂(DME) (13 mg, 0.04 mmol), t-Bubpycamcn (15 mg, 0.04 mmol) and Zn (19 mg, 0.29 mmol) in DMA (4.5 mL) was stirred at 40° C. under nitrogen atmosphere in a sealed tube for 16 hours. The mixture was filtered and concentrated. The residue was purified by prep-TLC (PE/EA=3/1) to give ethyl 3-(N-(2-chloro-3-fluorophenyl)-5-(3-methyloxetan-3-yl)-1-((2-(trimethylsilyl) ethoxy)methyl)-1H-benzo[d]imidazole-2-carboxamido)-2,2-difluoropropanoate (10 mg, 10%) as white solids. MS (ESI) m/z 626 [M+H]+.

### Step 9. ethyl 3-(N-(2-chloro-3-fluorophenyl)-5-(3-methyloxetan-3-yl)-1H-benzo[d]imidazole-2-carboxamido)-2,2-difluoropropanoate

A solution of ethyl 3-(N-(2-chloro-3-fluorophenyl)-5-(3-methyloxetan-3-yl)-1-((2-(trimethylsilyl) ethoxy)methyl)-1H-benzo[d]imidazole-2-carboxamido)-2,2-difluoropropanoate (30 mg, 0.05 mmol) in TFA/DCM (1 mL/2 mL) was stirred at rt for 1 h. The mixture was concentrated to give crude ethyl 3-(N-(2-chloro-3-fluorophenyl)-5-(3-methyloxetan-3-yl)-1H-benzo[d]imidazole-2-carboxamido)-2,2-difluoropropanoate (20 mg, 84%) as a white solid. MS (ESI) m/z 496 [M+H]+.

### Step 10. N-(2-chloro-3-fluorophenyl)-N-(2,2-difluoro-3-hydroxypropyl)-5-(3-methyloxetan-3-yl)-1H-benzo[d]imidazole-2-carboxamide

To a solution of ethyl 3-(N-(2-chloro-3-fluorophenyl)-5-(3-methyloxetan-3-yl)-1H-benzo[d]imidazole-2-carboxamido)-2,2-difluoropropanoate (15 mg, 0.03 mmol) in MeOH (2 mL) was added NaBH₄ (5 mg, 0.15 mmol) at rt. The mixture was stirred at rt for 0.5 hours. The mixture was diluted with water (2 mL), extracted with EA (10 mL). The organic layer was dried over Na₂SO₄, filtered and concentrated. The residue was purified by prep-HPLC(NH₄HCO₃) to give N-(2-chloro-3-fluorophenyl)-N-(2,2-difluoro-3-hydroxypropyl)-5-(3-methyloxetan-3-yl)-1H-benzo[d]imidazole-2-carboxamide (2.0 mg, 14%) as a white solid. MS (ESI) m/z 454 [M+H]+.

¹HNMR (400 MHz, DMSO-d₆) δ 13.29 (brs, 1H), 7.50-7.01 (m, 6H), 5.63-5.61 (m, 1H), 4.89-4.48 (m, 6H), 3.84-3.79 (m, 2H), 1.71-1.61 (m, 3H).

Examples (Compounds) 195 were synthesized using a similar method to that used in Example 223.

| Example | | | | |
|---|---|---|---|---|
| No. | | | | |
| (Compound | | Appearance | | MS |
| No.) | | and | | (m/z) |
| Method | Structure and Name | Yield | ¹H NMR Data | [M + H]⁺ |
| Example 195 Method 11 | 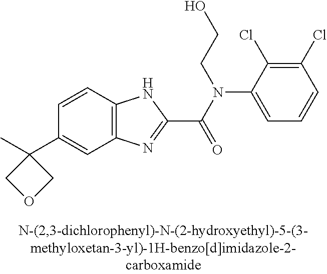 [SMILES: CC1(c2ccc3[nH]c(C(=O)N(CCO)c4cccc(Cl)c4Cl)nc3c2)COC1] | white solid, yield: 5% | ¹HNMR (400 MHz, DMSO-d₆) δ 7.64- 7.04 (m, 6H), 6.04 (s, 2H), 4.89-4.52 (m, 4H), 4.10-3.95 (m, 1H), 3.70-3.46 (m, 2H), 1.62 (s, 3H) | 420.1 |

### Preparation of N-(2-chloro-3-fluorophenyl)-N-(2,2-difluoro-3-hydroxypropyl)-5-(3-methyloxetan-3-yl)-1H-benzo[d]imidazole-2-carboxamide (232) Method 12

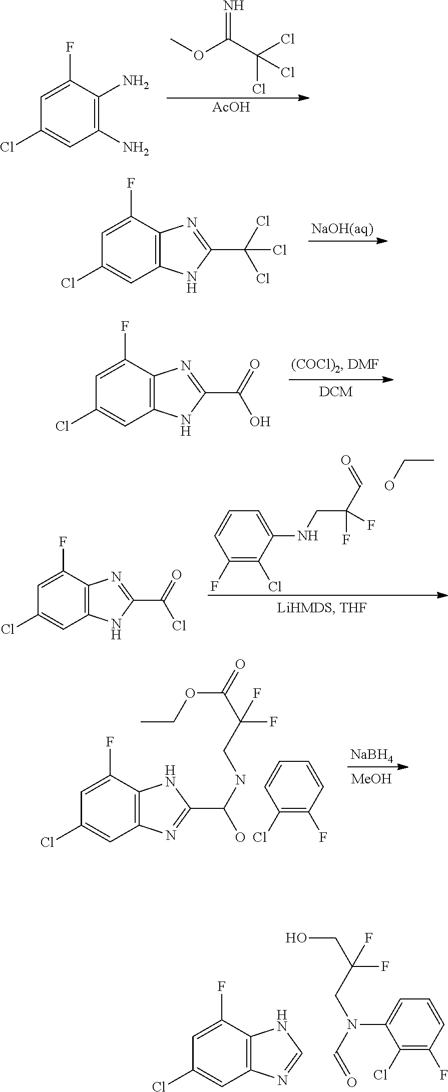

### Step 1. 6-chloro-4-fluoro-2-(trichloromethyl)-1H-benzo[d]imidazole

Methyl 2,2,2-trichloroethanimidate (991 mg, 5.62 mmol) was added to a solution of 5-chloro-3-fluoro-benzene-1,2-diamine (820 mg, 5.11 mmol) in acetic acid (25 mL) dropwise at 0° C. with vigorous stirring. The reaction mixture was stirred at room temperature for 5 h. Crushed ice was added into the reaction mixture and the reaction mixture was stirred for 20 min further after completion of the reaction (monitored by TLC). The precipitate was filtered, washed with water (3×100 ml), dried in an oven to give 6-chloro-4-fluoro-2-(trichloromethyl)-1H-benzo[d]imidazole (1.4 g) as a yellow solid, which was used in the next step without further purification. MS (ESI) m/z 287 [M+H]⁺.

### Step 2. 6-chloro-4-fluoro-1H-benzo[d]imidazole-2-carboxylic acid

6-chloro-4-fluoro-2-(trichloromethyl)-1H-benzo[d]imidazole (1.4 g, 5.10 mmol) was added to a cooled solution of sodium hydroxide (25 mL, 1 N). The resulting solution was filtered and the filtrate was acidified with hydrochloric acid (1 N) to pH 4. The precipitate was filtered off, washed twice with a solution of water and acetonitrile (ratio 3:1) and twice with ether. After drying, this led to 6-chloro-4-fluoro-1H-benzo[d]imidazole-2-carboxylic acid (1.1 g). MS (ESI) m/z 215 [M+H]+.

### Step 3. 6-chloro-4-fluoro-1H-benzo[d]imidazole-2-carbonyl chloride

To a suspension of 6-chloro-4-fluoro-1H-benzo[d]imidazole-2-carboxylic acid (1.1 g, 5.03 mmol) in DCM (25 ml) at 0° C. was added oxalyl chloride (1.3 g, 10.1 mmol) and 3 drops of dry DMF. After 30 minutes, the resulting clear solution was concentrated to dryness to give the title compound 6-chloro-4-fluoro-1H-benzo[d]imidazole-2-carbonyl chloride (1.1 g), which was immediately used without further purification. MS (ESI) m/z 233 [M+H]⁺.

### Step 4. ethyl 3-(5-chloro-N-(2-chloro-3-fluorophenyl)-7-fluoro-1H-benzo[d]imidazole-2-carboxamido)-2,2-difluoropropanoate

To a stirred solution of 6-chloro-4-fluoro-1H-benzo[d]imidazole-2-carbonyl chloride (215 mg, 0.9 mmol) and ethyl 3-(2-chloro-3-fluoro-anilino)-2,2-difluoropropanoate (200 mg, 0.7 mmol) in THF (5 mL) were added LiHMDS (1.1 mL, 1.1 mmol) dropwise at −10° C. The resulting mixture was stirred for 4 h. The reaction was quenched with water. The resulting mixture was extracted with DCM. The combined organic layers were washed with brine, and dried over anhydrous Na₂SO₄. After filtration, the filtrate was concentrated under reduced pressure. The residue was purified by prep-TLC (MeOH/DCM=1/5) to afford ethyl 3-(5-chloro-N-(2-chloro-3-fluorophenyl)-7-fluoro-1H-benzo[d]imidazole-2-carboxamido)-2,2-difluoropropanoate (157 mg) as a white solid. MS (ESI) m/z 478 [M+H]+.

### Step 5. 5-chloro-N-(2-chloro-3-fluorophenyl)-N-(2,2-difluoro-3-hydroxypropyl)-7-fluoro-1H-benzo[d]imidazole-2-carboxamide

To a cooled solution of ethyl 3-(5-chloro-N-(2-chloro-3-fluorophenyl)-7-fluoro-1H-benzo[d]imidazole-2-carboxamido)-2,2-difluoropropanoate (50 mg, 0.1 mmol) in methanol (3 mL) was added NaBH₄ (4.6 mg, 0.21 mmol). The mixture was stirred at 5° C. for 20 minutes and at room temperature for 2 hours. To the crude reaction was added water (1 mL) and the solvents were reduced under reduced pressure. The residue was dissolved in ethyl acetate (50 mL) and washed with brine (2×25 mL), dried (Na₂SO₄), and concentrated. Purification by column chromatography with silica gel eluting with ethyl acetate/n-hexane (1:2) gave the title compound 5-chloro-N-(2-chloro-3-fluorophenyl)-N-(2,2-difluoro-3-hydroxypropyl)-7-fluoro-1H-benzo[d]imidazole-2-carboxamide (13 mg, 29% yield) as a white solid MS (ESI) m/z 436 [M+H]⁺.

¹H NMR (400 MHz, Methanol-d₄) δ 7.56-7.35 (m, 2H), 7.32-7.13 (m, 2H), 6.97 (m, 1H), 4.50 (m, 1H), 4.20-4.03 (m, 1H), 3.84 (t, J=13.3 Hz, 2H).

Examples (Compounds) 130, 155, 160, 163, 164, 175, 191, 192, 201, 204, 205, 206, 208, 211, 231, 233, 234, 235, 236 were synthesized using a similar method to that used in Example 232.

| Example | | | | |
|---|---|---|---|---|
| No. | | | | |
| (Compound | | Appearance | | MS |
| No.) | | and | | (m/z) |
| Method | Structure and Name | Yield | ¹H NMR Data | [M + H]⁺ |
| Example 130 Method 12 | 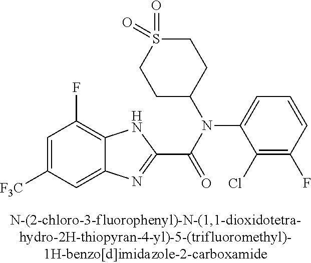 [SMILES: O=C(c1nc2cc(C(F)(F)F)cc(F)c2[nH]1)N(c1cccc(F)c1Cl)C1CCS(=O)(=O)CC1] | white solid, yield: 5% | ¹H NMR (400 MHz, Methanol-d₄) δ 7.73- 7.32 (m, 6H), 3.51- 3.35 (m, 5H), 3.19- 3.09 (m, 1H), 2.61- 2.57 (m, 1H), 2.37 (d, J = 13.2 Hz, 1H), 2.20 (ddd, J = 13.2, 11.2, 3.2 Hz, 1H). | 490.1 |
| Example 155 Method 12 | 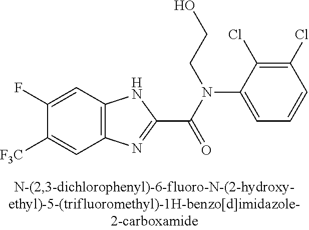 [SMILES: O=C(c1nc2cc(C(F)(F)F)c(F)cc2[nH]1)N(CCO)c1cccc(Cl)c1Cl] | white solid, yield: 30% | ¹H NMR (400 MHz, Methanol-d₄) δ 8.15- 7.26 (m, 5H), 4.39 (dt, J = 13.6, 6.0 Hz, 1H), 3.89 (ddt, J = 16.8, 10.8, 5.6 Hz, 1H), 3.78 (ddt, J = 17.2, 11.6, 5.6 Hz, 1H), 3.62 (dt, J = 13.6, 6.0 Hz, 1H). | 436.1 |
| Example 160 Method 12 | 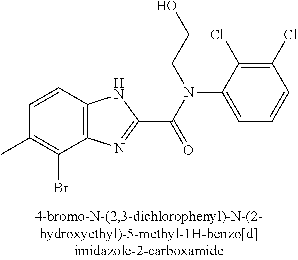 [SMILES: Cc1ccc2[nH]c(C(=O)N(CCO)c3cccc(Cl)c3Cl)nc2c1Br] | white solid, yield: 40% | ¹H NMR (400 MHz, DMSO-d₆) δ 7.60- 7.54 (m, 1H), 7.54- 7.46 (m, 1H), 7.39 (t, J = 8.0 Hz, 1H), 7.32 (t, J = 8.4 Hz, 1H), 7.19 (d, J = 8.3 Hz, 1H), 4.87 (s, 1H), 4.30-4.19 (m, 1H), 3.78-3.67 (m, 1H), 3.65-3.57 (m, 1H), 3.55-3.45 (m, 1H), 2.37 (s, 3H). | 442.0 |
| Example 163 Method 12 | 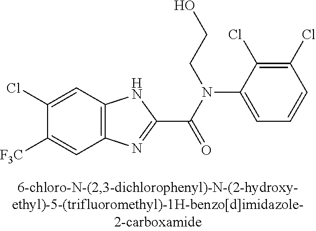 [SMILES: O=C(c1nc2cc(C(F)(F)F)c(Cl)cc2[nH]1)N(CCO)c1cccc(Cl)c1Cl] | white solid, yield: 63% | ¹H NMR (400 MHz, Methanol-d₄) δ 8.34- 7.22 (m, 5H), 4.40 (dt, J = 13.6, 6.0 Hz, 1H), 3.90 (tt, J = 12.4, 5.6 Hz, 1H), 3.78 (tt, J = 11.6, 5.6 Hz, 1H), 3.62 (dt, J = 13.6, 6.0 Hz, 1H). | 452.0 |
| Example 164 Method 12 | 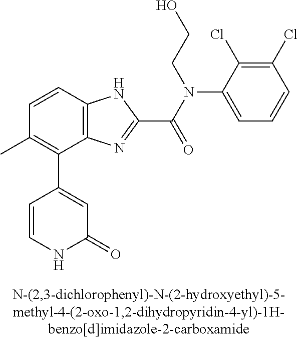 [SMILES: Cc1ccc2[nH]c(C(=O)N(CCO)c3cccc(Cl)c3Cl)nc2c1-c1cc[nH]c(=O)c1] | white solid, yield: 47.4% | ¹H NMR (400 MHz, DMSO-d₆) δ 13.22 (s, 1H), 11.55 (s, 1H), 7.70-7.43 (m, 3H), 7.38 (d, J = 8.3 Hz, 1H), 7.33-7.14 (m, 2H), 5.93 (s, 1H), 5.66 (d, J = 6.7 Hz, 1H), 4.85 (s, 1H), 4.26-4.14 (m, 1H), 3.76-3.65 (m, 1H), 3.62-3.53 (m, 1H), 3.51-3.42 (m, 1H), 2.24 (s, 3H). | 457.1 |
| Example 175 Method 12 | 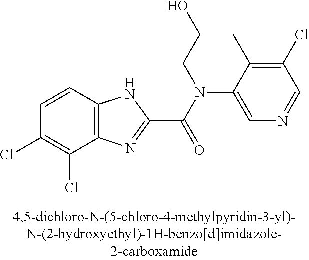 [SMILES: Cc1c(Cl)cncc1N(CCO)C(=O)c1nc2c(Cl)c(Cl)ccc2[nH]1] | white solid, yield: 24% | ¹H NMR (400 MHz, Chloroform-d) δ 8.60 (d, J = 11.7 Hz, 1H), 8.41 (d, J = 51.2 Hz, 1H), 7.44 (d, J = 8.6 Hz, 1H), 7.38-7.28 (m, 1H), 4.70-4.61 (m, 1H), 4.51-4.42 (m, 1H), 4.02-3.89 (m, 1H), 3.87-3.78 (m, 2H), 2.38 (d, J = 19.8 Hz, 3H). | 399.0 |
| Example 191 Method 12 Mixture of Enantiomers | 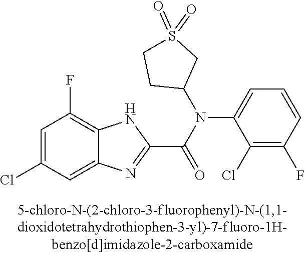 [SMILES: O=C(c1nc2cc(Cl)cc(F)c2[nH]1)N(c1cccc(F)c1Cl)C1CCS(=O)(=O)C1] | white solid, yield: 11% | ¹H NMR (400 MHz, DMSO-d₆) δ 7.48 (d, J = 16.0 Hz, 3H), 7.31 (s, 1H), 7.10 (d, J = 10.4 Hz, 1H), 5.15 (d, J = 46.4 Hz, 1H), 3.78-3.33 (m, 4H), 2.39-1.98 (m, 2H). | 460.1 |
| Example 192 Method 12 Mixture of Enantiomers | 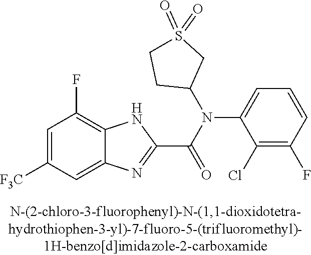 [SMILES: O=C(c1nc2cc(C(F)(F)F)cc(F)c2[nH]1)N(c1cccc(F)c1Cl)C1CCS(=O)(=O)C1] | white solid, yield: 18% | ¹HNMR (400 MHz, DMSO-d₆) δ 14.17 (s, 1H), 7.61-7.34 (m, 5H), 5.25-5.09 (m, 1H), 3.76-3.64 (m, 1H), 3.48-3.40 (m, 1H), 3.13-3.09 (m, 2H), 2.60-2.44 (m, 1H), 2.39-2.37 (m, 1H) | 494.1 |
| Example 201 Method 12 | 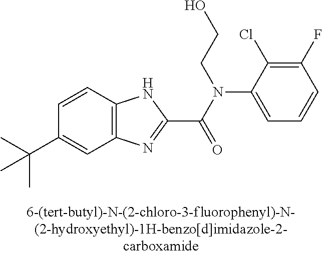 [SMILES: CC(C)(C)c1ccc2[nH]c(C(=O)N(CCO)c3cccc(F)c3Cl)nc2c1] | white solid, yield: 34% | ¹H NMR (400 MHz, DMSO-d₆) δ 13.01 (brs, 1H), 7.56-7.22 (m, 6H), 4.92-4.83 (m, 1H), 4.19-4.13 (m, 1H), 3.75-3.57 (m, 3H), 1.36 (s, 2.8H), 1.28 (s, 6.2H) | 390.1 |
| Example 204 Method 12 | 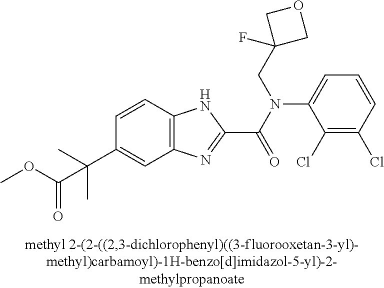 [SMILES: COC(=O)C(C)(C)c1ccc2[nH]c(C(=O)N(CC3(F)COC3)c3cccc(Cl)c3Cl)nc2c1] | white solid, yield: 56% | ¹H NMR (400 MHz, DMSO-d₆) δ 6.78- 6.25 (m, 6H), 3.88- 3.72 (m, 5H), 3.57- 3.44 (m, 1H), 2.74 (s, 3H), 0.71 (s, 6H). | 494.3 |
| Example 205 Method 12 | 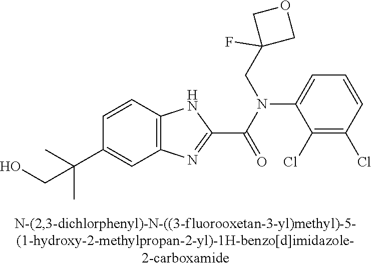 [SMILES: CC(C)(CO)c1ccc2[nH]c(C(=O)N(CC3(F)COC3)c3cccc(Cl)c3Cl)nc2c1] | white solid, yield: 53% | ¹H NMR (400 MHz, DMSO-d₆) δ 13.15 (d, J = 35.0 Hz, 1H), 7.91-7.07 (m, 6H), 5.57-5.19 (m, 1H), 4.78-4.24 (m, 6H), 3.53-3.36 (m, 2H), 1.21 (d, J = 14.5 Hz, 6H) | 466.2 |
| Example 206 Method 12 | 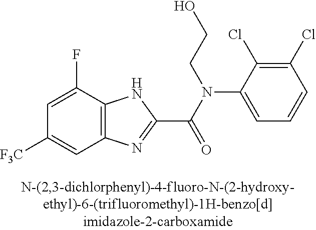 [SMILES: O=C(c1nc2cc(C(F)(F)F)cc(F)c2[nH]1)N(CCO)c1cccc(Cl)c1Cl] | white solid, yield: 24% | ¹H NMR (400 MHz, DMSO-d₆) δ 14.43- 13.98 (m, 1H), 7.77- 7.50 (m, 4H), 7.45- 7.32 (m, 1H), 4.88 (s, 1H), 4.33-4.19 (m, 1H), 3.80-3.70 (m, 1H), 3.68-3.46 (m, 2H). | 436.1 |
| Example 208 Method 12 | 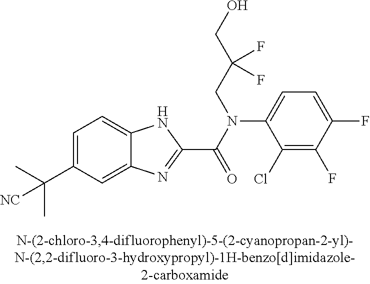 [SMILES: CC(C)(C#N)c1ccc2[nH]c(C(=O)N(CC(F)(F)CO)c3ccc(F)c(F)c3Cl)nc2c1] | white solid, yield: 65% | ¹H NMR (400 MHz, DMSO-d₆) δ 13.42 (s, 1H), 7.61-7.52 (m, 3H), 7.49-7.30 (m, 2H), 4.73-4.56 (m, 1H), 4.29-4.11 (m, 1H), 3.79-3.68 (m, 2H), 1.71 (s, 3H), 1.68 (s, 3H). | 469.3 |
| Example 211 Method 12 | 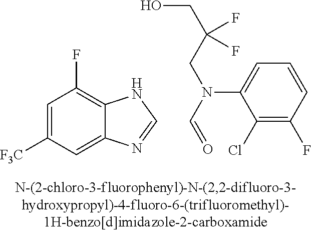 | white solid, yield: 22% | ¹H NMR (400 MHz, DMSO-d₆) δ 7.64 (s, 1H), 7.56-7.44 (m, 2H), 7.43-7.33 (m, 1H), 7.01 (d, J = 11.0 Hz, 1H), 4.85- 4.67 (m, 1H), 4.60- 4.44 (m, 1H), 4.25- 4.08 (m, 1H), 4.00- 3.62 (m, 2H). | 470.0 |
| Example 231 Method 12 | 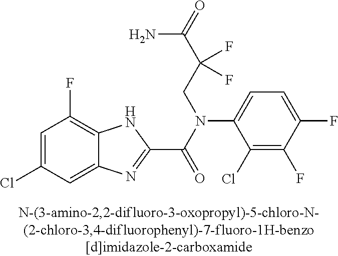 [SMILES: NC(=O)C(F)(F)CN(C(=O)c1nc2cc(Cl)cc(F)c2[nH]1)c1ccc(F)c(F)c1Cl] | white solid, yield: 51% | ¹H NMR (400 MHz, Methanol-d₄) δ 7.51 (m, 1H), 7.41-7.12 (m, 2H), 7.01 (m, 1H), 5.12-4.98 (m, 1H), 4.27-4.07 (m, 1H). | 467.2 |
| Example 233 Method 12 | 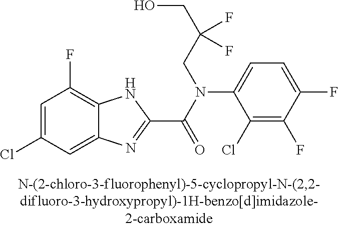 [SMILES: O=C(c1nc2cc(Cl)cc(F)c2[nH]1)N(CC(F)(F)CO)c1ccc(F)c(F)c1Cl] | white solid, yield: 36% | ¹H NMR (400 MHz, Methanol-d₄) δ 7.42- 7.25 (m, 2H), 7.16- 6.95 (m, 1H), 6.66 (m, 1H), 4.72-4.45 (m, 1H), 4.23-3.93 (m, 1H), 3.89-3.61 (m, 2H) | 454.3 |
| Example 234 Method 12 | 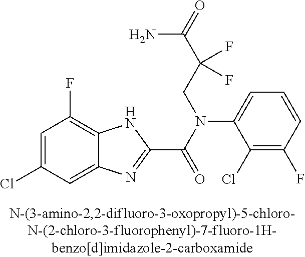 [SMILES: NC(=O)C(F)(F)CN(C(=O)c1nc2cc(Cl)cc(F)c2[nH]1)c1cccc(F)c1Cl] | white solid, yield: 42% | ¹H NMR (400 MHz, Methanol-d₄) δ 7.44- 7.12 (m, 4H), 6.96 (m, 1H), 4.97-4.85 (m, 1H), 4.18-3.88 (m, 1H). | 449.1 |
| Example 235 Method 12 | 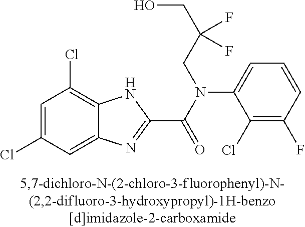 [SMILES: O=C(c1nc2cc(Cl)cc(Cl)c2[nH]1)N(CC(F)(F)CO)c1cccc(F)c1Cl] | white solid, yield: 29% | ¹H NMR (400 MHz, DMSO-d₆) δ 13.78 (s, 1H), 7.59-7.31 (m, 5H), 5.58 (t, J = 6.3 Hz, 1H), 4.75- 4.55 (m, 1H), 4.31- 4.14 (m, 1H), 3.83- 3.61 (m, 2H). | 452.0 |
| Example 236 Method 12 | 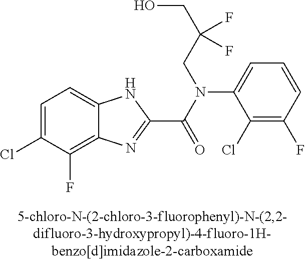 [SMILES: O=C(c1nc2c(F)c(Cl)ccc2[nH]1)N(CC(F)(F)CO)c1cccc(F)c1Cl] | white solid, yield: 12% | ¹HNMR (400 MHz, DMSO-d₆) δ 13.88 (br s, 1H), 7.58-7.28 (m, 5H), 5.64-5.61 (m, 1H), 4.70-4.58 (m, 1H), 4.30-4.17 (m, 1H), 3.80-3.65 (m, 2H) | 436.0 |

### Preparation of 5-(tert-butyl)-N-(3-chloro-6-oxo-1,6-dihydropyridin-2-yl)-N-((3-fluorooxetan-3-yl)methyl)-1H-benzo[d]imidazole-2-carboxamide (221)

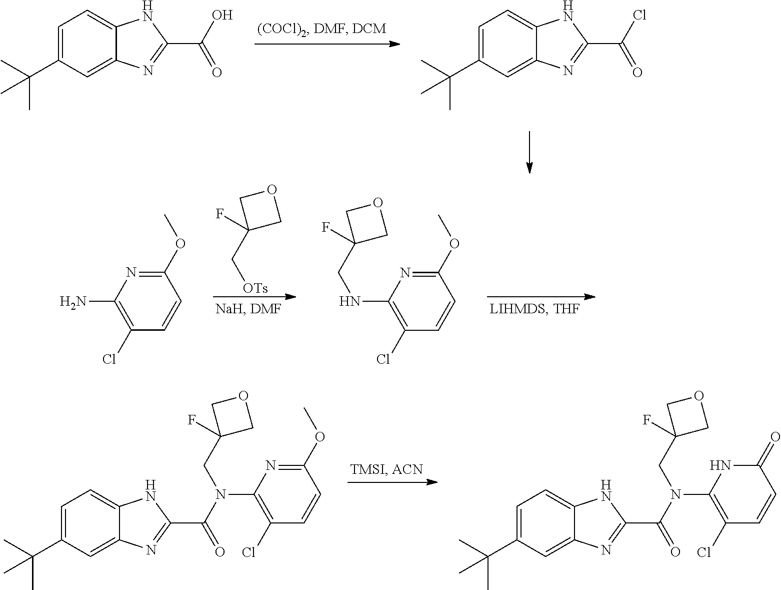

### Step 1. 5-(tert-butyl)-1H-benzo[d]imidazole-2-carbonyl chloride

To a solution of 5-(tert-butyl)-1H-benzo[d]imidazole-2-carboxylic acid (500 mg, 2.29 mmol) in DCM (20 mL) was added (COCl)₂ (0.39 mL, 4.58 mmol) and DMF (2 drop) at 0° C. The reaction was stirred at RT for 1 hour. The mixture was concentrated to give 5-(tert-butyl)-1H-benzo[d]imidazole-2-carbonyl chloride (500 mg, crude) as a yellow solid.

### Step 2. 3-chloro-N-((3-fluorooxetan-3-yl)methyl)-6-methoxypyridin-2-amine

To a solution of 3-chloro-6-methoxypyridin-2-amine (500 mg, 3.15 mmol) in DMF (10 mL) was added NaH (151 mg, 3.78 mmol, 60%) at 0° C. and stirred for 30 mins. (3-fluorooxetan-3-yl)methyl 4-methylbenzenesulfonate (984 mg, 3.78 mmol) and KI (523 mg, 3.15 mmol) was added to the mixture at 0° C. The reaction was stirred at 80° C. for 16 hours. The mixture was quenched with ice-water (30 mL), and extracted with EA (10 mL*3). The combined organic layers were washed with water (20 mL*2) and brine (20 mL), dried over Na₂SO₄, filtered and concentrated. The residue was purified by FCC (PE/EA=5/1) to give 3-chloro-N-((3-fluorooxetan-3-yl)methyl)-6-methoxypyridin-2-amine (470 mg, 60%) as a yellow liquid. MS (ESI) m/z 247 [M+H]⁺.

### Step 3. 5-(tert-butyl)-N-(3-chloro-6-methoxypyridin-2-yl)-N-((3-fluorooxetan-3-yl)methyl)-1H-benzo[d]imidazole-2-carboxamide

To a solution of 3-chloro-N-((3-fluorooxetan-3-yl)methyl)-6-methoxypyridin-2-amine (470 mg, 1.91 mmol) in THF (10 mL) was added LiHMDS (3.4 mL, 3.4 mmol, 1.0M in THF) at 0° C. and stirred for 30 mins. 5-(tert-butyl)-1H-benzo[d]imidazole-2-carbonyl chloride (488 mg, 2.1 mmol) was added to the mixture and stirred at rt for 2 hours at 0° C. The mixture was diluted with water (20 mL) and extracted with EA (10 mL*3). The combined organic layers were washed with brine (10 mL), dried over Na₂SO₄, filtered and concentrated. The residue was purified by FCC (PE/EA=5/1) to give 5-(tert-butyl)-N-(3-chloro-6-methoxypyridin-2-yl)-N-((3-fluorooxetan-3-yl)methyl)-1H-benzo[d]imidazole-2-carboxamide (220 mg, 25%) as a white solid. MS (ESI) m/z 447 [M+H]⁺.

### Step 4. 5-(tert-butyl)-N-(3-chloro-6-oxo-1,6-dihydropyridin-2-yl)-N-((3-fluorooxetan-3-yl)methyl)-1H-benzo[d]imidazole-2-carboxamide

To a solution of 5-(tert-butyl)-N-(3-chloro-6-methoxypyridin-2-yl)-N-((3-fluorooxetan-3-yl)methyl)-1H-benzo[d]imidazole-2-carboxamide (100 mg, 0.22 mmol) in MeCN (3 mL) was added TMSI (44.7 mg, 0.22 mmol) at 0° C. The mixture was stirred at rt for 16 hours. The mixture was diluted with water (10 mL) and extracted with EA (10 mL*2). The combined organic layers were washed with sat. NaHCO₃ (10 mL) and brine (10 mL), dried over Na₂SO₄, filtered and concentrated. The residue was purified by prep-HPLC (NH₄HCO₃) to give 5-(tert-butyl)-N-(3-chloro-6-oxo-1,6-dihydropyridin-2-yl)-N-((3-fluorooxetan-3-yl)methyl)-1H-benzo[d]imidazole-2-carboxamide (5.2 mg, 5.3%) as a white solid. MS (ESI) m/z 433 [M+H]⁺. ¹H NMR (400 MHz, DMSO-d₆) δ 13.44-13.39 (m, 1H), 7.76-7.32 (m, 4.5H), 6.47-6.43 (m, 0.5H), 5.56 (d, J=9.2 Hz, 1H), 4.83-4.78 (m, 2H), 3.81-3.51 (m, 4H), 1.36-1.30 (m, 9H).

### Example II. SARM1 (50-724) Enzymatic Assay

An enzymatic assay was performed in a 384-well plate using Dulbecco's phosphate-buffered saline (PBS) as the reaction buffer. Purified SARM1 (50-724) with a final concentration of 2 nM was pre-incubated with a test compound at 1% DMSO final assay concentration for 15 min at room temperature. The reaction was initiated by adding a mixture of 200 μM nicotinamide mononucleotide (NMN) as activator and 100 μM NAD⁺ as substrate. After 1 h of incubation at room temperature, the reaction was terminated with 10 times volume of 70% acetonitrile, then centrifuged at 3800 rpm for 10 min. The samples were analyzed using LC-MS/MS after diluted to a proper concentration by 10 mM ammonium acetate (pH 9.75).

SARM1 inhibitory activity of Compounds 1-80 is summarized in Table 2. In Table 2, activity is provided as follows: A=IC₅₀≤100 nM; B=101 nM≤IC₅₀≤500 nM; C=501 nM≤IC₅₀≤1000 nM; D=IC₅₀≥1001 nM. ND means “not determined.”

TABLE 2

| Cmpd No. | IC50 | Cmpd No. | IC50 | Cmpd No. | IC50 |
|---|---|---|---|---|---|
| 1 | A | 2 | C | 3 | D |
| 4 | D | 5 | B | 6 | B |
| 7 | D | 8 | D | 9 | D |
| 10 | D | 11 | D | 12 | D |
| 13 | D | 14 | C | 15 | C |
| 16 | C | 17 | D | 18 | D |
| 19 | D | 20 | A | 21 | D |
| 22 | D | 23 | D | 24 | D |
| 25 | D | 26 | D | 27 | B |
| 28 | B | 29 | D | 30 | D |
| 31 | D | 32 | D | 33 | D |
| 34 | D | 35 | D | 36 | B |
| 37 | B | 38 | D | 39 | D |
| 40 | D | 41 | D | 42 | D |
| 43 | D | 44 | D | 45 | D |
| 46 | B | 47 | B | 48 | A |
| 49 | B | 50 | D | 51 | D |
| 52 | D | 53 | D | 54 | D |
| 55 | D | 56 | D | 57 | D |
| 58 | D | 59 | D | 60 | B |
| 61 | D | 62 | B | 63 | D |
| 64 | D | 65 | D | 66 | ND |
| 67 | ND | 68 | D | 69 | A |
| 70 | B | 71 | A | 72 | A |
| 73 | B | 74 | A | 75 | C |
| 76 | B | 77 | D | 78 | B |
| 79 | A | 80 | A | 81 | D |
| 82 | B | 83 | B | 84 | D |
| 85 | D | 86 | D | 87 | D |
| 88 | D | 89 | C | 90 | D |
| 91 | B | 92 | B | 93 | C |
| 94 | D | 95 | D | 96 | A |
| 97 | B | 98 | A | 99 | D |
| 100 | A | 101 | D | 102 | A |
| 103 | A | 104 | B | 105 | A |
| 106 | A | 107 | A | 108 | B |
| 109 | B | 110 | A | 111 | A |
| 112 | A | 113 | D | 114 | B |
| 115 | A | 116 | A | 117 | D |
| 118 | A | 119 | B | 120 | A |
| 121 | B | 122 | B | 123 | A |
| 124 | D | 125 | A | 126 | D |
| 127 | A | 128 | A | 129 | A |
| 130 | A | 131 | A | 132 | A |
| 133 | A | 134 | B | 135 | A |
| 136 | A | 137 | A | 138 | B |
| 139 | B | 140 | A | 141 | A |
| 142 | D | 143 | D | 144 | B |
| 145 | B | 146 | A | 147 | A |
| 148 | A | 149 | B | 150 | A |
| 151 | A | 152 | A | 153 | B |
| 154 | D | 155 | B | 156 | B |
| 157 | A | 158 | A | 159 | B |
| 160 | A | 161 | A | 162 | D |
| 163 | B | 164 | B | 165 | C |
| 166 | A | 167 | A | 168 | A |
| 169 | B | 170 | A | 171 | B |
| 172 | C | 173 | A | 174 | A |
| 175 | C | 176 | D | 177 | A |
| 178 | A | 179 | A | 180 | A |
| 181 | A | 182 | A | 183 | A |
| 184 | B | 185 | A | 186 | A |
| 187 | A | 188 | B | 189 | A |
| 190 | A | 191 | A | 192 | A |
| 193 | A | 194 | A | 195 | A |
| 196 | A | 197 | A | 198 | A |
| 199 | A | 200 | A | 201 | A |
| 202 | A | 203 | A | 204 | A |
| 205 | A | 206 | A | 207 | B |
| 208 | A | 209 | A | 210 | A |
| 211 | A | 212 | A | 213 | A |
| 214 | B | 215 | B | 216 | A |
| 217 | B | 218 | A | 219 | B |
| 220 | B | 221 | A | 222 | A |
| 223 | A | 224 | A | 225 | A |
| 226 | A | 227 | A | 228 | A |
| 229 | A | 230 | A | 231 | A |
| 232 | A | 233 | A | 234 | A |
| 235 | B | 236 | A | 237 | C |
| 238 | A | 239 | B | 240 | A |

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. A compound of the following structural Formula 1: 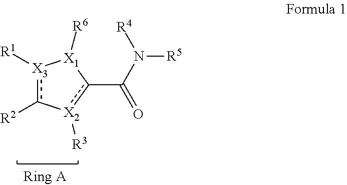
a tautomer thereof, a solvate or stereoisomer of the compound or the tautomer, or a pharmaceutically acceptable salt of the foregoing, wherein:
X₁ is C, N, or O; X₂ is C or N; X₃ is C or N;
R¹ and R²:
are independently selected from H, optionally substituted C₁-C₄ alkyl, and optionally substituted C₃-C₆ cycloalkyl, or
join to form a 5- to 7-membered ring such that the 5- to 7-membered ring and Ring A together form Fused Ring A selected from Fused Ring A-1, Fused Ring A-2, Fused Ring A-3, and Fused Ring A-4 shown below: 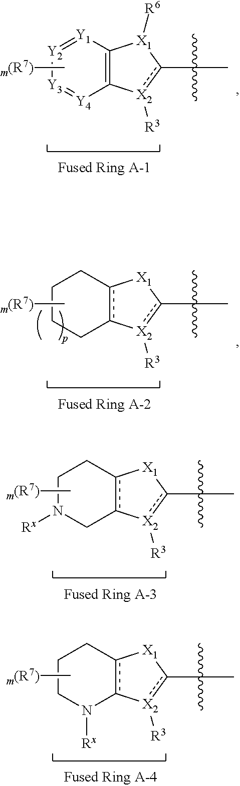
wherein:
Y₁, Y₂, Y₃, and Y₄ are each independently selected from C and N;
p is an integer selected from 0, 1, and 2;
R⁷ is selected from halogen, —CN, —CONH₂, —SO₂Me, optionally substituted 3-6 membered cycloalkyl, optionally substituted 4-6 membered heterocyclyl, optionally substituted phenyl, optionally substituted 5-6 membered heteroaryl, optionally substituted C₁-C₄ alkyl, ═O (provided that Fused Ring A is not Fused Ring A-1), and —OR^{s}, wherein R^{s} is optionally substituted C₁-C₄ alkyl, and m is an integer selected from 0, 1, 2, and 3;
R^{x} is selected from H, optionally substituted 3- to 5-membered cycloalkyl, optionally substituted C₁-C₄ alkyl, and —C(═O)R^{y}, wherein R^{y} is optionally substituted 3-5 membered cycloalkyl or optionally substituted C₁-C₄ alkyl;
R³:
is selected from absent, H, —CN, —C(═O)NH₂, and optionally substituted C₁-C₄ alkyl;
R⁴:
is selected from H, optionally substituted C₁-C₆ alkyl, optionally substituted 3- to 5-membered cycloalkyl, optionally substituted 3- to 6-membered heterocyclyl, optionally substituted 5- to 6-membered heteroaryl,
—R^{a}C(═O)R^{b}, —R^{a}NHC(═O)R^{b}, —R^{a}NR^{c}R^{d}, —R^{a}S(═O)R^{b}, —R^{a}S(═O)₂R^{b}, 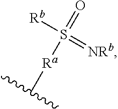
—R^{a}NHS(═O)₂R^{b}, and —R^{a}C(═O)NR^{c}R^{d}, wherein:
R^{a}, for each occurrence, is independently selected from C₁-C₈ alkyl optionally substituted by 1-3 groups selected from —OH and halogen;
R^{b}, for each occurrence, is independently selected from H, —OH, —O(C₁-C₃ alkyl), 4- to 6-membered heterocyclyl, and C₁-C₆ alkyl optionally substituted by 1-3 groups selected from —OH and halogen;
R^{c} and R^{d}, for each occurrence, are independently selected from H and C₁-C₆ alkyl, or
R^{c} and R^{d} join to form a 3- to 6-membered cycloalkyl or a 3- to 6-membered heterocyclyl wherein the 3- to 6-membered cycloalkyl or 3- to 6-membered heterocyclyl of R^{c} and R^{d} is optionally substituted by 1-3 groups selected from —OH and halogen;
R⁵:
is selected from optionally substituted C₁-C₇ alkyl, optionally substituted phenyl, optionally substituted 5- to 6-membered heteroaryl, optionally substituted 5- to 6-membered cycloalkyl, and optionally substituted 5- to 6-membered heterocyclyl; and
R⁶ is H, optionally substituted C₁-C₄ alkyl, or CH₂CONH₂, provided that R⁶ is absent when X₁ is O.

2. The compound of claim 1, wherein the compound has the following structural Formula 2: 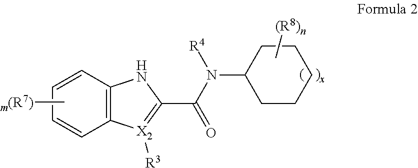
a tautomer thereof, a solvate or stereoisomer of the compound or the tautomer, or a pharmaceutically acceptable salt of the foregoing, wherein x is an integer selected from 0 and 1; R⁸, for each occurrence, is independently selected from C₁-C₄ alkyl (optionally substituted with 1 to 3 groups selected from —OH and halogen), halogen, CN, —O(C₁-C₄ alkyl), and —C(═O)O(C₁-C₄ alkyl); and n is an integer selected from 0, 1, 2, and 3.

3. The compound of claim 1, wherein the compound has the following structural Formula 2-1: 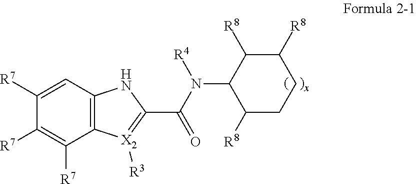
a tautomer thereof, a solvate or stereoisomer of the compound or the tautomer, or a pharmaceutically acceptable salt of the foregoing, wherein
X₂ is C or N;
R³ is selected from absent, H, —CN, —C(═O)NH₂, and C₁-C₂ alkyl, wherein
the C₁-C₂ alkyl of R³ is optionally substituted with a group selected from OH, —O(C₁-C₂ alkyl), CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —CN, —C(═O)OH, and —C(═O)NH₂;
R⁴ is selected from H, 3- to 5-membered cycloalkyl, 3- to 5-membered heterocyclyl, and C₁-C₃ alkyl optionally substituted with a group selected from OH, CN, and 3- to 5-membered heterocyclyl;
R⁷, for each occurrence, is independently selected from H, halogen, 3-4 membered cycloalkyl, and C₁-C₃ alkyl optionally substituted with 1 to 3 groups selected from —OH, halogen, and O(C₁-C₄ alkyl);
R⁸, for each occurrence, is independently selected from H, C₁-C₂ alkyl (optionally substituted with 1 to 3 groups selected from —OH and halogen), halogen, CN, —O(C₁-C₄ alkyl), and —C(═O)O(C₁-C₄ alkyl); and
x is an integer selected from 0 and 1.

4. The compound of claim 1, wherein the compound has the following structural Formula 2-2: 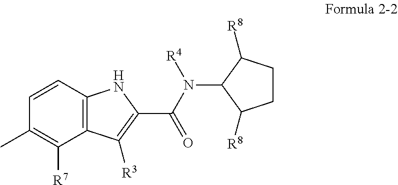
a tautomer thereof, a solvate or stereoisomer of the compound or the tautomer, or a pharmaceutically acceptable salt of the foregoing, wherein
R³ is selected from H, —CN, —C(═O)NH₂, and C₁-C₂ alkyl, wherein
the C₁-C₂ alkyl of R³ is optionally substituted with a group selected from OH, —O(C₁-C₂ alkyl), —CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —CN, —C(═O)OH, and —C(═O)NH₂;
R⁴ is methyl or CH₂CH₂OH;
R⁷ is selected from H, methyl, 3-4 membered cycloalkyl, and C₁-C₃ alkyl optionally substituted with 1 to 3 groups selected from —OH and halogen; and
R⁸, for each occurrence, is independently selected from H and C₁-C₂ alkyl optionally substituted with 1 to 3 groups selected from —OH and halogen.

5. The compound of claim 1, wherein the compound has the following structural Formula 2-3: 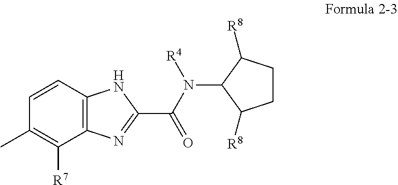
a tautomer thereof, a solvate or stereoisomer of the compound or the tautomer, or a pharmaceutically acceptable salt of the foregoing, wherein
R⁴ is methyl or CH₂CH₂OH;
R⁷ is selected from H, methyl, 3-4 membered cycloalkyl, and C₁-C₃ alkyl optionally substituted with 1 to 3 groups selected from —OH and halogen;
R⁸, for each occurrence, is independently selected from H and C₁-C₂ alkyl optionally substituted with 1 to 3 groups selected from —OH and halogen.

6. The compound of claim 1, wherein the compound has the following structural Formula 2-4: 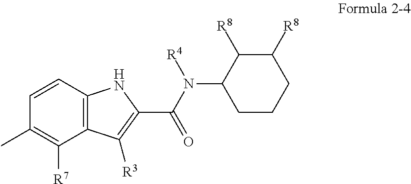
a tautomer thereof, a solvate or stereoisomer of the compound or the tautomer, or a pharmaceutically acceptable salt of the foregoing, wherein
R³ is selected from H, CN, —C(═O)NH₂, and C₁-C₂ alkyl, wherein
the C₁-C₂ alkyl of R³ is optionally substituted with a group selected from OH, —O(C₁-C₂ alkyl), CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —C(═O)OH, and —C(═O)NH₂;
R⁴ is methyl or CH₂CH₂OH;
R⁷ is selected from H, methyl, and 3-4 membered cycloalkyl optionally substituted with 1 to 3 groups selected from —OH and halogen, and
R⁸, for each occurrence, is independently selected from H and C₁-C₂ alkyl optionally substituted with 1 to 3 groups selected from —OH and halogen.

7. The compound of claim 1, wherein the compound has the following structural Formula 3: 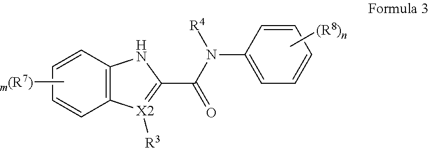
a tautomer thereof, a solvate or stereoisomer of the compound or the tautomer, or a pharmaceutically acceptable salt of the foregoing, wherein R⁸, for each occurrence, is independently selected from C₁-C₄ alkyl (optionally substituted with 1 to 3 groups selected from —OH and halogen), halogen, —CN, —O(C₁-C₄ alkyl), and —C(═O)O(C₁-C₄ alkyl); and n is an integer selected from 0, 1, 2, and 3.

8. The compound of claim 1, wherein the compound has the following structural Formula 3-1: 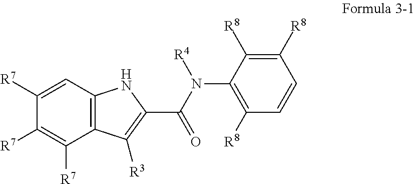
a tautomer thereof, a solvate or stereoisomer of the compound or the tautomer, or a pharmaceutically acceptable salt of the foregoing, wherein
R³ is selected from H, —CN, —C(═O)NH₂, and C₁-C₃ alkyl, wherein
the C₁-C₃ alkyl of R³ is optionally substituted with a group selected from OH, —O(C₁-C₂ alkyl), CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —C(═O)OH, and —C(═O)NH₂;
R⁴ is selected from H, 3- to 5-membered cycloalkyl, 3- to 5-membered heterocyclyl, and C₁-C₃ alkyl optionally substituted with a group selected from OH, CN, and 3- to 5-membered heterocyclyl;
R⁷, for each occurrence, is independently selected from H, halogen, 3-4 membered cycloalkyl, and C₁-C₃ alkyl optionally substituted with a group selected from —OH, halogen, and O(C₁-C₄ alkyl); and
R⁸, for each occurrence, is independently selected from H, C₁-C₂ alkyl (optionally substituted with 1 to 3 groups selected from —OH and halogen), halogen, CN, —C(═O)O(C₁-C₄ alkyl), and O(C₁-C₄ alkyl).

9. The compound of claim 1, wherein the compound has the following structural Formula 3-2: 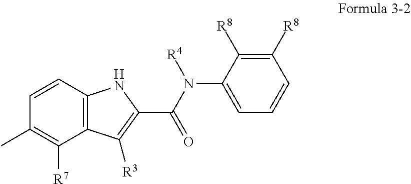
a tautomer thereof, a solvate or stereoisomer of the compound or the tautomer, or a pharmaceutically acceptable salt of the foregoing, wherein
R³ is selected from H, —CN, —C(═O)NH₂, and C₁-C₂ alkyl, wherein
the C₁-C₂ alkyl of R³ is optionally substituted with a group selected from OH, —O(C₁-C₂ alkyl), CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —C(═O)OH, and —C(═O)NH₂;
R⁴ is selected from H, 3- to 5-membered cycloalkyl, 3- to 5-membered heterocyclyl, and C₁-C₃ alkyl optionally substituted with a group selected from OH, CN, and 3- to 5-membered heterocyclyl;
R⁷ is selected from H, methyl, and 3-4 membered cycloalkyl, and
R⁸, for each occurrence, is independently selected from H, C₁-C₂ alkyl (optionally substituted with 1 to 3 groups selected from —OH and halogen), halogen, and O(C₁-C₄ alkyl).

10. The compound of claim 1, wherein the compound has the following structural Formula 3-3: 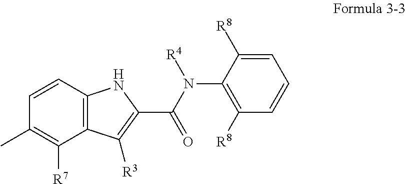
a tautomer thereof, a solvate or stereoisomer of the compound or the tautomer, or a pharmaceutically acceptable salt of the foregoing, wherein
R³ is selected from H, —CN, —C(═O)NH₂, and C₁-C₂ alkyl, wherein
the C₁-C₂ alkyl of R³ is optionally substituted with a group selected from OH, —O(C₁-C₂ alkyl), —CN, —C(═O)NH₂, and —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —C(═O)OH, and —C(═O)NH₂;
R⁴ is selected from H, 3- to 5-membered cycloalkyl, 3- to 5-membered heterocyclyl, and C₁-C₃ alkyl optionally substituted with a group selected from OH, CN, and 3- to 5-membered heterocyclyl;
R⁷ is selected from H, methyl, and 3-5 membered cycloalkyl, and
R⁸, for each occurrence, is independently selected from H, C₁-C₂ alkyl (optionally substituted with 1 to 3 groups selected from —OH and halogen), halogen, and O(C₁-C₄ alkyl).

11. The compound of claim 1, wherein the compound has the following structural Formula 4: 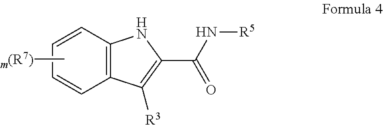
a tautomer thereof, a solvate or stereoisomer of the compound or the tautomer, or a pharmaceutically acceptable salt of the foregoing.

12. The compound of claim 1, wherein the compound has the following structural Formula 4-1: 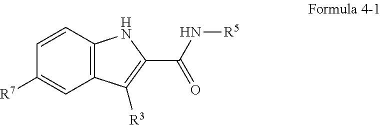
a tautomer thereof, a solvate or stereoisomer of the compound or the tautomer, or a pharmaceutically acceptable salt of the foregoing, wherein
R³ is C₁-C₂ alkyl optionally substituted with —NHR^{p1}, wherein
R^{p1} is selected from H, —C(═O)(C₁-C₂ alkyl), and 5- to 6-membered heteroaryl, wherein the 5- to 6-membered heteroaryl of R^{p1} is optionally substituted with 1-2 groups selected from C₁-C₃ alkyl, —C(═O)OH, —C(═O)NH₂, and phenyl optionally substituted with halogen;
R⁵ is selected from C₁-C₆ alkyl, wherein the C₁-C₆ alkyl of R⁵ is optionally substituted with —C(═O)NHR^{p2}, wherein R^{p2} is selected from C₁-C₃ alkyl and phenyl; and
R⁷ is selected from C₁-C₂ alkyl optionally substituted with 1 to 3 groups selected from —OH, halogen, and O(C₁-C₄ alkyl).

13. The compound, tautomer, solvate, stereoisomer, or pharmaceutically acceptable salt of claim 1, wherein R¹ and R² do not join to form Fused Ring A, and R¹ and R² are each independently selected from H, C₁-C₄ alkyl, and 3- to 6-membered cycloalkyl.

14. The compound, tautomer, solvate, stereoisomer, or pharmaceutically acceptable salt of claim 1, wherein R¹ and R² do not join to form Fused Ring A, and R¹ and R² are each independently selected from H, methyl, and —CH(CH₃)₂.

15. The compound of claim 1, wherein the compound has the following structural Formula 5: 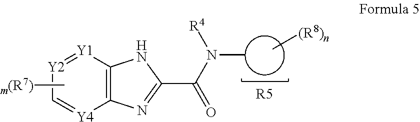
a tautomer thereof, a solvate or stereoisomer of the compound or the tautomer, or a pharmaceutically acceptable salt of the foregoing, wherein
Y₁, Y₂, and Y₄ are each independently selected from C and N;
R⁵ is phenyl, 5- to 6-membered heteroaryl, 5- to 6-membered cycloalkyl, or 5- to 6-membered heterocyclyl, wherein R⁵ is substituted with n groups of R⁸; and
R⁸, for each occurrence, is independently selected from halogen, —CN, —CONH₂, —SO₂Me, —C(═O)CH₃, C₁-C₄ alkyl (optionally substituted with 1-3 groups selected from —OH, halogen, —SO₂Me, and 4- to 6-membered heterocyclyl), 4- to 6-membered heterocyclyl (optionally substituted with 1-3 groups selected from —OH and halogen), ═O, and —O(C₁-C₃ alkyl), wherein the C₁-C₃ alkyl of the —O(C₁-C₃alkyl) is optionally substituted with 1-3 groups selected from —OH and halogen; and n is an integer selected from 0, 1, 2, 3, and 4.

16. The compound of claim 1, wherein the compound has the following structural Formula 6-1, 6-2, 6-3, or 6-4: 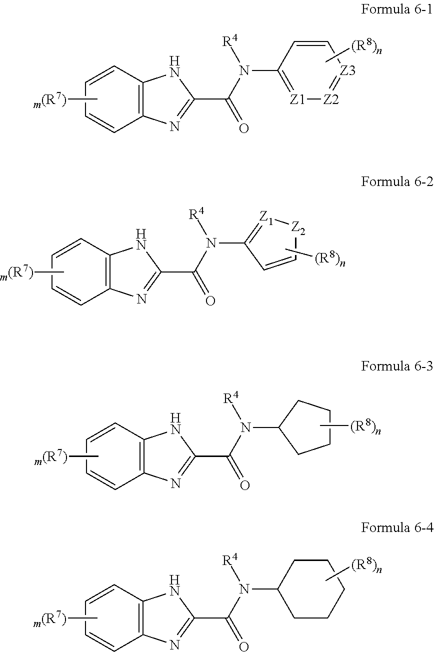
a tautomer thereof, a solvate or stereoisomer of the compound or the tautomer, or a pharmaceutically acceptable salt of the foregoing, wherein Z₁, Z₂, and Z₃ are each independently selected from C and N; R⁸, for each occurrence, is independently selected from halogen, —CN, C₁-C₂ alkyl, —O(C₁-C₂ alkyl), and ═O, wherein the C₁-C₂ alkyl of the —O(C₁-C₂ alkyl) is optionally substituted with 1-3 groups selected from halogen.

17. The compound of claim 1, wherein the compound has the following structural Formula 6-1a: 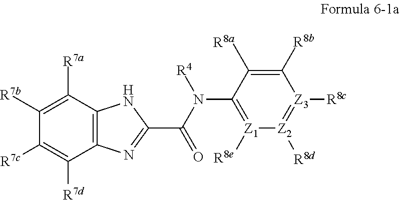
a tautomer thereof, a solvate or stereoisomer of the compound or the tautomer, or a pharmaceutically acceptable salt of the foregoing, wherein
Z₁, Z₂, and Z₃ are each independently selected from C and N;
R^{7a} is selected from H and halogen;
R^{7b} is selected from H and halogen;
R^{7c} is selected from H, halogen, C₁-C₄ alkyl (optionally substituted by 1-3 groups selected from OH, —CN, —CONH₂ and halogen), 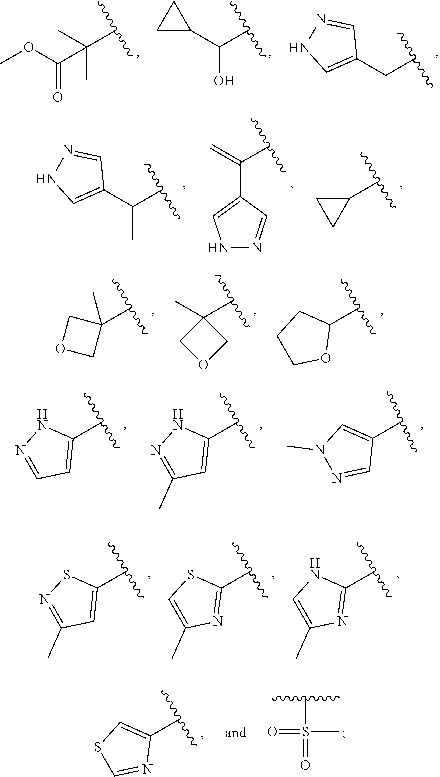
R^{7d} is selected from H, halogen, C₁-C₂ alkyl, —CN, 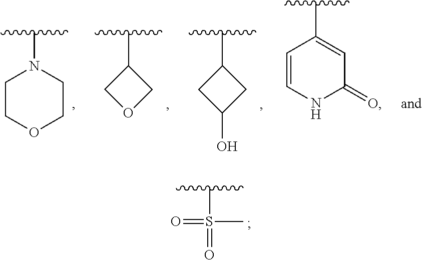
R^{8a}, R^{8b}, R^{8c}, R^{8d}, and R^{8e} are each independently selected from H, halogen, —CN, C₁-C₂ alkyl, —O(C₁-C₂ alkyl), ═O, and —OCF₃.

18. The compound of claim 1, wherein the compound has the following structural Formula 6-2a, 6-2b, 6-2c, 6-2d, 6-2e, or 6-2f: 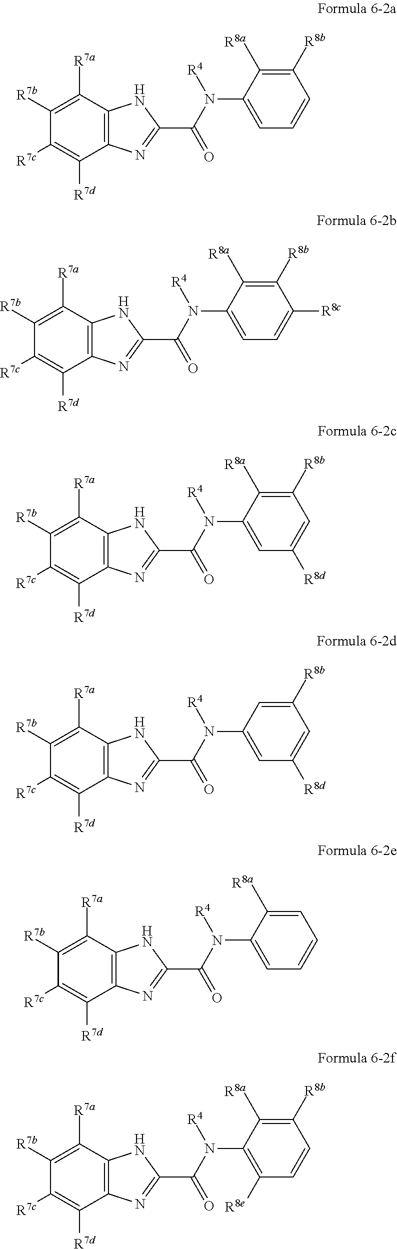
a tautomer thereof, a solvate or stereoisomer of the compound or the tautomer, or a pharmaceutically acceptable salt of the foregoing, wherein R^{8a}, R^{8b}, R^{8c}, R^{8d}, and R^{8e} are independently selected from F, Cl, CN, CH₃, ═O, —OCH₃, and —OCF₃.

19. The compound, tautomer, solvate, stereoisomer, or pharmaceutically acceptable salt of claim 1, wherein 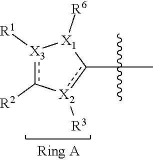
is selected from:

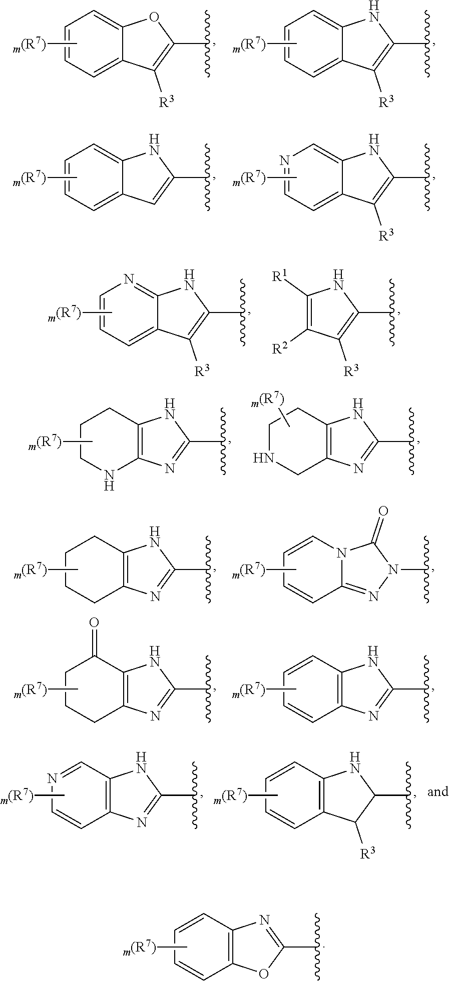

20. The compound, tautomer, solvate, stereoisomer, or pharmaceutically acceptable salt of claim 1, wherein 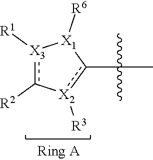
is selected from:

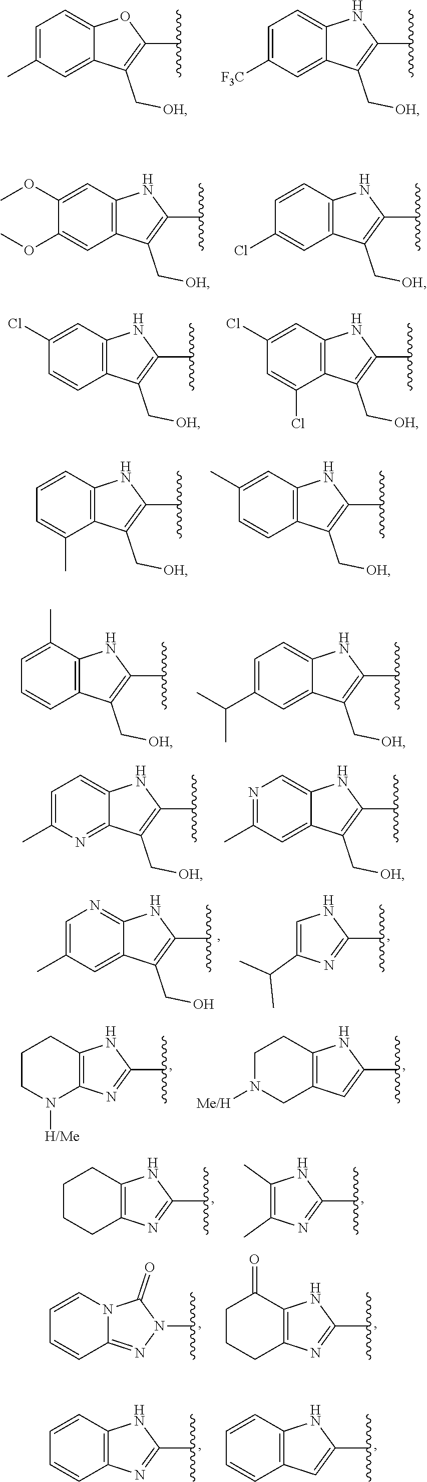

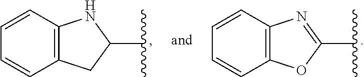 [SMILES: *C1Cc2ccccc2N1 ; *c1nc2ccccc2o1]

21. The compound, tautomer, solvate, stereoisomer, or pharmaceutically acceptable salt of claim 1, wherein

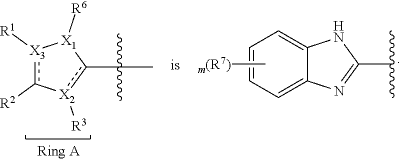

22. The compound, tautomer, solvate, stereoisomer, or pharmaceutically acceptable salt of claim 1, wherein 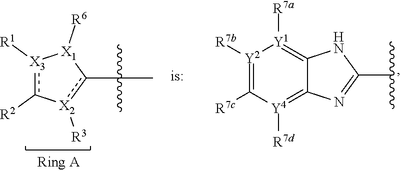
wherein:
Y₁, Y₂, and Y₄ are each independently selected from C and N;
R^{7a} and R^{7b} are each independently selected from H, CH₃, Cl, F, Br, and —OCH₃;
R^{7c} is selected from H, Me, Cl, F, Br, —OMe, CF₃, CHF₂, CH₂CF₃, CH₂CHF₂, —OCF₃, —OCHF₂, —SO₂Me, —CN, OH, CH₂OH, —COOH, —CONH₂, —CO₂Me, 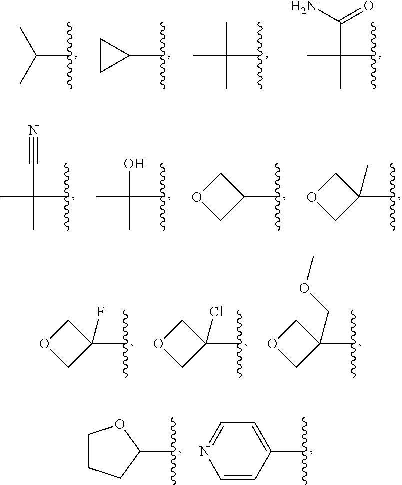 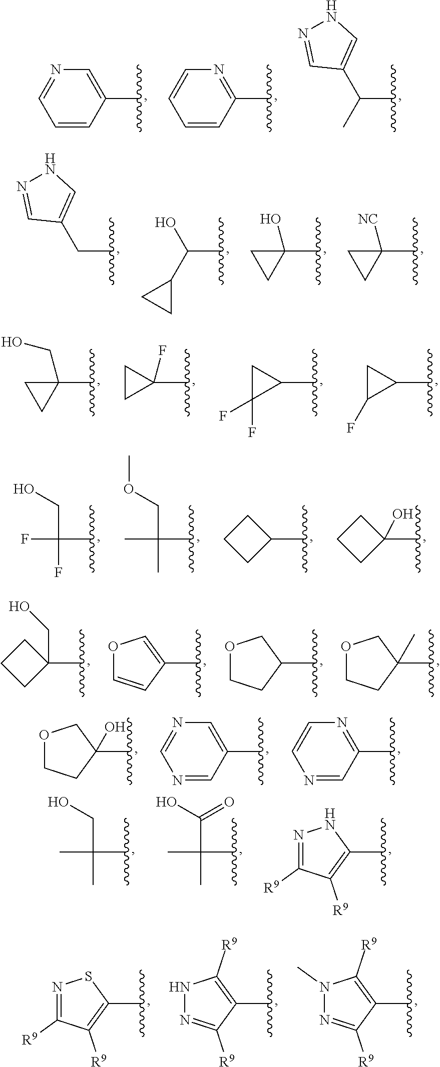 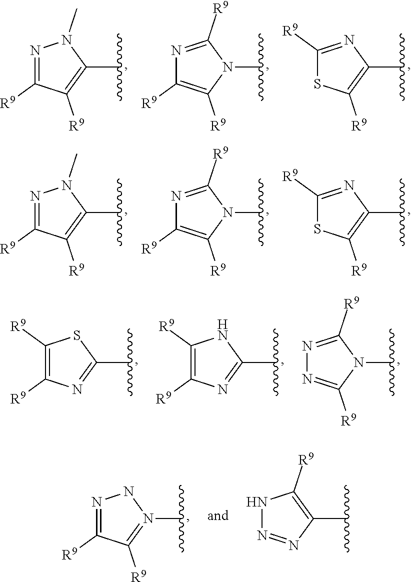
wherein R⁹, for each occurrence, is independently selected from H, Me, Cl, F, and —CN; and
R^{7d} is selected from H, Me, Cl, F, Br, —OMe, —CN, —CONH₂, —SO₂Me,

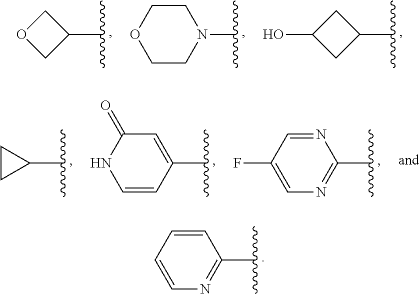

23. The compound, tautomer, solvate, stereoisomer, or pharmaceutically acceptable salt of claim 1, wherein 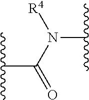
of Formula I is selected from:

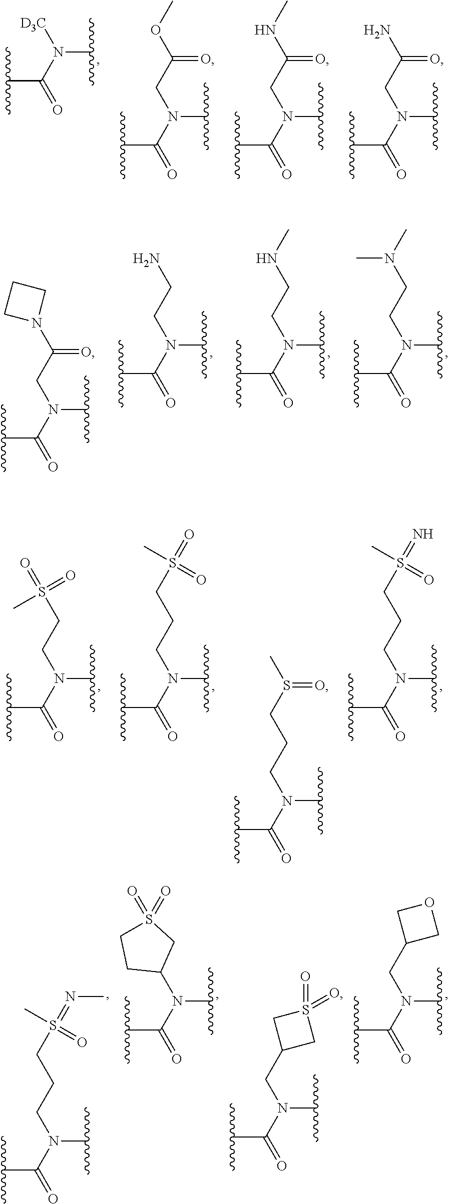

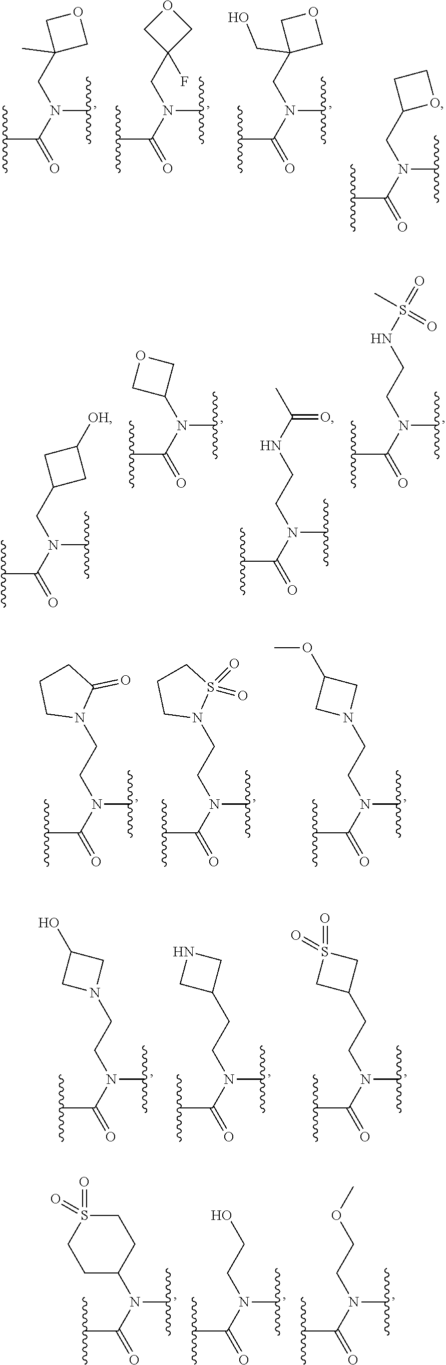

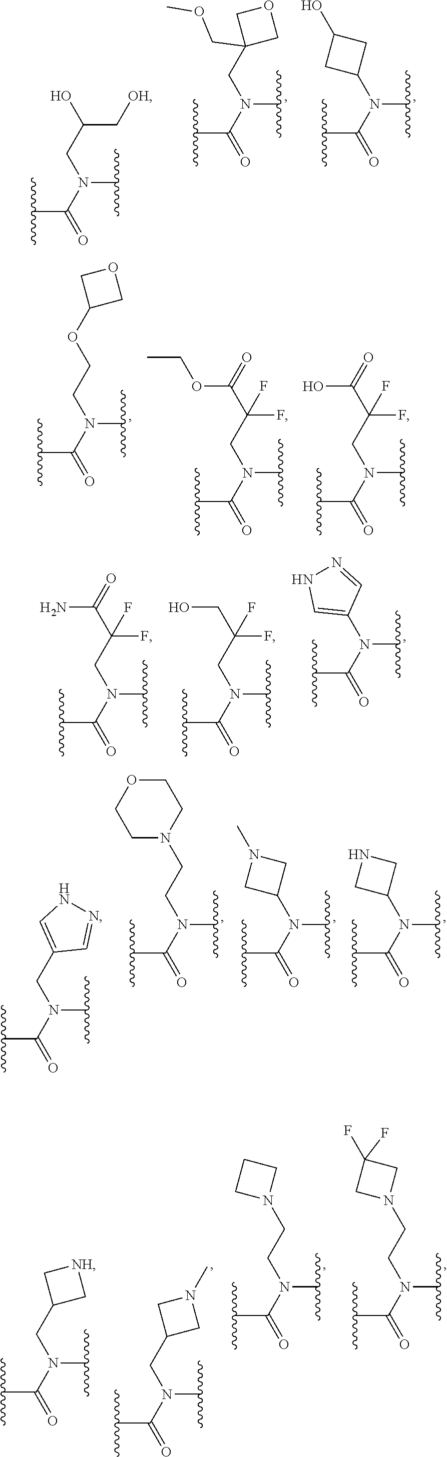

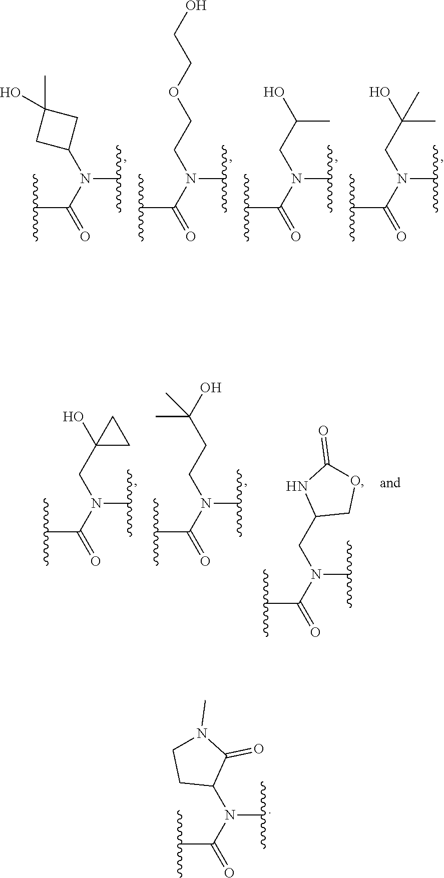

24. The compound, tautomer, solvate, stereoisomer, or pharmaceutically acceptable salt of claim 1, wherein R³ is selected from absent, H, CN, —C(═O)NH₂, and C₁-C₄ alkyl, wherein
the C₁-C₄ alkyl of R³ is optionally substituted with 1 to 2 groups selected from —OH, —O(C₁-C₃ alkyl), CN, —C(═O)NH₂, —NH₂, and —NHR^{p1}, wherein
R^{p1} is selected from —C(═O)(C₁-C₃ alkyl) and 5- to 6-membered heteroaryl, wherein the 5- to 6-membered heteroaryl of R^{p1} is optionally substituted with 1-3 groups selected from C₁-C₃ alkyl, —C(═O)OH, —C(═O)NH₂, and optionally substituted phenyl.

25. The compound, tautomer, solvate, stereoisomer, or pharmaceutically acceptable salt of claim 1, wherein R³ is selected from absent, H, CN, —C(═O)NH₂, and C₁-C₃ alkyl, wherein
the C₁-C₃ alkyl of R³ is optionally substituted with a group selected from —OH, —O(C₁-C₂ alkyl), CN, —C(═O)NH₂, —NH₂, and —NHR^{p1}, wherein
R^{p1} is selected from —C(═O)(C₁-C₂ alkyl) and 5- to 6-membered heteroaryl, wherein the 5- to 6-membered heteroaryl of R^{p1} is optionally substituted with 1-2 groups selected from C₁-C₃ alkyl, —C(═O)OH, —C(═O)NH₂, and phenyl optionally substituted with 1 to 2 groups selected from halogen.

26. The compound, tautomer, solvate, stereoisomer, or pharmaceutically acceptable salt of claim 1, wherein R³ is selected from absent, H, CN, —C(═O)NH₂, CH₂OH, and 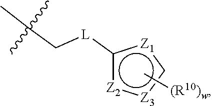
wherein L is N or O, Z1, Z2, and Z3 are independent selected from C, N, and O, R¹⁰ is selected from C₁-C₃ alkyl, —C(═O)NH₂, —C(═O)OH, and phenyl optionally substituted with 1 to 3 groups selected from halogen, and w is an integer selected from 0, 1, and 2.

27. The compound, tautomer, solvate, stereoisomer, or pharmaceutically acceptable salt of claim 1, wherein R³ is selected from absent, H, CH₂CH₂CH₃, CN, —C(═O)NH₂, CH₂OH, CH₂CH₂OH, CH₂OCH₃, CH₂CH₂OCH₃, CH₂NH₂, CH₂CH₂NH₂, CH₂CN, CH₂C(═O)NH₂,

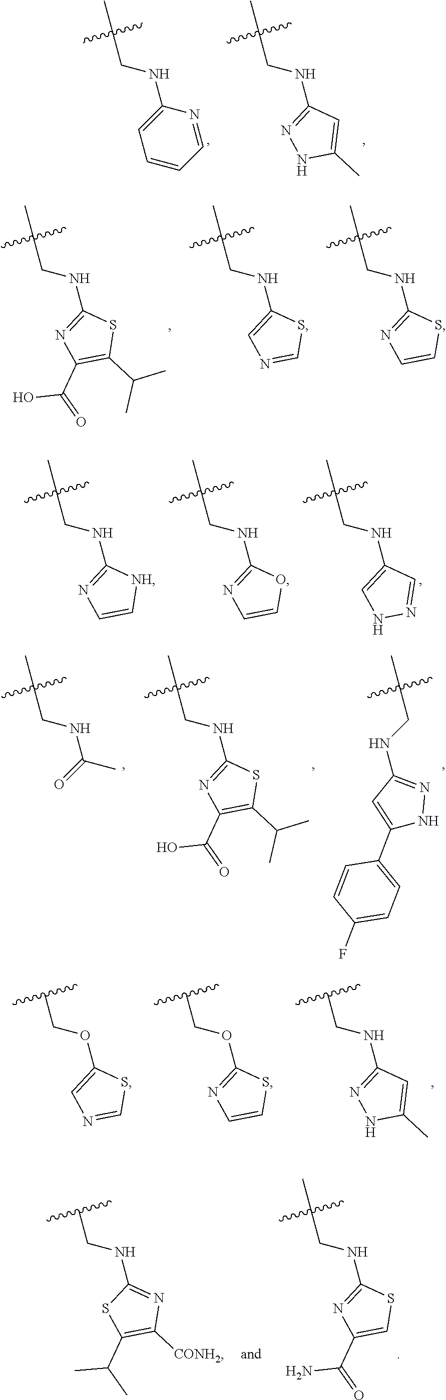

28. The compound, tautomer, solvate, stereoisomer, or pharmaceutically acceptable salt of claim 1, wherein R⁴ is selected from H, C₁-C₃ alkyl, and 3- to 4-membered heterocyclyl,
wherein the C₁-C₃ alkyl of R⁴ is optionally substituted with a group selected from OH, O(C₁-C₃ alkyl), 3- to 4-membered heterocyclyl, and —NR^{p}R^{q}, and wherein the 3- to 4-membered heterocyclyl of the C₁-C₃ alkyl of R⁴ is optionally substituted with a group selected from C₁-C₃ alkyl, and
wherein R^{p} and R^{q} are each independently selected from H and C₁-C₂ alkyl.

29. The compound, tautomer, solvate, stereoisomer, or pharmaceutically acceptable salt of claim 1, wherein R⁴ is selected from H, methyl, CH₂CH₂OH, CH₂CH₂OCH₃, CH₂CH₂NH₂, CH₂CH₂NHCH₃, CH₂CH₂N(CH₃)₂,

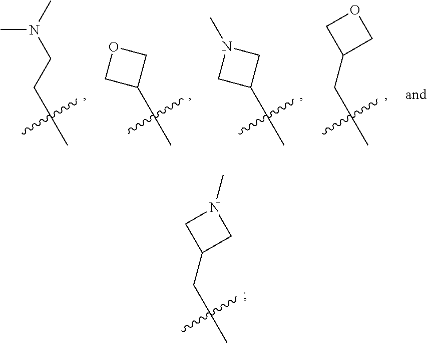

30. The compound, tautomer, solvate, stereoisomer, or pharmaceutically acceptable salt of claim 1, wherein R⁴ is selected from:
C₁-C₆ alkyl optionally substituted by 1-3 groups selected from D, halogen, —OR^{b}, 3- to 5-membered cycloalkyl (optionally substituted by 1-3 groups selected from —OH, C₁-C₃ alkyl, and halogen), 4- to 6-membered heterocyclyl (optionally substituted by 1-3 groups selected from —OR^{b}, halogen, ═O, and C₁-C₃ alkyl optionally substituted by —OR^{b}), and 5- to 6-membered heteroaryl (optionally substituted by 1-3 groups selected from —OR^{b}, halogen, and C₁-C₃ alkyl optionally substituted by —OR^{b});
4- to 6-membered heterocyclyl optionally substituted by 1-3 groups selected from —OR^{b}, halogen, ═O, and C₁-C₃alkyl optionally substituted by —OR^{b};
5- to 6-membered heteroaryl optionally substituted by 1-3 groups selected from —OR^{b}, halogen, and C₁-C₃ alkyl optionally substituted by —OR^{b};
—R^{a}C(═O)R^{b}; —R^{a}NHC(═O)R^{b}; —R^{a}NR^{c}R^{d}; —R^{a}S(═O)R^{b}; —R^{a}S(═O)₂R^{b}; 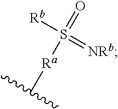
R^{a}NHS(═O)₂R^{b}; and —R^{a}C(═O)NR^{c}R^{d};
wherein:
R^{a}, for each occurrence, is independently selected from C₁-C₆ alkyl optionally substituted by 1-3 groups selected from —OH and halogen;
R^{b}, for each occurrence, is independently selected from H, —OH, —O(C₁-C₃ alkyl), 4- to 5-membered heterocyclyl, and C₁-C₃ alkyl optionally substituted by 1-3 groups selected from —OH and halogen;
R^{c} and R^{d}, for each occurrence, are independently selected from H, C₁-C₃ alkyl, or
R^{c} and R^{d} join to form a 3- to 6-membered cycloalkyl or 4- to 6-membered heterocyclyl optionally substituted by 1-3 groups selected from —OH and halogen.

31. The compound, tautomer, solvate, stereoisomer, or pharmaceutically acceptable salt of claim 1 wherein R⁴ is selected from:

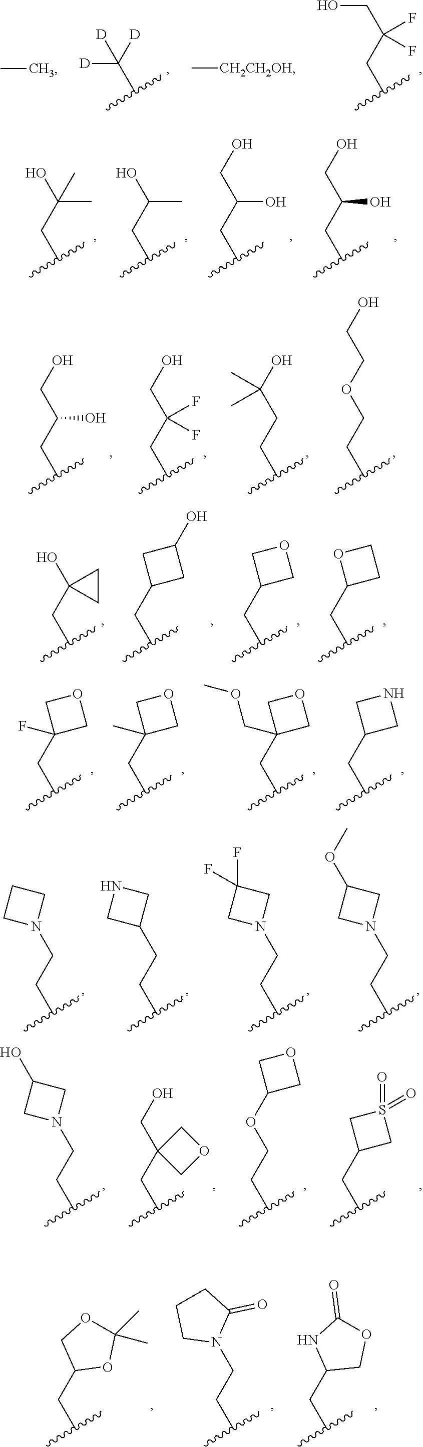

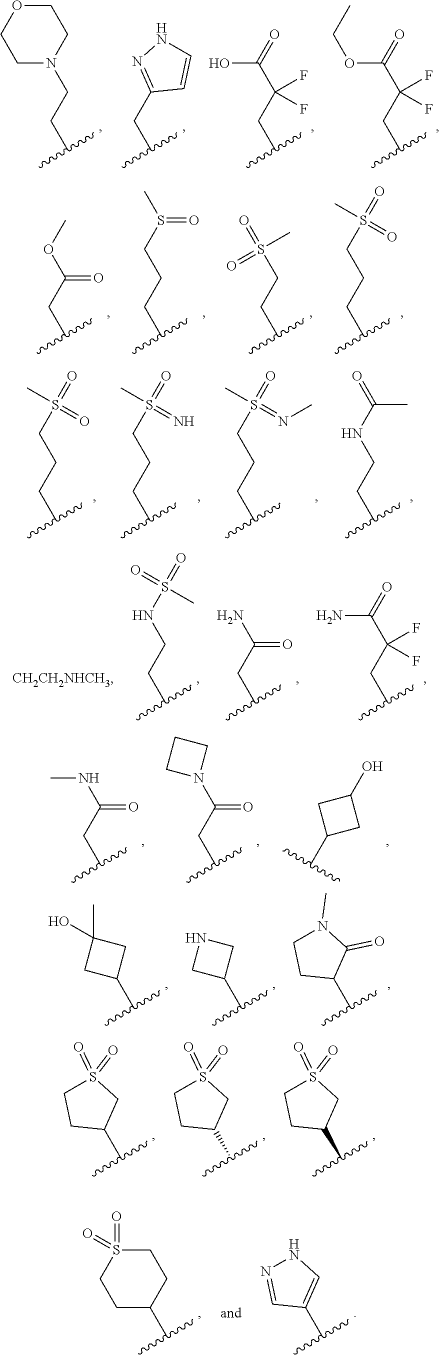

32. The compound, tautomer, solvate, stereoisomer, or pharmaceutically acceptable salt of claim 1, wherein R⁵ is selected from: C₁-C₆ alkyl, phenyl, 5- to 6-membered heteroaryl, 5- to 6-membered cycloalkyl, and 5- to 6-membered heterocyclyl,
wherein:
the C₁-C₆ alkyl of R⁵ is optionally substituted with —C(═O)NHR^{p2}, wherein R^{p2} is selected from C₁-C₃ alkyl and phenyl; and
the phenyl, 5- to 6-membered heteroaryl, 5- to 6-membered cycloalkyl, and 5- to 6-membered heterocyclyl of R⁵ is each optionally substituted with 1-3 groups selected from C₁-C₂ alkyl (optionally substituted with 1-3 groups selected from halogen), halogen, —CN, —O(C₁-C₂ alkyl), and —C(═O)O(C₁-C₄ alkyl), wherein the C₁-C₂ alkyl of —O(C₁-C₂ alkyl) is optionally substituted with 1-3 groups selected from halogen.

33. The compound, tautomer, solvate, stereoisomer, or pharmaceutically acceptable salt of claim 1, wherein R⁵ is selected from: methyl, —CH(CH₃)₂,

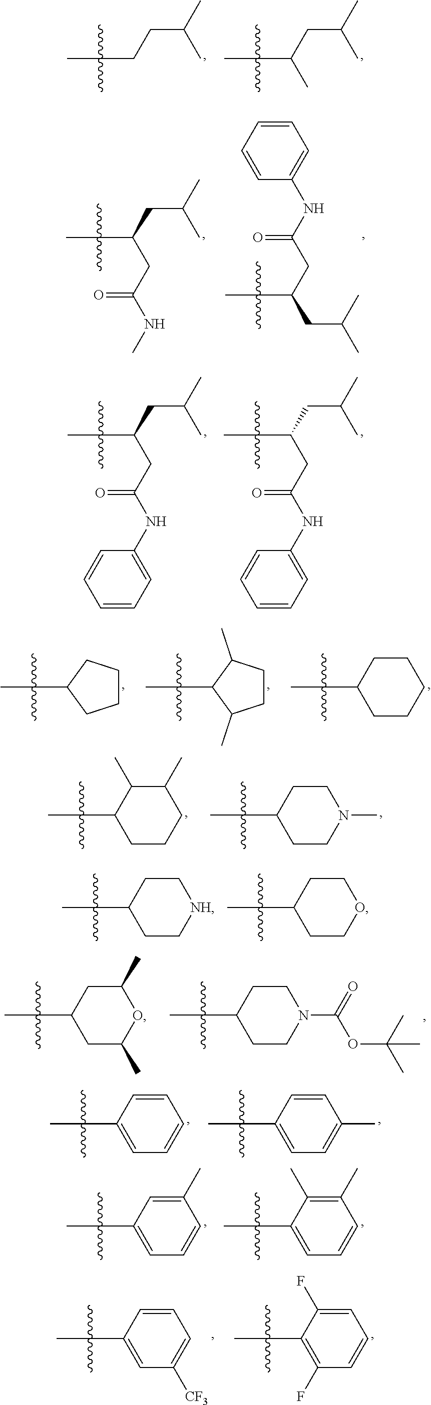

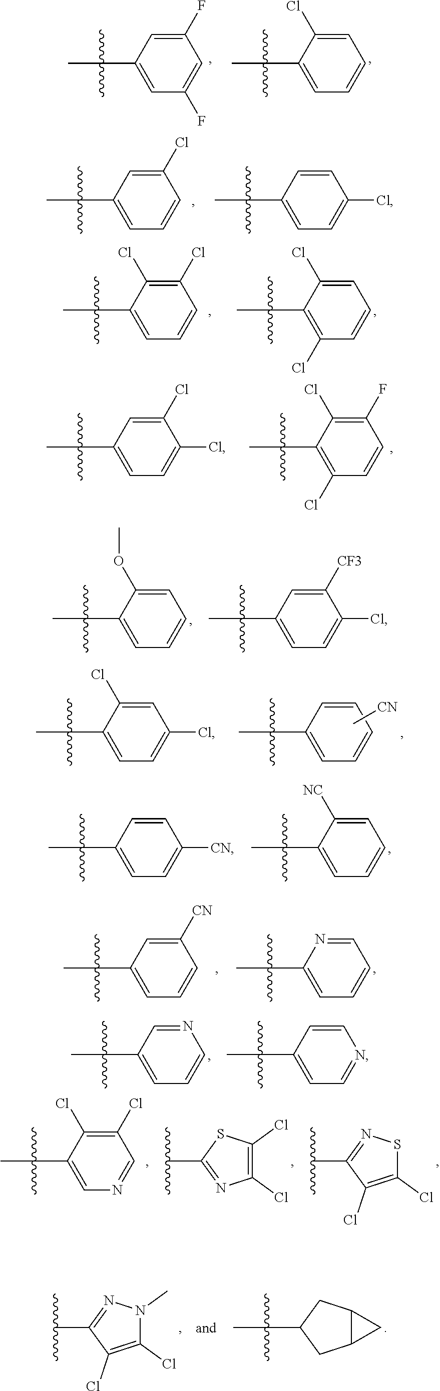

34. The compound, tautomer, solvate, stereoisomer, or pharmaceutically acceptable salt of claim 1, wherein R⁵ is selected from phenyl, 5- to 6-membered heteroaryl, 5- to 6-membered cycloalkyl, and 5- to 6-membered heterocyclyl, wherein the phenyl, heteroaryl, cycloalkyl, and heterocyclyl are optionally substituted with 1-3 groups selected from halogen, —CN, C₁-C₂ alkyl (optionally substituted with 1-3 groups selected from halogen), ═O, and —O(C₁-C₂ alkyl), wherein the C₁-C₂ alkyl of the —O(C₁-C₂ alkyl), is optionally substituted with 1-3 groups selected from halogen.

35. The compound, tautomer, solvate, stereoisomer, or pharmaceutically acceptable salt of claim 1, wherein R⁵ is selected from: 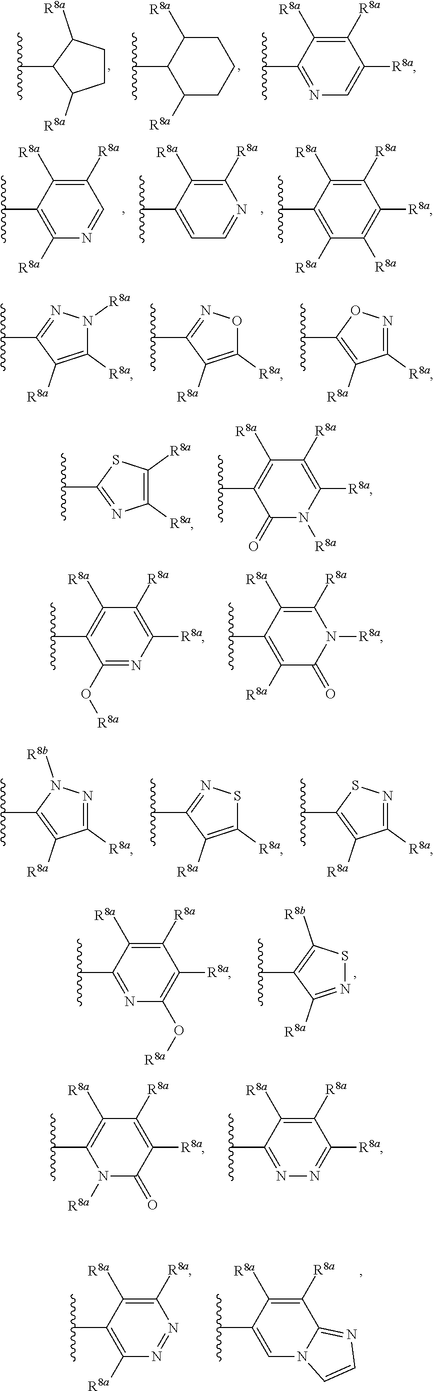 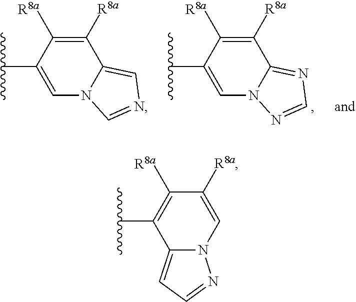
wherein: R^{8a}, for each occurrence, is independently selected from H, F, Cl, Me, OMe, —OCF₃, —OCHF₂, CHF₂, CF₃, —CN, —SO₂Me, —SMe, CH₂CF₃, CH₂SO₂Me, —CONH₂, CH₂OH, 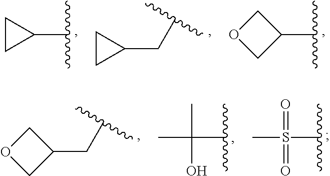
R^{8b}, for each occurrence, is independently selected from:
Me, CF₃, CHF₂, CH₂CF₃, Acetyl, SO₂Me,

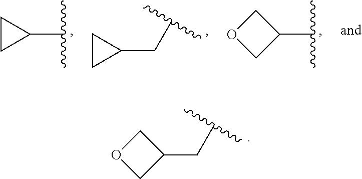

36. The compound, tautomer, solvate, stereoisomer, or pharmaceutically acceptable salt of claim 1, wherein R⁵ is selected from:

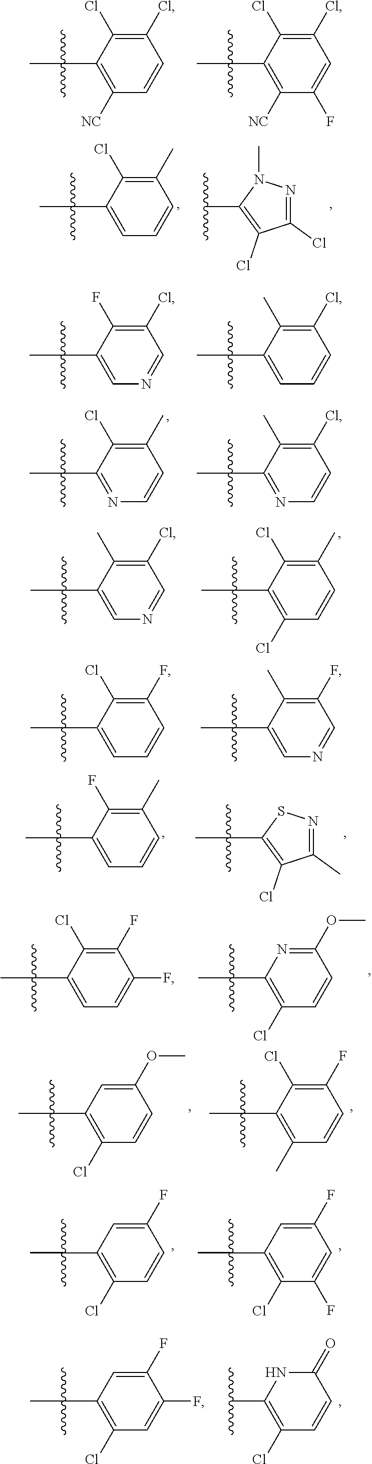

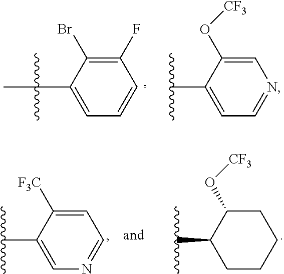

37. The compound, tautomer, solvate, stereoisomer, or pharmaceutically acceptable salt of claim 1, for each occurrence, is independently selected from halogen, —CN, —CONH₂, 4- to 6-membered heterocyclyl, 5- to 6-membered heteroaryl, phenyl, 3-5 membered cycloalkyl, C₁-C₄ alkyl (optionally substituted by 1-3 groups selected from halogen and OH), ═O (provided that Fused Ring A is not Fused Ring A-1), O(C₁-C₄ alkyl), and m is an integer selected from 0, 1, and 2.

38. The compound, tautomer, solvate, stereoisomer, or pharmaceutically acceptable salt of claim 1, wherein R⁷, for each occurrence, is independently selected from methyl, ethyl, —CH(CH₃)₂, OCH₃, F, Cl, Br, —CN, —CONH₂, —C(CH₃)₂OH, CF₃, and, and m is an integer selected from 0, 1, and 2.

39. The compound, tautomer, solvate, stereoisomer, or pharmaceutically acceptable salt of claim 1, wherein R⁷, for each occurrence, is independently selected from:
C₁-C₆ alkyl (optionally substituted by 1-3 groups selected from halogen, —OH, —CN, —C(═O)OCH₃, 3- to 4-membered cycloalkyl, and 5- to 6-membered heteroaryl),
C₁-C₆ alkenyl (optionally substituted by 1-3 groups selected from halogen, —OH, —CN, —C(═O)OCH₃, 3- to 4-membered cycloalkyl, and 5- to 6-membered heteroaryl),
3- to 5-membered cycloalkyl (optionally substituted by 1-3 groups selected from halogen, —OH, and C₁-C₂ alkyl),
4- to 6-membered heterocyclyl (optionally substituted by 1-3 groups selected from halogen, —OH, C₁-C₄ alkyl, and ═O),
5- to 6-membered heteroaryl (optionally substituted by 1-3 groups selected from halogen and C₁-C₂ alkyl), and
—S(═O)₂(C₁-C₂ alkyl), and
m is an integer selected from 0, 1, and 2.

40. The compound, tautomer, solvate, stereoisomer, or pharmaceutically acceptable salt of claim 1, wherein R⁷, for each occurrence, is independently selected from

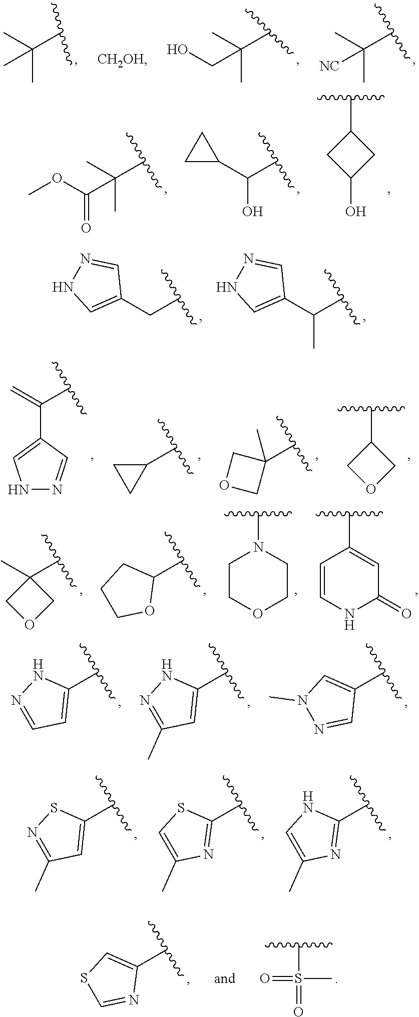

41. The compound according to claim 1, wherein the compound is selected from Compounds 1 to 240, a tautomer thereof, a solvate or stereoisomer of the compound or the tautomer, or a pharmaceutically acceptable salt of the foregoing.

42. A pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

43. A method of treating a disease or condition, comprising administering to a subject in need thereof, a therapeutically effective amount of a compound according to claim 1, a tautomer thereof, a solvate or stereoisomer of the compound or the tautomer, or a pharmaceutically acceptable salt of the foregoing, or the pharmaceutical composition comprising the compound of claim 1, a tautomer thereof, a solvate or stereoisomer of the compound or the tautomer, or a pharmaceutically acceptable salt of the foregoing and at least one pharmaceutically acceptable carrier, wherein the disease or condition is selected from ALS, Parkinson's disease, multiple sclerosis, traumatic brain injury, diabetic neuropathy, and CIPN.

44. A method of treating a disease or condition caused by axonal degeneration, comprising administering to a subject in need thereof, a therapeutically effective amount of a compound according to claim 1, a tautomer thereof, a solvate or stereoisomer of the compound or the tautomer, or a pharmaceutically acceptable salt of the foregoing, or a pharmaceutical composition comprising the compound of claim 1, a tautomer thereof, a solvate or stereoisomer of the compound or the tautomer, or a pharmaceutically acceptable salt of the foregoing and at least one pharmaceutically acceptable carrier.

45. A method of modulating SARM1, comprising contacting a subject in need thereof with a compound according to claim 1, a tautomer thereof, a solvate or stereoisomer of the compound or the tautomer, or a pharmaceutically acceptable salt of the foregoing, or a pharmaceutical composition comprising the compound of claim 1, a tautomer thereof, a solvate or stereoisomer of the compound or the tautomer, or a pharmaceutically acceptable salt of the foregoing and at least one pharmaceutically acceptable carrier.

46. A method of inhibiting or preventing axonal degeneration, comprising contacting a subject in need thereof with a compound a compound according to claim 1, a tautomer thereof, a solvate or stereoisomer of the compound or the tautomer, or a pharmaceutically acceptable salt of the foregoing, or a pharmaceutical composition comprising the compound of claim 1, a tautomer thereof, a solvate or stereoisomer of the compound or the tautomer, or a pharmaceutically acceptable salt of the foregoing and at least one pharmaceutically acceptable carrier.
